(19) United States
(12) Patent Application Publication
(10) Pub. No.: US 2025/0098527 A1
(43) Pub. Date: Mar. 20, 2025

(54) ORGANIC COMPOUND, ELECTRONIC ELEMENT AND ELECTRONIC APPARATUS

(71) Applicant: SHAANXI LIGHTE OPTOELECTRONICS MATERIAL CO., LTD., Xi'an, Shaanxi (CN)

(72) Inventors: Qiqi NIE, Xi'an, Shaanxi (CN); Youngkook KIM, Xi'an, Shaanxi (CN); Yun LIU, Xi'an, Shaanxi (CN)

(21) Appl. No.: 18/552,937

(22) PCT Filed: Dec. 7, 2022
(86) PCT No.: PCT/CN2022/137296

§ 371 Date: Sep. 28, 2023

(30) Foreign Application Priority Data
Mar. 29, 2022 (CN) 202210316982.X

(51) Int. Cl. H10K 85/60; C07D 209/88; C07D 405/14; C07D 409/14; H10K 30/00; H10K 50/10

(52) U.S. Cl. CPC H10K 85/636; C07D 209/88; C07D 405/14; C07D 409/14; H10K 85/633; H10K 30/00; H10K 50/10; H10K 85/615; H10K 85/6572; H10K 85/6574; H10K 85/6576

(57) ABSTRACT

The present disclosure relates to an organic compound, an electronic element and an electronic apparatus. The structure of the organic compound is as shown in Formula 1. The organic compound can improve the performance of the electronic element and the electronic apparatus.

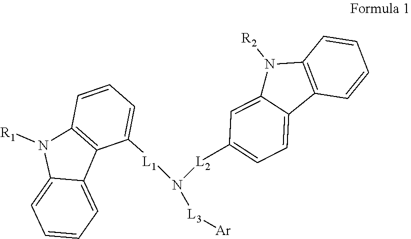

### CROSS-REFERENCE TO RELATED APPLICATIONS

The present disclosure claims the priority of Chinese patent application CN202210316982.X filed on Mar. 29, 2022, the whole content of which is incorporated herein by reference in its entirety.

### TECHNICAL FIELD

The present disclosure relates to the field of organic light-emitting materials, in particular relates to an organic compound, an electronic element and an electronic apparatus.

### BACKGROUND

With the development of electronic technology and the advancement of material science, the application range of devices used to achieve electroluminescence or photoelectric conversion is becoming more and more wide. Such devices typically include a cathode and an anode disposed relative to each other, and a functional layer disposed between the cathode and the anode. This functional layer consists of multiple layers of organic or inorganic film, and generally includes an energy conversion layer, a hole transport layer between the energy conversion layer and the anode, and an electron transport layer between the energy conversion layer and the cathode.

Taking an organic electroluminescent device as an example, it generally includes an anode, a hole transport layer, an electroluminescent layer as an energy conversion layer, an electron transport layer and a cathode, which are disposed sequentially. When the voltage is applied to the cathode and the anode, the two electrodes generate an electric field, under the action of the electric field, the electrons on the cathode side move to the electroluminescent layer, the holes on the anode side also move to the luminescent layer, the electrons and holes combine in the electroluminescent layer to form excitons, and the excitons in the excited state release energy outward, which in turn makes the electroluminescent layer emit light to the outside.

At present, in the use of organic electroluminescent devices, problems in related to poor performance still exit, such as short life, and incapability of having a good driving voltage, luminous efficiency and service life, which limit the use of electroluminescent devices. Therefore, it is still necessary to further study this field, especially the functional layer materials used, to improve the performance of organic electroluminescent devices.

### SUMMARY

For the above problems existing in the prior art, the object of the present disclosure is to provide an organic compound, an electronic element and an electronic apparatus. The organic compound of the present disclosure can improve the performance of the electronic device effectively.

In the first aspect, the present disclosure provides an organic compound, the structure of the organic compound is as shown in Formula 1:

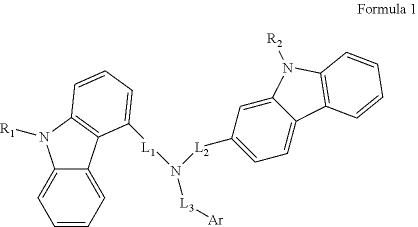

wherein, R₁ and R₂ are the same or different, and are each independently selected from a substituted or unsubstituted aryl with 6 to 25 carbon atoms, a substituted or unsubstituted heteroaryl with 5 to 25 carbon atoms, an alkyl with 1 to 10 carbon atoms, or a cycloalkyl with 3 to 10 carbon atoms;
Ar is selected from a substituted or unsubstituted aryl with 6 to 21 carbon atoms, or a substituted or unsubstituted heteroaryl with 5 to 20 carbon atoms;
L₁, L₂ and L₃ are the same or different, and are each independently selected from a single bond, a substituted or unsubstituted arylene with 6 to 15 carbon atoms, or a heteroarylene with 5 to 15 carbon atoms;
the substituents of R₁, R₂, Ar, L₁, L₂ and L₃ are the same or different, and are each independently selected from deuterium, cyano, an alkyl with 1 to 10 carbon atoms, a deuterated alkyl with 1 to 10 carbon atoms, an alkoxy with 1 to 10 carbon atoms, an alkylthio with 1 to 10 carbon atoms, an aryl with 6 to 12 carbon atoms, a heteroaryl with 5 to 12 carbon atoms, or a cycloalkyl with 3 to 10 carbon atoms;
and the total number of carbon atoms of L; and Ar is not more than 21.

In the second aspect, the present disclosure provides an electronic element, comprising an anode, a cathode, and a functional layer disposed between the anode and the cathode, wherein, the functional layer comprises the organic compound of the first aspect according to the present disclosure.

In the third aspect, the present disclosure provides an electronic apparatus, comprising the electronic element of the second aspect of the present disclosure.

In the organic compound of the present disclosure, two carbazole groups are introduced into the triarylamine structure, and these two carbazole groups are connected to the aromatic amine through positions 2 and 4, respectively. This can provide the molecule with a suitable HOMO energy level, and can also effectively enhance the first triplet energy level of the material. Meanwhile, the -L₃-Ar structure introduced in the triarylamine structure is a simple and small aromatic group, which can effectively avoid the stacking between molecules and provide the material with excellent film-forming properties. The organic compound is used as a hole transport layer material in an organic electroluminescent device, and can improve the service life of the device.

Other features and advantages of the present disclosure will be described in detail in the subsequent detailed description.

### BRIEF DESCRIPTION OF THE DRAWINGS

FIG. 1 shows a schematic diagram of the structure of an organic electroluminescent device of an embodiment according to the present disclosure.

FIG. 2 shows a schematic diagram of the structure of a photoelectric conversion device of an embodiment according to the present disclosure.

FIG. 3 shows a schematic diagram of an electronic apparatus of an embodiment according to the present disclosure.

FIG. 4 shows a schematic diagram of an electronic apparatus of another embodiment according to the present disclosure.

### DESCRIPTION OF THE REFERENCE SIGNS

100, anode; 200, cathode; 300, functional layer; 310, hole injection layer; 320, hole transport layer; 321, electron barrier layer; 330, organic light-emitting layer; 340, electron transport layer; 350, electron injection layer; 360: photoelectric conversion layer; 400: a first electronic apparatus; and 500: a second electronic apparatus.

### DETAILED DESCRIPTION

The specific embodiments of the present disclosure are described in detail in combination with the drawings. It is to be understood that the specific embodiments described herein are intended only to illustrate and explain the present invention and are not intended to limit the present invention.

The first aspect of the present disclosure is to provide an organic compound having the structure shown in Formula 1:

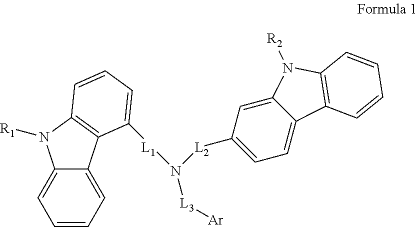

wherein, R₁ and R₂ are the same or different, and are each independently selected from a substituted or unsubstituted aryl with 6 to 25 carbon atoms, a substituted or unsubstituted heteroaryl with 5 to 25 carbon atoms, an alkyl with 1 to 10 carbon atoms, or a cycloalkyl with 3 to 10 carbon atoms;
Ar is selected from a substituted or unsubstituted aryl with 6 to 21 carbon atoms, or a substituted or unsubstituted heteroaryl with 5 to 20 carbon atoms;
L₁, L₂ and L₃ are the same or different, and are each independently selected from a single bond, a substituted or unsubstituted arylene with 6 to 15 carbon atoms, or a heteroarylene with 5 to 15 carbon atoms;
the substituents of R₁, R₂, Ar, L₁, L₂ and L₃ are the same or different, and are each independently selected from deuterium, cyano, an alkyl with 1 to 10 carbon atoms, a deuterated alkyl with 1 to 10 carbon atoms, an alkoxy with 1 to 10 carbon atoms, an alkylthio with 1 to 10 carbon atoms, an aryl with 6 to 12 carbon atoms, a heteroaryl with 5 to 12 carbon atoms, or a cycloalkyl with 3 to 10 carbon atoms;
and the total number of carbon atoms of L₃ and Ar is not more than 21. That is, the total number of carbon atoms of group

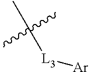

is not more than 21.

The expressions “each . . . is independently selected from” and “ . . . is independently selected from” in the present disclosure can be used interchangeably and should be understood in a broad sense. It can mean that the specific options expressed between the same symbols in different groups do not affect each other, or it can mean that the specific options expressed between the same symbols in the same group do not affect each other. For example,

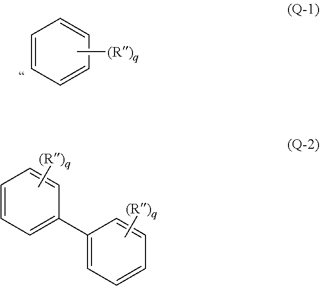

wherein, each q is independently selected from the group consisting of 0, 1, 2 or 3, and each R″ is independently selected from the group consisting of hydrogen, deuterium, fluorine, and chlorine” means that: Formula Q-1 represents that there are q substituents of R″ in the benzene ring, each R″ can be the same or different, and options for each R″ do not affect each other; and Formula Q-2 represents that there are q substituents of R″ in each benzene ring of the biphenyl, the number q of the substituents of R″ in the two benzene rings can be the same or different, each R″ can be the same or different, and options for each R″ do not affect each other.

In the present disclosure, the term “substituted or unsubstituted” means that the functional group described after the term may or may not have a substituent (hereinafter referred to as R_{c} for ease of description). For example, “substituted or unsubstituted aryl” refers to an aryl with a substituent Re or a non-substituted aryl. The above-mentioned substituent, i.e., R_{c}, for example, can be deuterium, cyano, heteroaryl, aryl, deuterated aryl, alkyl, deuterated alkyl, cycloalkyl, alkoxy, alkylthio, etc. When two substituents R_{c} are attached to the same atom, the two substituents R_{c} may exist independently or be connected to each other to form a ring with the atom; when there are two adjacent substituents Re on a functional group, the adjacent substituents R_{c} can exist independently or be fused to form a ring with the functional group to which they are attached.

In the present disclosure, the number of carbon atoms of a substituted or unsubstituted functional group refers to the number of all carbon atoms. For example, if Ar is a substituted aryl with a number of carbon atoms of 12, then the number of all carbon atoms of the aryl and the substituents thereon is 12.

In the present disclosure, aryl refers to any functional group or substituent derived from an aromatic carbon ring. The aryl may be a monocyclic aryl (e.g., phenyl) or a polycyclic aryl, in other words, the aryl may be a monocyclic aryl, a fused aryl, two or more monocyclic aryl connected by a carbon-carbon conjugate linkage, a monocyclic aryl and a fused aryl connected by a carbon-carbon conjugate linkage, two or more fused aryl connected by a carbon-carbon conjugate linkage. That is, unless otherwise specified, two or more aromatic groups connected by a carbon-carbon conjugate linkage may also be regarded as aryl according to the present disclosure. Wherein the fused aryl may include, for example, a bicyclic fused aryl (e.g., naphthyl), a tricyclic fused aryl (e.g., phenanthryl, fluorenyl, anthryl) and the like. The aryl does not contain heteroatoms such as B, N, O, S, P. Se and Si. It should be noted that biphenyl, terphenyl, 9,9-dimethylfluorenyl are all treated as aryl in the present disclosure. Examples of aryl may include, but not limited to, phenyl, naphthyl, fluorenyl, anthryl, phenanthryl, biphenyl, terphenyl, benzo[9,10]phenanthryl, pyrenyl, benzofluoranthryl, chrysenyl and the like.

In the present disclosure, the substituted aryl may be that one or more hydrogen atoms of the aryl are substituted by a group such as deuterium, cyano, aryl, heteroaryl, alkyl, cycloalkyl, deuterated alkyl, alkoxy, alkylthio, etc. Specific examples of heteroaryl-substituted aryl include, but not limited to, dibenzofuranyl-substituted phenyl, dibenzothienyl-substituted phenyl, etc. It should be understood that the number of carbon atoms of substituted aryl refers to the total number of carbon atoms of an aryl and the substituent on the aryl, for example, the substituted aryl with a number of carbon atoms of 18 refers to the total number of carbon atoms of the aryl and the substituent group is 18.

In the present disclosure, heteroaryl refers to a monovalent aromatic ring comprising 1, 2, 3, 4, 5, 6 or more heteroatoms or derivatives thereof, and the heteroatoms may be at least one of B, O, N, P, Si, Se, and S. Heteroaryl can be monocyclic heteroaryl or polycyclic heteroaryl. In other words, heteroaryl may be a single aromatic ring system, or a plurality of aromatic ring systems connected by a carbon-carbon conjugate linkage, and any one of the aromatic ring systems is an aromatic single ring or an aromatic fused ring. For example, heteroaryl includes but not limited to thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzoimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, etc. Among them, thienyl, furyl, phenanthrolinyl, etc. are heteroaryl with single aromatic ring system, and N-phenylcarbazolyl and N-pyridyl are heteroaryl with multiple aromatic ring systems connected by a carbon-carbon conjugate linkage. In the present disclosure, the involved heteroarylene refers to the divalent group formed by a further loss of a hydrogen atom of a heteroaryl.

In the present disclosure, the substituted heteroaryl may be that one or more hydrogen atoms of the heteroaryl are substituted with a group such as deuterium, cyano, aryl, heteroaryl, alkyl, cycloalkyl, deuterated alkyl, alkoxy, alkylthio, etc. Specific examples of aryl-substituted heteroaryl include, but not limited to, phenyl-substituted dibenzofuranyl, phenyl-substituted dibenzothienyl, phenyl-substituted pyridinyl, etc. It should be understood that the number of carbon atoms of substituted heteroaryl refers to the total number of carbon atoms of a heteroaryl and substituents on the heteroaryl.

In the present disclosure, the non-positional bond refers to a single bond

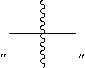

extending from a ring system, which represents that one end of the bond can be attached to any position in the ring system through which the bond penetrates, and the other end is attached to the remaining part in the compound molecule. For example, as shown in Formula (f) below, the naphthylene group represented by Formula (f) is attached to the remaining part in the molecule by two non-positional bonds penetrating through the bicyclic ring, and the meaning expressed therein includes any of the possible connection manners shown in Formula (f-1) to Formula (f-10):

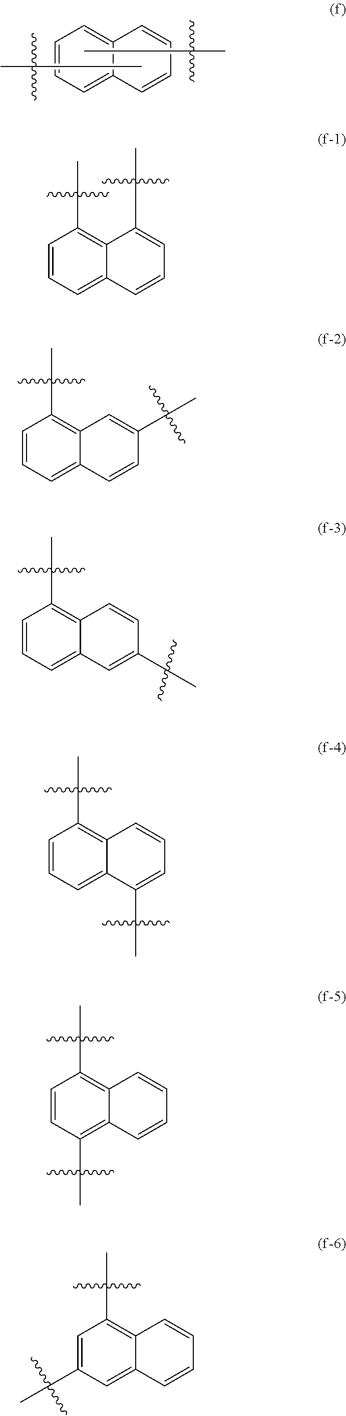

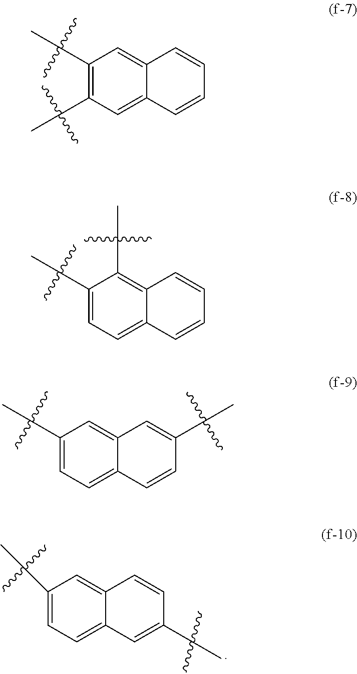 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the phenanthryl represented by Formula (X′) is attached to the remaining part in the molecule by a non-positioned connecting bond extending from the center of one benzene ring, and the meaning expressed therein, includes any of the possible connection manners shown in Formula (X′-1) to Formula (X′-4):

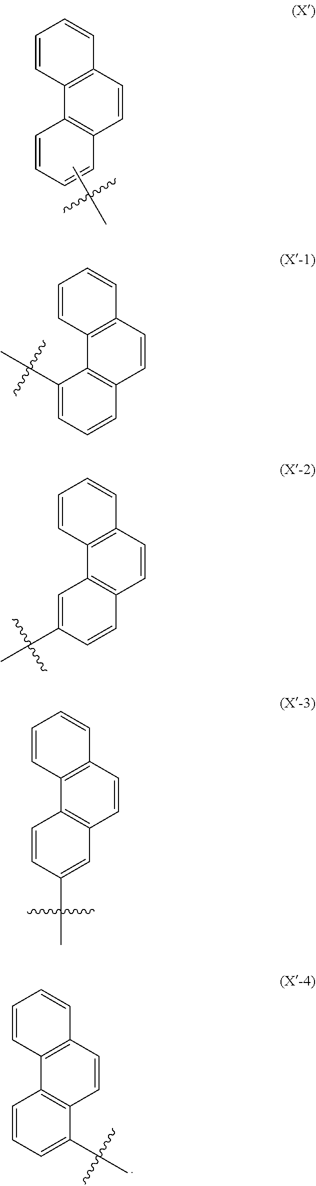

The non-positioned substituent in the present disclosure refers to a substituent linked by a single bond extending from the center of a ring system, which represents that the substituent may be linked to any possible position in the ring system. For example, as shown in Formula (Y) below, the substituent R′ presented in Formula (Y) is connected to a quinoline ring by an non-positioned connection bond, and the meaning expressed therein, includes any possible connection manners as shown in Formula (Y-1) to Formula (Y-7):

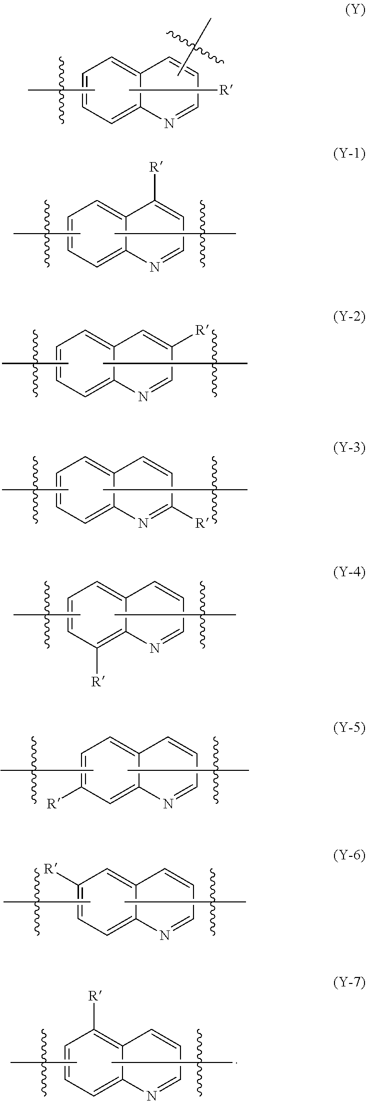

In the present disclosure, the alkyl with 1 to 10 carbon atoms includes a straight-chain alkyl with 1 to 10 carbon atoms and a branched-chain alkyl with 1 to 10 carbon atoms, and the number of carbon atoms may be 1, 2, 3, 4, 5, 6, 7, 8, 9, or 10. Specific examples of alkyl with 1 to 10 carbon atoms include, but not limited to, methyl, ethyl, n-propyl, isopropyl, n-butyl, isobutyl, tert-butyl, n-pentyl, isoamyl, neopentyl, cyclopentyl, n-hexyl, heptyl, n-octyl, 2-ethylhexyl, nonyl, decyl, 3,7-dimethyloctyl and the like.

In the present disclosure, specific examples of deuterated alkyl with 1 to 10 carbon atoms include, but not limited to, trideuterated methyl.

In the present disclosure, the number of carbon atoms of aryl as a substituent may be 6-12, for example, 6, 10, 12, etc., Specific examples of aryl as a substituent include, phenyl, naphthyl, and biphenyl.

In the present disclosure, the number of carbon atoms of heteroaryl as a substituent may be 5-12. The number of carbon atoms is for example, 5, 8, 9, 10, 12, etc., Specific examples of heteroaryl as a substituent include, but not limited to, pyridinyl, quinolyl, dibenzofuranyl, dibenzothienyl, carbazolyl, etc.

In the present disclosure, the number of carbon atoms of cycloalkyl as a substituent may be 3-10, preferably 5-8. Specific examples of cycloalkyl include, but not limited to, cyclopentyl, cyclohexyl, etc.

Optionally, R₁ and R₂ are the same or different, and are each independently selected from a substituted or unsubstituted aryl with 6 to 18 carbon atoms, a substituted or unsubstituted heteroaryl with 5 to 15 carbon atoms, an alkyl with 1 to 5 carbon atoms, or a cycloalkyl with 3 to 8 carbon atoms. For example, each of R₁ and R₂ is independently selected from: a substituted or unsubstituted aryl with a number of carbon atoms of 6, 7, 8, 9, 10, 11, 12, 13, 14, 15, 16, 17, or 18, a substituted or unsubstituted heteroaryl with a number of carbon atoms of 5, 6, 7, 8, 9, 10, 11, 12, 13, 14, 15, 16, 17, or 18, an alkyl with a number of carbon atoms of 1, 2, 3, 4, or 5, or a cycloalkyl with a number of carbon atoms of 3, 4, 5, 6, 7, or 8.

Optionally, R₁ and R₂ are the same or different, and are each independently selected from a substituted or unsubstituted aryl with 6 to 18 carbon atoms, a substituted or unsubstituted heteroaryl with 5 to 18 carbon atoms of 5-18, an alkyl with 1 to 5 carbon atoms, or a cycloalkyl with 3 to 8 carbon atoms.

In an embodiment, R₁ and R₂ are the same or different, and are each independently selected from a substituted or unsubstituted aryl with 6 to 18 carbon atoms, a substituted or unsubstituted heteroaryl with 12 to 15 carbon atoms, an alkyl with 1 to 5 carbon atoms, or a cycloalkyl group with 5 to 8 carbon atoms.

Optionally, each of R₁ and R₂ is independently selected from a methyl, an ethyl, a n-propyl, an isopropyl, a n-butyl, an isobutyl, a tert-butyl, a cyclopentyl, a cyclohexyl, a substituted or unsubstituted phenyl, a substituted or unsubstituted biphenyl, a substituted or unsubstituted naphthyl, a substituted or unsubstituted fluorenyl, a substituted or unsubstituted phenanthryl, a substituted or unsubstituted terphenyl, a substituted or unsubstituted pyridyl, a substituted or unsubstituted dibenzofuranyl, a substituted or unsubstituted dibenzothienyl, or a substituted or unsubstituted carbazolyl.

Optionally, each of the substituents of R₁ and R₂ is independently selected from deuterium, a cyano, an alkyl with 1 to 4 carbon atoms, a deuterated alkyl with 1 to 4 carbon atoms, an alkoxy with 1 to 4 carbon atoms, an alkylthio with 1 to 4 carbon atoms, an aryl with 6 to 10 carbon atoms, or a cycloalkyl with 5-10 carbon atoms.

Optionally, each of the substituents of R₁ and R₂ is independently selected from deuterium, a cyano, a methyl, an ethyl, a n-propyl, an isopropyl, a tert-butyl, a trideuterated methyl, a methoxy, an ethoxy, a phenyl, a naphthyl, a cyclopentyl or a cyclohexyl.

In an embodiment, each of R₁ and R₂ is independently selected from a methyl, an ethyl, a n-propyl, an isopropyl, a n-butyl, an isobutyl, a tert-butyl, a cyclopentyl, a cyclohexyl, a substituted or unsubstituted group W, wherein the unsubstituted group W is selected from the group consisting of the following groups:

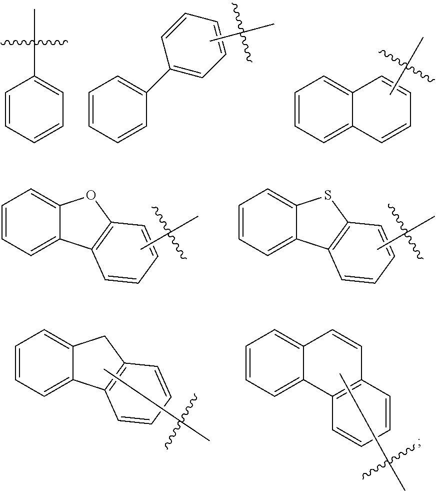

the substituted group W has one or more substituents, each of the substituents is independently selected from deuterium, a cyano, a methyl, an ethyl, a n-propyl, an isopropyl, a tert-butyl, a trideuterated methyl, or a phenyl; when the number of the substituents is more than 1, the substituents are the same or different.

Optionally, each of R₁ and R₂ is independently selected from the group consisting of a methyl, an ethyl, a n-propyl, an isopropyl, a n-butyl, an isobutyl, a tert-butyl and the following groups:

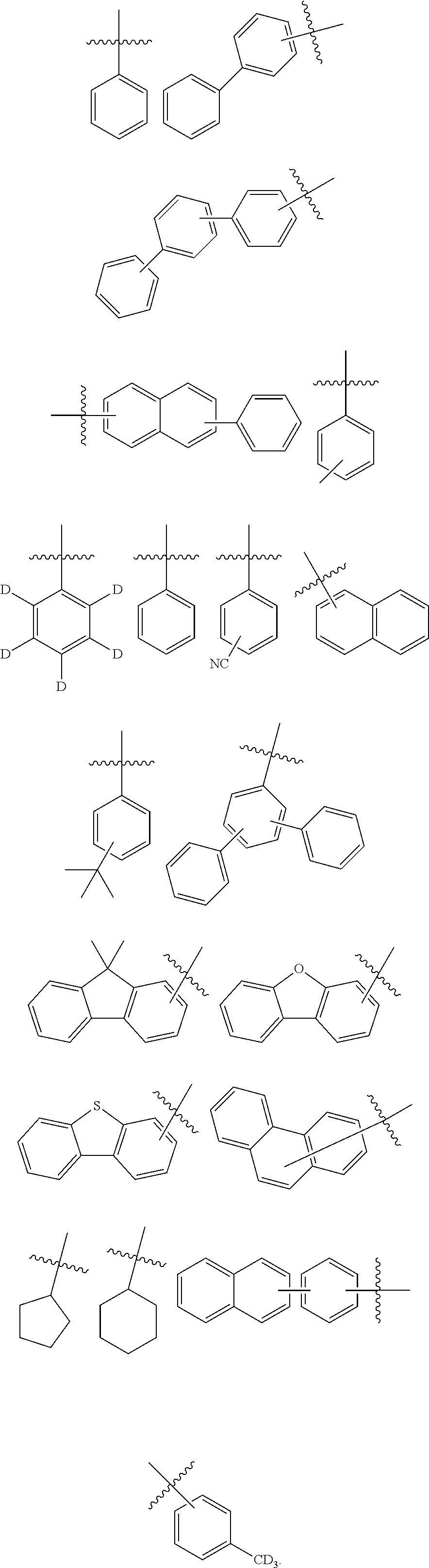

Further optionally, each of R₁ and R₂ is independently selected from the group consisting of a methyl, an ethyl, a n-propyl, an isopropyl, a n-butyl, an isobutyl, a tert-butyl and the following groups:

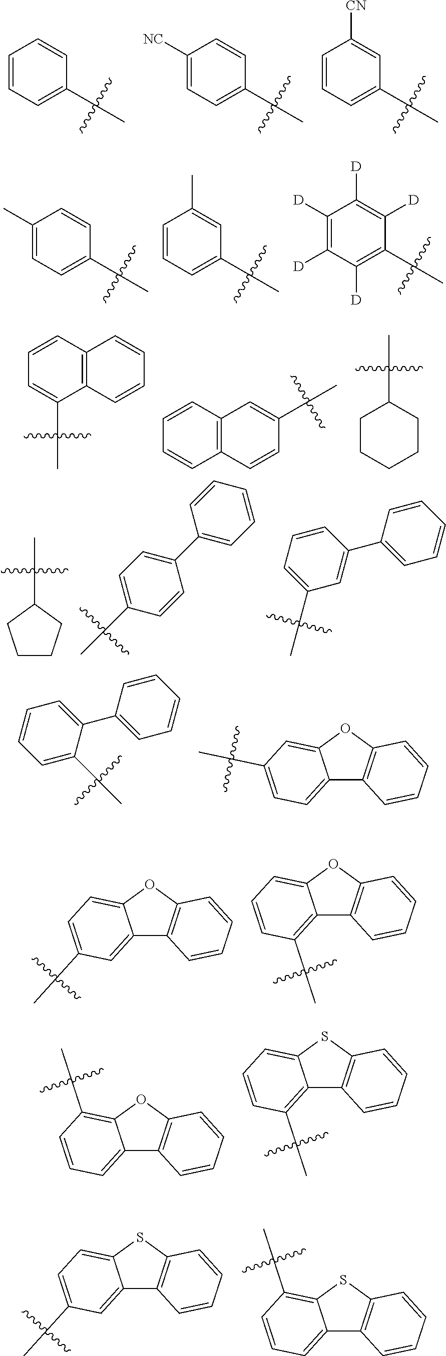

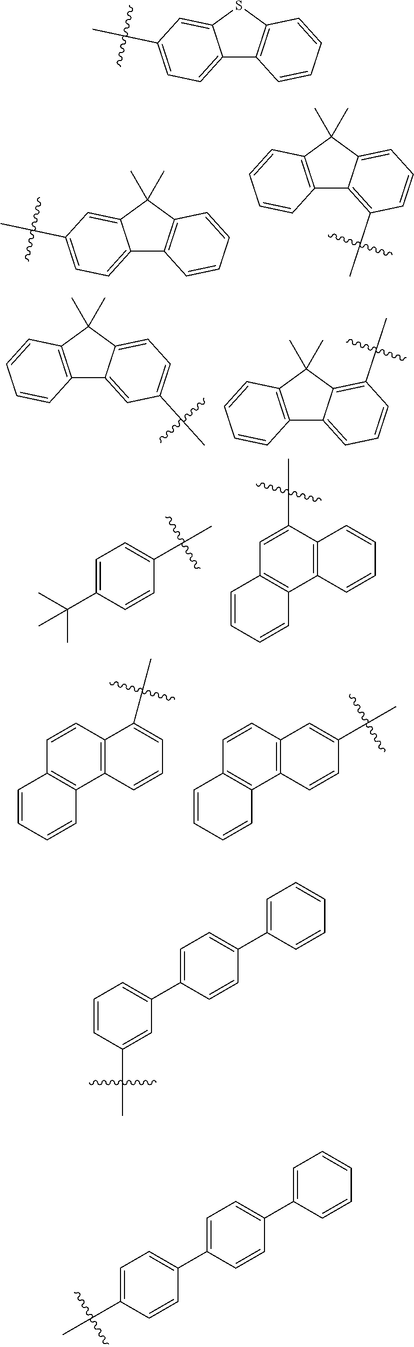

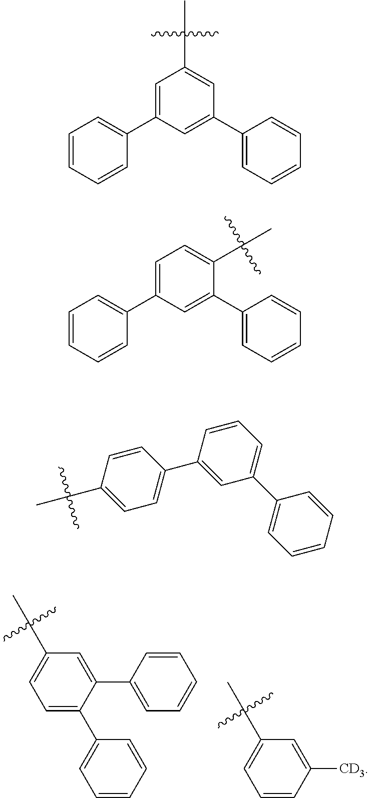 [SMILES: *c1cc(-c2ccccc2)cc(-c2ccccc2)c1 ; *c1ccc(-c2ccccc2)cc1-c1ccccc1 ; *c1ccc(-c2cccc(-c3ccccc3)c2)cc1 ; *c1ccc(-c2ccccc2)c(-c2ccccc2)c1 ; *c1cccc(C([2H])([2H])[2H])c1]

In some embodiments, the structure of the organic compound is as shown in one of the followings:

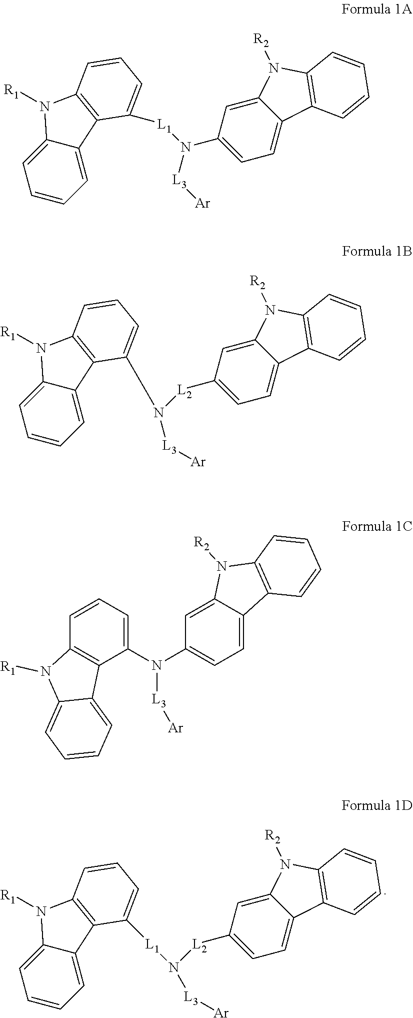

In an embodiment, R₁, R₂, Ar, L₁, L₂ and L; in Formula 1A, Formula 1B, Formula 1C and Formula 1D have the same definitions as those in Formula 1.

In another embodiment of the present disclosure, none of L₁, L₂ and L₃ in Formula 1A to Formula 1D is a single bond. Preferably, the structure of the organic compound is as shown in Formula 1B.

Optionally, L₁, L₂ and L₃ are the same or different, and are each independently selected from a single bond, a substituted or unsubstituted arylene with 6 to 12 carbon atoms, or a substituted or unsubstituted heteroarylene with 5 to 12 carbon atoms. For example, each of L₁, L₂ and L₃ is independently selected from: a single bond, a substituted or unsubstituted arylene with a carbon number of 6, 7, 8, 9, 10, 11, or 12, or selected from a substituted or unsubstituted heteroarylene with a number of carbon atoms of 5, 6, 7, 8, 9, 10, 11, or 12.

In an embodiment, L₁, L₂ and L₃ are the same or different, and are each independently selected from a single bond, a substituted or unsubstituted phenylene, a substituted or unsubstituted naphthylene, a substituted or unsubstituted biphenylene, a substituted or unsubstituted pyridylidene, a substituted or unsubstituted dibenzofuranylene, or a substituted or unsubstituted dibenzothienylene.

Optionally, the substituents of L₁, L₂ and L₃ are the same or different, and are each independently selected from deuterium, a cyano, an alkyl with 1 to 4 carbon atoms, a deuterated alkyl with 1 to 4 carbon atoms, or a phenyl.

Further optionally, each of the substituents of L₁, L₂ and L₃ is independently selected deuterium, a cyano, a methyl, an ethyl, an isopropyl, a tert-butyl, a trideuterated methyl or a phenyl.

In an embodiment, L₁, L₂ and L₃ are the same or different, and are each independently selected from a single bond or a substituted or unsubstituted group V, wherein, the unsubstituted group V is selected from the group consisting of the following groups:

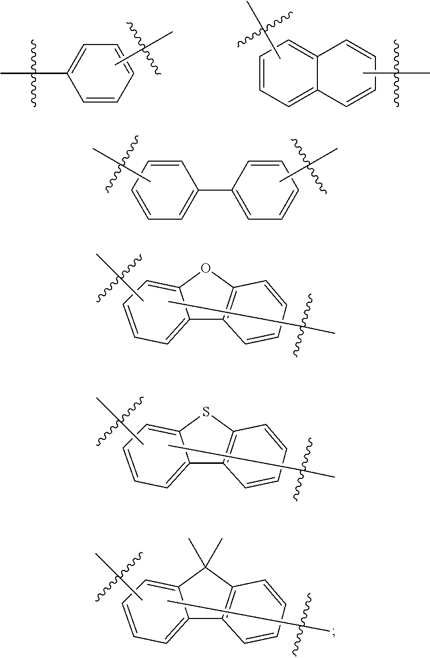

the substituted group V has one or more substituents, each of the substituents is independently selected from deuterium, a cyano, a methyl, an ethyl, an isopropyl, a tert-butyl, a trideuterated methyl or a phenyl; when the number of the substituents is more than 1, the substituents are the same or different.

Optionally, L₁, L₂ and L₃ are the same or different, and are each independently selected from the group consisting of a bond and the following groups:

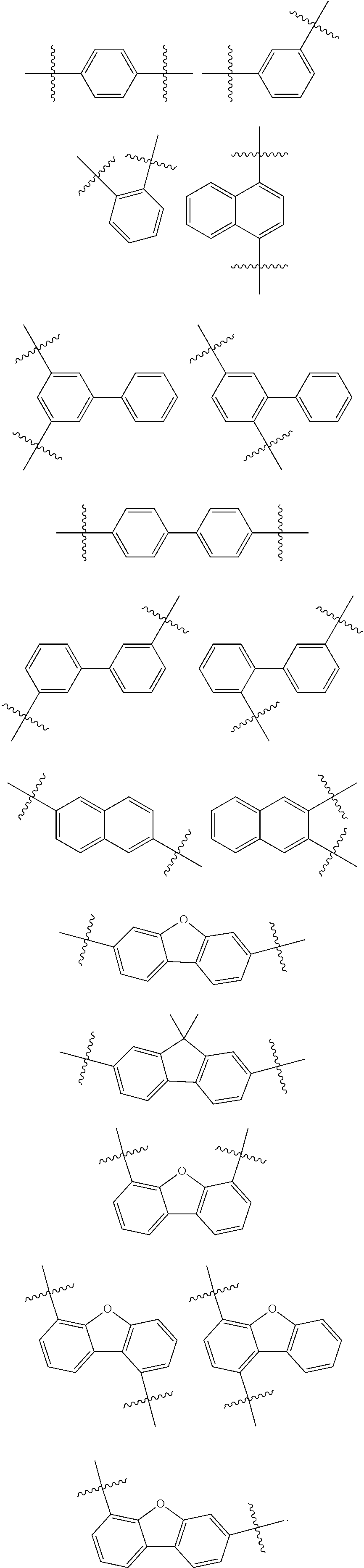

In a specific embodiment, L₁ and L₂ are each independently selected from the group consisting of a single bond and the following groups:

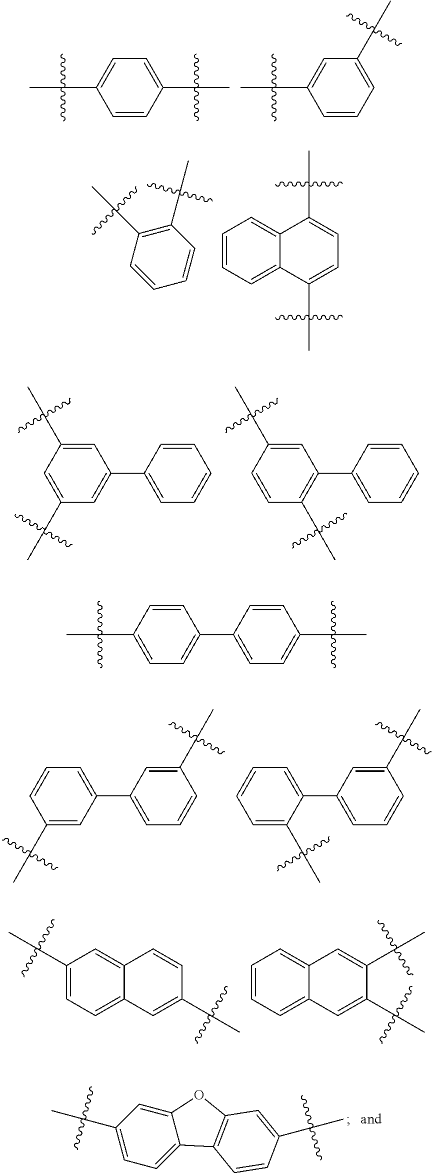

L₃ is selected from the group consisting of a single bond and the following groups:

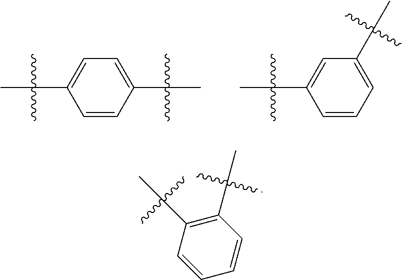 [SMILES: *c1ccc(*)cc1 ; *c1cccc(*)c1 ; *c1ccccc1*]

In a preferable embodiment, L₁ is a single bond, L₂ is selected from a substituted or unsubstituted arylene with 6 to 12 carbon atoms, L₃ is selected from a single bond, a substituted or unsubstituted arylene with 6 to 12 carbon atoms. In this embodiment, the organic compound has higher hole mobility and energy transfer efficiency, and more stable spatial configuration. The organic compound as a hole transport layer material, can further increase the service life of the organic electroluminescent device, and further improve the comprehensive performance of the device. More preferably, L₂ is selected from a substituted or unsubstituted phenylene, a substituted or unsubstituted naphthylene, or a substituted or unsubstituted biphenylene, the substituent of L₂ is defined as above.

In the present disclosure, Ar can be selected from a substituted or unsubstituted aryl with a number of carbon atoms of 6, 7, 8, 9, 10, 11, 12, 13, 14, 15, 16, 17, or 18, or a substituted or unsubstituted heteroaryl with a number of carbon atoms of 5, 6, 7, 8, 9, 10, 11, 12, 13, 14, 15, 16, 17, or 18.

In a specific embodiment, Ar is selected from a substituted or unsubstituted aryl with 6 to 18 carbon atoms, or a substituted or unsubstituted heteroaryl with 12 to 18 carbon atoms.

Optionally, each of the substituents of Ar is independently selected from deuterium, a cyano, an alkyl with 1 to 4 carbon atoms, a deuterated alkyl with 1 to 4 carbon atoms, or an aryl with 6 to 10 carbon atoms.

Further optionally, each of the substituents of Ar is independently selected from deuterium, a cyano, a methyl, an ethyl, a n-propyl, an isopropyl, a tert-butyl, a trideuterated methyl, a phenyl or a naphthyl.

In an embodiment, Ar is selected from a substituted or unsubstituted group Z, wherein, the unsubstituted group Z is selected from the group consisting of the following groups:

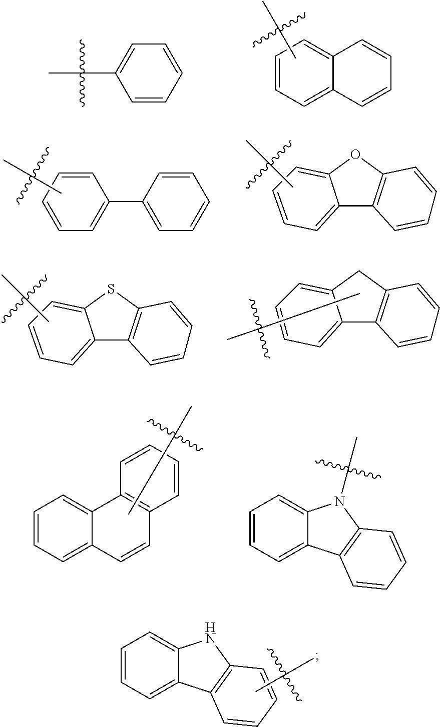

the substituted group Z has one or more substituents, each of the substituents is independently selected from deuterium, a cyano, a methyl, an ethyl, an isopropyl, a tert-butyl, a trideuterated methyl, a phenyl or a naphthyl; when the number of the substituents is more than 1, the substituents are the same or different.

Optionally, Ar is selected from the group consisting of the following groups:

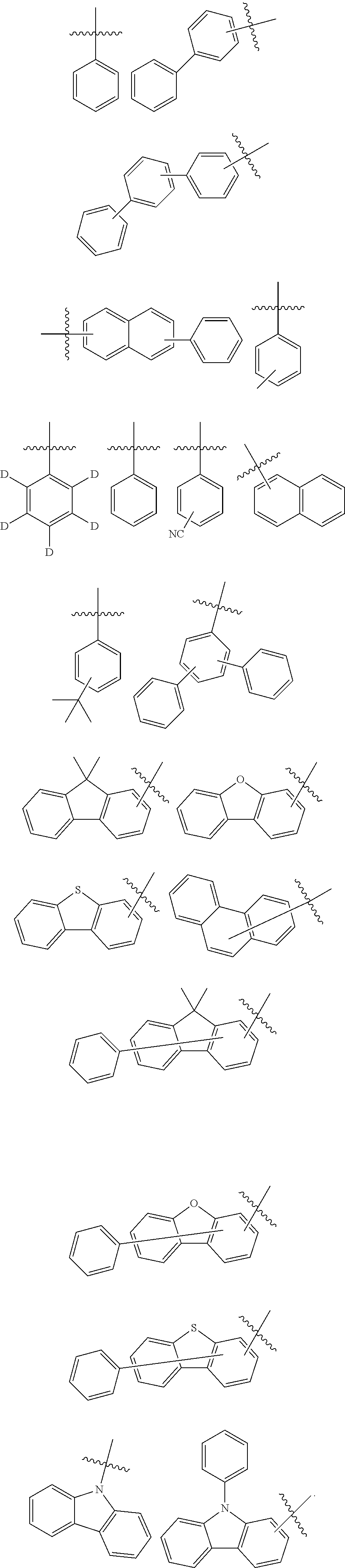

Further optionally, Ar is selected from the group consisting of the following groups:

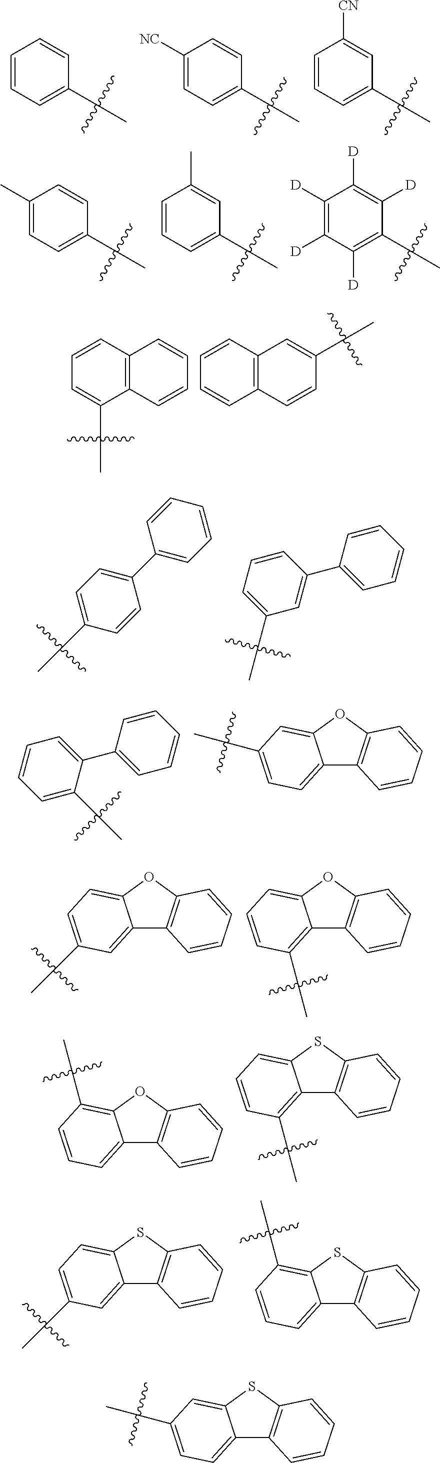

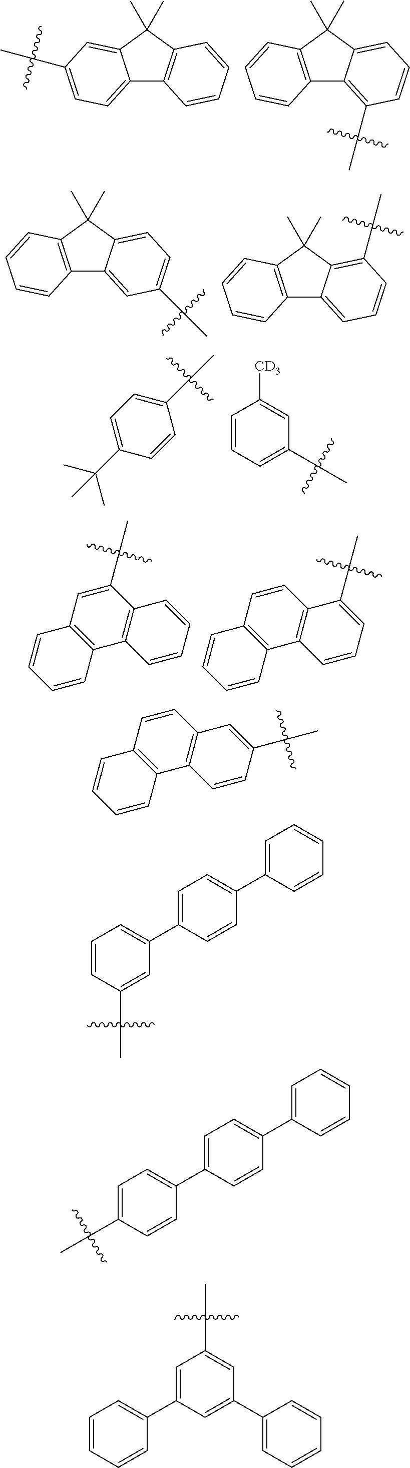

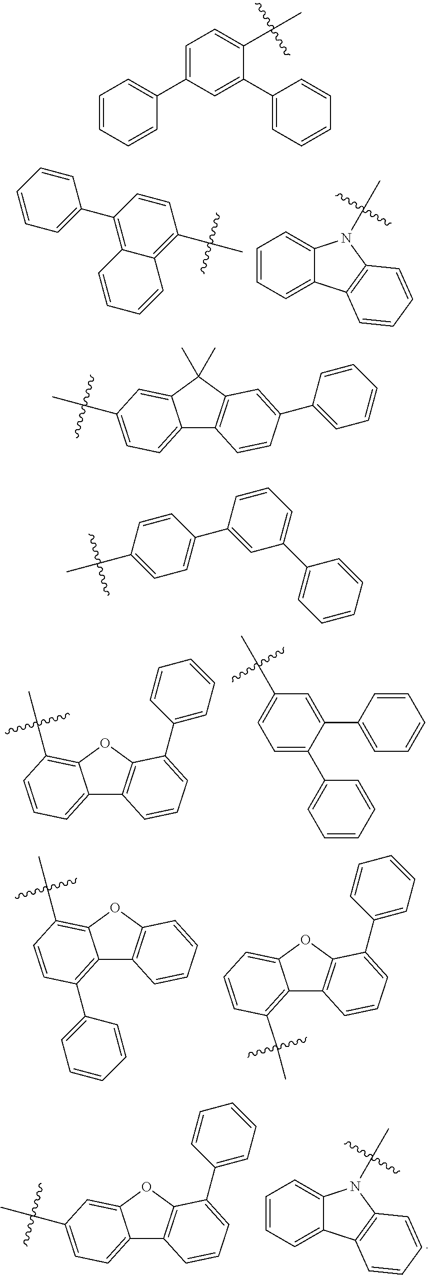

In an embodiment,

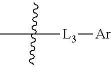

is selected from the group consisting of the following-L-A groups:

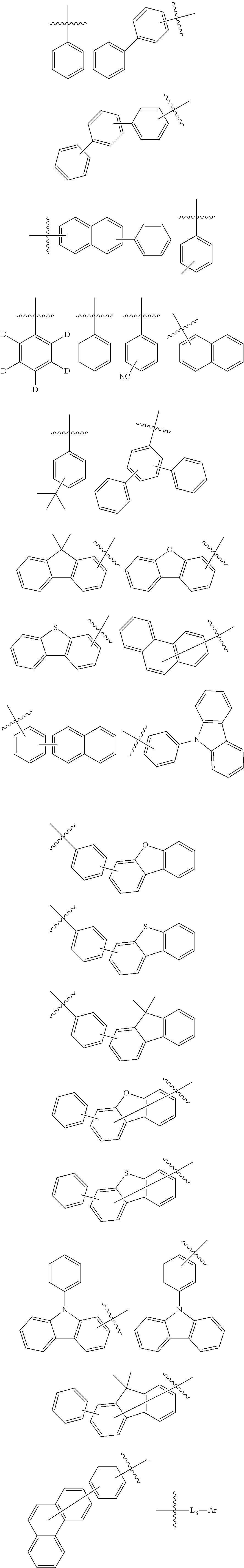

is an aromatic group or two aromatic groups connected by a bond, which can maintain a fixed dihedral angle between the two carbazolyl groups on the aromatic amine, and maintain the steric configuration of the compound in the best state.

Optionally, the organic compound is selected from the group consisting of the following compounds:

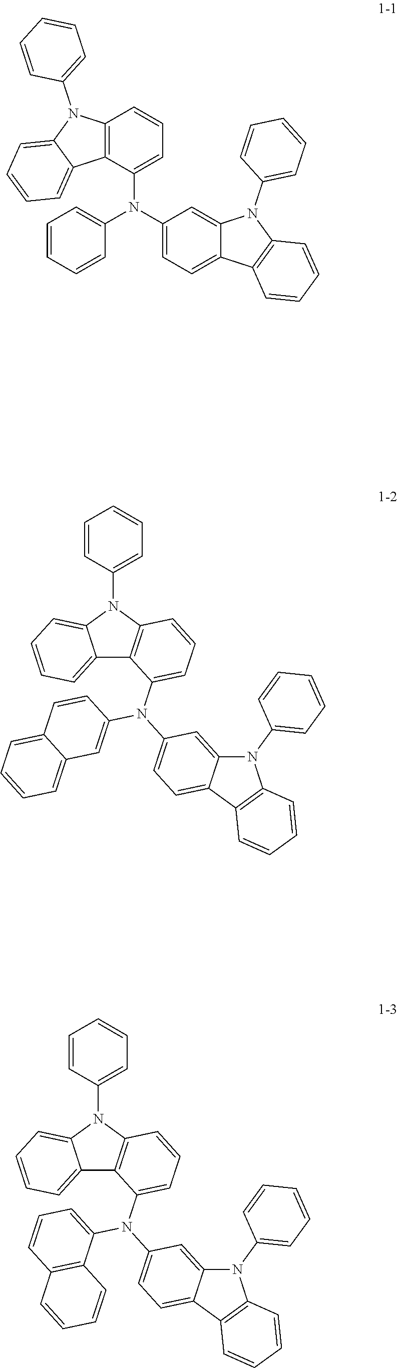 [SMILES: c1ccc(N(c2ccc3c4ccccc4n(-c4ccccc4)c3c2)c2cccc3c2c2ccccc2n3-c2ccccc2)cc1 ; c1ccc(-n2c3ccccc3c3ccc(N(c4ccc5ccccc5c4)c4cccc5c4c4ccccc4n5-c4ccccc4)cc32)cc1 ; c1ccc(-n2c3ccccc3c3ccc(N(c4cccc5ccccc45)c4cccc5c4c4ccccc4n5-c4ccccc4)cc32)cc1]

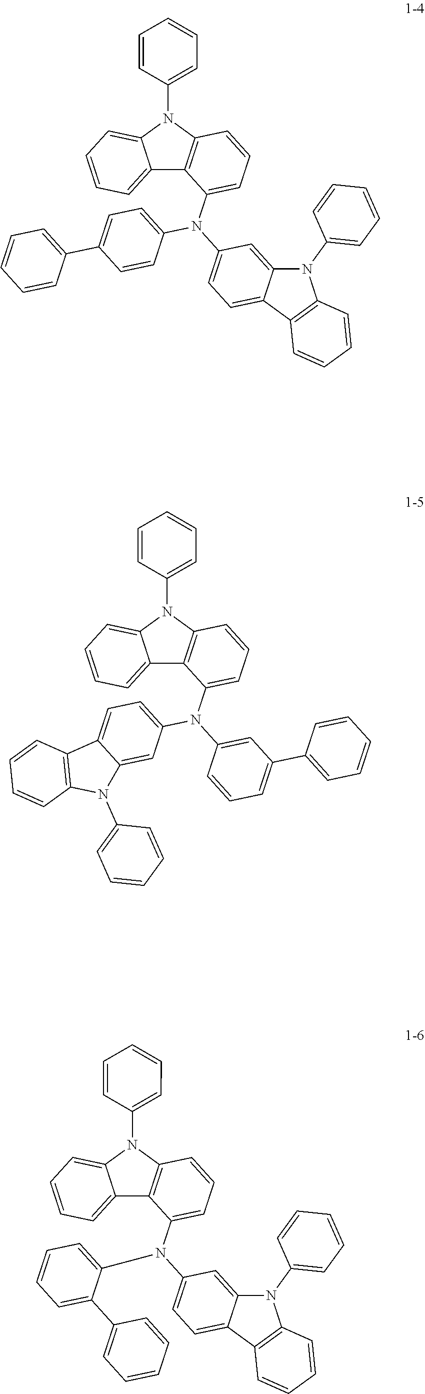 [SMILES: c1ccc(-c2ccc(N(c3ccc4c5ccccc5n(-c5ccccc5)c4c3)c3cccc4c3c3ccccc3n4-c3ccccc3)cc2)cc1 ; c1ccc(-c2cccc(N(c3ccc4c5ccccc5n(-c5ccccc5)c4c3)c3cccc4c3c3ccccc3n4-c3ccccc3)c2)cc1 ; c1ccc(-c2ccccc2N(c2ccc3c4ccccc4n(-c4ccccc4)c3c2)c2cccc3c2c2ccccc2n3-c2ccccc2)cc1]

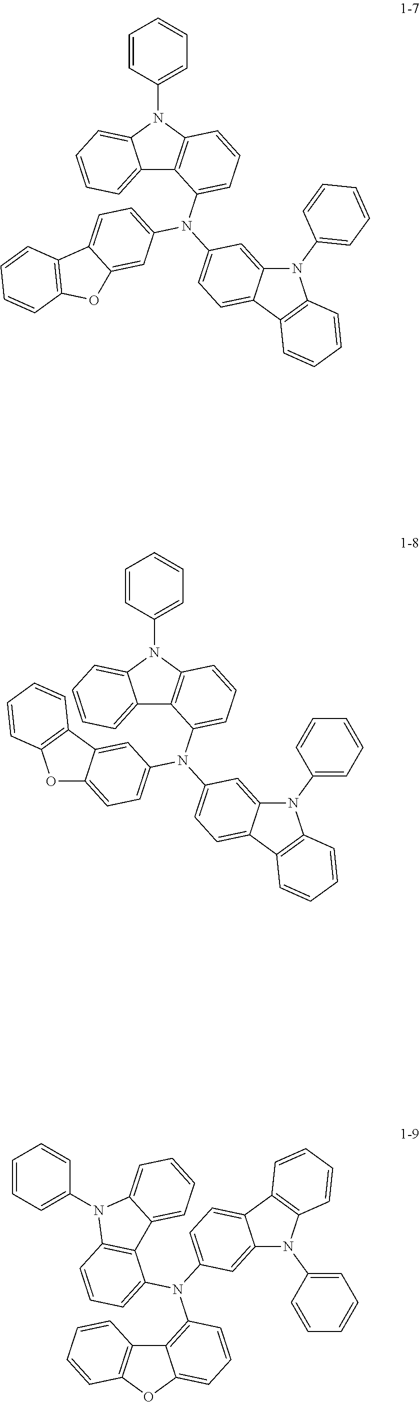 [SMILES: c1ccc(-n2c3ccccc3c3ccc(N(c4ccc5c(c4)oc4ccccc45)c4cccc5c4c4ccccc4n5-c4ccccc4)cc32)cc1 ; c1ccc(-n2c3ccccc3c3ccc(N(c4ccc5oc6ccccc6c5c4)c4cccc5c4c4ccccc4n5-c4ccccc4)cc32)cc1 ; c1ccc(-n2c3ccccc3c3ccc(N(c4cccc5oc6ccccc6c45)c4cccc5c4c4ccccc4n5-c4ccccc4)cc32)cc1]

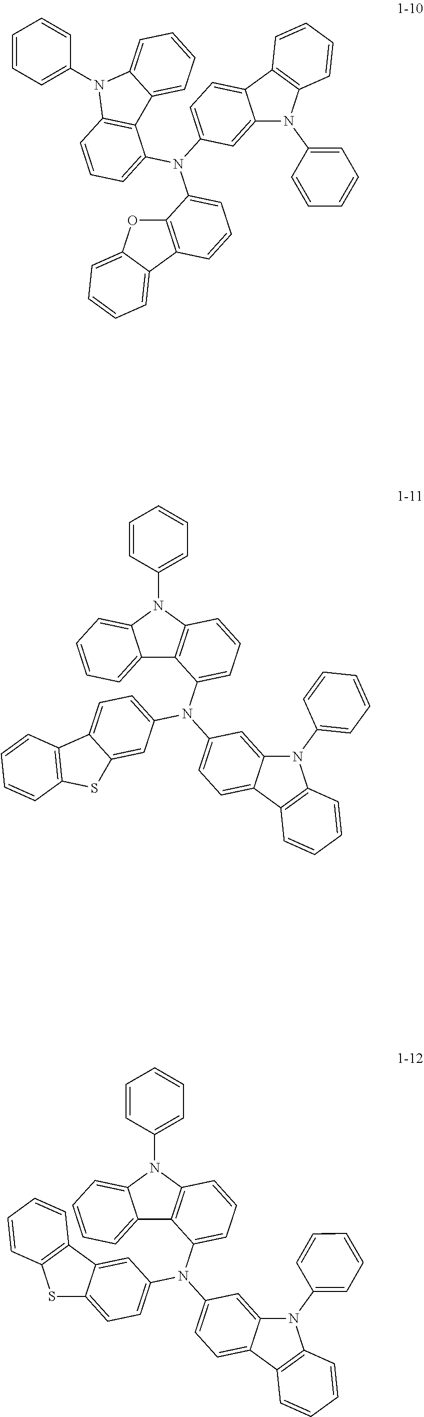 [SMILES: c1ccc(-n2c3ccccc3c3ccc(N(c4cccc5c4oc4ccccc45)c4cccc5c4c4ccccc4n5-c4ccccc4)cc32)cc1 ; c1ccc(-n2c3ccccc3c3ccc(N(c4ccc5c(c4)sc4ccccc45)c4cccc5c4c4ccccc4n5-c4ccccc4)cc32)cc1 ; c1ccc(-n2c3ccccc3c3ccc(N(c4ccc5sc6ccccc6c5c4)c4cccc5c4c4ccccc4n5-c4ccccc4)cc32)cc1]

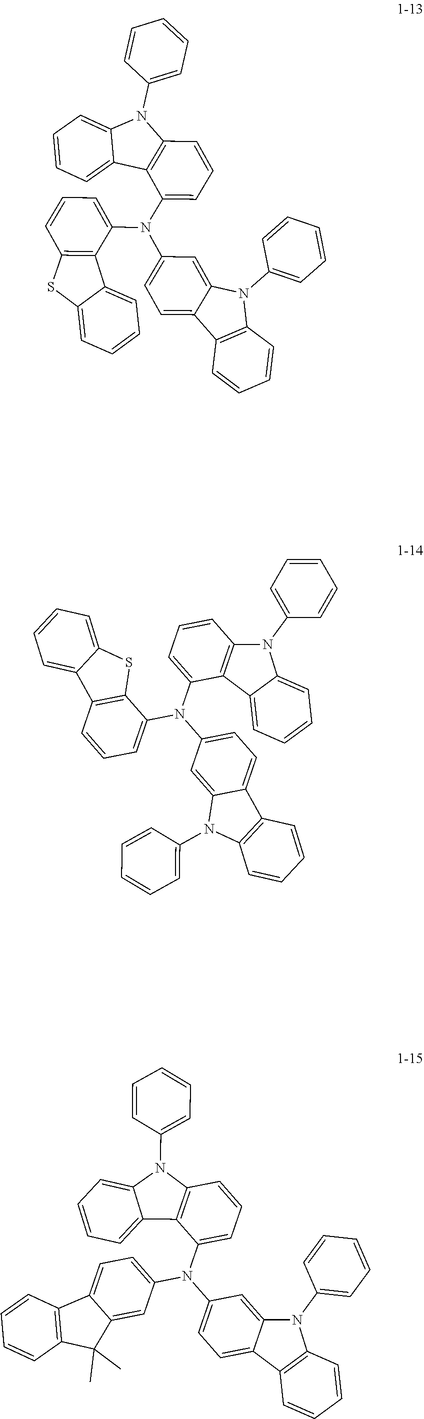 [SMILES: c1ccc(-n2c3ccccc3c3ccc(N(c4cccc5sc6ccccc6c45)c4cccc5c4c4ccccc4n5-c4ccccc4)cc32)cc1 ; c1ccc(-n2c3ccccc3c3ccc(N(c4cccc5c4sc4ccccc45)c4cccc5c4c4ccccc4n5-c4ccccc4)cc32)cc1 ; CC1(C)c2ccccc2-c2ccc(N(c3ccc4c5ccccc5n(-c5ccccc5)c4c3)c3cccc4c3c3ccccc3n4-c3ccccc3)cc21]

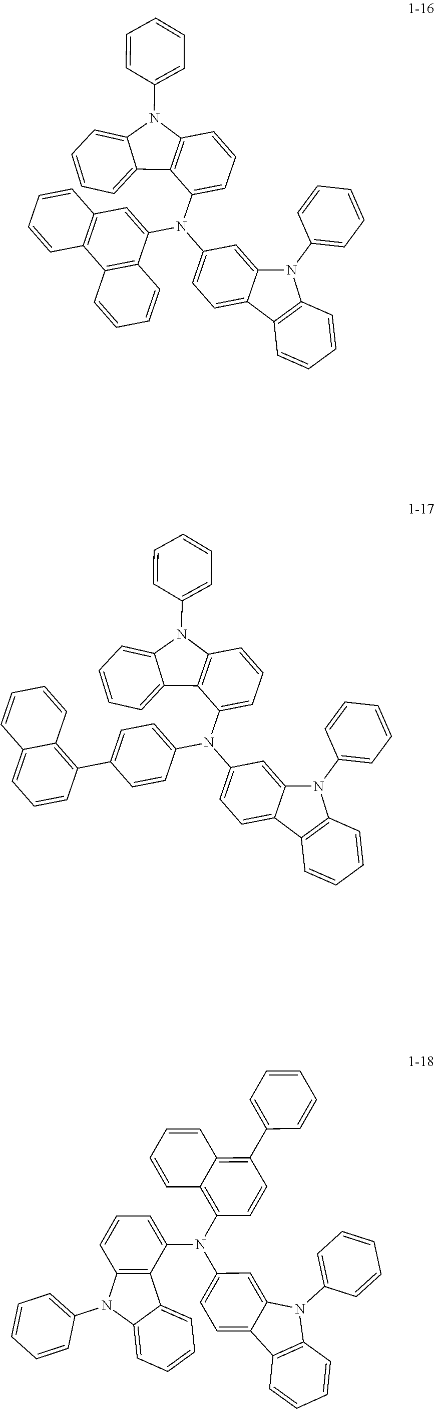 [SMILES: c1ccc(-n2c3ccccc3c3ccc(N(c4cc5ccccc5c5ccccc45)c4cccc5c4c4ccccc4n5-c4ccccc4)cc32)cc1 ; c1ccc(-n2c3ccccc3c3ccc(N(c4ccc(-c5cccc6ccccc56)cc4)c4cccc5c4c4ccccc4n5-c4ccccc4)cc32)cc1 ; c1ccc(-c2ccc(N(c3ccc4c5ccccc5n(-c5ccccc5)c4c3)c3cccc4c3c3ccccc3n4-c3ccccc3)c3ccccc23)cc1]

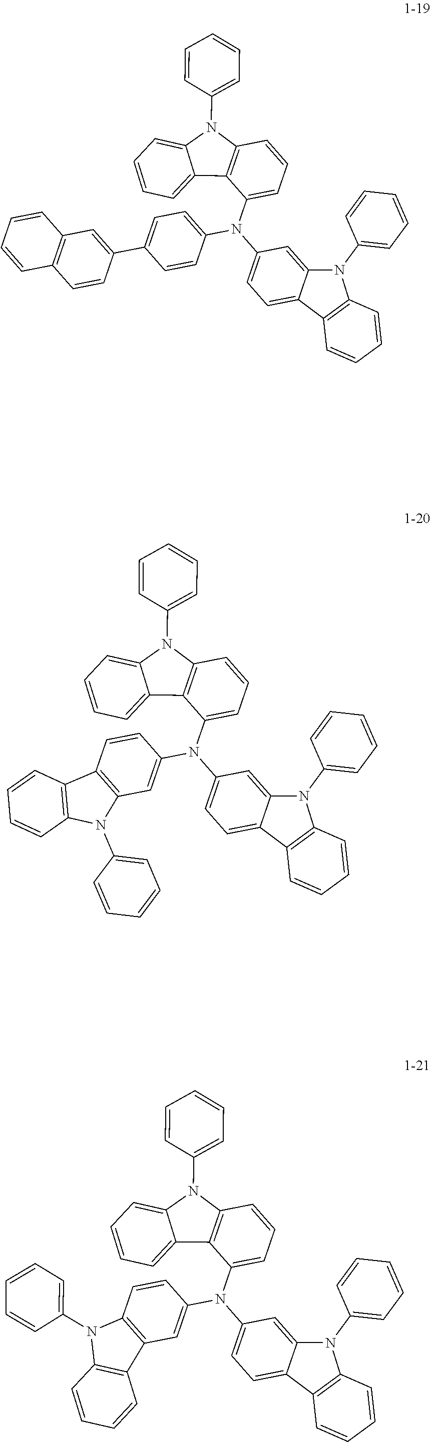 [SMILES: c1ccc(-n2c3ccccc3c3ccc(N(c4ccc(-c5ccc6ccccc6c5)cc4)c4cccc5c4c4ccccc4n5-c4ccccc4)cc32)cc1 ; c1ccc(-n2c3ccccc3c3ccc(N(c4ccc5c6ccccc6n(-c6ccccc6)c5c4)c4cccc5c4c4ccccc4n5-c4ccccc4)cc32)cc1 ; c1ccc(-n2c3ccccc3c3cc(N(c4ccc5c6ccccc6n(-c6ccccc6)c5c4)c4cccc5c4c4ccccc4n5-c4ccccc4)ccc32)cc1]

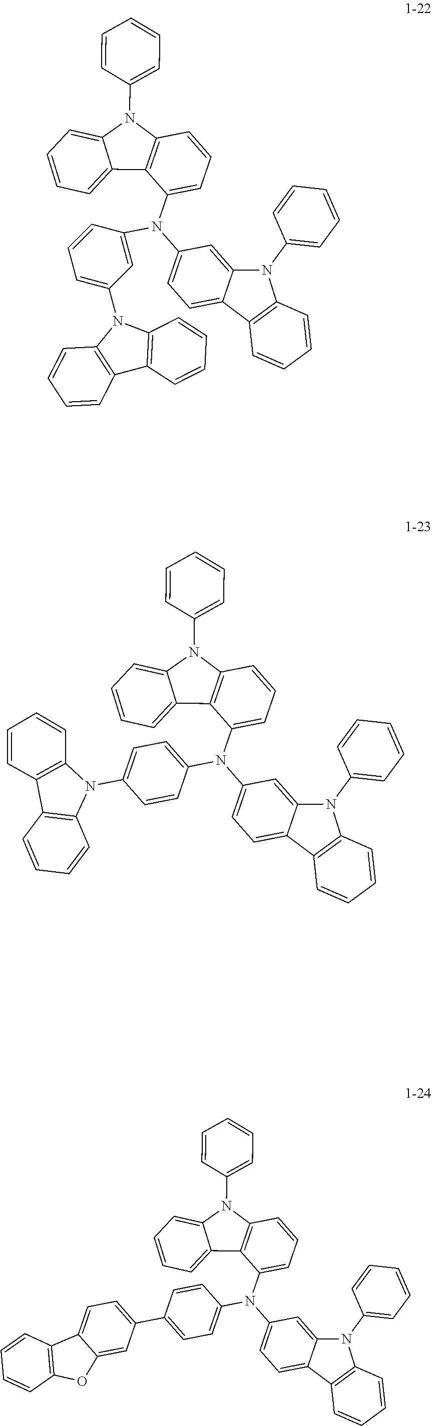 [SMILES: c1ccc(-n2c3ccccc3c3ccc(N(c4cccc(-n5c6ccccc6c6ccccc65)c4)c4cccc5c4c4ccccc4n5-c4ccccc4)cc32)cc1 ; c1ccc(-n2c3ccccc3c3ccc(N(c4ccc(-n5c6ccccc6c6ccccc65)cc4)c4cccc5c4c4ccccc4n5-c4ccccc4)cc32)cc1 ; c1ccc(-n2c3ccccc3c3ccc(N(c4ccc(-c5ccc6c(c5)oc5ccccc56)cc4)c4cccc5c4c4ccccc4n5-c4ccccc4)cc32)cc1]

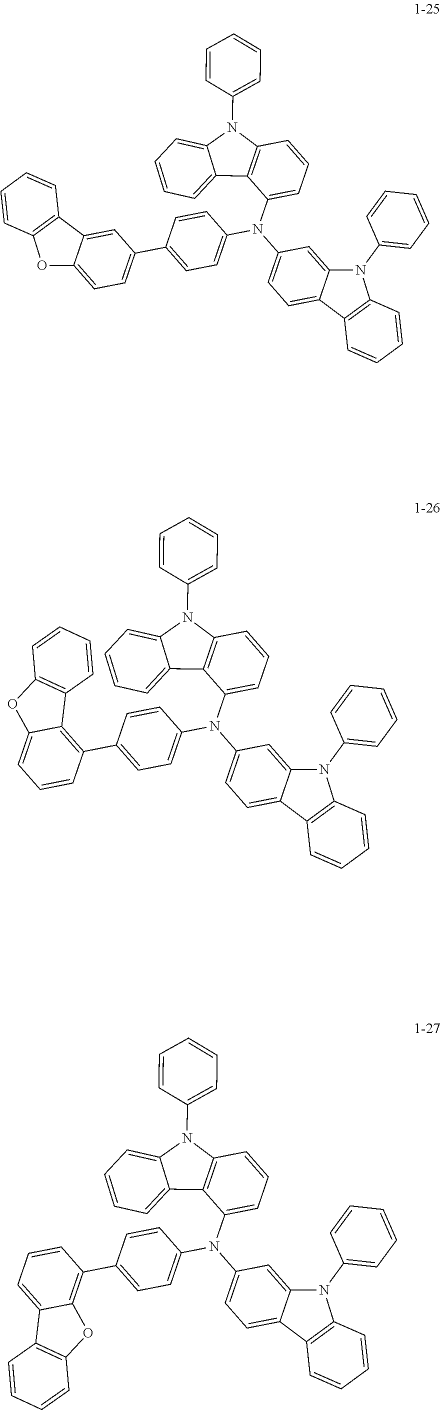 [SMILES: c1ccc(-n2c3ccccc3c3ccc(N(c4ccc(-c5ccc6oc7ccccc7c6c5)cc4)c4cccc5c4c4ccccc4n5-c4ccccc4)cc32)cc1 ; c1ccc(-n2c3ccccc3c3ccc(N(c4ccc(-c5cccc6oc7ccccc7c56)cc4)c4cccc5c4c4ccccc4n5-c4ccccc4)cc32)cc1 ; c1ccc(-n2c3ccccc3c3ccc(N(c4ccc(-c5cccc6c5oc5ccccc56)cc4)c4cccc5c4c4ccccc4n5-c4ccccc4)cc32)cc1]

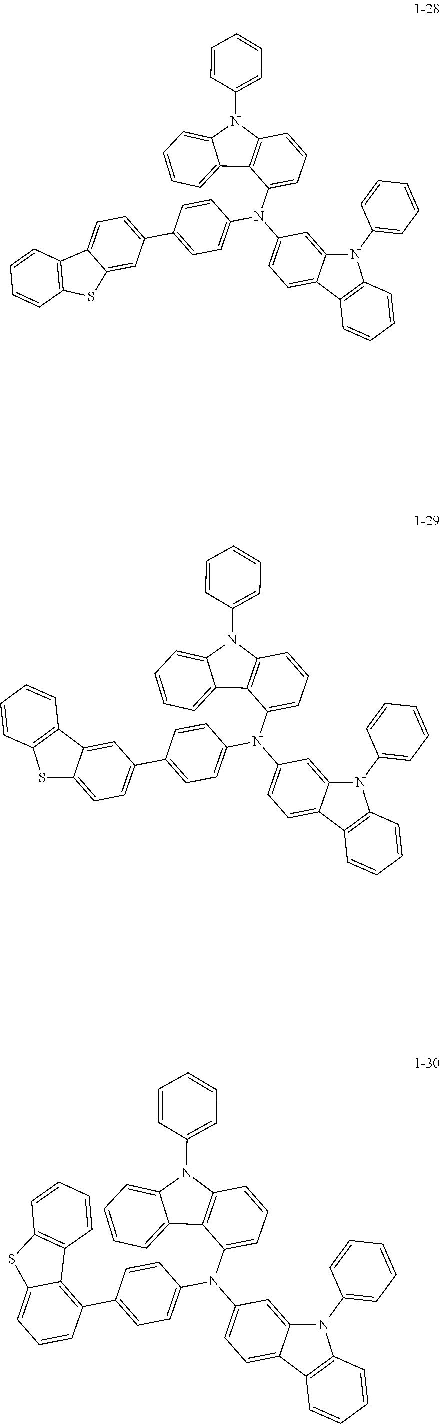 [SMILES: c1ccc(-n2c3ccccc3c3ccc(N(c4ccc(-c5ccc6c(c5)sc5ccccc56)cc4)c4cccc5c4c4ccccc4n5-c4ccccc4)cc32)cc1 ; c1ccc(-n2c3ccccc3c3ccc(N(c4ccc(-c5ccc6sc7ccccc7c6c5)cc4)c4cccc5c4c4ccccc4n5-c4ccccc4)cc32)cc1 ; c1ccc(-n2c3ccccc3c3ccc(N(c4ccc(-c5cccc6sc7ccccc7c56)cc4)c4cccc5c4c4ccccc4n5-c4ccccc4)cc32)cc1]

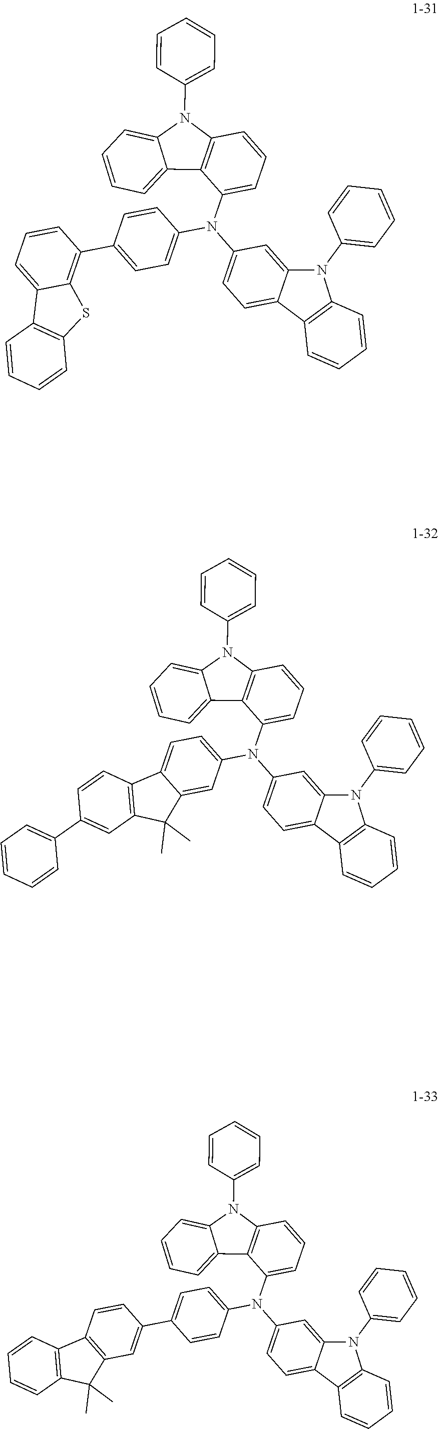 [SMILES: c1ccc(-n2c3ccccc3c3ccc(N(c4ccc(-c5cccc6c5sc5ccccc56)cc4)c4cccc5c4c4ccccc4n5-c4ccccc4)cc32)cc1 ; CC1(C)c2cc(-c3ccccc3)ccc2-c2ccc(N(c3ccc4c5ccccc5n(-c5ccccc5)c4c3)c3cccc4c3c3ccccc3n4-c3ccccc3)cc21 ; CC1(C)c2ccccc2-c2ccc(-c3ccc(N(c4ccc5c6ccccc6n(-c6ccccc6)c5c4)c4cccc5c4c4ccccc4n5-c4ccccc4)cc3)cc21]

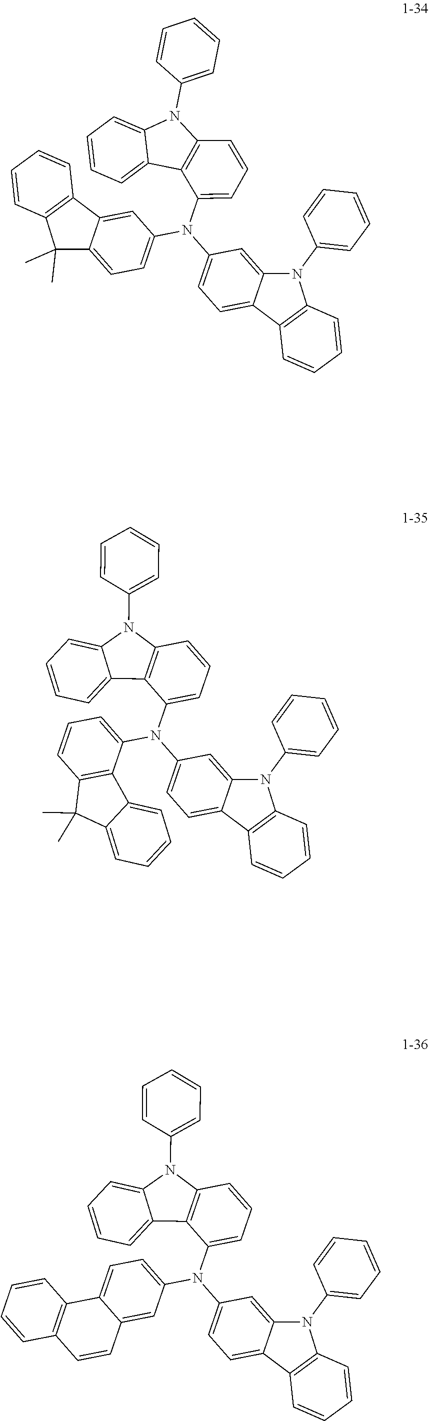 [SMILES: CC1(C)c2ccccc2-c2cc(N(c3ccc4c5ccccc5n(-c5ccccc5)c4c3)c3cccc4c3c3ccccc3n4-c3ccccc3)ccc21 ; CC1(C)c2ccccc2-c2c(N(c3ccc4c5ccccc5n(-c5ccccc5)c4c3)c3cccc4c3c3ccccc3n4-c3ccccc3)cccc21 ; c1ccc(-n2c3ccccc3c3ccc(N(c4ccc5c(ccc6ccccc65)c4)c4cccc5c4c4ccccc4n5-c4ccccc4)cc32)cc1]

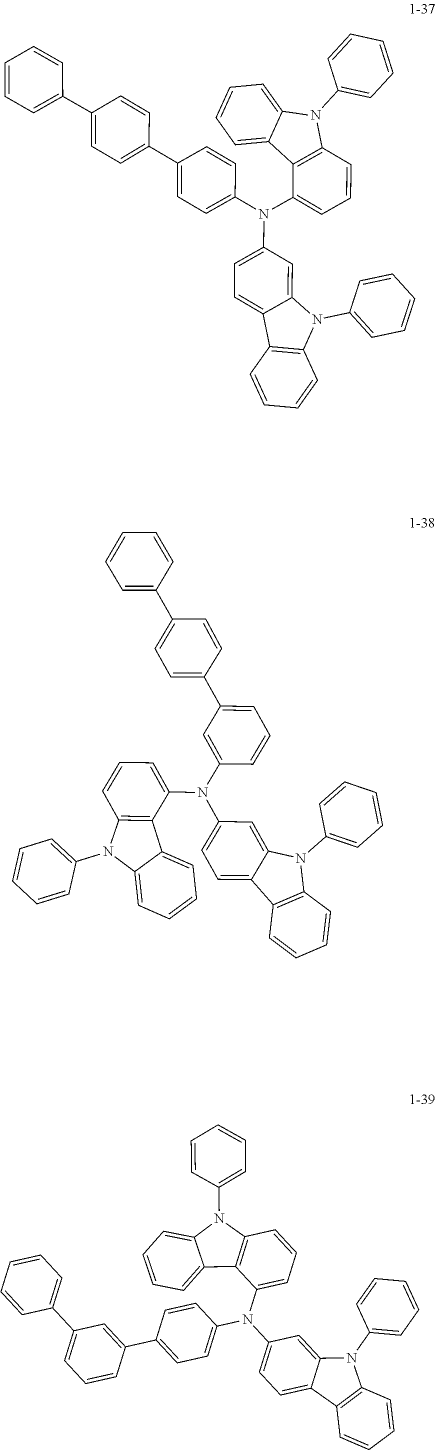 [SMILES: c1ccc(-c2ccc(-c3ccc(N(c4ccc5c6ccccc6n(-c6ccccc6)c5c4)c4cccc5c4c4ccccc4n5-c4ccccc4)cc3)cc2)cc1 ; c1ccc(-c2ccc(-c3cccc(N(c4ccc5c6ccccc6n(-c6ccccc6)c5c4)c4cccc5c4c4ccccc4n5-c4ccccc4)c3)cc2)cc1 ; c1ccc(-c2cccc(-c3ccc(N(c4ccc5c6ccccc6n(-c6ccccc6)c5c4)c4cccc5c4c4ccccc4n5-c4ccccc4)cc3)c2)cc1]

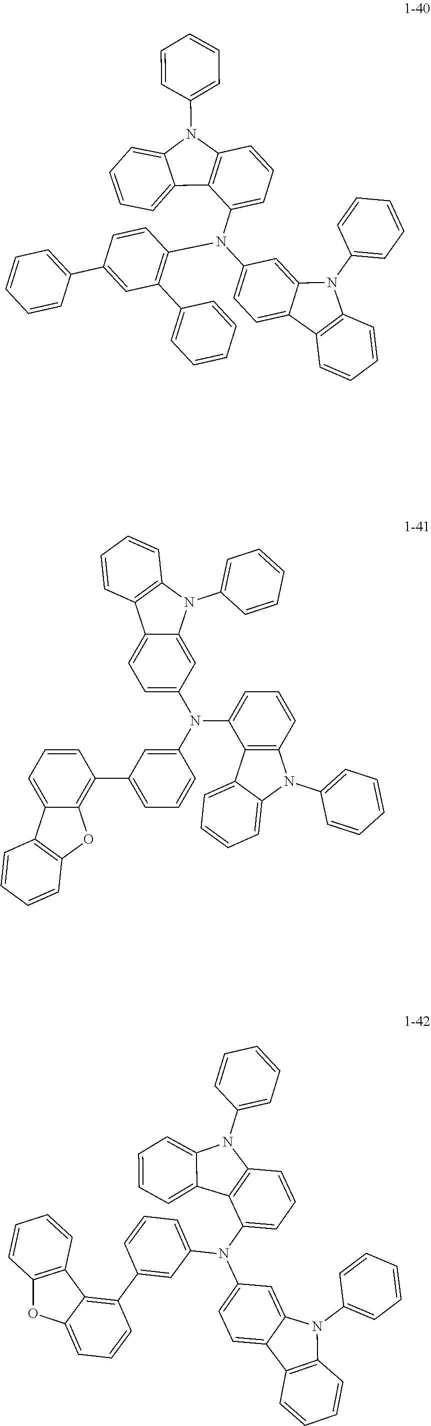 [SMILES: c1ccc(-c2ccc(N(c3ccc4c5ccccc5n(-c5ccccc5)c4c3)c3cccc4c3c3ccccc3n4-c3ccccc3)c(-c3ccccc3)c2)cc1 ; c1ccc(-n2c3ccccc3c3ccc(N(c4cccc(-c5cccc6c5oc5ccccc56)c4)c4cccc5c4c4ccccc4n5-c4ccccc4)cc32)cc1 ; c1ccc(-n2c3ccccc3c3ccc(N(c4cccc(-c5cccc6oc7ccccc7c56)c4)c4cccc5c4c4ccccc4n5-c4ccccc4)cc32)cc1]

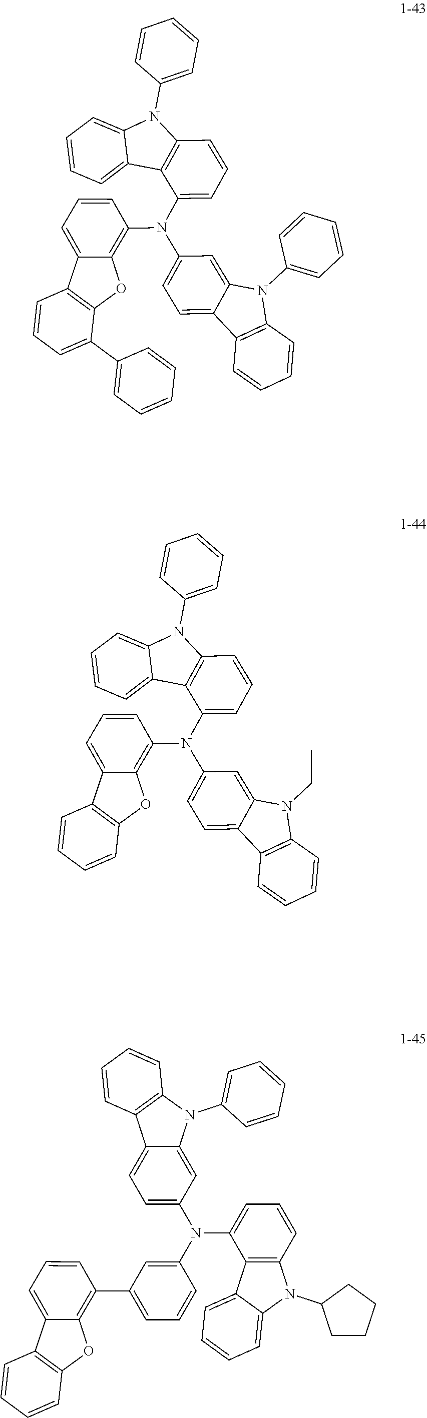 [SMILES: c1ccc(-c2cccc3c2oc2c(N(c4ccc5c6ccccc6n(-c6ccccc6)c5c4)c4cccc5c4c4ccccc4n5-c4ccccc4)cccc23)cc1 ; CCn1c2ccccc2c2ccc(N(c3cccc4c3oc3ccccc34)c3cccc4c3c3ccccc3n4-c3ccccc3)cc21 ; c1ccc(-n2c3ccccc3c3ccc(N(c4cccc(-c5cccc6c5oc5ccccc56)c4)c4cccc5c4c4ccccc4n5C4CCCC4)cc32)cc1]

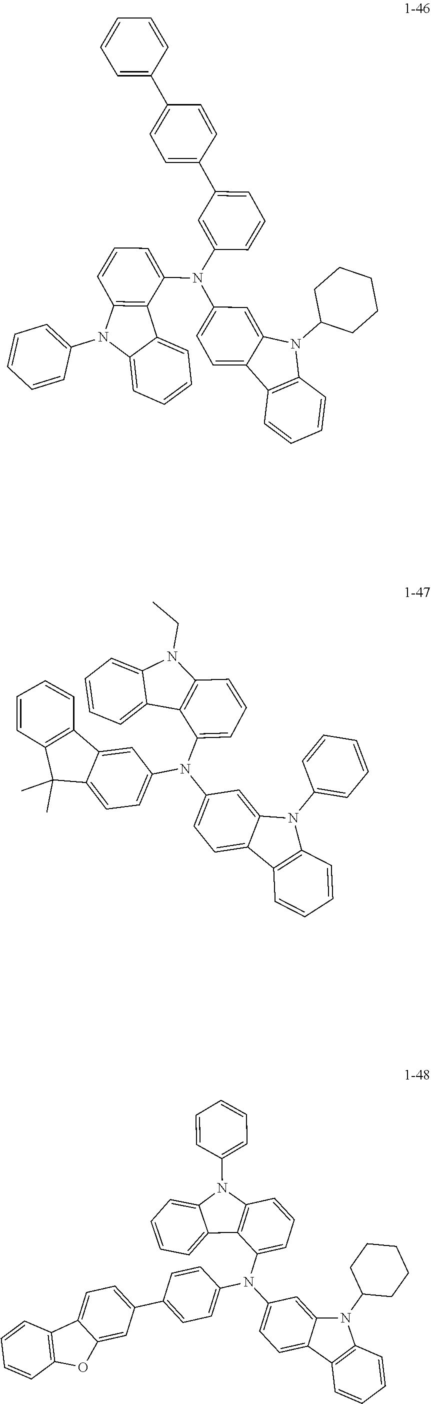 [SMILES: c1ccc(-c2ccc(-c3cccc(N(c4ccc5c6ccccc6n(C6CCCCC6)c5c4)c4cccc5c4c4ccccc4n5-c4ccccc4)c3)cc2)cc1 ; CCn1c2ccccc2c2c(N(c3ccc4c(c3)-c3ccccc3C4(C)C)c3ccc4c5ccccc5n(-c5ccccc5)c4c3)cccc21 ; c1ccc(-n2c3ccccc3c3c(N(c4ccc(-c5ccc6c(c5)oc5ccccc56)cc4)c4ccc5c6ccccc6n(C6CCCCC6)c5c4)cccc32)cc1]

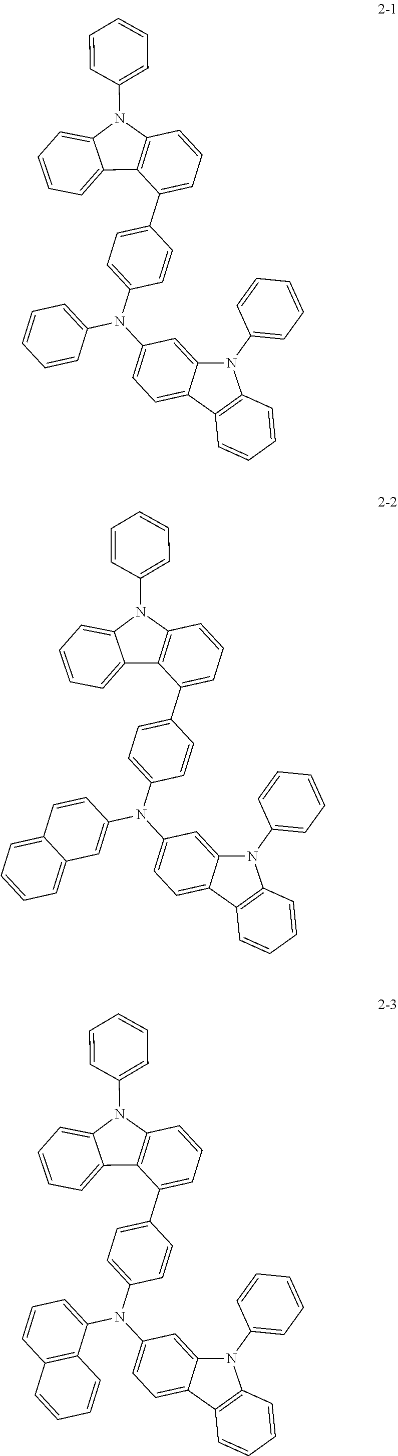 [SMILES: c1ccc(N(c2ccc(-c3cccc4c3c3ccccc3n4-c3ccccc3)cc2)c2ccc3c4ccccc4n(-c4ccccc4)c3c2)cc1 ; c1ccc(-n2c3ccccc3c3ccc(N(c4ccc(-c5cccc6c5c5ccccc5n6-c5ccccc5)cc4)c4ccc5ccccc5c4)cc32)cc1 ; c1ccc(-n2c3ccccc3c3ccc(N(c4ccc(-c5cccc6c5c5ccccc5n6-c5ccccc5)cc4)c4cccc5ccccc45)cc32)cc1]

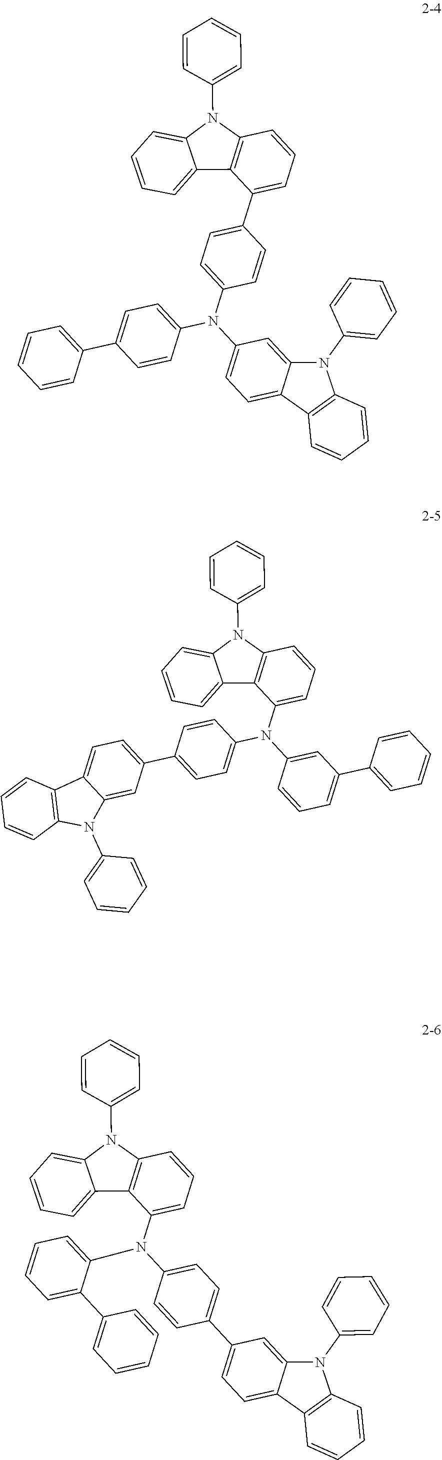 [SMILES: c1ccc(-c2ccc(N(c3ccc(-c4cccc5c4c4ccccc4n5-c4ccccc4)cc3)c3ccc4c5ccccc5n(-c5ccccc5)c4c3)cc2)cc1 ; c1ccc(-c2cccc(N(c3ccc(-c4ccc5c6ccccc6n(-c6ccccc6)c5c4)cc3)c3cccc4c3c3ccccc3n4-c3ccccc3)c2)cc1 ; c1ccc(-c2ccccc2N(c2ccc(-c3ccc4c5ccccc5n(-c5ccccc5)c4c3)cc2)c2cccc3c2c2ccccc2n3-c2ccccc2)cc1]

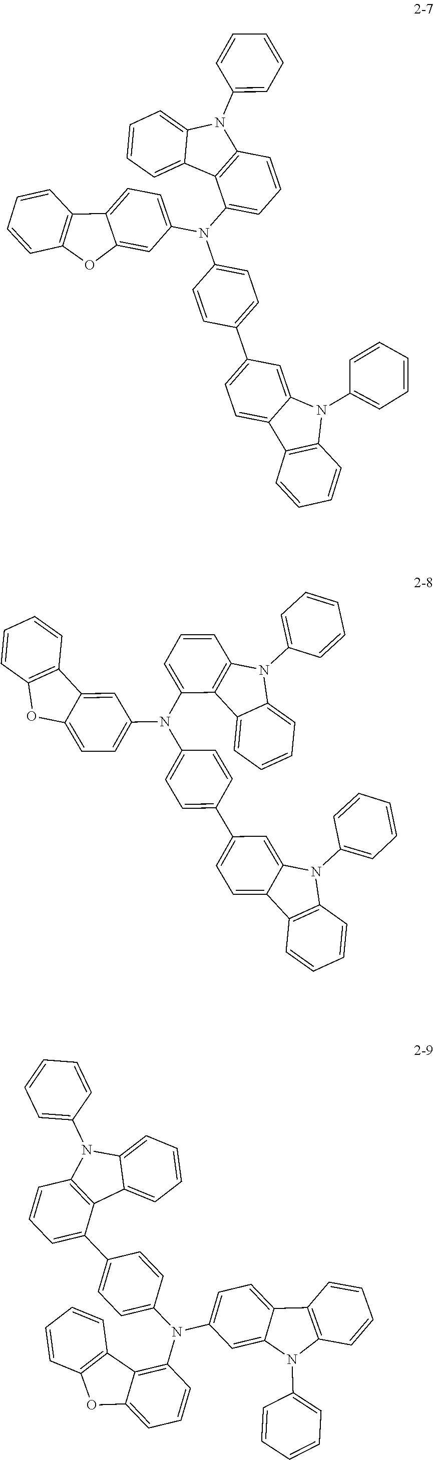 [SMILES: c1ccc(-n2c3ccccc3c3ccc(-c4ccc(N(c5ccc6c(c5)oc5ccccc56)c5cccc6c5c5ccccc5n6-c5ccccc5)cc4)cc32)cc1 ; c1ccc(-n2c3ccccc3c3ccc(-c4ccc(N(c5ccc6oc7ccccc7c6c5)c5cccc6c5c5ccccc5n6-c5ccccc5)cc4)cc32)cc1 ; c1ccc(-n2c3ccccc3c3ccc(N(c4ccc(-c5cccc6c5c5ccccc5n6-c5ccccc5)cc4)c4cccc5oc6ccccc6c45)cc32)cc1]

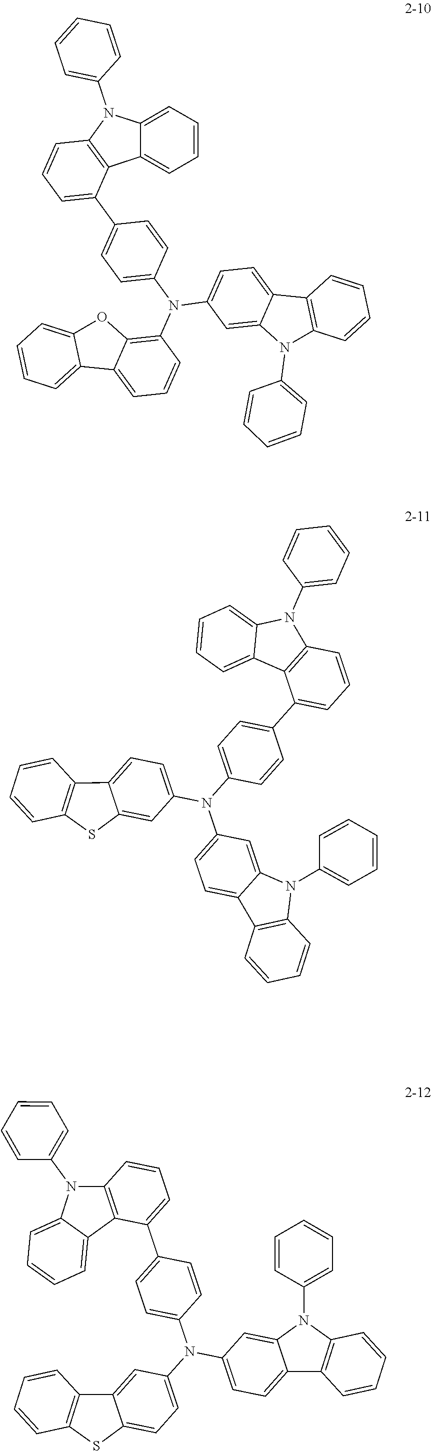 [SMILES: c1ccc(-n2c3ccccc3c3ccc(N(c4ccc(-c5cccc6c5c5ccccc5n6-c5ccccc5)cc4)c4cccc5c4oc4ccccc45)cc32)cc1 ; c1ccc(-n2c3ccccc3c3ccc(N(c4ccc(-c5cccc6c5c5ccccc5n6-c5ccccc5)cc4)c4ccc5c(c4)sc4ccccc45)cc32)cc1 ; c1ccc(-n2c3ccccc3c3ccc(N(c4ccc(-c5cccc6c5c5ccccc5n6-c5ccccc5)cc4)c4ccc5sc6ccccc6c5c4)cc32)cc1]

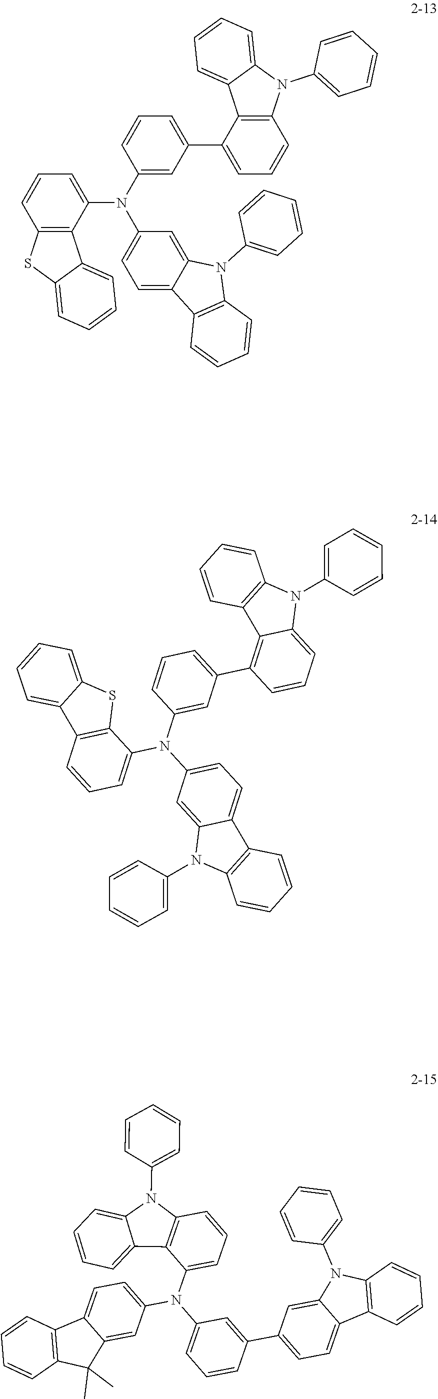 [SMILES: c1ccc(-n2c3ccccc3c3ccc(N(c4cccc(-c5cccc6c5c5ccccc5n6-c5ccccc5)c4)c4cccc5sc6ccccc6c45)cc32)cc1 ; c1ccc(-n2c3ccccc3c3ccc(N(c4cccc(-c5cccc6c5c5ccccc5n6-c5ccccc5)c4)c4cccc5c4sc4ccccc45)cc32)cc1 ; CC1(C)c2ccccc2-c2ccc(N(c3cccc(-c4ccc5c6ccccc6n(-c6ccccc6)c5c4)c3)c3cccc4c3c3ccccc3n4-c3ccccc3)cc21]

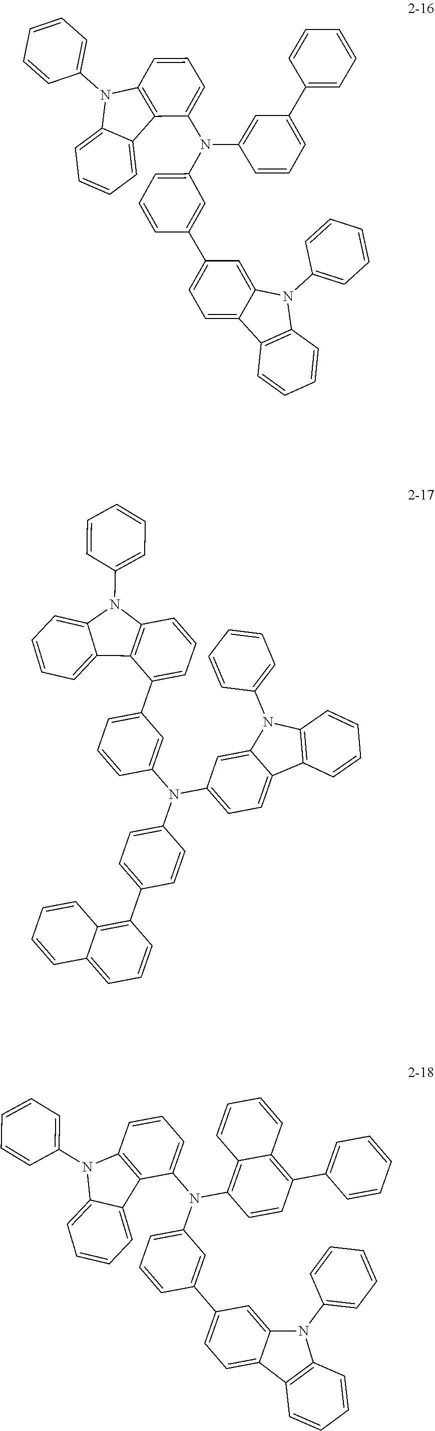 [SMILES: c1ccc(-c2cccc(N(c3cccc(-c4ccc5c6ccccc6n(-c6ccccc6)c5c4)c3)c3cccc4c3c3ccccc3n4-c3ccccc3)c2)cc1 ; c1ccc(-n2c3ccccc3c3ccc(N(c4ccc(-c5cccc6ccccc56)cc4)c4cccc(-c5cccc6c5c5ccccc5n6-c5ccccc5)c4)cc32)cc1 ; c1ccc(-c2ccc(N(c3cccc(-c4ccc5c6ccccc6n(-c6ccccc6)c5c4)c3)c3cccc4c3c3ccccc3n4-c3ccccc3)c3ccccc23)cc1]

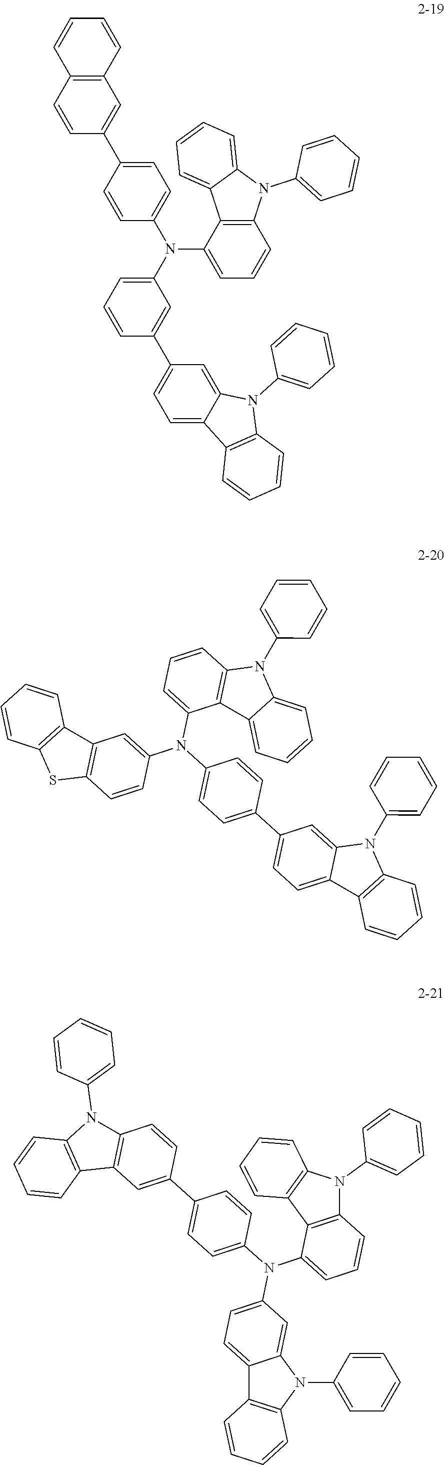 [SMILES: c1ccc(-n2c3ccccc3c3ccc(-c4cccc(N(c5ccc(-c6ccc7ccccc7c6)cc5)c5cccc6c5c5ccccc5n6-c5ccccc5)c4)cc32)cc1 ; c1ccc(-n2c3ccccc3c3ccc(-c4ccc(N(c5ccc6sc7ccccc7c6c5)c5cccc6c5c5ccccc5n6-c5ccccc5)cc4)cc32)cc1 ; c1ccc(-n2c3ccccc3c3cc(-c4ccc(N(c5ccc6c7ccccc7n(-c7ccccc7)c6c5)c5cccc6c5c5ccccc5n6-c5ccccc5)cc4)ccc32)cc1]

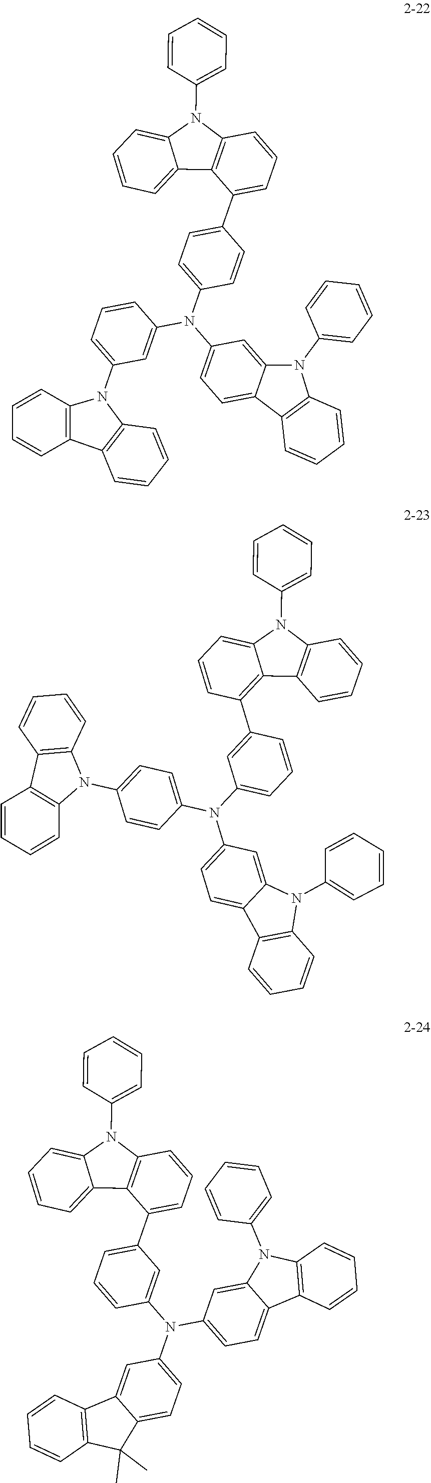 [SMILES: c1ccc(-n2c3ccccc3c3ccc(N(c4ccc(-c5cccc6c5c5ccccc5n6-c5ccccc5)cc4)c4cccc(-n5c6ccccc6c6ccccc65)c4)cc32)cc1 ; c1ccc(-n2c3ccccc3c3ccc(N(c4ccc(-n5c6ccccc6c6ccccc65)cc4)c4cccc(-c5cccc6c5c5ccccc5n6-c5ccccc5)c4)cc32)cc1 ; CC1(C)c2ccccc2-c2cc(N(c3cccc(-c4cccc5c4c4ccccc4n5-c4ccccc4)c3)c3ccc4c5ccccc5n(-c5ccccc5)c4c3)ccc21]

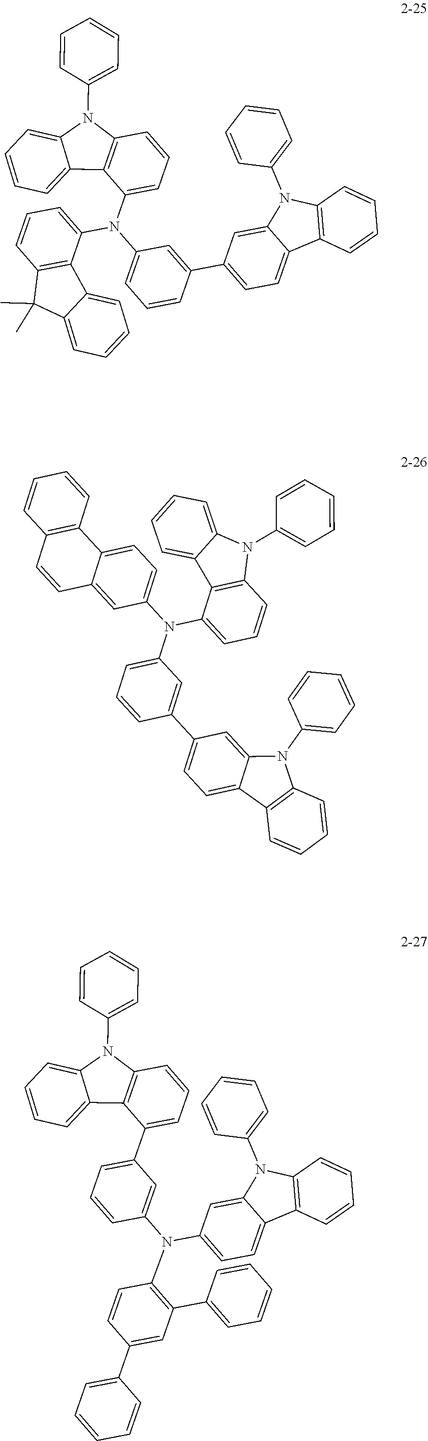 [SMILES: CC1(C)c2ccccc2-c2c(N(c3cccc(-c4ccc5c6ccccc6n(-c6ccccc6)c5c4)c3)c3cccc4c3c3ccccc3n4-c3ccccc3)cccc21 ; c1ccc(-n2c3ccccc3c3ccc(-c4cccc(N(c5ccc6c(ccc7ccccc76)c5)c5cccc6c5c5ccccc5n6-c5ccccc5)c4)cc32)cc1 ; c1ccc(-c2ccc(N(c3cccc(-c4cccc5c4c4ccccc4n5-c4ccccc4)c3)c3ccc4c5ccccc5n(-c5ccccc5)c4c3)c(-c3ccccc3)c2)cc1]

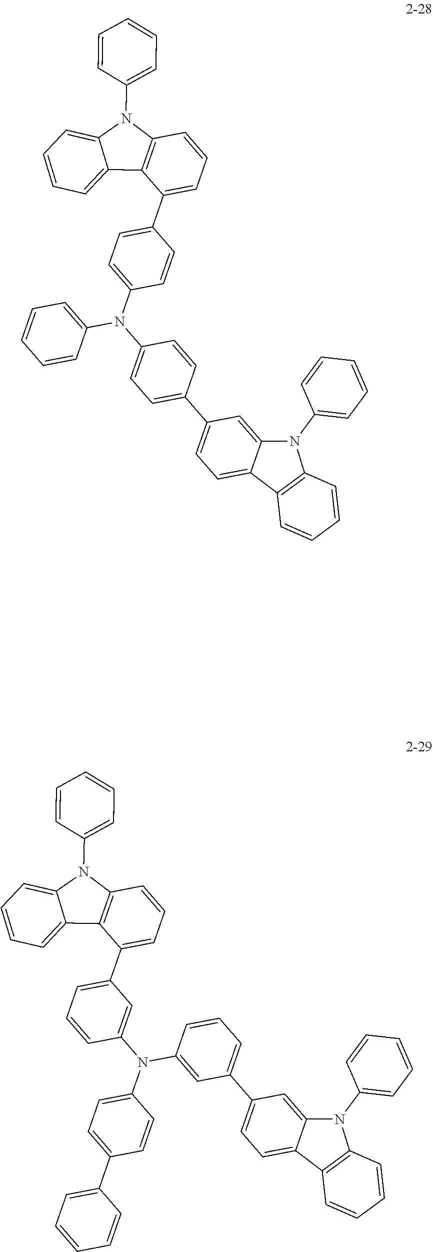 [SMILES: c1ccc(N(c2ccc(-c3ccc4c5ccccc5n(-c5ccccc5)c4c3)cc2)c2ccc(-c3cccc4c3c3ccccc3n4-c3ccccc3)cc2)cc1 ; c1ccc(-c2ccc(N(c3cccc(-c4ccc5c6ccccc6n(-c6ccccc6)c5c4)c3)c3cccc(-c4cccc5c4c4ccccc4n5-c4ccccc4)c3)cc2)cc1]

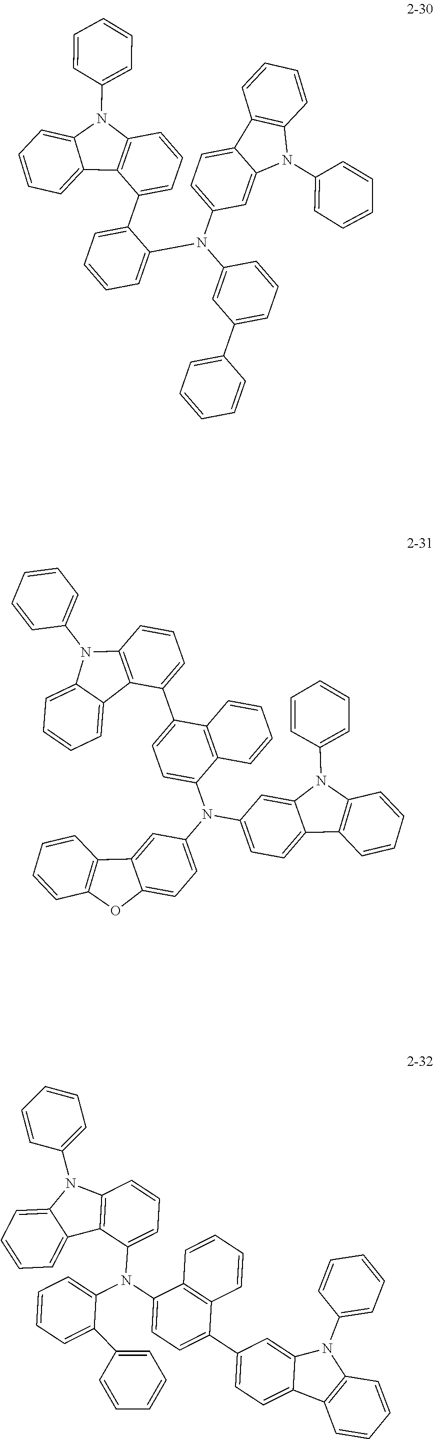 [SMILES: c1ccc(-c2cccc(N(c3ccc4c5ccccc5n(-c5ccccc5)c4c3)c3ccccc3-c3cccc4c3c3ccccc3n4-c3ccccc3)c2)cc1 ; c1ccc(-n2c3ccccc3c3ccc(N(c4ccc5oc6ccccc6c5c4)c4ccc(-c5cccc6c5c5ccccc5n6-c5ccccc5)c5ccccc45)cc32)cc1 ; c1ccc(-c2ccccc2N(c2ccc(-c3ccc4c5ccccc5n(-c5ccccc5)c4c3)c3ccccc23)c2cccc3c2c2ccccc2n3-c2ccccc2)cc1]

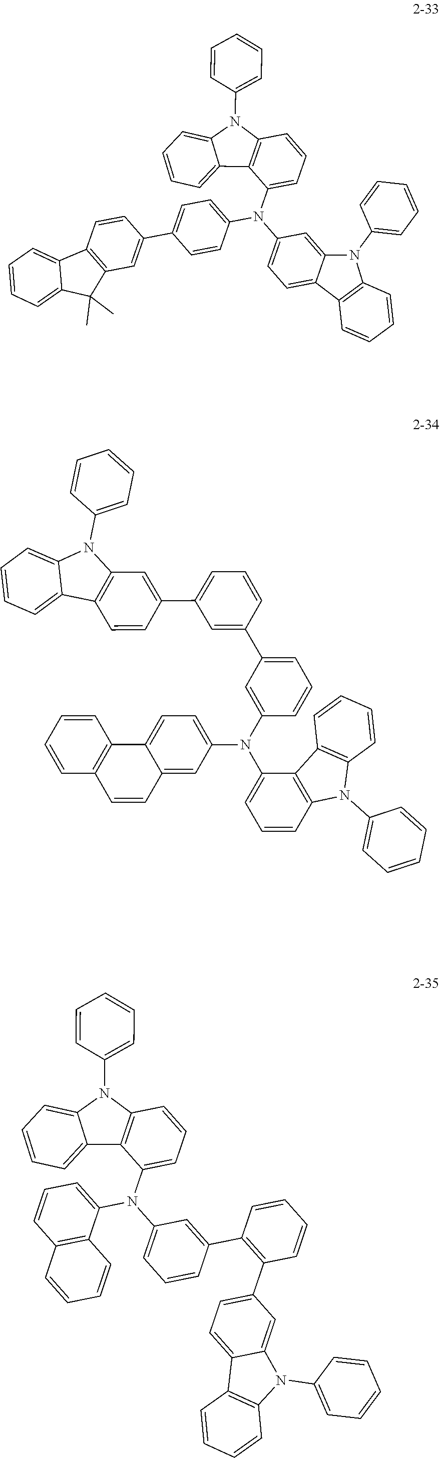 [SMILES: CC1(C)c2ccccc2-c2ccc(-c3ccc(N(c4ccc5c6ccccc6n(-c6ccccc6)c5c4)c4cccc5c4c4ccccc4n5-c4ccccc4)cc3)cc21 ; c1ccc(-n2c3ccccc3c3ccc(-c4cccc(-c5cccc(N(c6ccc7c(ccc8ccccc87)c6)c6cccc7c6c6ccccc6n7-c6ccccc6)c5)c4)cc32)cc1 ; c1ccc(-n2c3ccccc3c3ccc(-c4ccccc4-c4cccc(N(c5cccc6ccccc56)c5cccc6c5c5ccccc5n6-c5ccccc5)c4)cc32)cc1]

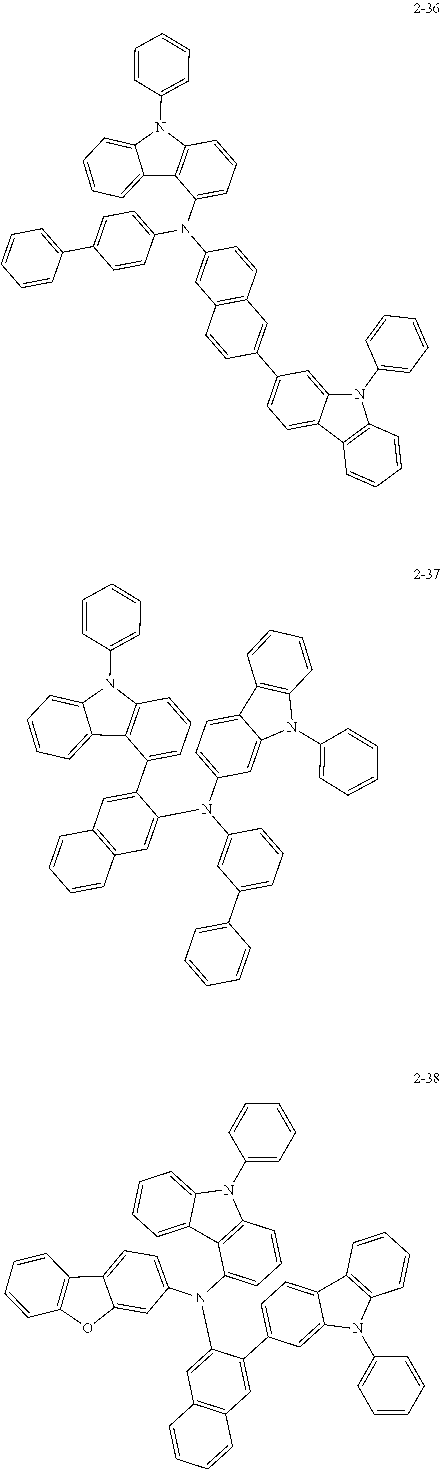 [SMILES: c1ccc(-c2ccc(N(c3ccc4cc(-c5ccc6c7ccccc7n(-c7ccccc7)c6c5)ccc4c3)c3cccc4c3c3ccccc3n4-c3ccccc3)cc2)cc1 ; c1ccc(-c2cccc(N(c3ccc4c5ccccc5n(-c5ccccc5)c4c3)c3cc4ccccc4cc3-c3cccc4c3c3ccccc3n4-c3ccccc3)c2)cc1 ; c1ccc(-n2c3ccccc3c3ccc(-c4cc5ccccc5cc4N(c4ccc5c(c4)oc4ccccc45)c4cccc5c4c4ccccc4n5-c4ccccc4)cc32)cc1]

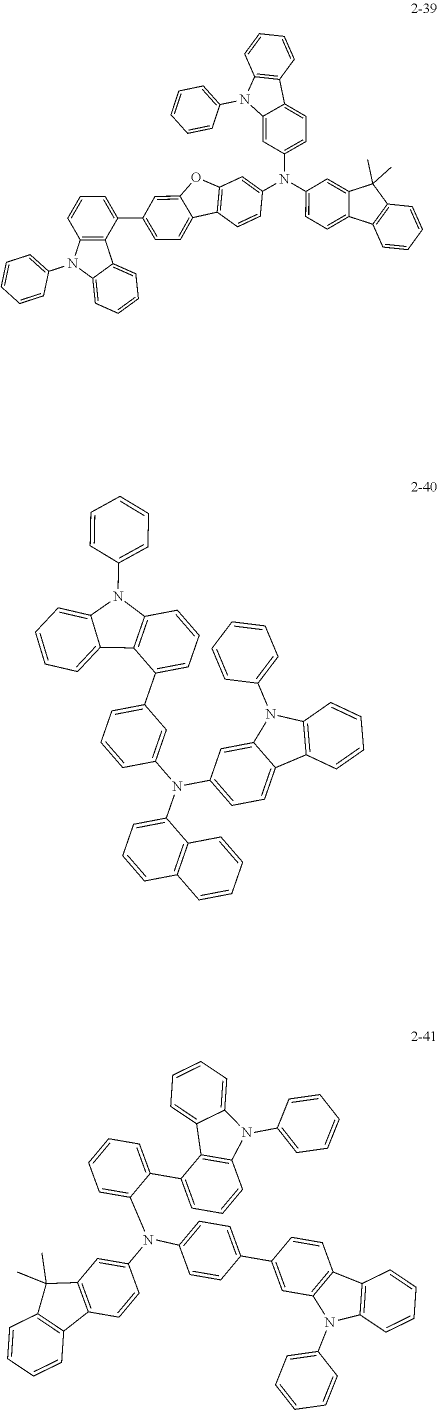 [SMILES: CC1(C)c2ccccc2-c2ccc(N(c3ccc4c(c3)oc3cc(-c5cccc6c5c5ccccc5n6-c5ccccc5)ccc34)c3ccc4c5ccccc5n(-c5ccccc5)c4c3)cc21 ; c1ccc(-n2c3ccccc3c3ccc(N(c4cccc(-c5cccc6c5c5ccccc5n6-c5ccccc5)c4)c4cccc5ccccc45)cc32)cc1 ; CC1(C)c2ccccc2-c2ccc(N(c3ccc(-c4ccc5c6ccccc6n(-c6ccccc6)c5c4)cc3)c3ccccc3-c3cccc4c3c3ccccc3n4-c3ccccc3)cc21]

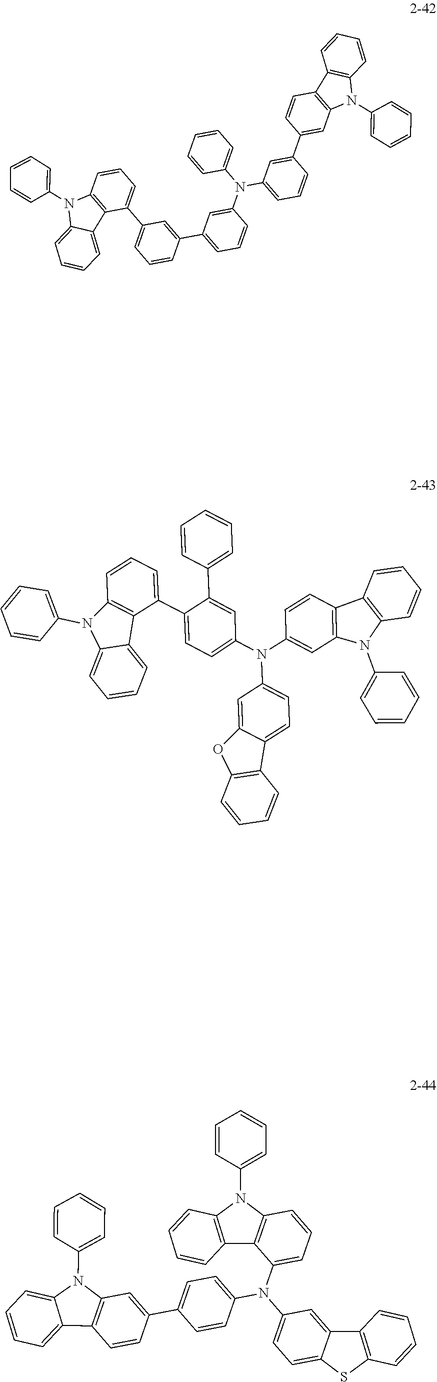 [SMILES: c1ccc(N(c2cccc(-c3cccc(-c4cccc5c4c4ccccc4n5-c4ccccc4)c3)c2)c2cccc(-c3ccc4c5ccccc5n(-c5ccccc5)c4c3)c2)cc1 ; c1ccc(-c2cc(N(c3ccc4c(c3)oc3ccccc34)c3ccc4c5ccccc5n(-c5ccccc5)c4c3)ccc2-c2cccc3c2c2ccccc2n3-c2ccccc2)cc1 ; c1ccc(-n2c3ccccc3c3ccc(-c4ccc(N(c5ccc6sc7ccccc7c6c5)c5cccc6c5c5ccccc5n6-c5ccccc5)cc4)cc32)cc1]

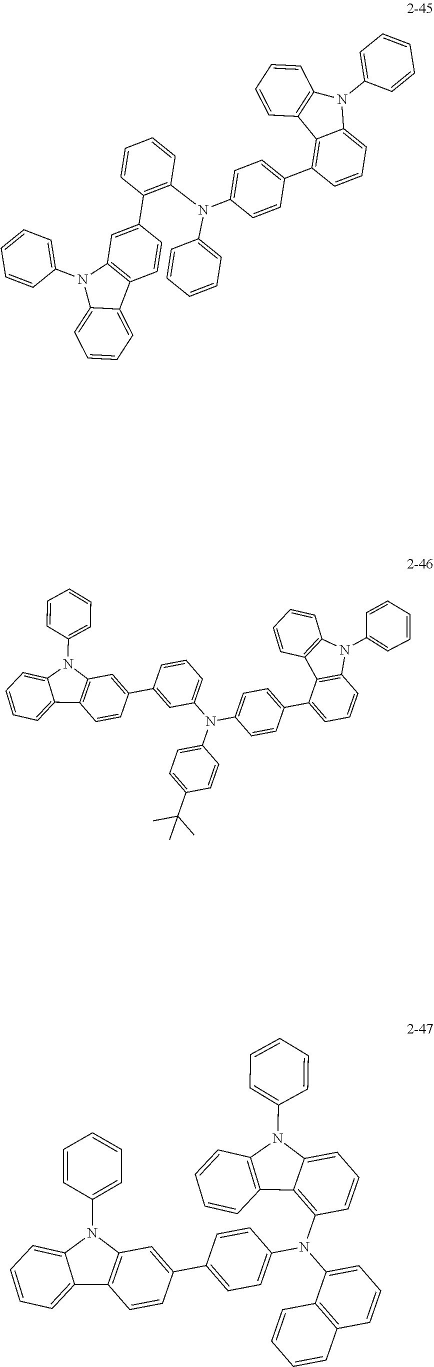 [SMILES: c1ccc(N(c2ccc(-c3cccc4c3c3ccccc3n4-c3ccccc3)cc2)c2ccccc2-c2ccc3c4ccccc4n(-c4ccccc4)c3c2)cc1 ; CC(C)(C)c1ccc(N(c2ccc(-c3cccc4c3c3ccccc3n4-c3ccccc3)cc2)c2cccc(-c3ccc4c5ccccc5n(-c5ccccc5)c4c3)c2)cc1 ; c1ccc(-n2c3ccccc3c3ccc(-c4ccc(N(c5cccc6ccccc56)c5cccc6c5c5ccccc5n6-c5ccccc5)cc4)cc32)cc1]

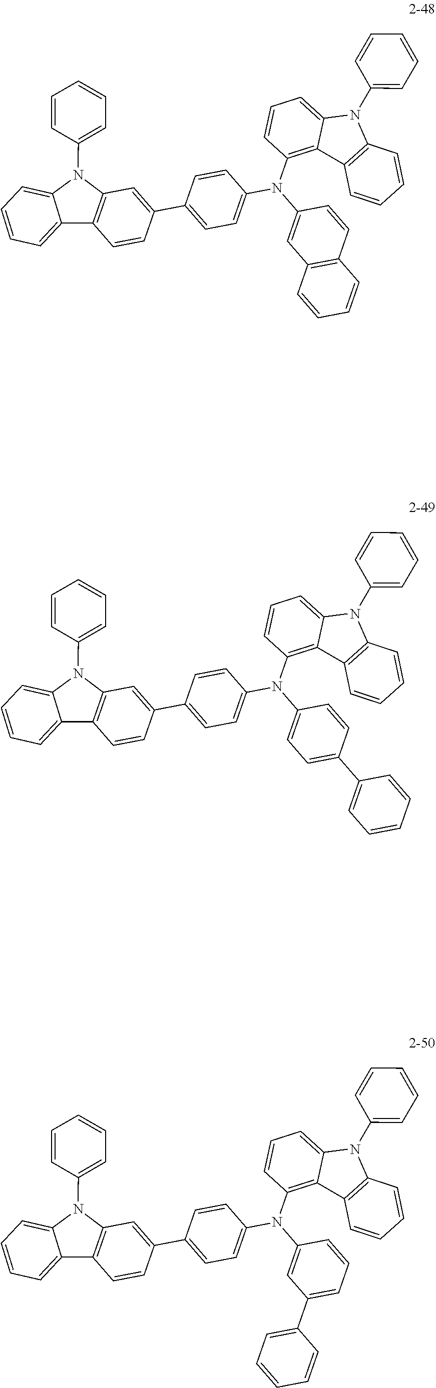 [SMILES: c1ccc(-n2c3ccccc3c3ccc(-c4ccc(N(c5ccc6ccccc6c5)c5cccc6c5c5ccccc5n6-c5ccccc5)cc4)cc32)cc1 ; c1ccc(-c2ccc(N(c3ccc(-c4ccc5c6ccccc6n(-c6ccccc6)c5c4)cc3)c3cccc4c3c3ccccc3n4-c3ccccc3)cc2)cc1 ; c1ccc(-c2cccc(N(c3ccc(-c4ccc5c6ccccc6n(-c6ccccc6)c5c4)cc3)c3cccc4c3c3ccccc3n4-c3ccccc3)c2)cc1]

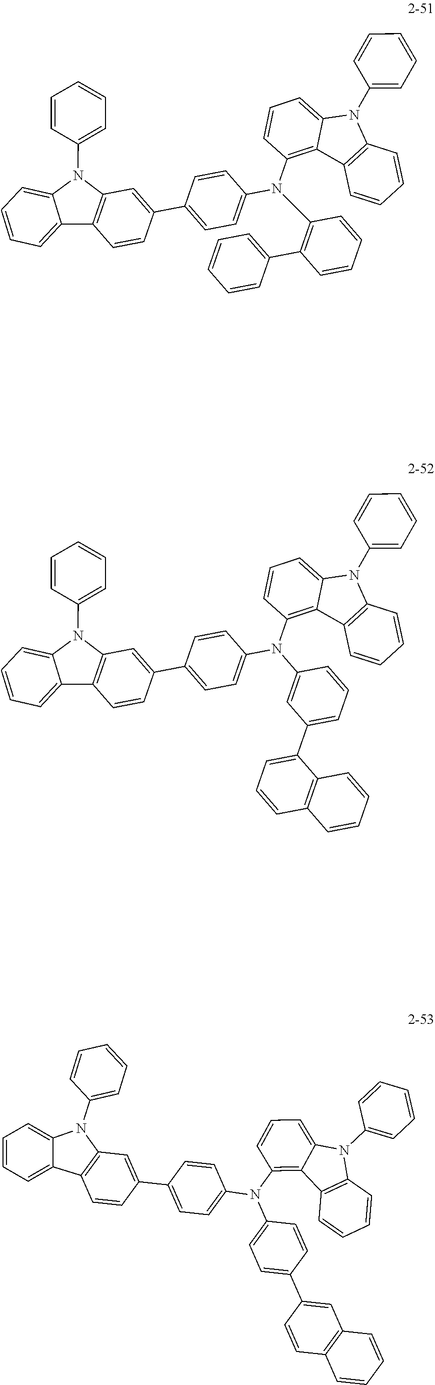 [SMILES: c1ccc(-c2ccccc2N(c2ccc(-c3ccc4c5ccccc5n(-c5ccccc5)c4c3)cc2)c2cccc3c2c2ccccc2n3-c2ccccc2)cc1 ; c1ccc(-n2c3ccccc3c3ccc(-c4ccc(N(c5cccc(-c6cccc7ccccc67)c5)c5cccc6c5c5ccccc5n6-c5ccccc5)cc4)cc32)cc1 ; c1ccc(-n2c3ccccc3c3ccc(-c4ccc(N(c5ccc(-c6ccc7ccccc7c6)cc5)c5cccc6c5c5ccccc5n6-c5ccccc5)cc4)cc32)cc1]

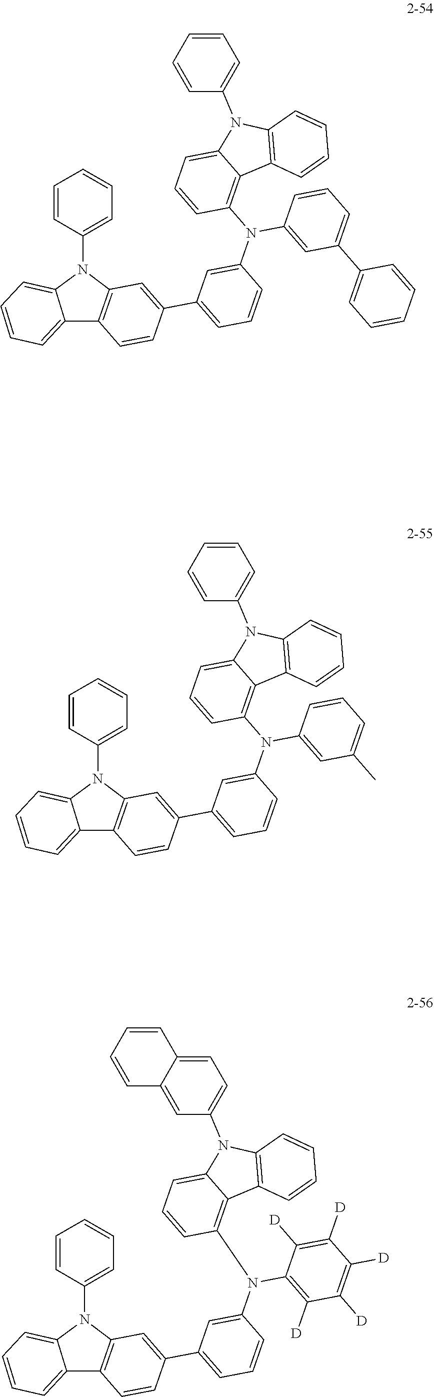 [SMILES: c1ccc(-c2cccc(N(c3cccc(-c4ccc5c6ccccc6n(-c6ccccc6)c5c4)c3)c3cccc4c3c3ccccc3n4-c3ccccc3)c2)cc1 ; Cc1cccc(N(c2cccc(-c3ccc4c5ccccc5n(-c5ccccc5)c4c3)c2)c2cccc3c2c2ccccc2n3-c2ccccc2)c1 ; [2H]c1c([2H])c([2H])c(N(c2cccc(-c3ccc4c5ccccc5n(-c5ccccc5)c4c3)c2)c2cccc3c2c2ccccc2n3-c2ccc3ccccc3c2)c([2H])c1[2H]]

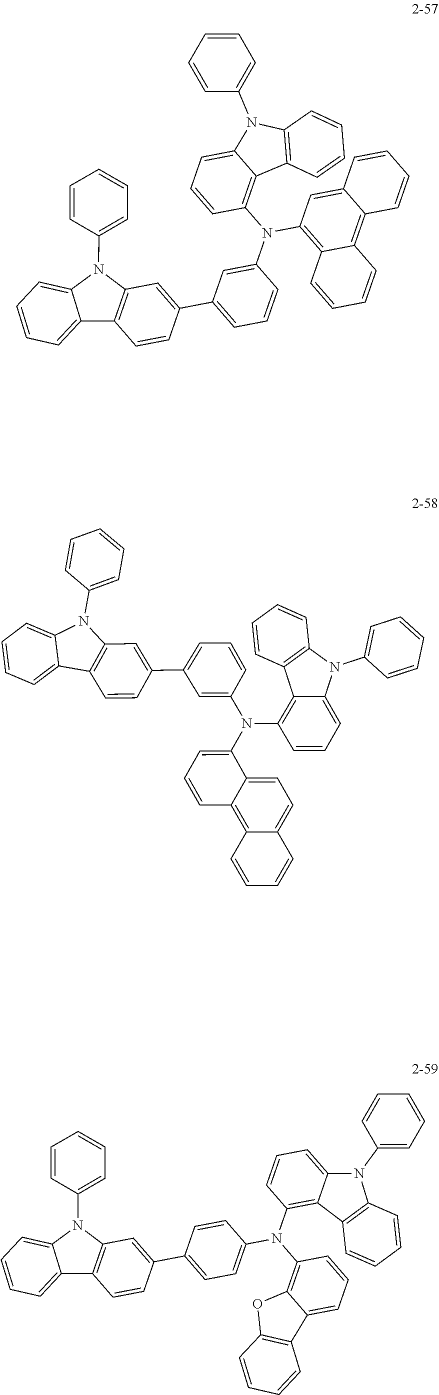 [SMILES: c1ccc(-n2c3ccccc3c3ccc(-c4cccc(N(c5cc6ccccc6c6ccccc56)c5cccc6c5c5ccccc5n6-c5ccccc5)c4)cc32)cc1 ; c1ccc(-n2c3ccccc3c3ccc(-c4cccc(N(c5cccc6c5ccc5ccccc56)c5cccc6c5c5ccccc5n6-c5ccccc5)c4)cc32)cc1 ; c1ccc(-n2c3ccccc3c3ccc(-c4ccc(N(c5cccc6c5oc5ccccc56)c5cccc6c5c5ccccc5n6-c5ccccc5)cc4)cc32)cc1]

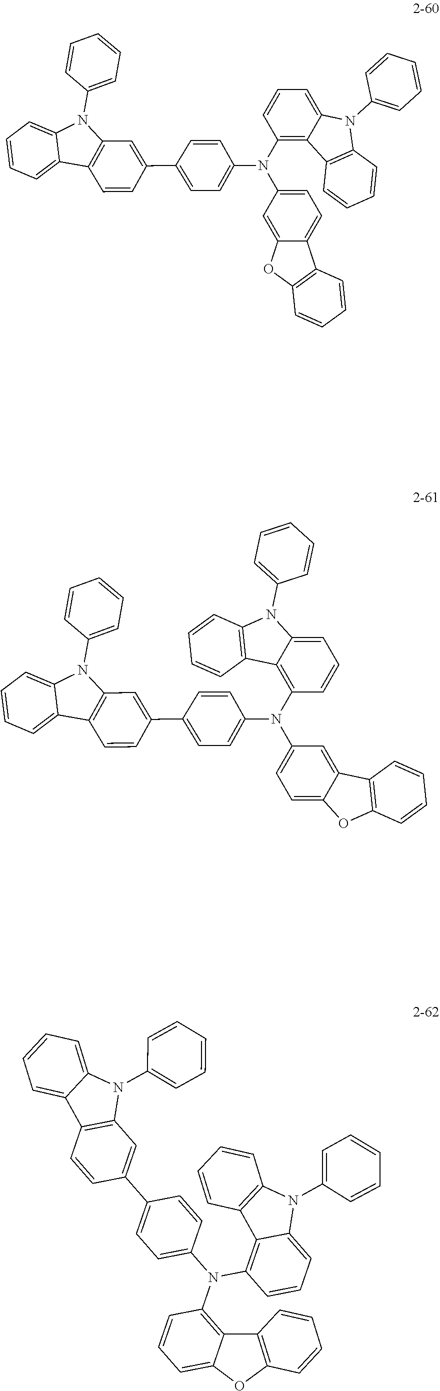 [SMILES: c1ccc(-n2c3ccccc3c3ccc(-c4ccc(N(c5ccc6c(c5)oc5ccccc56)c5cccc6c5c5ccccc5n6-c5ccccc5)cc4)cc32)cc1 ; c1ccc(-n2c3ccccc3c3ccc(-c4ccc(N(c5ccc6oc7ccccc7c6c5)c5cccc6c5c5ccccc5n6-c5ccccc5)cc4)cc32)cc1 ; c1ccc(-n2c3ccccc3c3ccc(-c4ccc(N(c5cccc6oc7ccccc7c56)c5cccc6c5c5ccccc5n6-c5ccccc5)cc4)cc32)cc1]

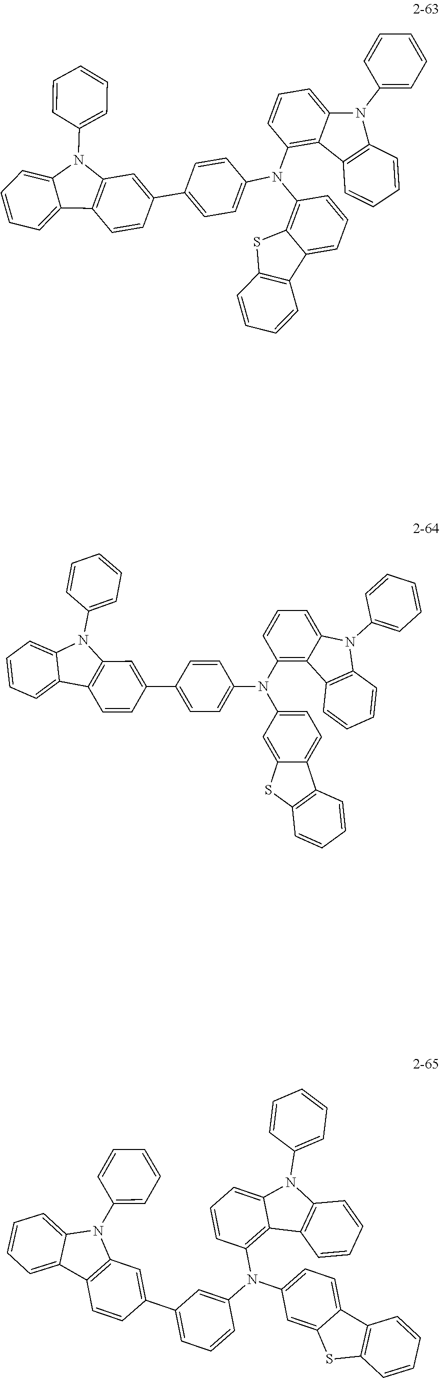 [SMILES: c1ccc(-n2c3ccccc3c3ccc(-c4ccc(N(c5cccc6c5sc5ccccc56)c5cccc6c5c5ccccc5n6-c5ccccc5)cc4)cc32)cc1 ; c1ccc(-n2c3ccccc3c3ccc(-c4ccc(N(c5ccc6c(c5)sc5ccccc56)c5cccc6c5c5ccccc5n6-c5ccccc5)cc4)cc32)cc1 ; c1ccc(-n2c3ccccc3c3ccc(-c4cccc(N(c5ccc6c(c5)sc5ccccc56)c5cccc6c5c5ccccc5n6-c5ccccc5)c4)cc32)cc1]

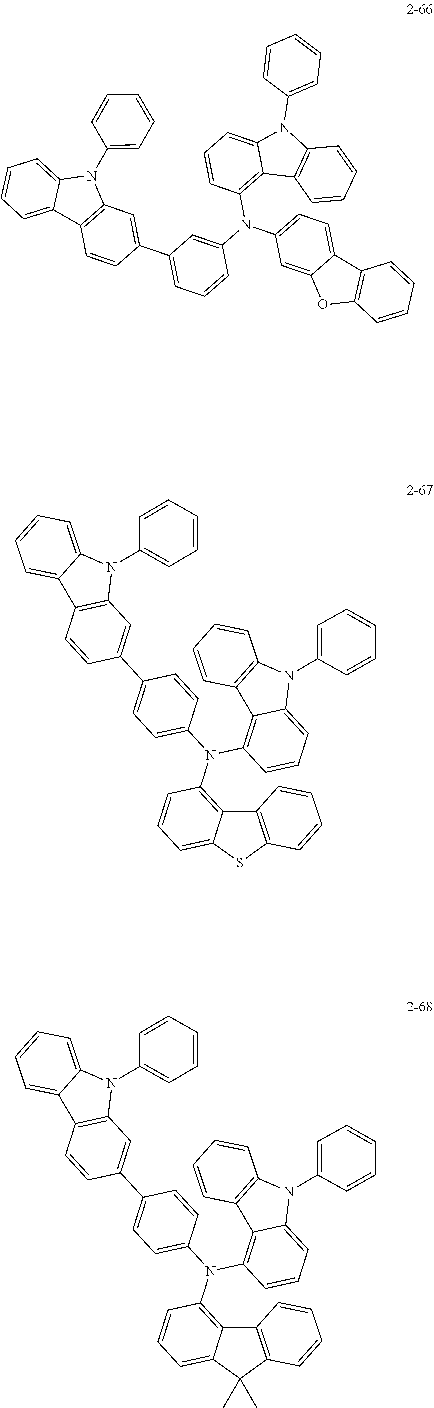 [SMILES: c1ccc(-n2c3ccccc3c3ccc(-c4cccc(N(c5ccc6c(c5)oc5ccccc56)c5cccc6c5c5ccccc5n6-c5ccccc5)c4)cc32)cc1 ; c1ccc(-n2c3ccccc3c3ccc(-c4ccc(N(c5cccc6sc7ccccc7c56)c5cccc6c5c5ccccc5n6-c5ccccc5)cc4)cc32)cc1 ; CC1(C)c2ccccc2-c2c(N(c3ccc(-c4ccc5c6ccccc6n(-c6ccccc6)c5c4)cc3)c3cccc4c3c3ccccc3n4-c3ccccc3)cccc21]

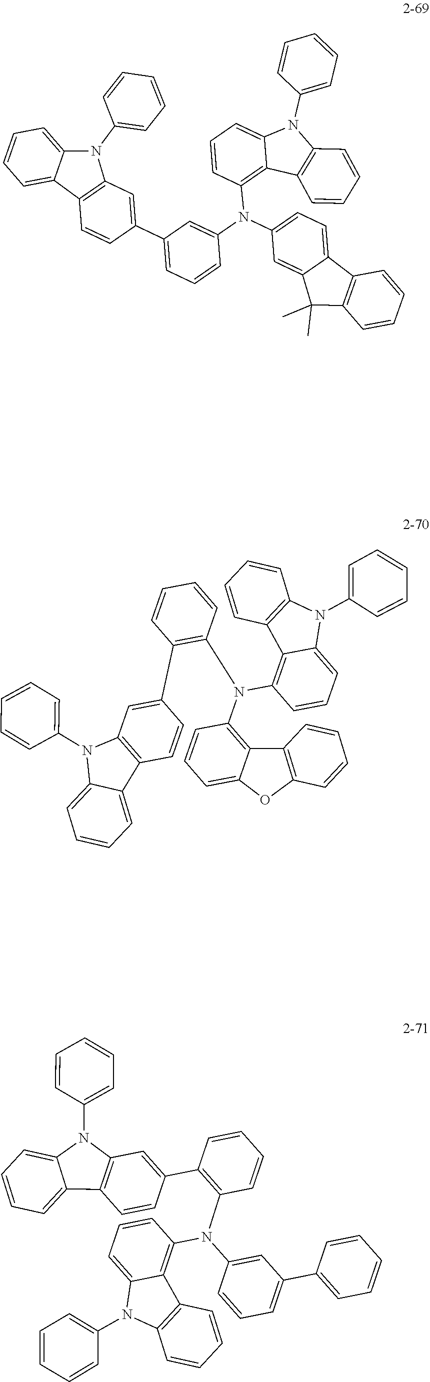 [SMILES: CC1(C)c2ccccc2-c2ccc(N(c3cccc(-c4ccc5c6ccccc6n(-c6ccccc6)c5c4)c3)c3cccc4c3c3ccccc3n4-c3ccccc3)cc21 ; c1ccc(-n2c3ccccc3c3ccc(-c4ccccc4N(c4cccc5oc6ccccc6c45)c4cccc5c4c4ccccc4n5-c4ccccc4)cc32)cc1 ; c1ccc(-c2cccc(N(c3ccccc3-c3ccc4c5ccccc5n(-c5ccccc5)c4c3)c3cccc4c3c3ccccc3n4-c3ccccc3)c2)cc1]

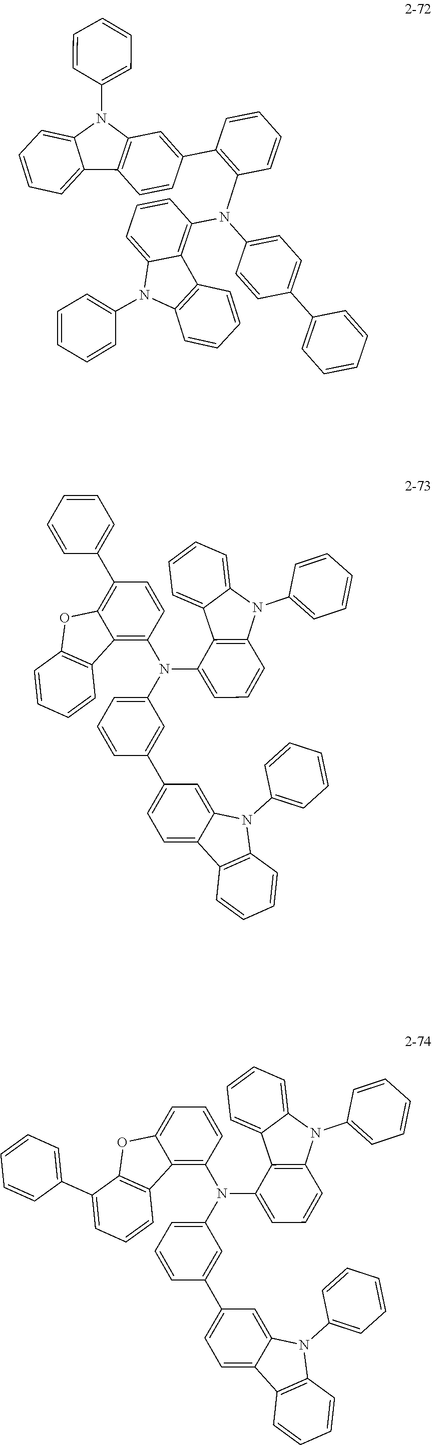 [SMILES: c1ccc(-c2ccc(N(c3ccccc3-c3ccc4c5ccccc5n(-c5ccccc5)c4c3)c3cccc4c3c3ccccc3n4-c3ccccc3)cc2)cc1 ; c1ccc(-c2ccc(N(c3cccc(-c4ccc5c6ccccc6n(-c6ccccc6)c5c4)c3)c3cccc4c3c3ccccc3n4-c3ccccc3)c3c2oc2ccccc23)cc1 ; c1ccc(-c2cccc3c2oc2cccc(N(c4cccc(-c5ccc6c7ccccc7n(-c7ccccc7)c6c5)c4)c4cccc5c4c4ccccc4n5-c4ccccc4)c23)cc1]

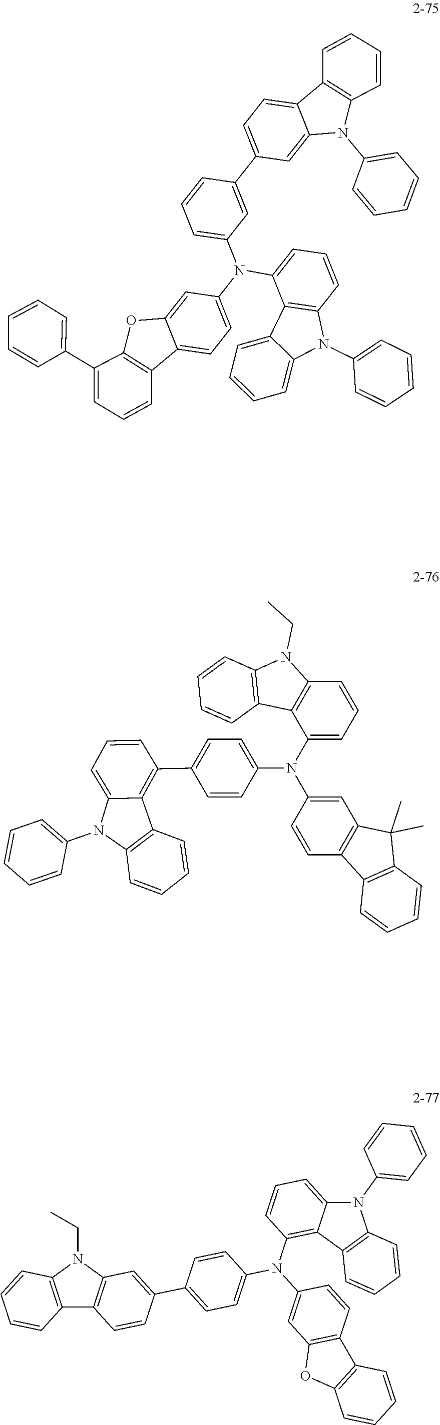

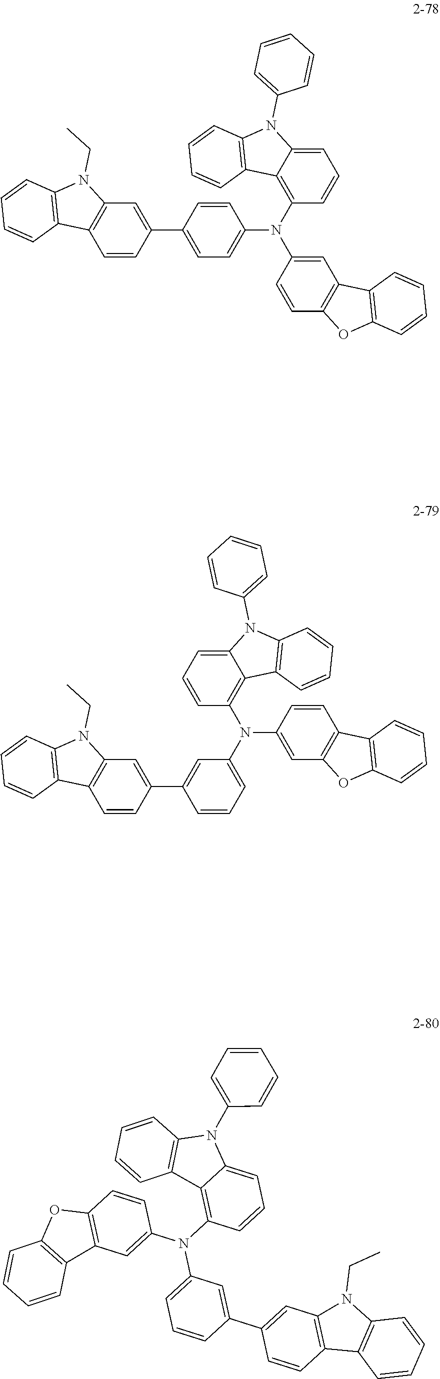 [SMILES: CCn1c2ccccc2c2ccc(-c3ccc(N(c4ccc5oc6ccccc6c5c4)c4cccc5c4c4ccccc4n5-c4ccccc4)cc3)cc21 ; CCn1c2ccccc2c2ccc(-c3cccc(N(c4ccc5c(c4)oc4ccccc45)c4cccc5c4c4ccccc4n5-c4ccccc4)c3)cc21 ; CCn1c2ccccc2c2ccc(-c3cccc(N(c4ccc5oc6ccccc6c5c4)c4cccc5c4c4ccccc4n5-c4ccccc4)c3)cc21]

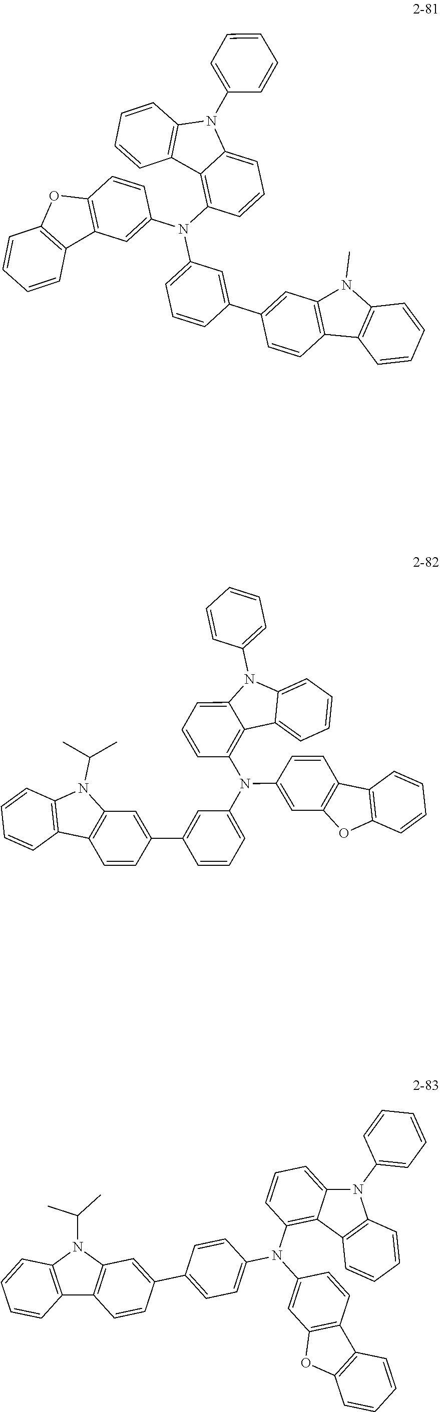 [SMILES: Cn1c2ccccc2c2ccc(-c3cccc(N(c4ccc5oc6ccccc6c5c4)c4cccc5c4c4ccccc4n5-c4ccccc4)c3)cc21 ; CC(C)n1c2ccccc2c2ccc(-c3cccc(N(c4ccc5c(c4)oc4ccccc45)c4cccc5c4c4ccccc4n5-c4ccccc4)c3)cc21 ; CC(C)n1c2ccccc2c2ccc(-c3ccc(N(c4ccc5c(c4)oc4ccccc45)c4cccc5c4c4ccccc4n5-c4ccccc4)cc3)cc21]

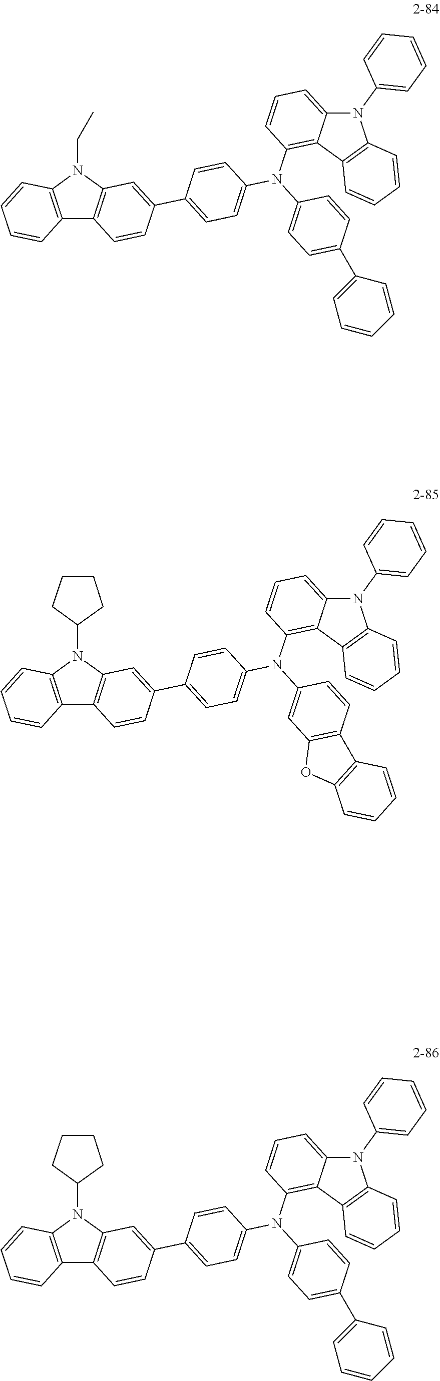 [SMILES: CCn1c2ccccc2c2ccc(-c3ccc(N(c4ccc(-c5ccccc5)cc4)c4cccc5c4c4ccccc4n5-c4ccccc4)cc3)cc21 ; c1ccc(-n2c3ccccc3c3c(N(c4ccc(-c5ccc6c7ccccc7n(C7CCCC7)c6c5)cc4)c4ccc5c(c4)oc4ccccc45)cccc32)cc1 ; c1ccc(-c2ccc(N(c3ccc(-c4ccc5c6ccccc6n(C6CCCC6)c5c4)cc3)c3cccc4c3c3ccccc3n4-c3ccccc3)cc2)cc1]

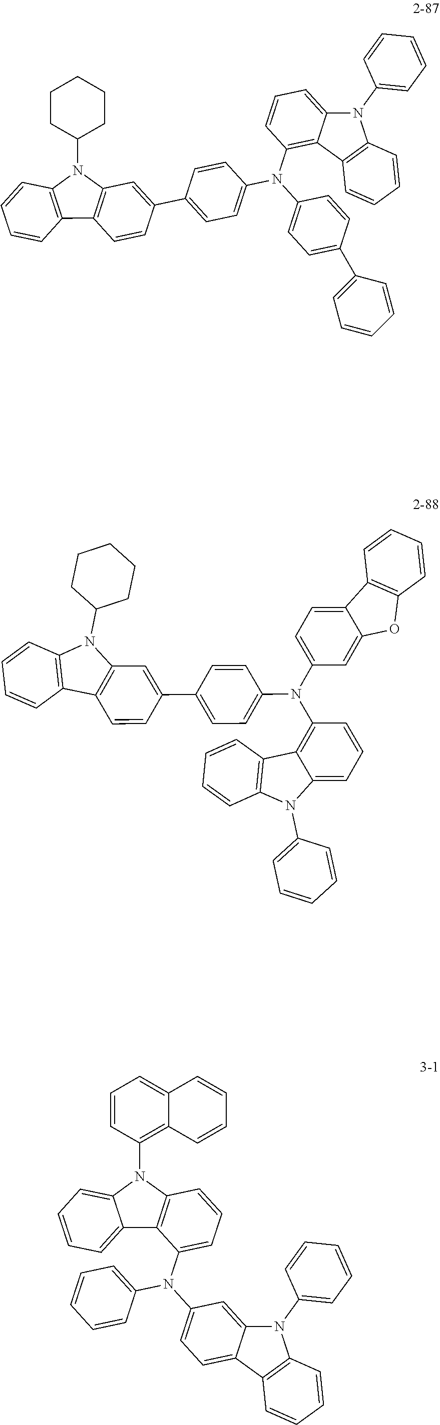 [SMILES: c1ccc(-c2ccc(N(c3ccc(-c4ccc5c6ccccc6n(C6CCCCC6)c5c4)cc3)c3cccc4c3c3ccccc3n4-c3ccccc3)cc2)cc1 ; c1ccc(-n2c3ccccc3c3c(N(c4ccc(-c5ccc6c7ccccc7n(C7CCCCC7)c6c5)cc4)c4ccc5c(c4)oc4ccccc45)cccc32)cc1 ; c1ccc(N(c2ccc3c4ccccc4n(-c4ccccc4)c3c2)c2cccc3c2c2ccccc2n3-c2cccc3ccccc23)cc1]

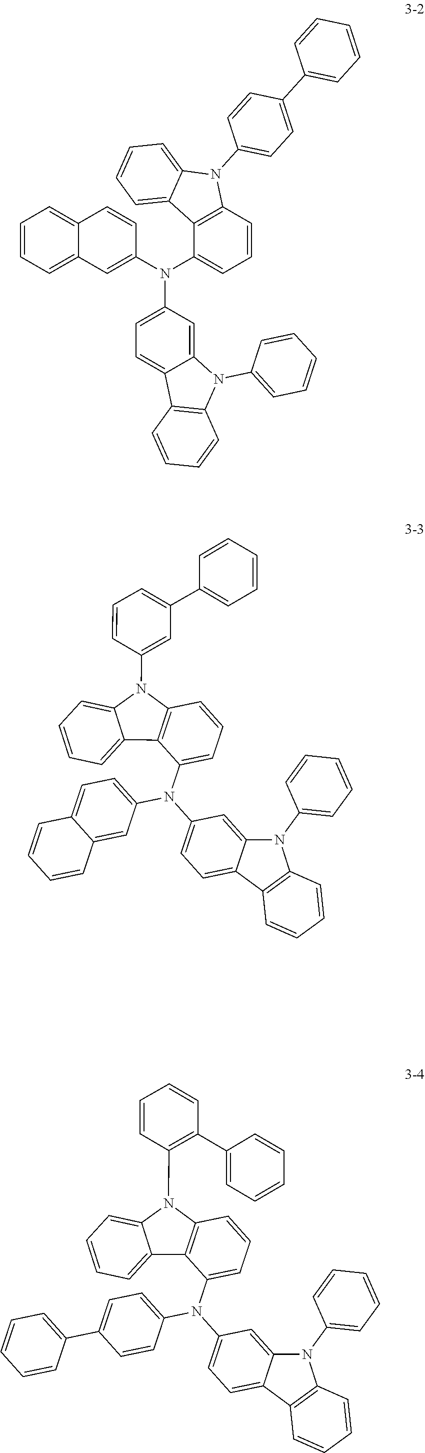 [SMILES: c1ccc(-c2ccc(-n3c4ccccc4c4c(N(c5ccc6ccccc6c5)c5ccc6c7ccccc7n(-c7ccccc7)c6c5)cccc43)cc2)cc1 ; c1ccc(-c2cccc(-n3c4ccccc4c4c(N(c5ccc6ccccc6c5)c5ccc6c7ccccc7n(-c7ccccc7)c6c5)cccc43)c2)cc1 ; c1ccc(-c2ccc(N(c3ccc4c5ccccc5n(-c5ccccc5)c4c3)c3cccc4c3c3ccccc3n4-c3ccccc3-c3ccccc3)cc2)cc1]

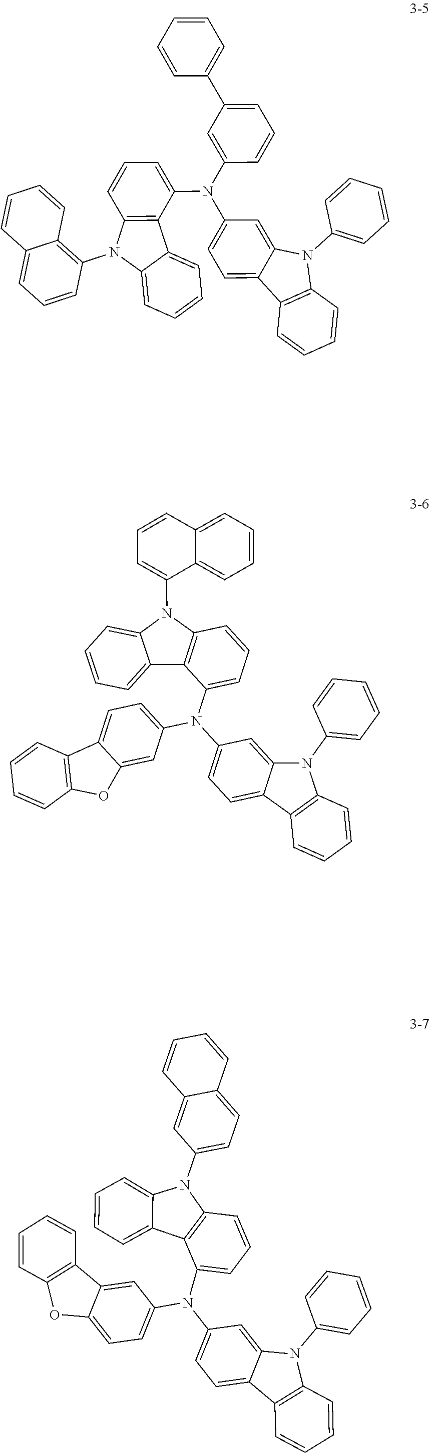 [SMILES: c1ccc(-c2cccc(N(c3ccc4c5ccccc5n(-c5ccccc5)c4c3)c3cccc4c3c3ccccc3n4-c3cccc4ccccc34)c2)cc1 ; c1ccc(-n2c3ccccc3c3ccc(N(c4ccc5c(c4)oc4ccccc45)c4cccc5c4c4ccccc4n5-c4cccc5ccccc45)cc32)cc1 ; c1ccc(-n2c3ccccc3c3ccc(N(c4ccc5oc6ccccc6c5c4)c4cccc5c4c4ccccc4n5-c4ccc5ccccc5c4)cc32)cc1]

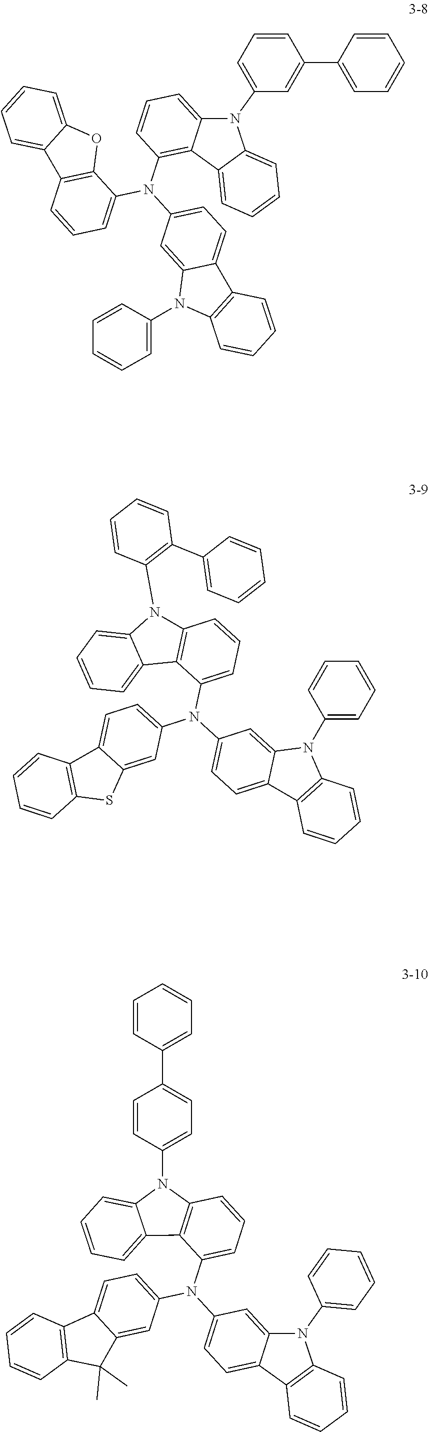 [SMILES: c1ccc(-c2cccc(-n3c4ccccc4c4c(N(c5ccc6c7ccccc7n(-c7ccccc7)c6c5)c5cccc6c5oc5ccccc56)cccc43)c2)cc1 ; c1ccc(-c2ccccc2-n2c3ccccc3c3c(N(c4ccc5c(c4)sc4ccccc45)c4ccc5c6ccccc6n(-c6ccccc6)c5c4)cccc32)cc1 ; CC1(C)c2ccccc2-c2ccc(N(c3ccc4c5ccccc5n(-c5ccccc5)c4c3)c3cccc4c3c3ccccc3n4-c3ccc(-c4ccccc4)cc3)cc21]

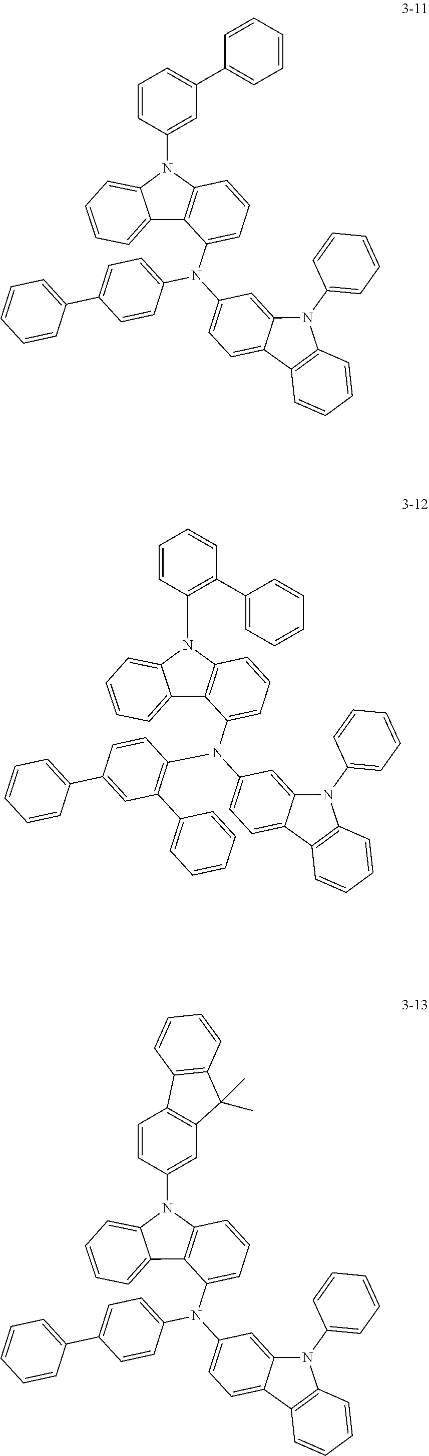 [SMILES: c1ccc(-c2ccc(N(c3ccc4c5ccccc5n(-c5ccccc5)c4c3)c3cccc4c3c3ccccc3n4-c3cccc(-c4ccccc4)c3)cc2)cc1 ; c1ccc(-c2ccc(N(c3ccc4c5ccccc5n(-c5ccccc5)c4c3)c3cccc4c3c3ccccc3n4-c3ccccc3-c3ccccc3)c(-c3ccccc3)c2)cc1 ; CC1(C)c2ccccc2-c2ccc(-n3c4ccccc4c4c(N(c5ccc(-c6ccccc6)cc5)c5ccc6c7ccccc7n(-c7ccccc7)c6c5)cccc43)cc21]

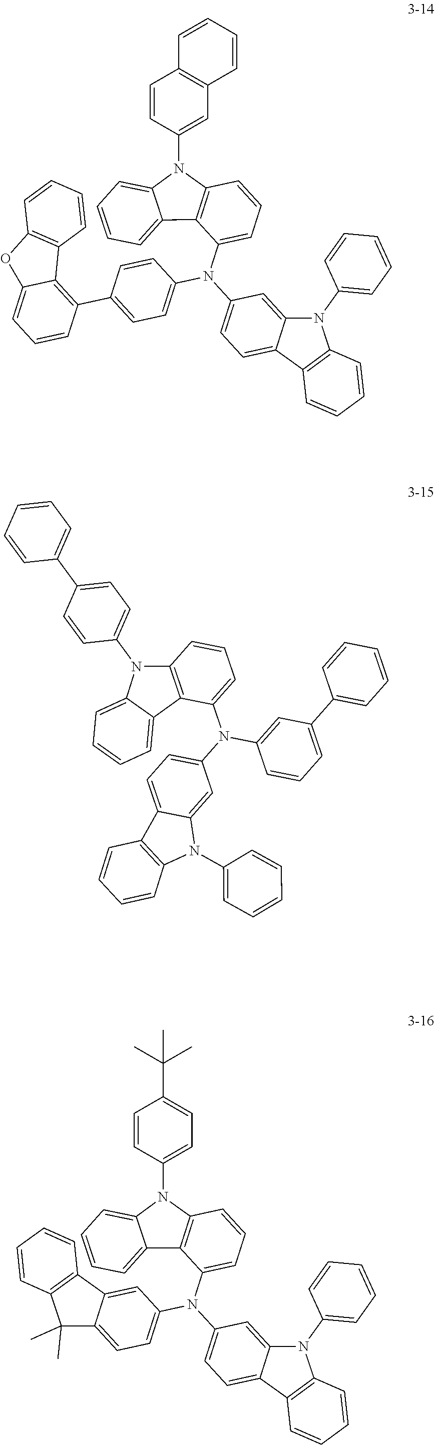 [SMILES: c1ccc(-n2c3ccccc3c3ccc(N(c4ccc(-c5cccc6oc7ccccc7c56)cc4)c4cccc5c4c4ccccc4n5-c4ccc5ccccc5c4)cc32)cc1 ; c1ccc(-c2ccc(-n3c4ccccc4c4c(N(c5cccc(-c6ccccc6)c5)c5ccc6c7ccccc7n(-c7ccccc7)c6c5)cccc43)cc2)cc1 ; CC(C)(C)c1ccc(-n2c3ccccc3c3c(N(c4ccc5c(c4)-c4ccccc4C5(C)C)c4ccc5c6ccccc6n(-c6ccccc6)c5c4)cccc32)cc1]

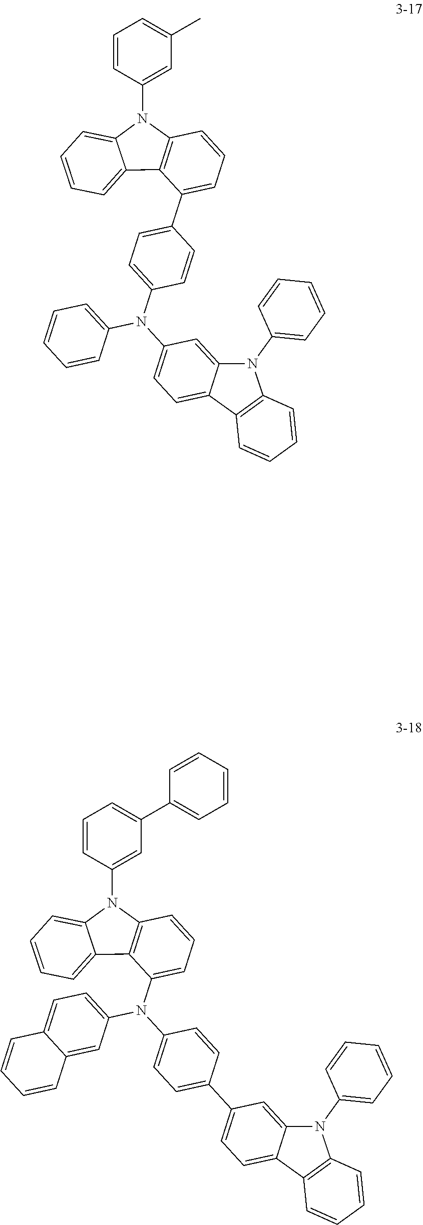 [SMILES: Cc1cccc(-n2c3ccccc3c3c(-c4ccc(N(c5ccccc5)c5ccc6c7ccccc7n(-c7ccccc7)c6c5)cc4)cccc32)c1 ; c1ccc(-c2cccc(-n3c4ccccc4c4c(N(c5ccc(-c6ccc7c8ccccc8n(-c8ccccc8)c7c6)cc5)c5ccc6ccccc6c5)cccc43)c2)cc1]

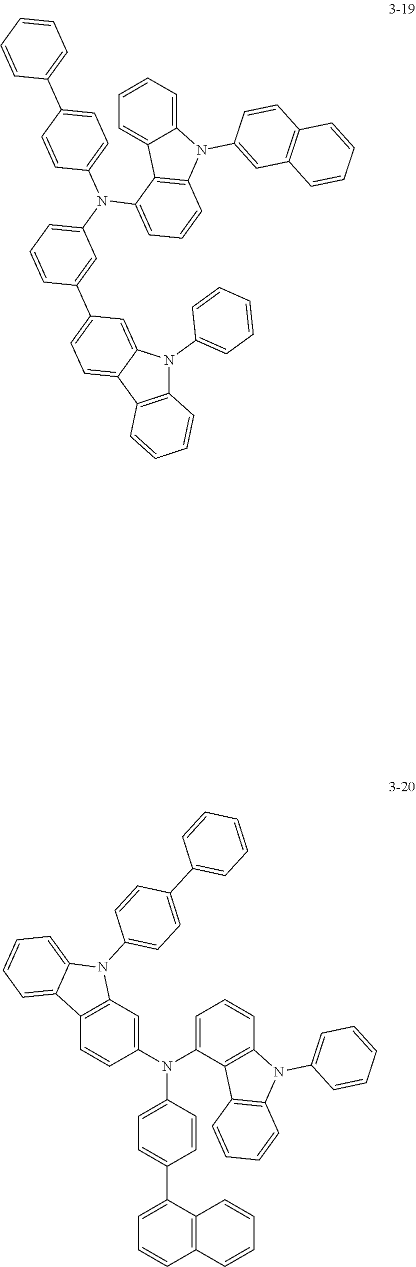 [SMILES: c1ccc(-c2ccc(N(c3cccc(-c4ccc5c6ccccc6n(-c6ccccc6)c5c4)c3)c3cccc4c3c3ccccc3n4-c3ccc4ccccc4c3)cc2)cc1 ; c1ccc(-c2ccc(-n3c4ccccc4c4ccc(N(c5ccc(-c6cccc7ccccc67)cc5)c5cccc6c5c5ccccc5n6-c5ccccc5)cc43)cc2)cc1]

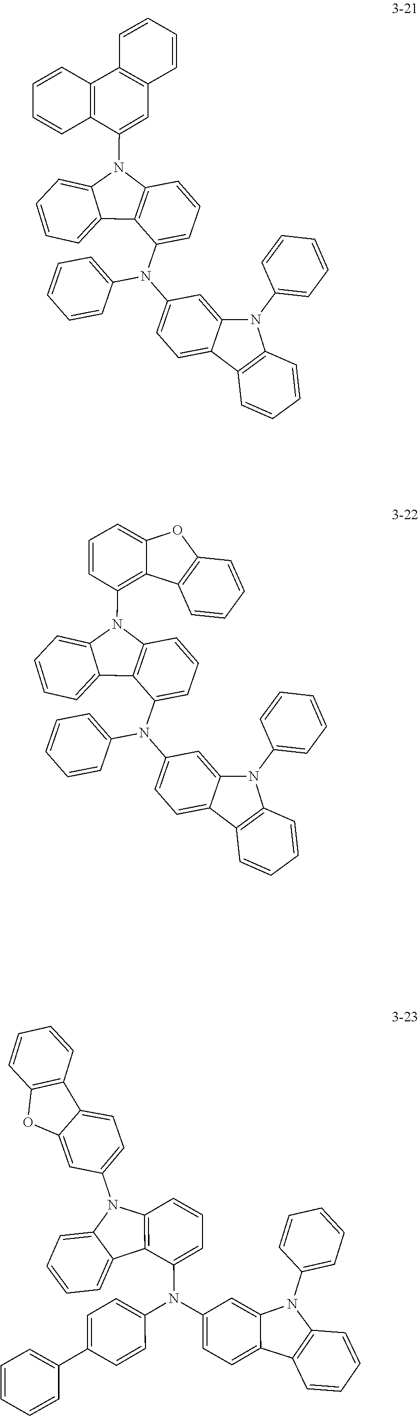 [SMILES: c1ccc(N(c2ccc3c4ccccc4n(-c4ccccc4)c3c2)c2cccc3c2c2ccccc2n3-c2cc3ccccc3c3ccccc23)cc1 ; c1ccc(N(c2ccc3c4ccccc4n(-c4ccccc4)c3c2)c2cccc3c2c2ccccc2n3-c2cccc3oc4ccccc4c23)cc1 ; c1ccc(-c2ccc(N(c3ccc4c5ccccc5n(-c5ccccc5)c4c3)c3cccc4c3c3ccccc3n4-c3ccc4c(c3)oc3ccccc34)cc2)cc1]

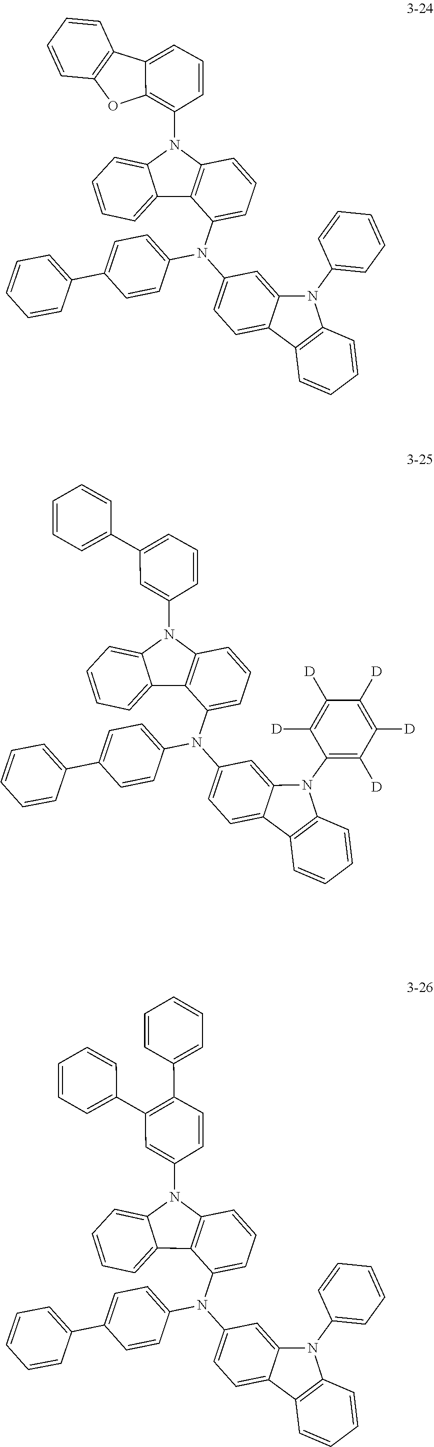 [SMILES: c1ccc(-c2ccc(N(c3ccc4c5ccccc5n(-c5ccccc5)c4c3)c3cccc4c3c3ccccc3n4-c3cccc4c3oc3ccccc34)cc2)cc1 ; [2H]c1c([2H])c([2H])c(-n2c3ccccc3c3ccc(N(c4ccc(-c5ccccc5)cc4)c4cccc5c4c4ccccc4n5-c4cccc(-c5ccccc5)c4)cc32)c([2H])c1[2H] ; c1ccc(-c2ccc(N(c3ccc4c5ccccc5n(-c5ccccc5)c4c3)c3cccc4c3c3ccccc3n4-c3ccc(-c4ccccc4)c(-c4ccccc4)c3)cc2)cc1]

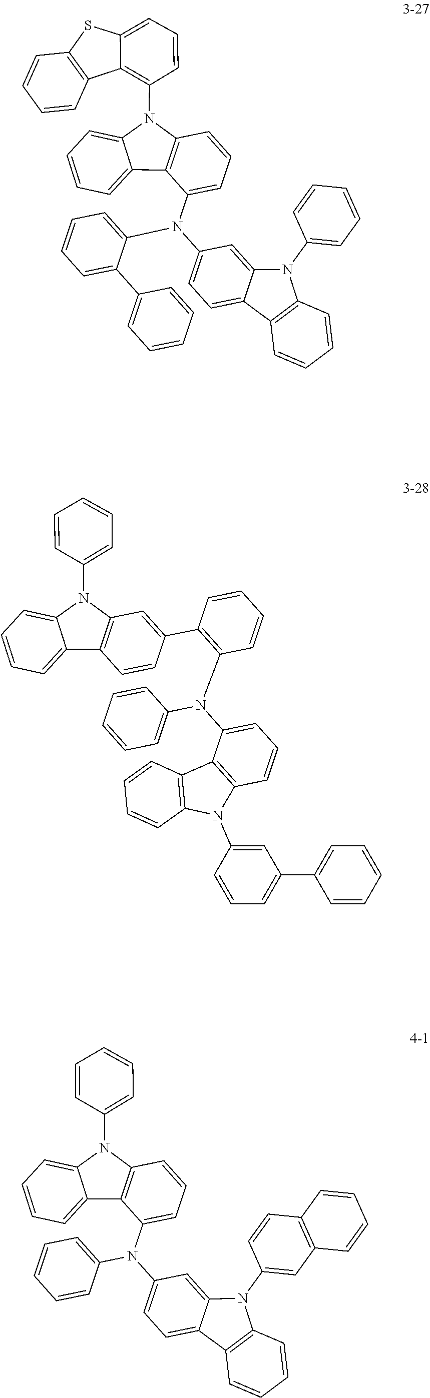 [SMILES: c1ccc(-c2ccccc2N(c2ccc3c4ccccc4n(-c4ccccc4)c3c2)c2cccc3c2c2ccccc2n3-c2cccc3sc4ccccc4c23)cc1 ; c1ccc(-c2cccc(-n3c4ccccc4c4c(N(c5ccccc5)c5ccccc5-c5ccc6c7ccccc7n(-c7ccccc7)c6c5)cccc43)c2)cc1 ; c1ccc(N(c2ccc3c4ccccc4n(-c4ccc5ccccc5c4)c3c2)c2cccc3c2c2ccccc2n3-c2ccccc2)cc1]

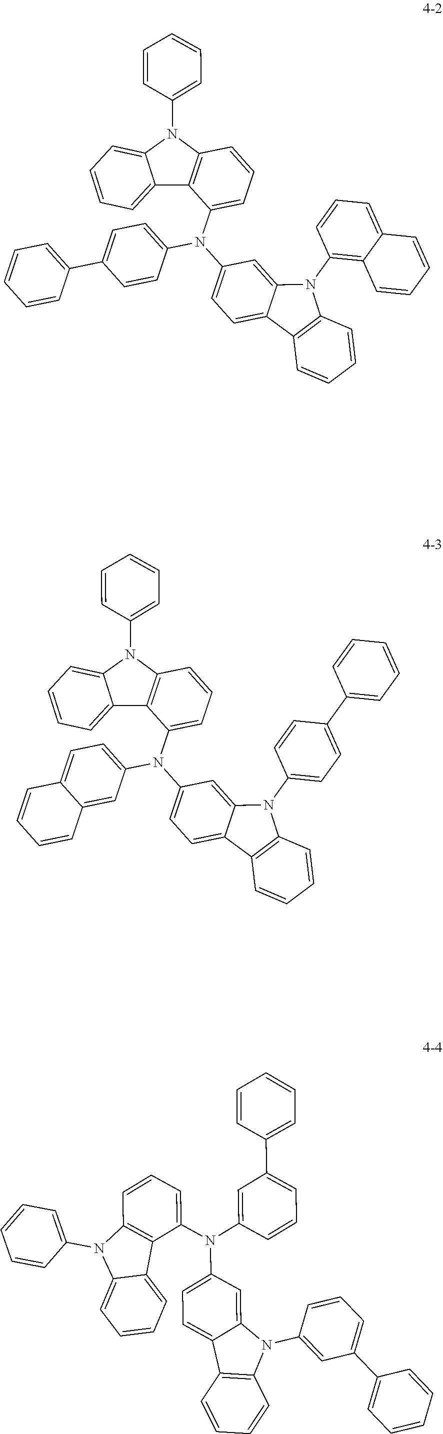 [SMILES: c1ccc(-c2ccc(N(c3ccc4c5ccccc5n(-c5cccc6ccccc56)c4c3)c3cccc4c3c3ccccc3n4-c3ccccc3)cc2)cc1 ; c1ccc(-c2ccc(-n3c4ccccc4c4ccc(N(c5ccc6ccccc6c5)c5cccc6c5c5ccccc5n6-c5ccccc5)cc43)cc2)cc1 ; c1ccc(-c2cccc(N(c3ccc4c5ccccc5n(-c5cccc(-c6ccccc6)c5)c4c3)c3cccc4c3c3ccccc3n4-c3ccccc3)c2)cc1]

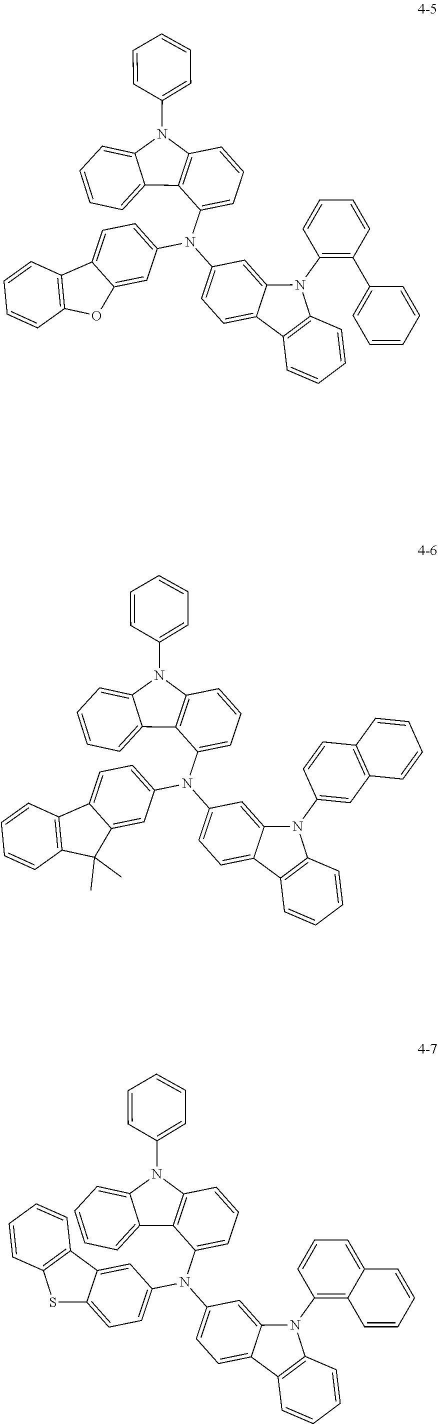 [SMILES: c1ccc(-c2ccccc2-n2c3ccccc3c3ccc(N(c4ccc5c(c4)oc4ccccc45)c4cccc5c4c4ccccc4n5-c4ccccc4)cc32)cc1 ; CC1(C)c2ccccc2-c2ccc(N(c3ccc4c5ccccc5n(-c5ccc6ccccc6c5)c4c3)c3cccc4c3c3ccccc3n4-c3ccccc3)cc21 ; c1ccc(-n2c3ccccc3c3c(N(c4ccc5sc6ccccc6c5c4)c4ccc5c6ccccc6n(-c6cccc7ccccc67)c5c4)cccc32)cc1]

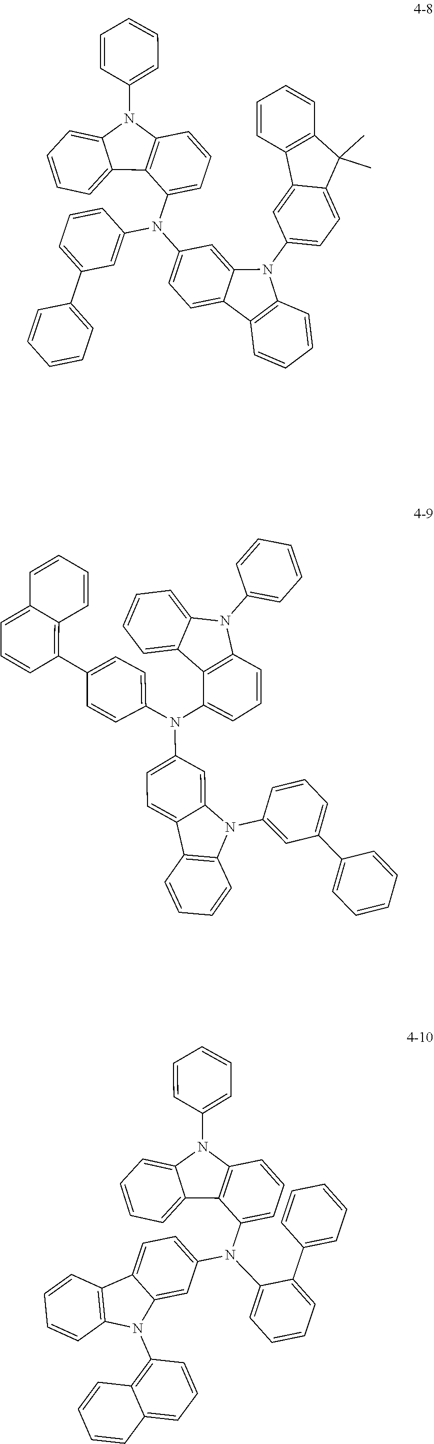 [SMILES: CC1(C)c2ccccc2-c2cc(-n3c4ccccc4c4ccc(N(c5cccc(-c6ccccc6)c5)c5cccc6c5c5ccccc5n6-c5ccccc5)cc43)ccc21 ; c1ccc(-c2cccc(-n3c4ccccc4c4ccc(N(c5ccc(-c6cccc7ccccc67)cc5)c5cccc6c5c5ccccc5n6-c5ccccc5)cc43)c2)cc1 ; c1ccc(-c2ccccc2N(c2ccc3c4ccccc4n(-c4cccc5ccccc45)c3c2)c2cccc3c2c2ccccc2n3-c2ccccc2)cc1]

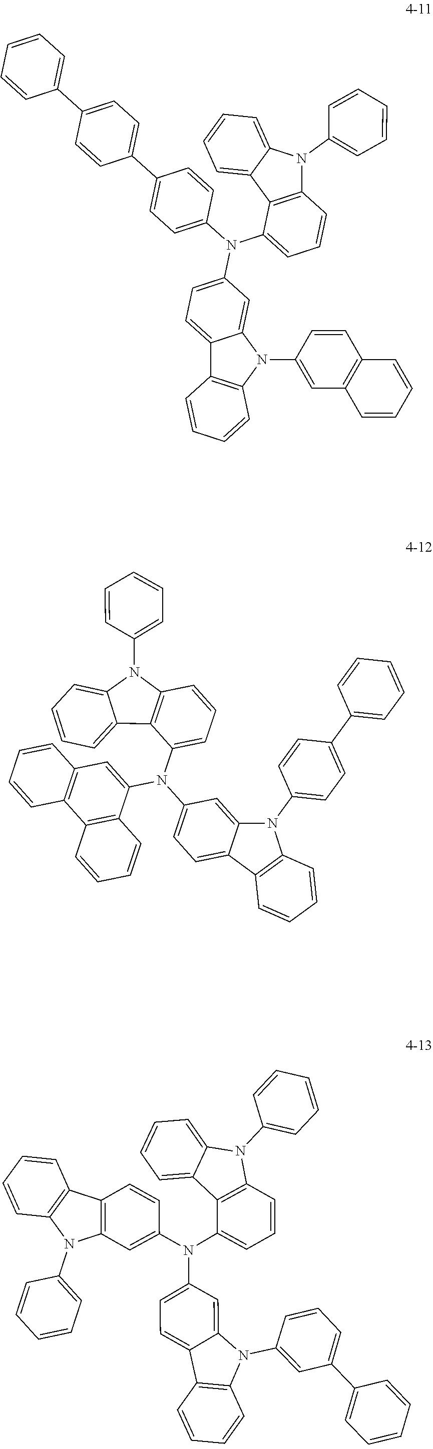

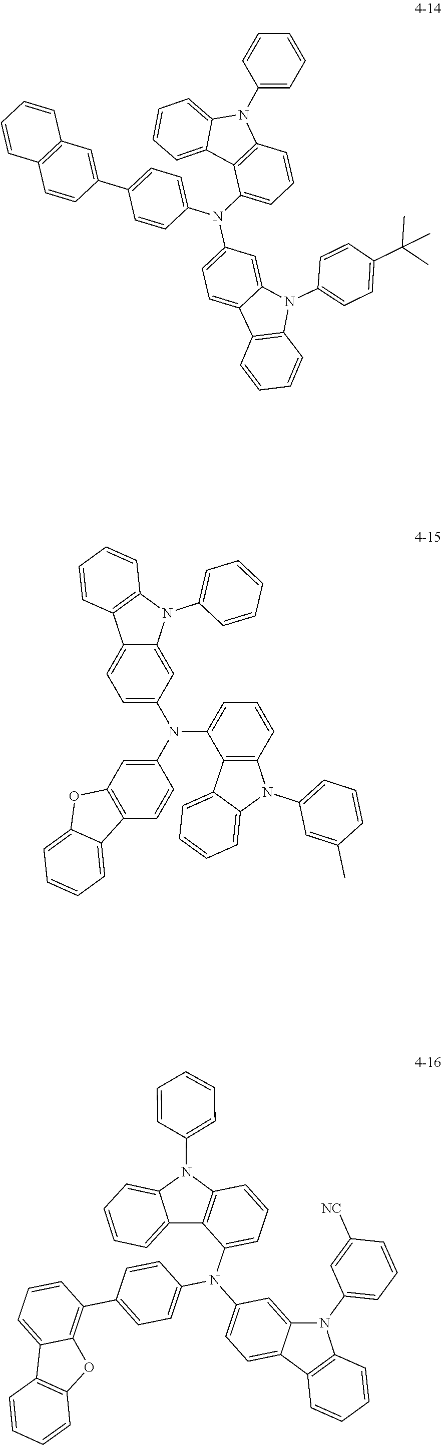

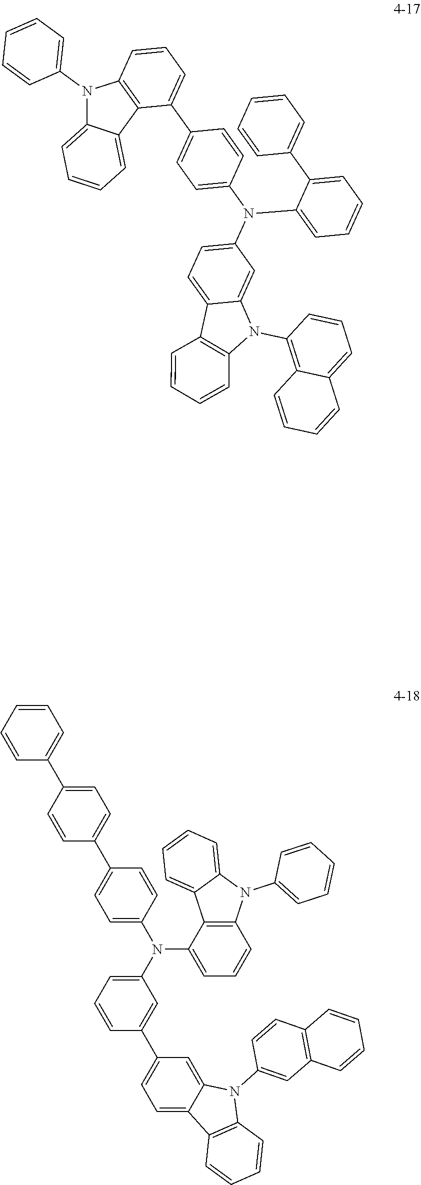 [SMILES: c1ccc(-c2ccccc2N(c2ccc(-c3cccc4c3c3ccccc3n4-c3ccccc3)cc2)c2ccc3c4ccccc4n(-c4cccc5ccccc45)c3c2)cc1 ; c1ccc(-c2ccc(-c3ccc(N(c4cccc(-c5ccc6c7ccccc7n(-c7ccc8ccccc8c7)c6c5)c4)c4cccc5c4c4ccccc4n5-c4ccccc4)cc3)cc2)cc1]

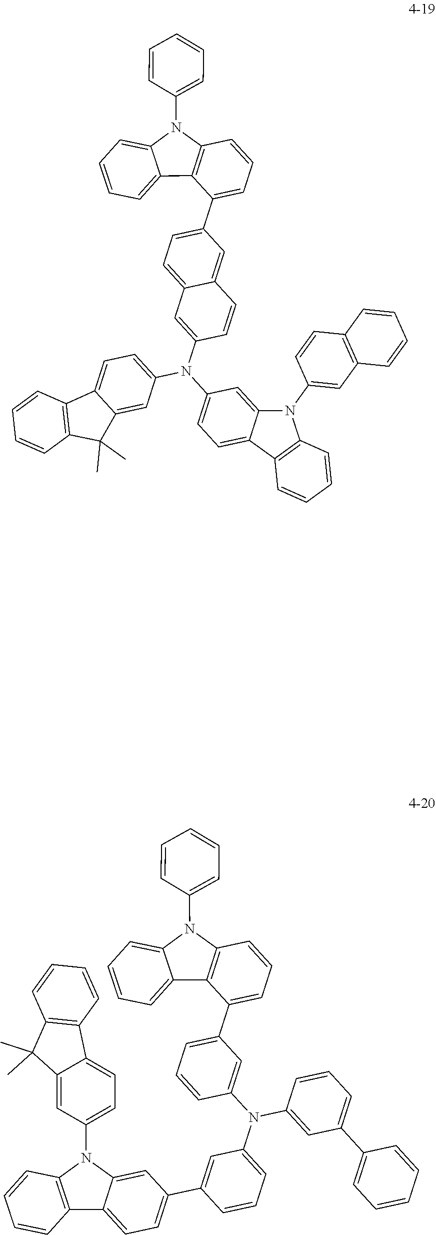 [SMILES: CC1(C)c2ccccc2-c2ccc(N(c3ccc4cc(-c5cccc6c5c5ccccc5n6-c5ccccc5)ccc4c3)c3ccc4c5ccccc5n(-c5ccc6ccccc6c5)c4c3)cc21 ; CC1(C)c2ccccc2-c2ccc(-n3c4ccccc4c4ccc(-c5cccc(N(c6cccc(-c7ccccc7)c6)c6cccc(-c7cccc8c7c7ccccc7n8-c7ccccc7)c6)c5)cc43)cc21]

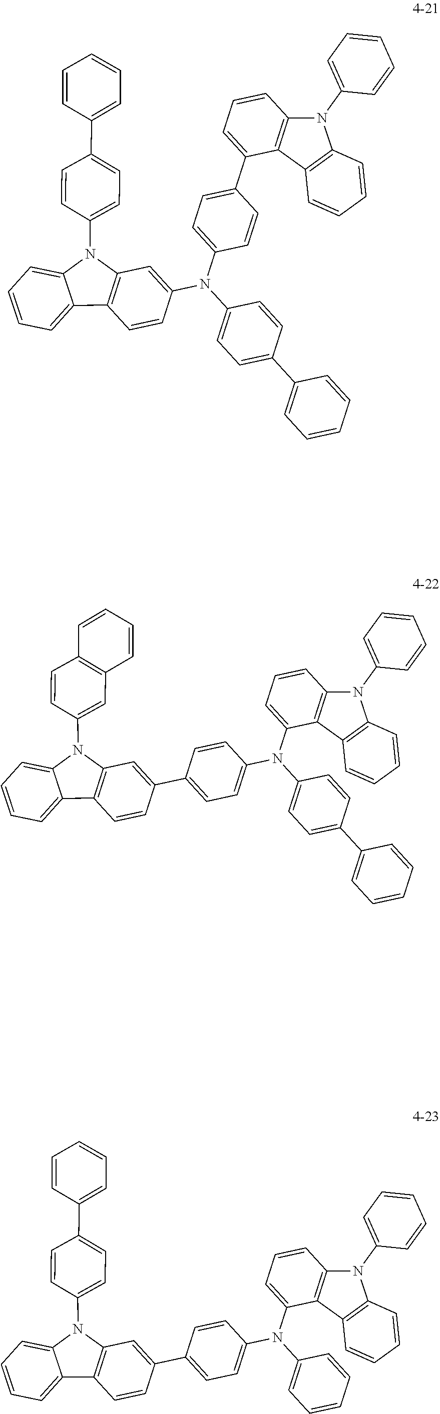 [SMILES: c1ccc(-c2ccc(N(c3ccc(-c4cccc5c4c4ccccc4n5-c4ccccc4)cc3)c3ccc4c5ccccc5n(-c5ccc(-c6ccccc6)cc5)c4c3)cc2)cc1 ; c1ccc(-c2ccc(N(c3ccc(-c4ccc5c6ccccc6n(-c6ccc7ccccc7c6)c5c4)cc3)c3cccc4c3c3ccccc3n4-c3ccccc3)cc2)cc1 ; c1ccc(-c2ccc(-n3c4ccccc4c4ccc(-c5ccc(N(c6ccccc6)c6cccc7c6c6ccccc6n7-c6ccccc6)cc5)cc43)cc2)cc1]

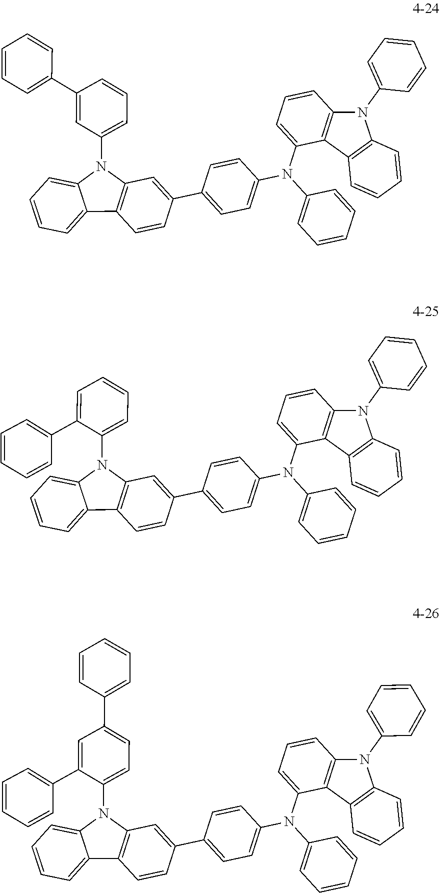 [SMILES: c1ccc(-c2cccc(-n3c4ccccc4c4ccc(-c5ccc(N(c6ccccc6)c6cccc7c6c6ccccc6n7-c6ccccc6)cc5)cc43)c2)cc1 ; c1ccc(-c2ccccc2-n2c3ccccc3c3ccc(-c4ccc(N(c5ccccc5)c5cccc6c5c5ccccc5n6-c5ccccc5)cc4)cc32)cc1 ; c1ccc(-c2ccc(-n3c4ccccc4c4ccc(-c5ccc(N(c6ccccc6)c6cccc7c6c6ccccc6n7-c6ccccc6)cc5)cc43)c(-c3ccccc3)c2)cc1]

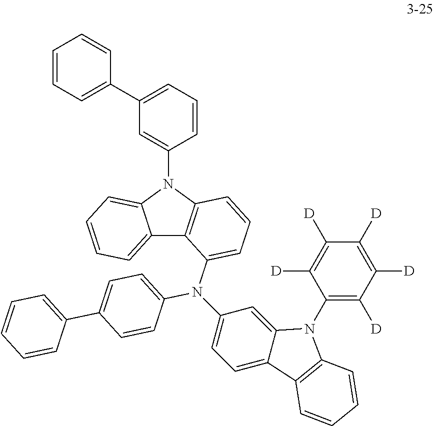 [SMILES: [2H]c1c([2H])c([2H])c(-n2c3ccccc3c3ccc(N(c4ccc(-c5ccccc5)cc4)c4cccc5c4c4ccccc4n5-c4cccc(-c5ccccc5)c4)cc32)c([2H])c1[2H]]

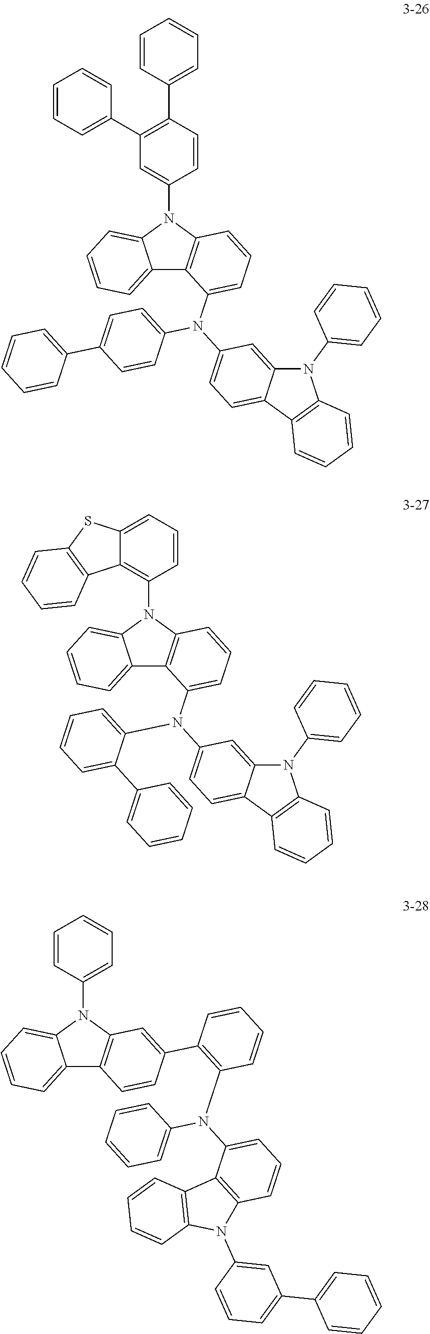 [SMILES: c1ccc(-c2ccc(N(c3ccc4c5ccccc5n(-c5ccccc5)c4c3)c3cccc4c3c3ccccc3n4-c3ccc(-c4ccccc4)c(-c4ccccc4)c3)cc2)cc1 ; c1ccc(-c2ccccc2N(c2ccc3c4ccccc4n(-c4ccccc4)c3c2)c2cccc3c2c2ccccc2n3-c2cccc3sc4ccccc4c23)cc1 ; c1ccc(-c2cccc(-n3c4ccccc4c4c(N(c5ccccc5)c5ccccc5-c5ccc6c7ccccc7n(-c7ccccc7)c6c5)cccc43)c2)cc1]

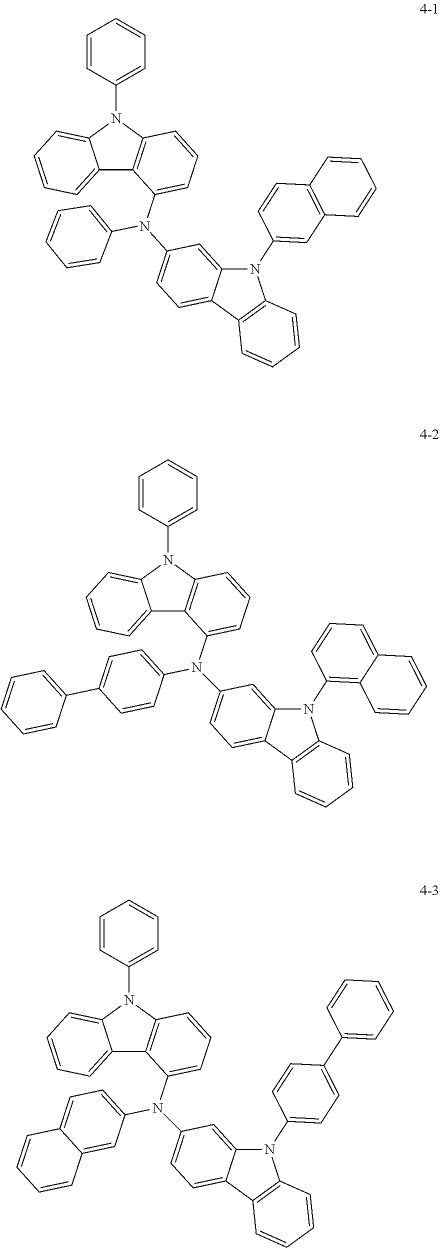 [SMILES: c1ccc(N(c2ccc3c4ccccc4n(-c4ccc5ccccc5c4)c3c2)c2cccc3c2c2ccccc2n3-c2ccccc2)cc1 ; c1ccc(-c2ccc(N(c3ccc4c5ccccc5n(-c5cccc6ccccc56)c4c3)c3cccc4c3c3ccccc3n4-c3ccccc3)cc2)cc1 ; c1ccc(-c2ccc(-n3c4ccccc4c4ccc(N(c5ccc6ccccc6c5)c5cccc6c5c5ccccc5n6-c5ccccc5)cc43)cc2)cc1]

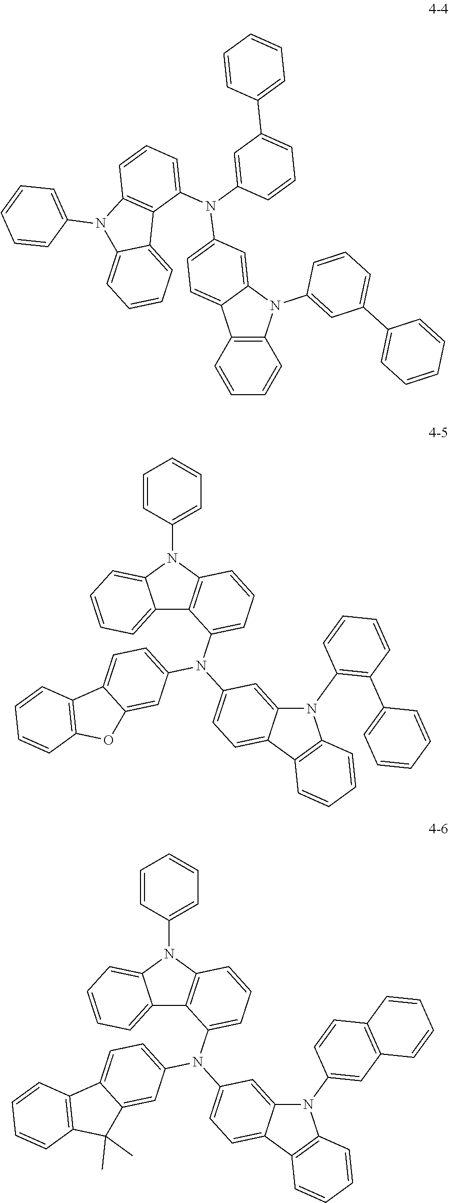 [SMILES: c1ccc(-c2cccc(N(c3ccc4c5ccccc5n(-c5cccc(-c6ccccc6)c5)c4c3)c3cccc4c3c3ccccc3n4-c3ccccc3)c2)cc1 ; c1ccc(-c2ccccc2-n2c3ccccc3c3ccc(N(c4ccc5c(c4)oc4ccccc45)c4cccc5c4c4ccccc4n5-c4ccccc4)cc32)cc1 ; CC1(C)c2ccccc2-c2ccc(N(c3ccc4c5ccccc5n(-c5ccc6ccccc6c5)c4c3)c3cccc4c3c3ccccc3n4-c3ccccc3)cc21]

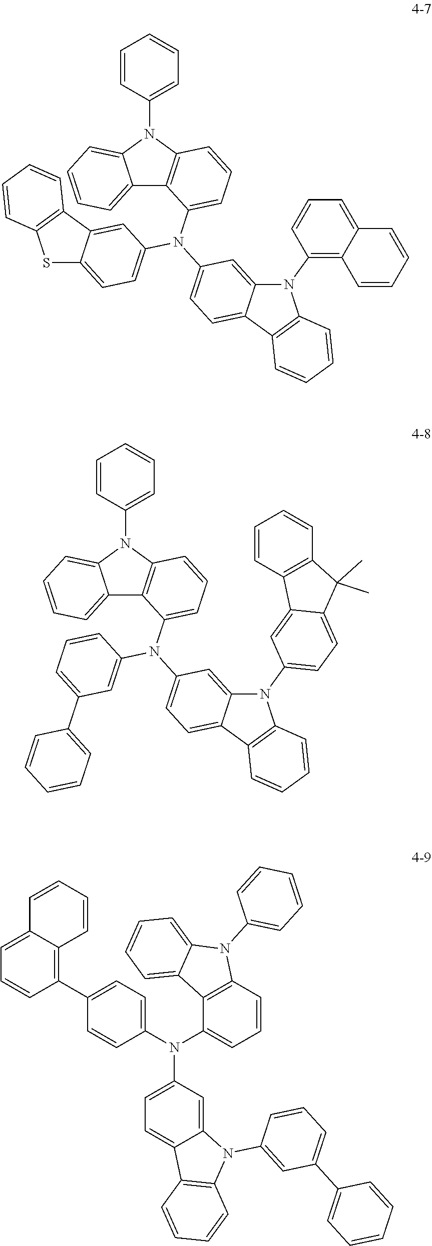 [SMILES: c1ccc(-n2c3ccccc3c3c(N(c4ccc5sc6ccccc6c5c4)c4ccc5c6ccccc6n(-c6cccc7ccccc67)c5c4)cccc32)cc1 ; CC1(C)c2ccccc2-c2cc(-n3c4ccccc4c4ccc(N(c5cccc(-c6ccccc6)c5)c5cccc6c5c5ccccc5n6-c5ccccc5)cc43)ccc21 ; c1ccc(-c2cccc(-n3c4ccccc4c4ccc(N(c5ccc(-c6cccc7ccccc67)cc5)c5cccc6c5c5ccccc5n6-c5ccccc5)cc43)c2)cc1]

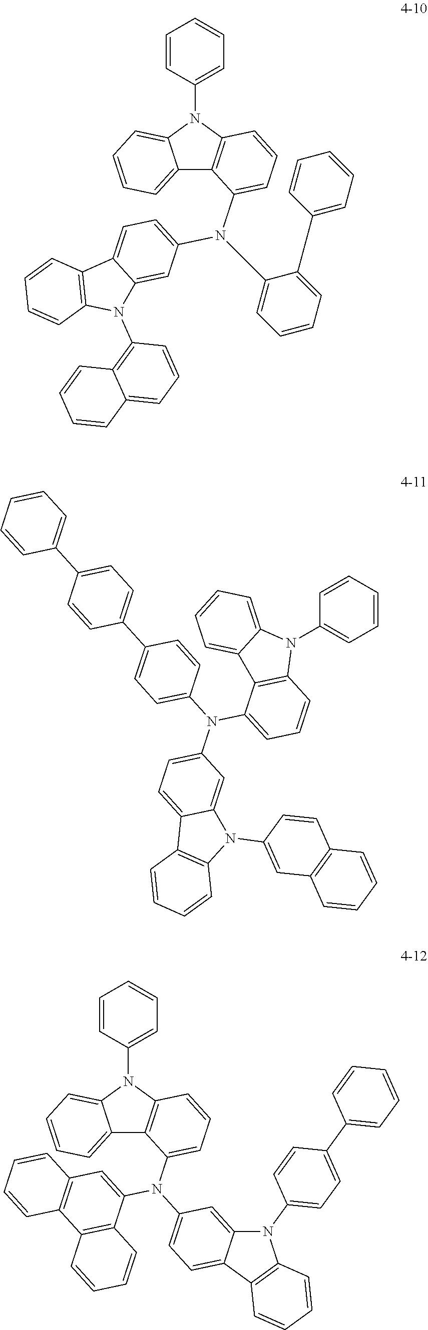 [SMILES: c1ccc(-c2ccccc2N(c2ccc3c4ccccc4n(-c4cccc5ccccc45)c3c2)c2cccc3c2c2ccccc2n3-c2ccccc2)cc1 ; c1ccc(-c2ccc(-c3ccc(N(c4ccc5c6ccccc6n(-c6ccc7ccccc7c6)c5c4)c4cccc5c4c4ccccc4n5-c4ccccc4)cc3)cc2)cc1 ; c1ccc(-c2ccc(-n3c4ccccc4c4ccc(N(c5cc6ccccc6c6ccccc56)c5cccc6c5c5ccccc5n6-c5ccccc5)cc43)cc2)cc1]

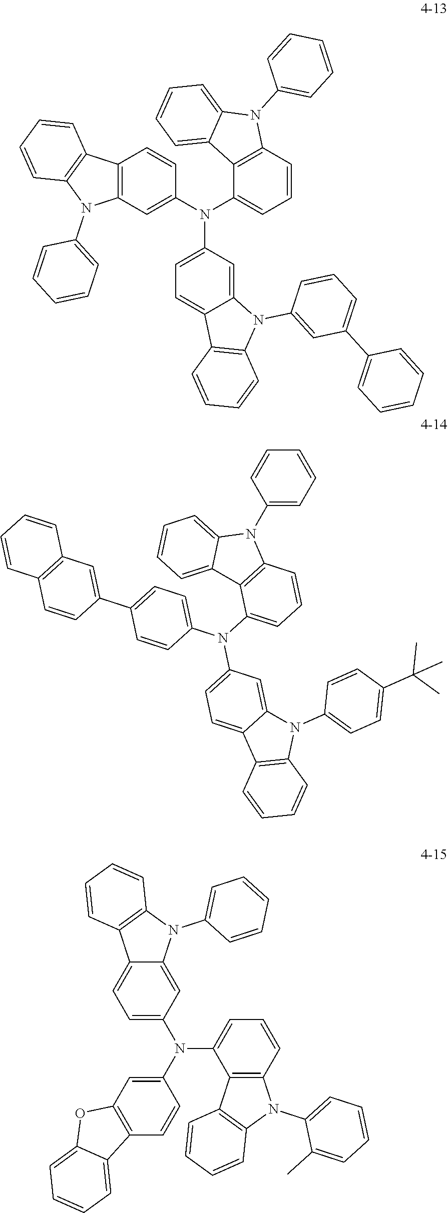 [SMILES: c1ccc(-c2cccc(-n3c4ccccc4c4ccc(N(c5ccc6c7ccccc7n(-c7ccccc7)c6c5)c5cccc6c5c5ccccc5n6-c5ccccc5)cc43)c2)cc1 ; CC(C)(C)c1ccc(-n2c3ccccc3c3ccc(N(c4ccc(-c5ccc6ccccc6c5)cc4)c4cccc5c4c4ccccc4n5-c4ccccc4)cc32)cc1 ; Cc1ccccc1-n1c2ccccc2c2c(N(c3ccc4c(c3)oc3ccccc34)c3ccc4c5ccccc5n(-c5ccccc5)c4c3)cccc21]

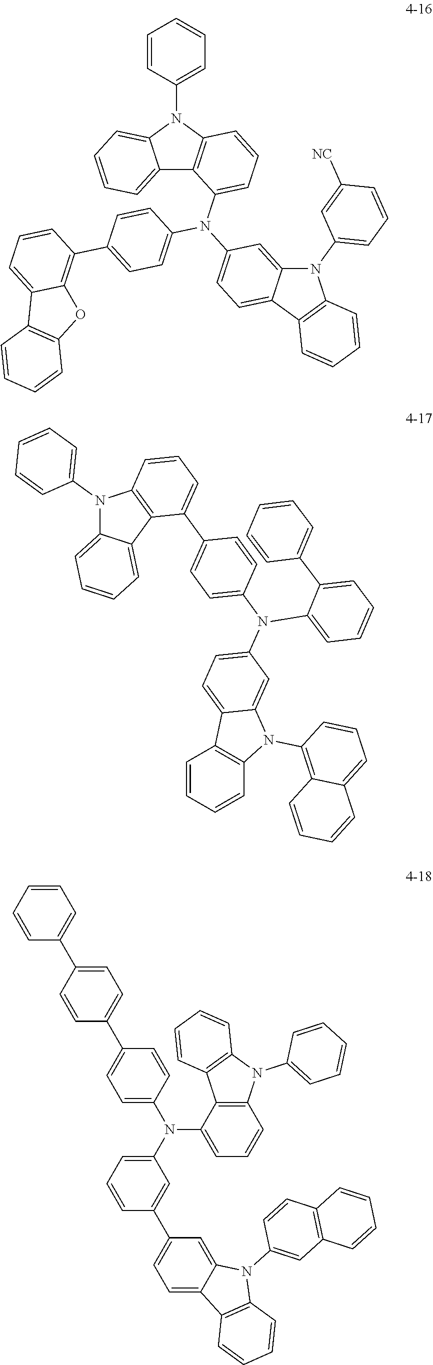

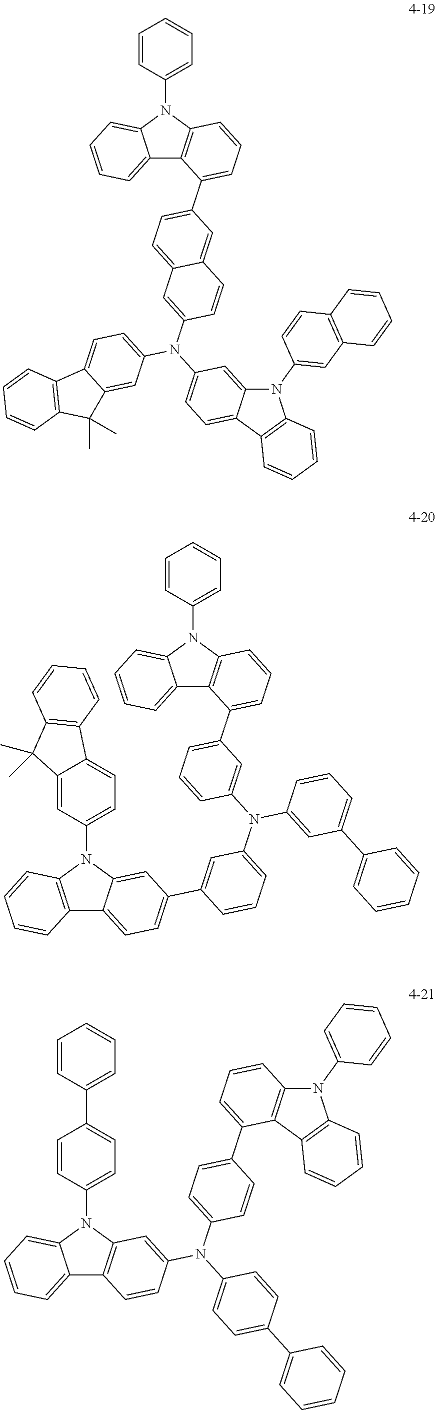 [SMILES: CC1(C)c2ccccc2-c2ccc(N(c3ccc4cc(-c5cccc6c5c5ccccc5n6-c5ccccc5)ccc4c3)c3ccc4c5ccccc5n(-c5ccc6ccccc6c5)c4c3)cc21 ; CC1(C)c2ccccc2-c2ccc(-n3c4ccccc4c4ccc(-c5cccc(N(c6cccc(-c7ccccc7)c6)c6cccc(-c7cccc8c7c7ccccc7n8-c7ccccc7)c6)c5)cc43)cc21 ; c1ccc(-c2ccc(N(c3ccc(-c4cccc5c4c4ccccc4n5-c4ccccc4)cc3)c3ccc4c5ccccc5n(-c5ccc(-c6ccccc6)cc5)c4c3)cc2)cc1]

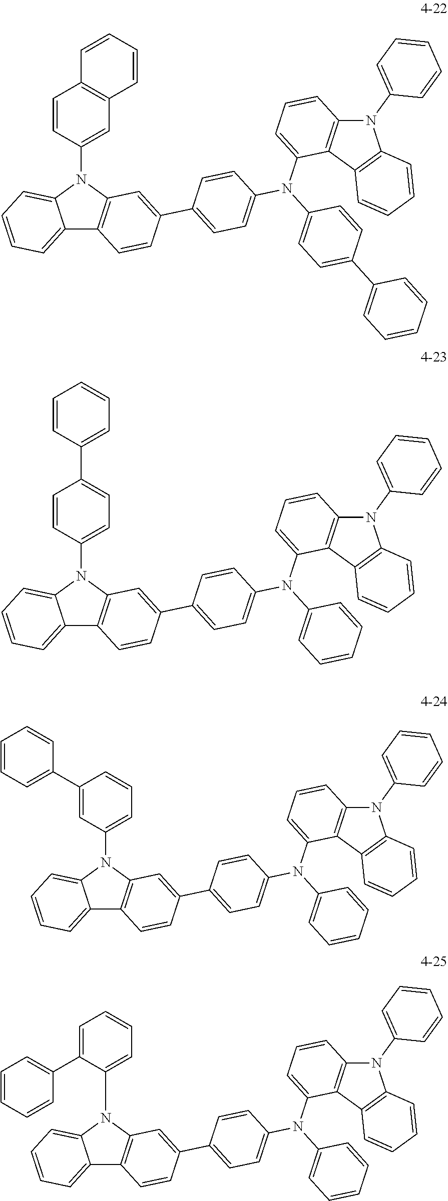 [SMILES: c1ccc(-c2ccc(N(c3ccc(-c4ccc5c6ccccc6n(-c6ccc7ccccc7c6)c5c4)cc3)c3cccc4c3c3ccccc3n4-c3ccccc3)cc2)cc1 ; c1ccc(-c2ccc(-n3c4ccccc4c4ccc(-c5ccc(N(c6ccccc6)c6cccc7c6c6ccccc6n7-c6ccccc6)cc5)cc43)cc2)cc1 ; c1ccc(-c2cccc(-n3c4ccccc4c4ccc(-c5ccc(N(c6ccccc6)c6cccc7c6c6ccccc6n7-c6ccccc6)cc5)cc43)c2)cc1 ; c1ccc(-c2ccccc2-n2c3ccccc3c3ccc(-c4ccc(N(c5ccccc5)c5cccc6c5c5ccccc5n6-c5ccccc5)cc4)cc32)cc1]

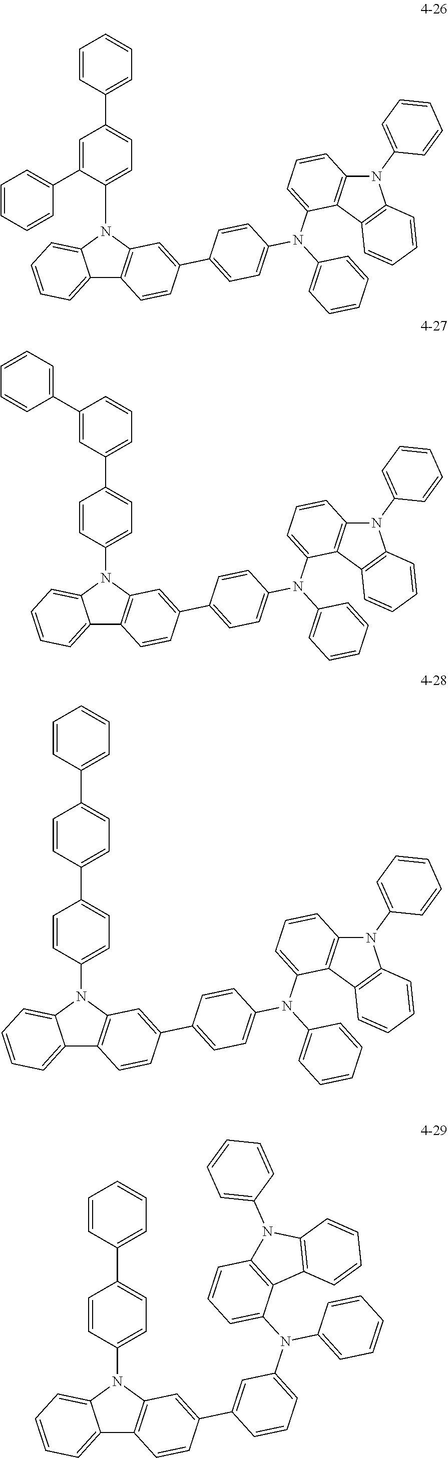 [SMILES: c1ccc(-c2ccc(-n3c4ccccc4c4ccc(-c5ccc(N(c6ccccc6)c6cccc7c6c6ccccc6n7-c6ccccc6)cc5)cc43)c(-c3ccccc3)c2)cc1 ; c1ccc(-c2cccc(-c3ccc(-n4c5ccccc5c5ccc(-c6ccc(N(c7ccccc7)c7cccc8c7c7ccccc7n8-c7ccccc7)cc6)cc54)cc3)c2)cc1 ; c1ccc(-c2ccc(-c3ccc(-n4c5ccccc5c5ccc(-c6ccc(N(c7ccccc7)c7cccc8c7c7ccccc7n8-c7ccccc7)cc6)cc54)cc3)cc2)cc1 ; c1ccc(-c2ccc(-n3c4ccccc4c4ccc(-c5cccc(N(c6ccccc6)c6cccc7c6c6ccccc6n7-c6ccccc6)c5)cc43)cc2)cc1]

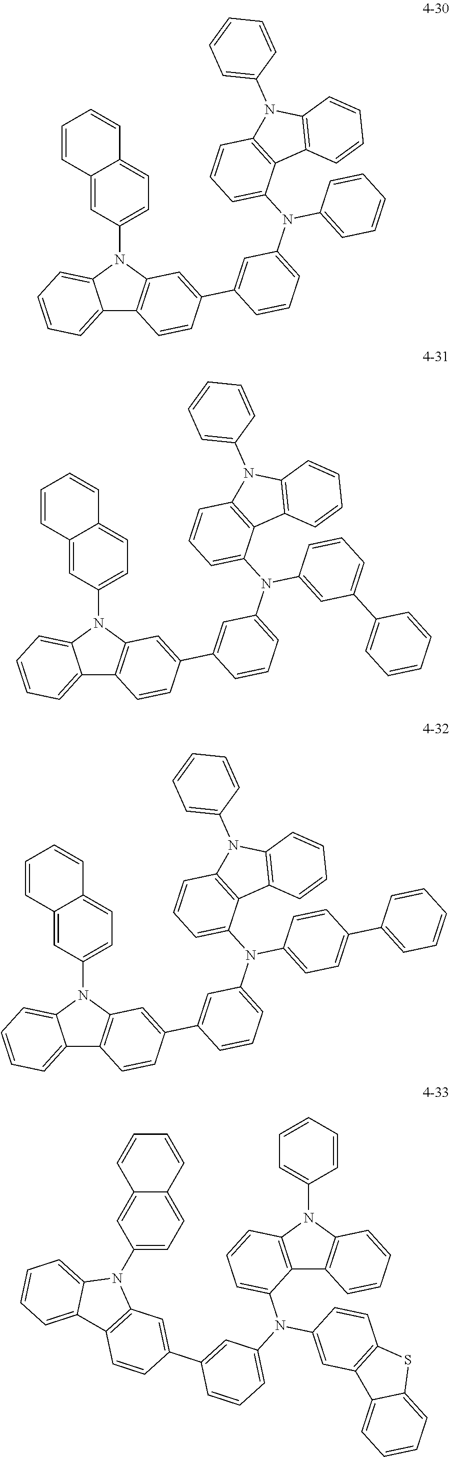 [SMILES: c1ccc(N(c2cccc(-c3ccc4c5ccccc5n(-c5ccc6ccccc6c5)c4c3)c2)c2cccc3c2c2ccccc2n3-c2ccccc2)cc1 ; c1ccc(-c2cccc(N(c3cccc(-c4ccc5c6ccccc6n(-c6ccc7ccccc7c6)c5c4)c3)c3cccc4c3c3ccccc3n4-c3ccccc3)c2)cc1 ; c1ccc(-c2ccc(N(c3cccc(-c4ccc5c6ccccc6n(-c6ccc7ccccc7c6)c5c4)c3)c3cccc4c3c3ccccc3n4-c3ccccc3)cc2)cc1 ; c1ccc(-n2c3ccccc3c3c(N(c4cccc(-c5ccc6c7ccccc7n(-c7ccc8ccccc8c7)c6c5)c4)c4ccc5sc6ccccc6c5c4)cccc32)cc1]

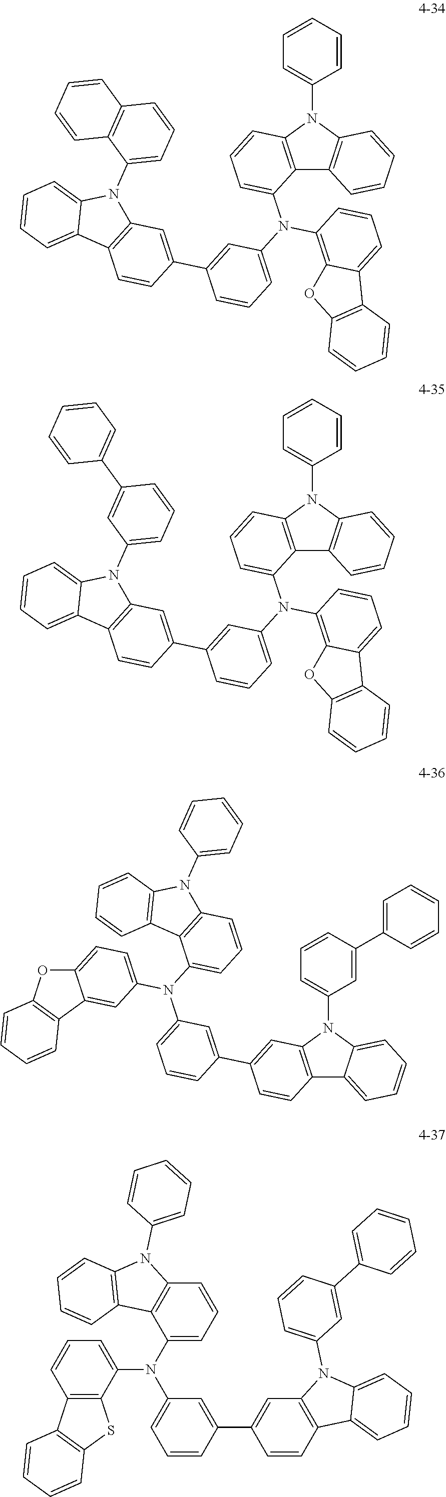 [SMILES: c1ccc(-n2c3ccccc3c3c(N(c4cccc(-c5ccc6c7ccccc7n(-c7cccc8ccccc78)c6c5)c4)c4cccc5c4oc4ccccc45)cccc32)cc1 ; c1ccc(-c2cccc(-n3c4ccccc4c4ccc(-c5cccc(N(c6cccc7c6oc6ccccc67)c6cccc7c6c6ccccc6n7-c6ccccc6)c5)cc43)c2)cc1 ; c1ccc(-c2cccc(-n3c4ccccc4c4ccc(-c5cccc(N(c6ccc7oc8ccccc8c7c6)c6cccc7c6c6ccccc6n7-c6ccccc6)c5)cc43)c2)cc1 ; c1ccc(-c2cccc(-n3c4ccccc4c4ccc(-c5cccc(N(c6cccc7c6sc6ccccc67)c6cccc7c6c6ccccc6n7-c6ccccc6)c5)cc43)c2)cc1]

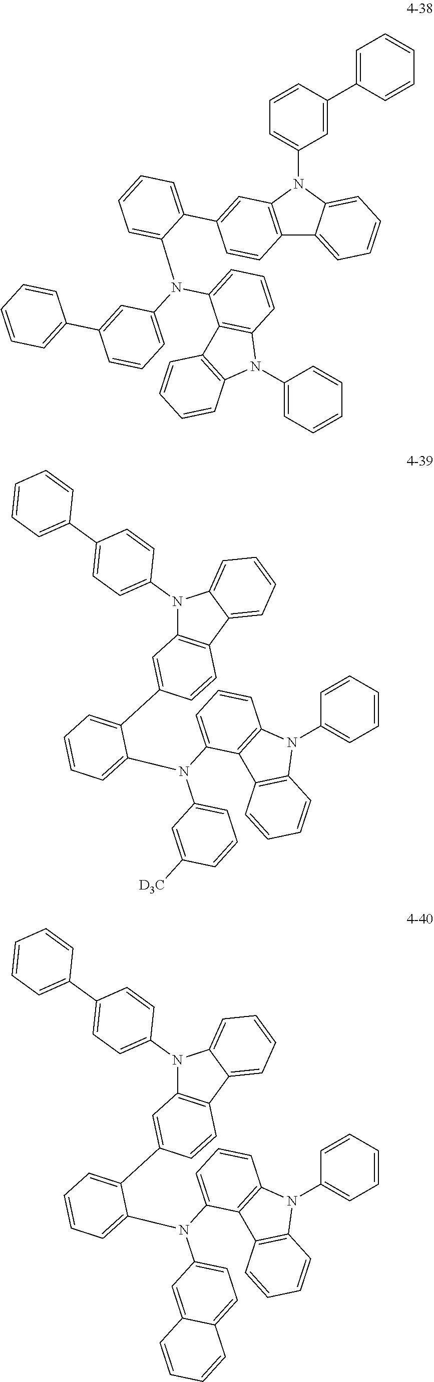 [SMILES: c1ccc(-c2cccc(N(c3ccccc3-c3ccc4c5ccccc5n(-c5cccc(-c6ccccc6)c5)c4c3)c3cccc4c3c3ccccc3n4-c3ccccc3)c2)cc1 ; [2H]C([2H])([2H])c1cccc(N(c2ccccc2-c2ccc3c4ccccc4n(-c4ccc(-c5ccccc5)cc4)c3c2)c2cccc3c2c2ccccc2n3-c2ccccc2)c1 ; c1ccc(-c2ccc(-n3c4ccccc4c4ccc(-c5ccccc5N(c5ccc6ccccc6c5)c5cccc6c5c5ccccc5n6-c5ccccc5)cc43)cc2)cc1]

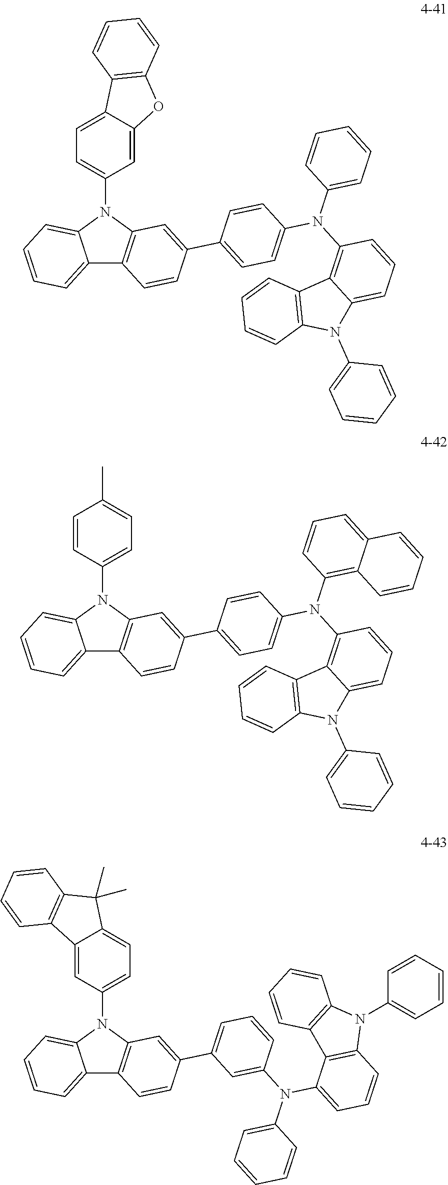 [SMILES: c1ccc(N(c2ccc(-c3ccc4c5ccccc5n(-c5ccc6c(c5)oc5ccccc56)c4c3)cc2)c2cccc3c2c2ccccc2n3-c2ccccc2)cc1 ; Cc1ccc(-n2c3ccccc3c3ccc(-c4ccc(N(c5cccc6ccccc56)c5cccc6c5c5ccccc5n6-c5ccccc5)cc4)cc32)cc1 ; CC1(C)c2ccccc2-c2cc(-n3c4ccccc4c4ccc(-c5cccc(N(c6ccccc6)c6cccc7c6c6ccccc6n7-c6ccccc6)c5)cc43)ccc21]

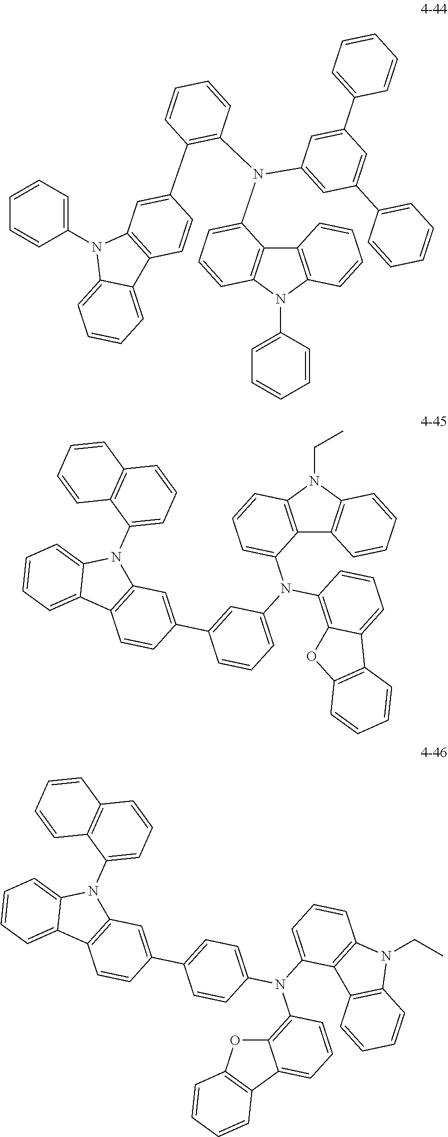 [SMILES: c1ccc(-c2cc(-c3ccccc3)cc(N(c3ccccc3-c3ccc4c5ccccc5n(-c5ccccc5)c4c3)c3cccc4c3c3ccccc3n4-c3ccccc3)c2)cc1 ; CCn1c2ccccc2c2c(N(c3cccc(-c4ccc5c6ccccc6n(-c6cccc7ccccc67)c5c4)c3)c3cccc4c3oc3ccccc34)cccc21 ; CCn1c2ccccc2c2c(N(c3ccc(-c4ccc5c6ccccc6n(-c6cccc7ccccc67)c5c4)cc3)c3cccc4c3oc3ccccc34)cccc21]

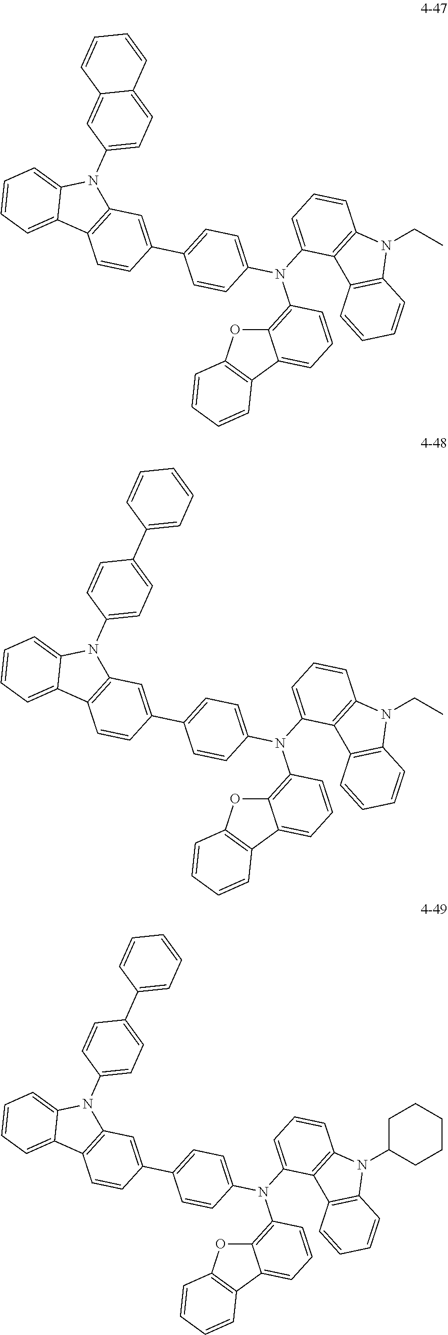 [SMILES: CCn1c2ccccc2c2c(N(c3ccc(-c4ccc5c6ccccc6n(-c6ccc7ccccc7c6)c5c4)cc3)c3cccc4c3oc3ccccc34)cccc21 ; CCn1c2ccccc2c2c(N(c3ccc(-c4ccc5c6ccccc6n(-c6ccc(-c7ccccc7)cc6)c5c4)cc3)c3cccc4c3oc3ccccc34)cccc21 ; c1ccc(-c2ccc(-n3c4ccccc4c4ccc(-c5ccc(N(c6cccc7c6oc6ccccc67)c6cccc7c6c6ccccc6n7C6CCCCC6)cc5)cc43)cc2)cc1]

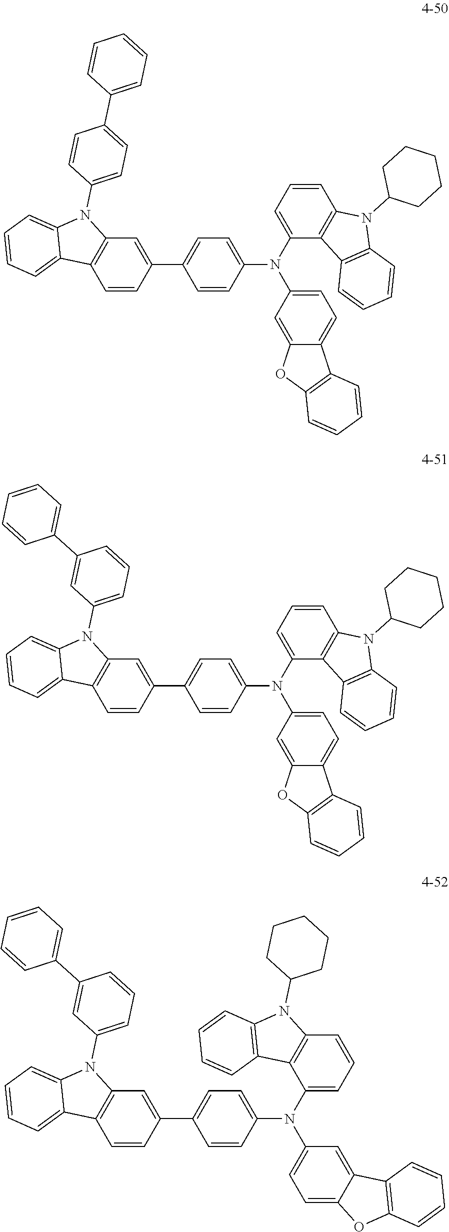 [SMILES: c1ccc(-c2ccc(-n3c4ccccc4c4ccc(-c5ccc(N(c6ccc7c(c6)oc6ccccc67)c6cccc7c6c6ccccc6n7C6CCCCC6)cc5)cc43)cc2)cc1 ; c1ccc(-c2cccc(-n3c4ccccc4c4ccc(-c5ccc(N(c6ccc7c(c6)oc6ccccc67)c6cccc7c6c6ccccc6n7C6CCCCC6)cc5)cc43)c2)cc1 ; c1ccc(-c2cccc(-n3c4ccccc4c4ccc(-c5ccc(N(c6ccc7oc8ccccc8c7c6)c6cccc7c6c6ccccc6n7C6CCCCC6)cc5)cc43)c2)cc1]

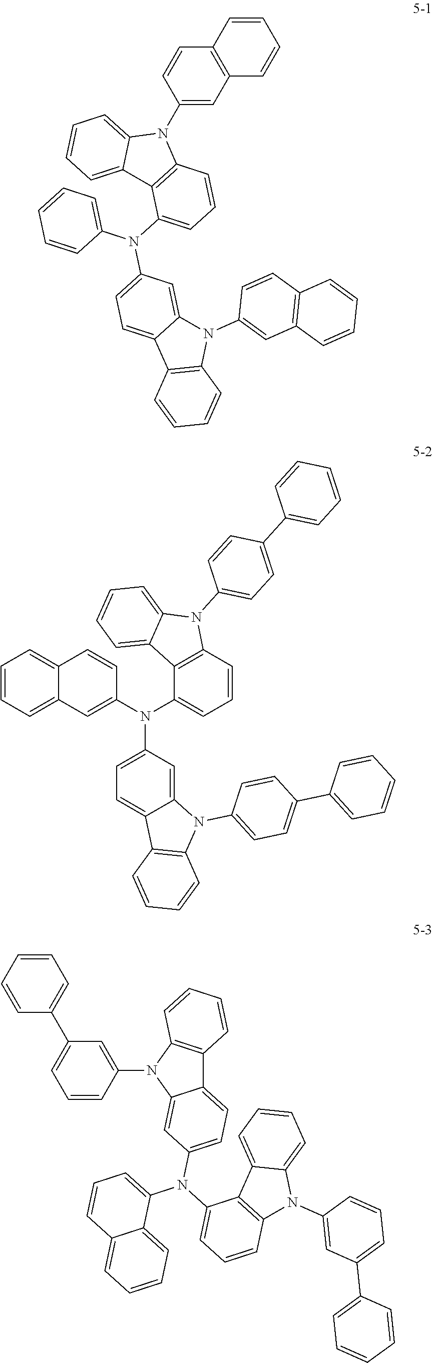 [SMILES: c1ccc(N(c2ccc3c4ccccc4n(-c4ccc5ccccc5c4)c3c2)c2cccc3c2c2ccccc2n3-c2ccc3ccccc3c2)cc1 ; c1ccc(-c2ccc(-n3c4ccccc4c4ccc(N(c5ccc6ccccc6c5)c5cccc6c5c5ccccc5n6-c5ccc(-c6ccccc6)cc5)cc43)cc2)cc1 ; c1ccc(-c2cccc(-n3c4ccccc4c4ccc(N(c5cccc6ccccc56)c5cccc6c5c5ccccc5n6-c5cccc(-c6ccccc6)c5)cc43)c2)cc1]

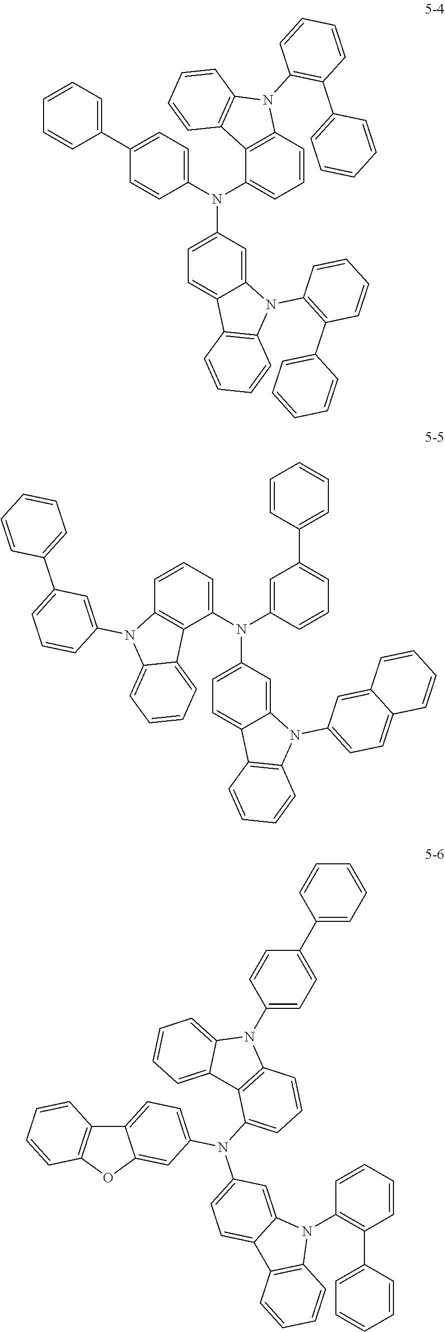

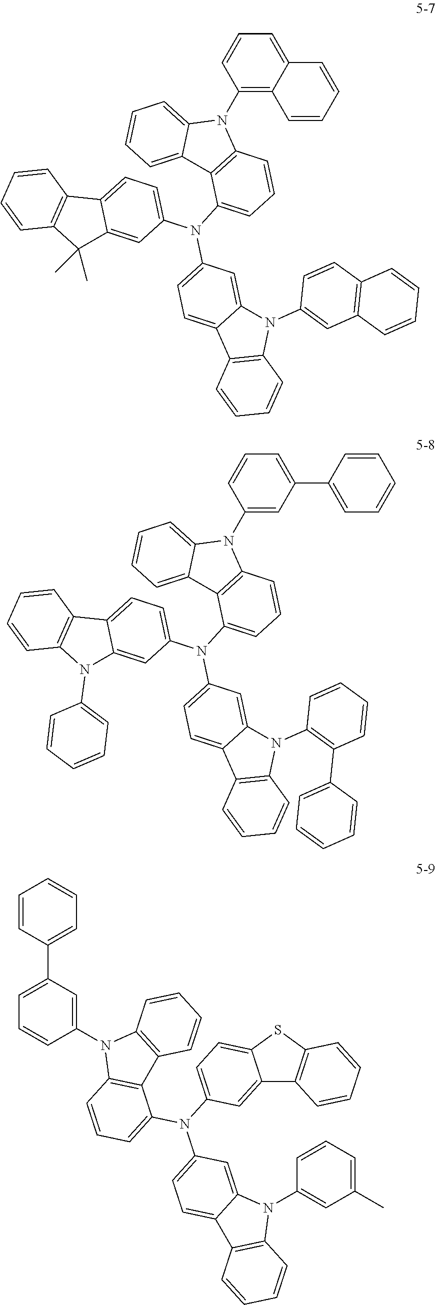

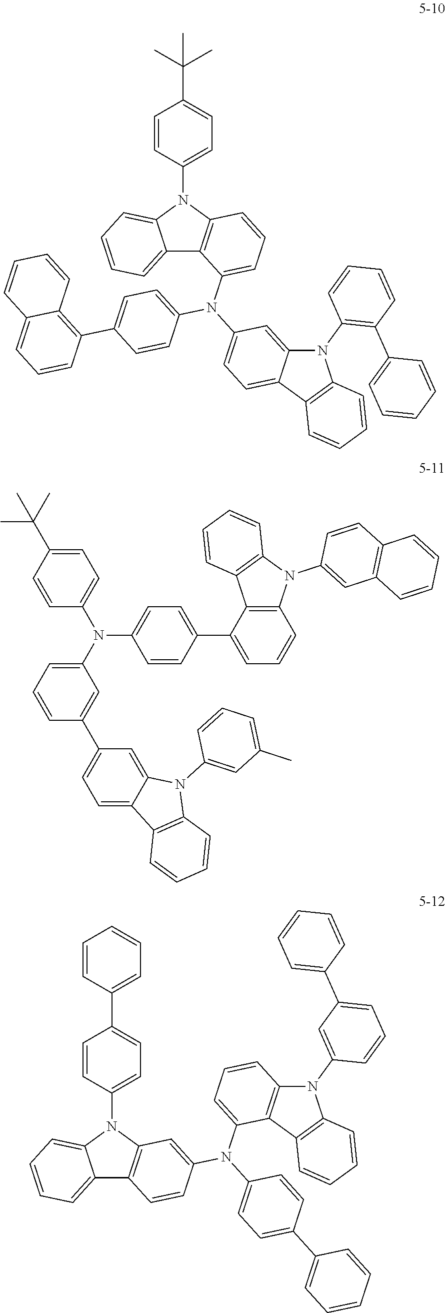 [SMILES: CC(C)(C)c1ccc(-n2c3ccccc3c3c(N(c4ccc(-c5cccc6ccccc56)cc4)c4ccc5c6ccccc6n(-c6ccccc6-c6ccccc6)c5c4)cccc32)cc1 ; Cc1cccc(-n2c3ccccc3c3ccc(-c4cccc(N(c5ccc(-c6cccc7c6c6ccccc6n7-c6ccc7ccccc7c6)cc5)c5ccc(C(C)(C)C)cc5)c4)cc32)c1 ; c1ccc(-c2ccc(N(c3ccc4c5ccccc5n(-c5ccc(-c6ccccc6)cc5)c4c3)c3cccc4c3c3ccccc3n4-c3cccc(-c4ccccc4)c3)cc2)cc1]

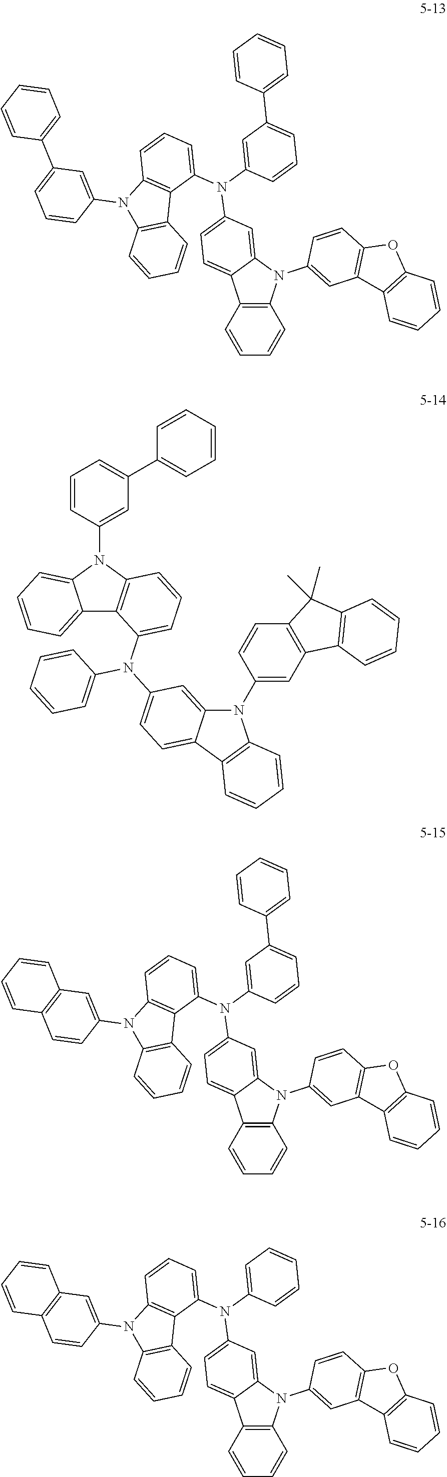 [SMILES: c1ccc(-c2cccc(N(c3ccc4c5ccccc5n(-c5ccc6oc7ccccc7c6c5)c4c3)c3cccc4c3c3ccccc3n4-c3cccc(-c4ccccc4)c3)c2)cc1 ; CC1(C)c2ccccc2-c2cc(-n3c4ccccc4c4ccc(N(c5ccccc5)c5cccc6c5c5ccccc5n6-c5cccc(-c6ccccc6)c5)cc43)ccc21 ; c1ccc(-c2cccc(N(c3ccc4c5ccccc5n(-c5ccc6oc7ccccc7c6c5)c4c3)c3cccc4c3c3ccccc3n4-c3ccc4ccccc4c3)c2)cc1 ; c1ccc(N(c2ccc3c4ccccc4n(-c4ccc5oc6ccccc6c5c4)c3c2)c2cccc3c2c2ccccc2n3-c2ccc3ccccc3c2)cc1]

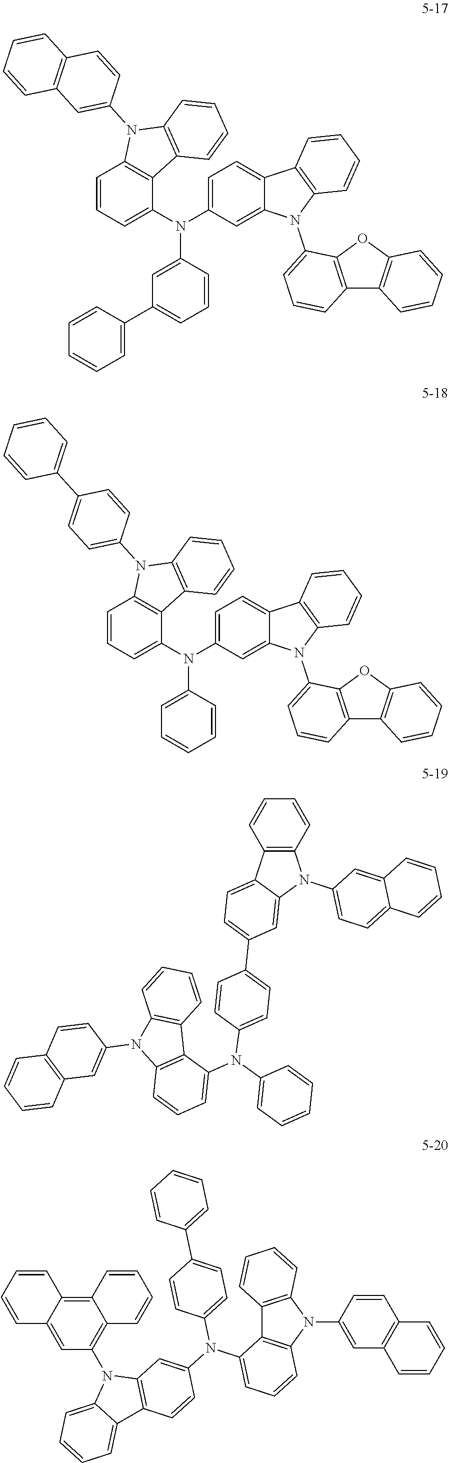 [SMILES: c1ccc(-c2cccc(N(c3ccc4c5ccccc5n(-c5cccc6c5oc5ccccc56)c4c3)c3cccc4c3c3ccccc3n4-c3ccc4ccccc4c3)c2)cc1 ; c1ccc(-c2ccc(-n3c4ccccc4c4c(N(c5ccccc5)c5ccc6c7ccccc7n(-c7cccc8c7oc7ccccc78)c6c5)cccc43)cc2)cc1 ; c1ccc(N(c2ccc(-c3ccc4c5ccccc5n(-c5ccc6ccccc6c5)c4c3)cc2)c2cccc3c2c2ccccc2n3-c2ccc3ccccc3c2)cc1 ; c1ccc(-c2ccc(N(c3ccc4c5ccccc5n(-c5cc6ccccc6c6ccccc56)c4c3)c3cccc4c3c3ccccc3n4-c3ccc4ccccc4c3)cc2)cc1]

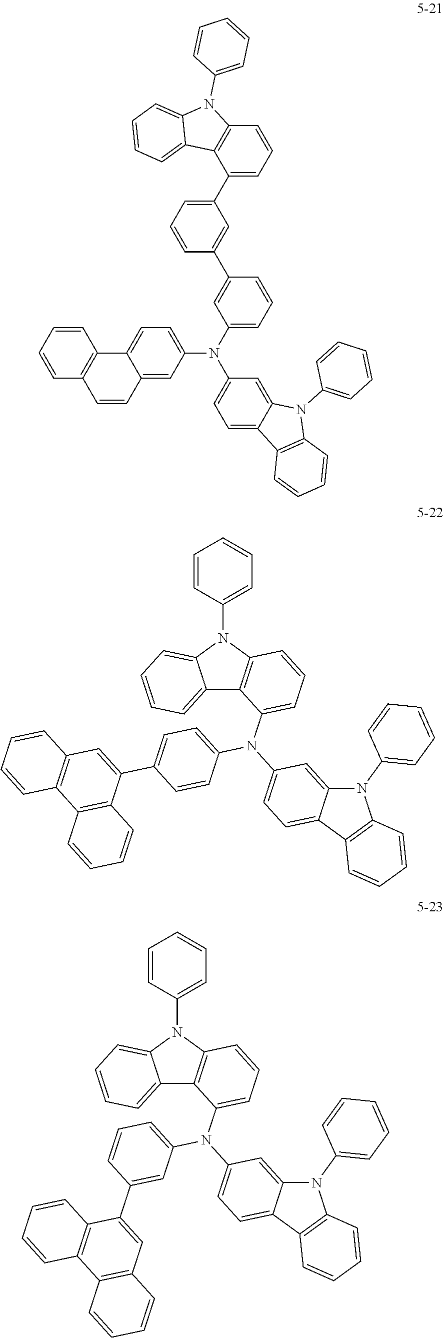 [SMILES: c1ccc(-n2c3ccccc3c3ccc(N(c4cccc(-c5cccc(-c6cccc7c6c6ccccc6n7-c6ccccc6)c5)c4)c4ccc5c(ccc6ccccc65)c4)cc32)cc1 ; c1ccc(-n2c3ccccc3c3ccc(N(c4ccc(-c5cc6ccccc6c6ccccc56)cc4)c4cccc5c4c4ccccc4n5-c4ccccc4)cc32)cc1 ; c1ccc(-n2c3ccccc3c3ccc(N(c4cccc(-c5cc6ccccc6c6ccccc56)c4)c4cccc5c4c4ccccc4n5-c4ccccc4)cc32)cc1]

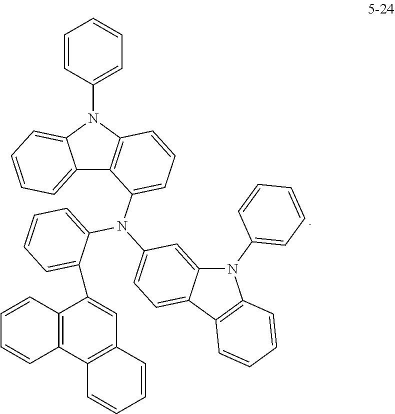 [SMILES: c1ccc(-n2c3ccccc3c3ccc(N(c4ccccc4-c4cc5ccccc5c5ccccc45)c4cccc5c4c4ccccc4n5-c4ccccc4)cc32)cc1]

The synthesis method of the provided organic compound of the present disclosure is not particularly limited, those skilled in the art may determine a suitable synthesis method according to the preparation method provided in the organic compound combination synthesis example portion of the present disclosure. In other words, the synthesis examples of the present disclosure provide an exemplary method for preparing the organic compounds, and the raw materials employed may be commercially available or obtained by methods well known in the art. Those skilled in the art may obtain all organic compounds provided in the present disclosure according to these exemplary preparation methods, and not all of the specific preparation methods for preparing the organic compound will be described in detail herein, and it should not be understood by those skilled in the art as limitations on the present disclosure.

The second aspect of the present disclosure provides an electronic element, including an anode, a cathode, and a functional layer disposed between the anode and the cathode, wherein the functional layer comprises an organic compound described in the first aspect of the present disclosure.

Optionally, the functional layer comprises a hole transport layer, where the hole transport layer comprises an organic compound of the present disclosure.

In the present disclosure, the electronic element may be an organic electroluminescent device or a photoelectric conversion device.

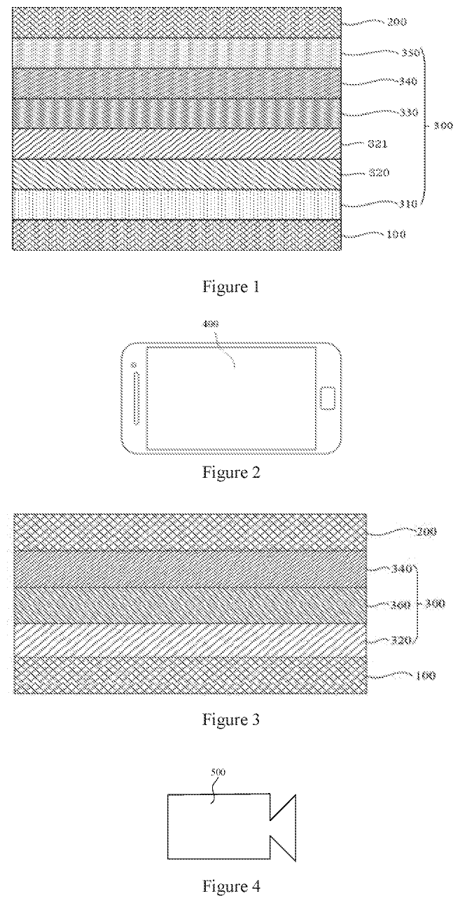

According to a specific embodiment, the electronic element is an organic electroluminescent device. As shown in FIG. 1, the organic electroluminescent device may include an anode 100, a hole transport layer 320, an organic light-emitting layer 330, an electron transport layer 340 and a cathode 200 disposed sequentially.

In the present disclosure, the anode 100 includes an anode material, which preferably has a large work function and promotes the hole injection into the functional layer 300. Specific examples of the anode material include, but not limited to: metals such as nickel, platinum, vanadium, chromium, copper, zinc and gold or alloys thereof; metal oxides such as zinc oxide, indium oxide, indium tin oxide (ITO) and zinc indium oxide (IZO); combinations of metals and oxides such as ZnO:Al or SnO₂:Sb; or conductive polymers such as poly(3-methylthiophene), poly[3,4-(ethylene-1,2-dioxy)thiophene] (PEDT), polypyrrole and polyaniline. Preferably a transparent electrode comprising indium tin oxide (ITO) is included as the anode.

Optionally, the hole transport layer 320 comprises an organic compound of the present disclosure.

Optionally, an electron barrier layer 321 (also referred to as a “hole-adjustment layer”) is further provided between the hole transport layer 320 and the organic light emitting-layer 330. The material of the electron barrier layer 321 may be selected from the group consisting of carbazole polymer, carbazole-linked aromatic amine compounds, substituted fluorene-linked aromatic amine compounds, and other types of compounds, and the present disclosure does not impose any special limitation on this. For example, the material of the electron barrier layer 321 is selected from the group consisting of the following compounds:

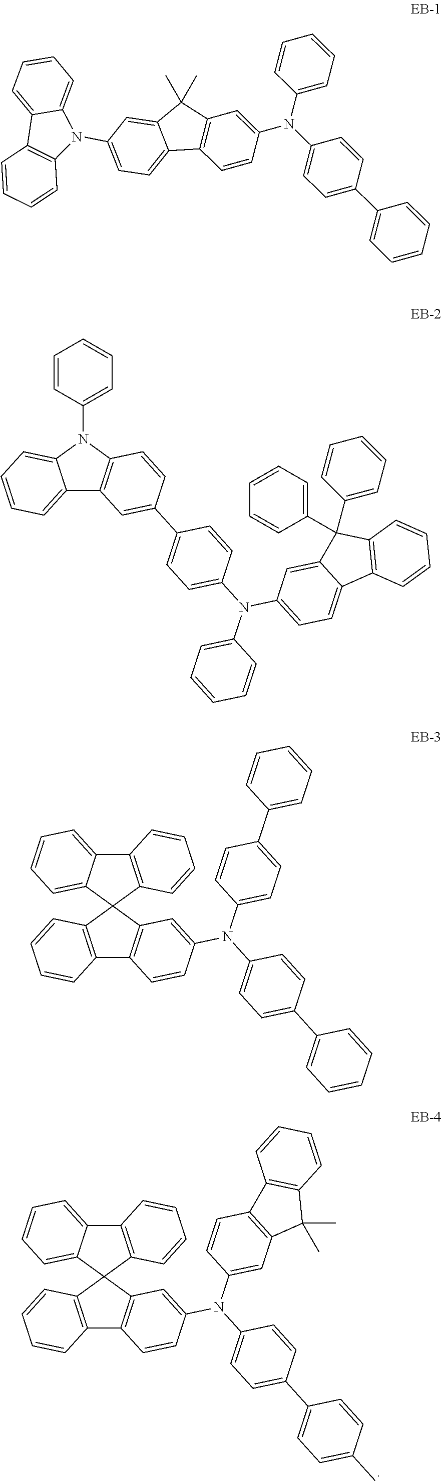

In a specific embodiment, the material of the electron barrier layer 321 is EB-3.

Organic light-emitting layer 330 may be composed of a single light-emitting material, or may include a host material and a dopant material. Optionally, the organic light-emitting layer 330 is composed of a host material and a dopant material. Holes injected into the organic light-emitting layer 330 and electrons injected into the organic light emitting-layer 330 may be combined within the organic light-emitting layer 330 to form excitons. The exciton transmits energy to the host material, and the host material transmits energy to the dopant material, thereby enabling the dopant material to emit light.

The host material of the organic light-emitting layer 330 may be metal chelating compounds, stilbene derivatives, aromatic amine derivatives, dibenzofuran derivatives or other types of materials, and the present disclosure does not impose any special limitation on this. For example, the host material is α, β-ADN or PCAN.

The dopant material of the organic light-emitting layer 330 may be a compound having a condensed aryl ring or derivatives thereof, a compound having a heteroaryl ring or derivatives thereof, aromatic amine derivatives or other materials, and the present disclosure does not impose any special limitation on this. For example, the dopant material is BD-1.

Electron transport layer 340 may be a single-layer structure, may also be a multilayer structure, which may include one or more electron transport materials. The electron transport material may typically comprise a metal complex and/or nitrogen-containing heterocyclic derivative, wherein the metal complex material may for example be selected from LiQ, Alq₃, Bepq₂, and the like; the nitrogen-containing heterocyclic derivative may be an aromatic ring having a nitrogen-containing six-membered ring or a five-membered ring skeleton, a fused aromatic ring compound having a nitrogen-containing six-membered ring or a five-membered ring skeleton, and the like. Specific examples include, but not limited to, BCP, Bphen, NBphen, DBimiBphen, BimiBphen and other 1,10-phenanthroline compounds. In a specific embodiment, the electron transport layer 340 is composed of BCP and LiQ.

Cathode 200 includes a cathode material, which is a material that helps electrons inject into the functional layer 300 with a small escape work. Specific examples of cathode materials include, but not limited to, metals such as magnesium, calcium, sodium, potassium, titanium, indium, yttrium, lithium, gadolinium, aluminum, silver, tin and lead or alloys thereof; or multilayer materials such as LiF/Al, Liq/Al, LiO₂/Al, LiF/Ca, LiF/Al and BaF₂/Ca. Preferably, a metal electrode comprising magnesium and silver is used as the cathode.

Optionally, as shown in FIG. 1, a hole injection layer 310 is further provided between the anode 100 and the hole transport layer 320 to enhance the ability to inject holes into the hole transport layer 320. The hole injection layer 310 may comprise a material selected from the group consisting of benzidine derivatives, starburst aryl amine compounds, phthalocyanine derivatives and other materials, and the present disclosure does not impose any special limitation on this. For example, the hole injection layer 310 consists of F4-TCNQ.

Optionally, as shown in FIG. 1, an electron injection layer 350 is further provided between the cathode 200 and the electron transport layer 340 to enhance the ability to inject electrons into the electron transport layer 340. The electron injection layer 350 may include inorganic materials such as alkali metal sulfides, alkali metal halides, or may include complexes of alkali metals with organic compounds. For example, the electron injection layer 350 contains LiQ or Yb.

In the present disclosure, the organic electroluminescent device may be a blue light device, a red light device or a green light device, preferably a blue light device.

According to another embodiment, the electronic element is a photoelectric conversion device. As shown in FIG. 3, the photoelectric conversion device may include an anode 100, a hole transport layer 320, a photoelectric conversion layer 360, an electron transport layer 340 and a cathode 200 disposed sequentially. Wherein the hole transport layer 320 comprises an organic compound of the present disclosure.

In the present disclosure, the photoelectric conversion device may be a solar cell, for example, an organic thin-film solar cell.

The third aspect of the present disclosure provides an electronic apparatus, comprising the electronic element described in the second aspect of the present disclosure.

According to an embodiment, as shown in FIG. 2, the electronic apparatus is a first electronic apparatus 400, comprising the above-mentioned organic electroluminescent device. The first electronic apparatus 400 may for example be a display apparatus, lighting apparatus, optical communication apparatus or other type of electronic apparatus, and for example may include, but not limited to, a computer screen, mobile phone screen, television, electronic paper, emergency lighting, optical module, etc.

According to another embodiment, as shown in FIG. 4, the electronic apparatus is a second electronic apparatus 500 comprising the above-mentioned photoelectric conversion device. The second electronic apparatus 500 may for example be a solar power generation equipment, a light detector, a fingerprint recognition equipment, an optical module, a CCD camera or other types of electronic apparatuses.

The present disclosure is further described in detail in Examples. However, the following examples are only illustration of the present disclosure, and do not intend to limit the present disclosure.

The compounds whose synthesis methods are not mentioned in this disclosure are commercially available raw material products.

### Synthesis Example 1: Synthesis of Compound 1-1

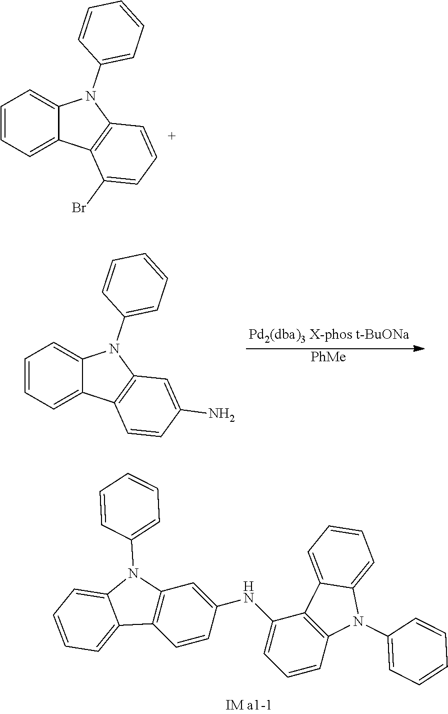

(1) 4-bromo-N-phenylcarbazole (50.0 g, 155.18 mmol), 9-phenyl-9H-carbazol-2-amine (44.1 g, 170.70 mmol), tris(dibenzylideneacetone)dipalladium (1.42 g, 1.55 mmol), 2-dicyclohexylphosphino-2′,4′,6′-triisopropylbiphenyl (1.48 g, 3.1 mmol), sodium tert-butoxide (22.37 g, 232.77 mmol) and toluene (500 mL) were added to a round bottom flask, and stirred at 108° C. to react for 4 h under the protection of nitrogen. After completion of the reaction, the resulting reaction solution was cooled to room temperature, washed with water and separated, obtaining an organic phase after separation. The obtained organic phase was dried with anhydrous magnesium sulfate, and then removed the solvent under reduced pressure to obtain a crude product. The obtained crude product was purified by silica gel column chromatography using a mixture of dichloromethane and n-heptane as an eluating agent, to obtain a white solid compound IM a1-1 (62.4 g, yield: 80.5%).

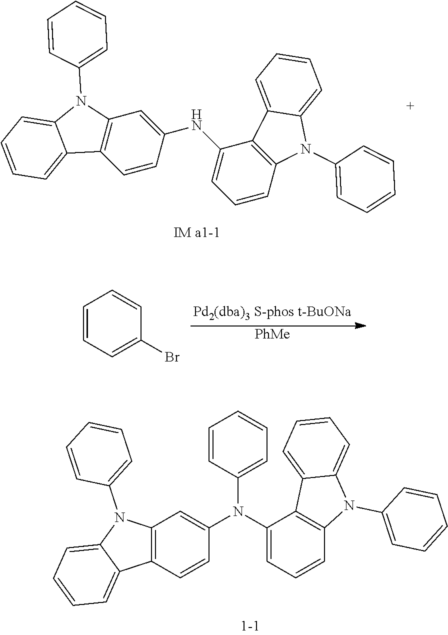 [SMILES: c1ccc(-n2c3ccccc3c3ccc(Nc4cccc5c4c4ccccc4n5-c4ccccc4)cc32)cc1 ; Brc1ccccc1 ; "PhMe" ; c1ccc(N(c2ccc3c4ccccc4n(-c4ccccc4)c3c2)c2cccc3c2c2ccccc2n3-c2ccccc2)cc1]

(2) IM a1-1 (5.0 g, 10.0 mmol), bromobenzene (1.7 g, 11.0 mmol), tris(dibenzylideneacetone)dipalladium (0.09 g, 0.10 mmol), 2-dicyclohexylphosphino-2′,6′-dimethoxy-biphenyl (0.08 g, 0.2 mmol), sodium tert-butoxide (1.44 g, 15.01 mol) and toluene (50 mL) were added to a round bottom flask, and stirred at 108° C. to react for 2 h under the protection of nitrogen. After completion of the reaction, the resulting reaction solution was cooled to room temperature, washed with water and then separated to obtain an organic phase. The obtained organic phase was dried with anhydrous magnesium sulfate, and then removed the solvent under reduced pressure to obtain a crude product. The obtained crude product was purified by silica gel column chromatography using a mixture of dichloromethane and n-heptane as an eluating agent, to obtain a white solid compound 1-1 (4.8 g, yield: 83%), mass spectrometry (m/z)=576.2 [M+H]⁺; NMR data for compound 1-1: ¹H NMR (400 MHZ, CD2Cl2): 8.19 (m, 3H), 8.02 (d, 1H), 7.77-7.41 (m, 18H), 7.31 (t, 1H), 7.20 (s, 1H), 7.05-7.01 (d, 2H), 6.88-6.79 (m, 2H), 6.65 (d, 1H).

### Synthesis Example 2 to Synthesis Example 10, and Synthesis Example 36

The following compounds were synthesized with reference to the synthesis method of compound 1-1, except that bromobenzene was replaced with reactant A. The reactant A, the synthesized compound, the yield and mass spectrometry result thereof are shown in Table 1.

TABLE 1

| | | | | Mass |
|---|---|---|---|---|
| | | | | spectrometry |
| Synthesis | | | | (m/z), |
| Example | Reactant A | Compound | Yield (%) | [M + H]⁺ |
| 2 | 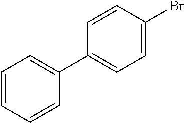 [SMILES: Brc1ccc(-c2ccccc2)cc1] | 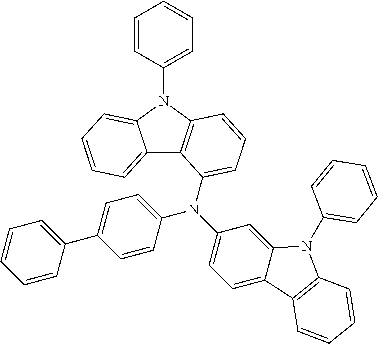 [SMILES: c1ccc(-c2ccc(N(c3ccc4c5ccccc5n(-c5ccccc5)c4c3)c3cccc4c3c3ccccc3n4-c3ccccc3)cc2)cc1] | 83 | 652.3 |
| 3 | 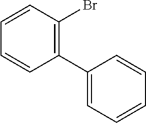 [SMILES: Brc1ccccc1-c1ccccc1] | 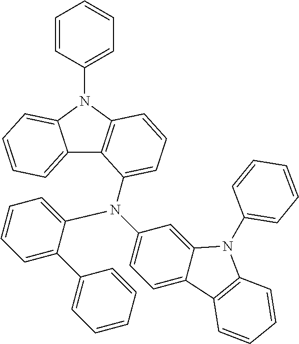 [SMILES: c1ccc(-c2ccccc2N(c2ccc3c4ccccc4n(-c4ccccc4)c3c2)c2cccc3c2c2ccccc2n3-c2ccccc2)cc1] | 79 | 652.3 |
| 4 | 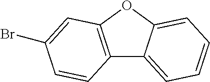 [SMILES: Brc1ccc2c(c1)oc1ccccc12] | 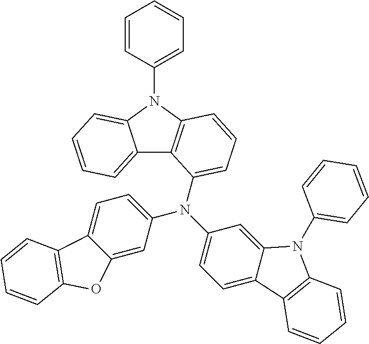 [SMILES: c1ccc(-n2c3ccccc3c3ccc(N(c4ccc5c(c4)oc4ccccc45)c4cccc5c4c4ccccc4n5-c4ccccc4)cc32)cc1] | 84 | 666.3 |
| 5 | 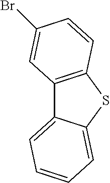 [SMILES: Brc1ccc2sc3ccccc3c2c1] | 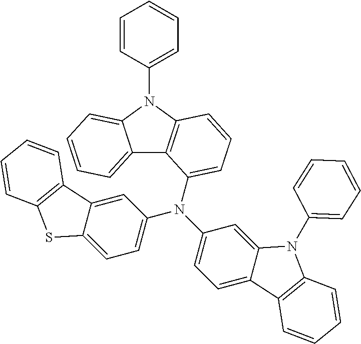 [SMILES: c1ccc(-n2c3ccccc3c3ccc(N(c4ccc5sc6ccccc6c5c4)c4cccc5c4c4ccccc4n5-c4ccccc4)cc32)cc1] | 76 | 682.2 |
| 6 | 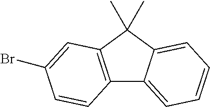 [SMILES: CC1(C)c2ccccc2-c2ccc(Br)cc21] | 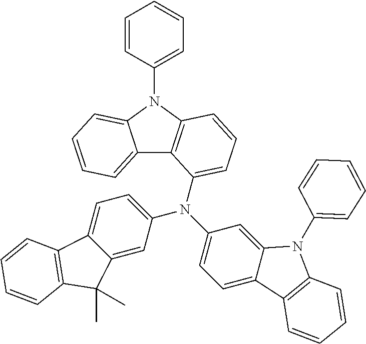 [SMILES: CC1(C)c2ccccc2-c2ccc(N(c3ccc4c5ccccc5n(-c5ccccc5)c4c3)c3cccc4c3c3ccccc3n4-c3ccccc3)cc21] | 87 | 692.3 |
| 7 | 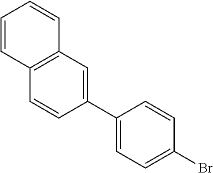 [SMILES: Brc1ccc(-c2ccc3ccccc3c2)cc1] | 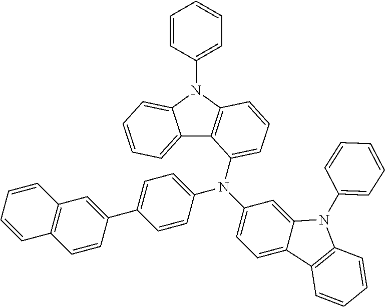 [SMILES: c1ccc(-n2c3ccccc3c3ccc(N(c4ccc(-c5ccc6ccccc6c5)cc4)c4cccc5c4c4ccccc4n5-c4ccccc4)cc32)cc1] | 85 | 702.3 |
| 8 | 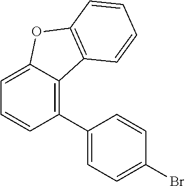 [SMILES: Brc1ccc(-c2cccc3oc4ccccc4c23)cc1] | 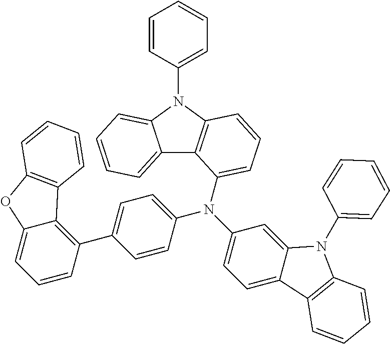 [SMILES: c1ccc(-n2c3ccccc3c3ccc(N(c4ccc(-c5cccc6oc7ccccc7c56)cc4)c4cccc5c4c4ccccc4n5-c4ccccc4)cc32)cc1] | 82 | 742.3 |
| 9 | 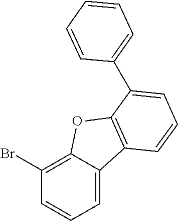 [SMILES: Brc1cccc2c1oc1c(-c3ccccc3)cccc12] | 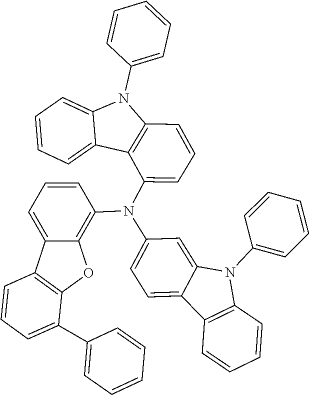 [SMILES: c1ccc(-c2cccc3c2oc2c(N(c4ccc5c6ccccc6n(-c6ccccc6)c5c4)c4cccc5c4c4ccccc4n5-c4ccccc4)cccc23)cc1] | 76 | 742.3 |
| 10 | 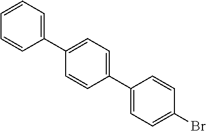 [SMILES: Brc1ccc(-c2ccc(-c3ccccc3)cc2)cc1] | 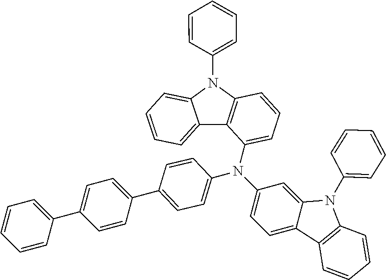 [SMILES: c1ccc(-c2ccc(-c3ccc(N(c4ccc5c6ccccc6n(-c6ccccc6)c5c4)c4cccc5c4c4ccccc4n5-c4ccccc4)cc3)cc2)cc1] | 80 | 728.3 |
| 36 | 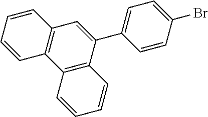 [SMILES: Brc1ccc(-c2cc3ccccc3c3ccccc23)cc1] | 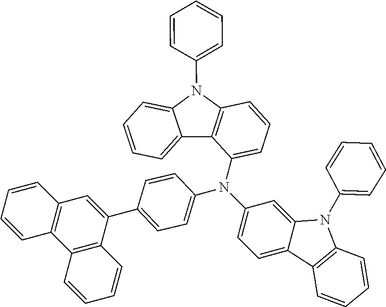 [SMILES: c1ccc(-n2c3ccccc3c3ccc(N(c4ccc(-c5cc6ccccc6c6ccccc56)cc4)c4cccc5c4c4ccccc4n5-c4ccccc4)cc32)cc1] | 77 | 752.3 |

### Synthesis of Intermediate IM b1-X

The synthesis of IM b1-X is illustrated by taking IM b1-1 as an example.

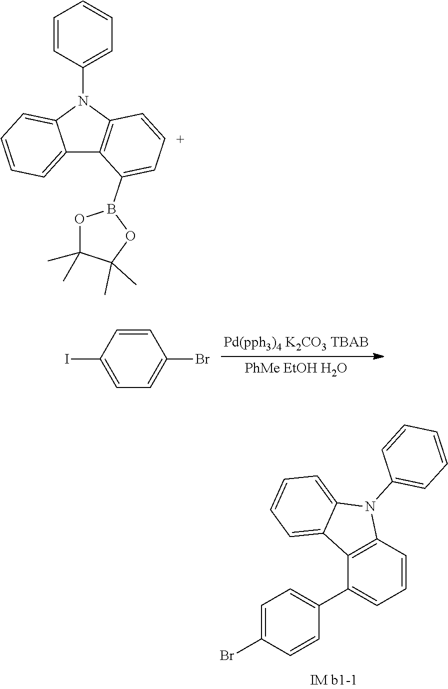

(9-phenyl-9H-carbazol-4-yl) boronic acid pinacol ester (10.0 g, 27.08 mmol), p-bromoiodobenzene (8.43 g, 29.79 mmol), tetrakis(triphenylphosphino) palladium (0.31 g, 0.27 mmol), potassium carbonate (7.47 g, 54.16 mmol), tetrabutylammonium bromide (1.75 g, 5.42 mmol), toluene (60 mL), anhydrous ethanol (20 mL) and deionized water (20 mL) were added to a round bottom flask, and stirred at 78° C. to react for 5 h under the protection of nitrogen. After completion of the reaction, the resulting reaction solution was cooled to room temperature, washed with water and then separated to obtain an organic phase. The obtained organic phase was dried with anhydrous magnesium sulfate, and then removed the solvent under reduced pressure to obtain a crude product. The obtained crude product was purified by using a mixture of dichloromethane and n-heptane (volume ratio 1/2) as the recrystallization solvent to gain a gray solid, namely IM b1-1 (7.4 g, yield: 68.6%).

The other IM b1-X compounds listed in Table 2 were synthesized with reference to the synthesis method of IM b1-1, except that (9-phenyl-9H-carbazol-4-yl) boronic acid pinacol ester was replaced with reactant B, and p-bromoiodobenzene was replaced with reactant C listed in Table 2.

TABLE 2

| Reactant B | Reactant C | IM b1-X | Yield (%) |
|---|---|---|---|
| 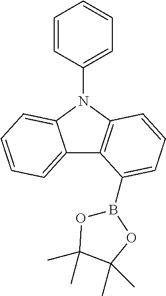 [SMILES: CC1(C)OB(c2cccc3c2c2ccccc2n3-c2ccccc2)OC1(C)C] | 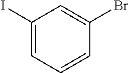 | 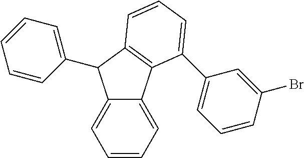 [SMILES: Brc1cccc(-c2cccc3c2-c2ccccc2C3c2ccccc2)c1] | 76 |
| | 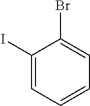 | 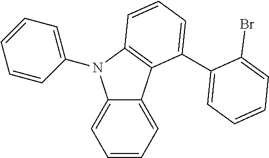 [SMILES: Brc1ccccc1-c1cccc2c1c1ccccc1n2-c1ccccc1] | 59 |
| | 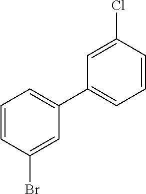 [SMILES: Clc1cccc(-c2cccc(Br)c2)c1] | 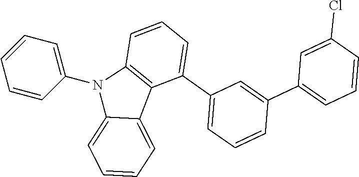 [SMILES: Clc1cccc(-c2cccc(-c3cccc4c3c3ccccc3n4-c3ccccc3)c2)c1] | 65 |
| | 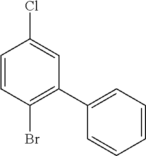 [SMILES: Clc1ccc(Br)c(-c2ccccc2)c1] | 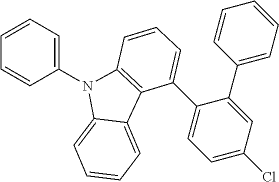 [SMILES: Clc1ccc(-c2cccc3c2c2ccccc2n3-c2ccccc2)c(-c2ccccc2)c1] | 71 |
| 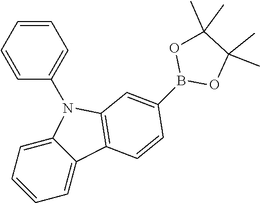 [SMILES: CC1(C)OB(c2ccc3c4ccccc4n(-c4ccccc4)c3c2)OC1(C)C] | 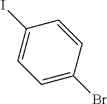 | 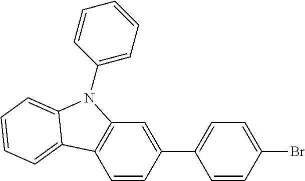 [SMILES: Brc1ccc(-c2ccc3c4ccccc4n(-c4ccccc4)c3c2)cc1] | 70 |
| | 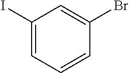 | 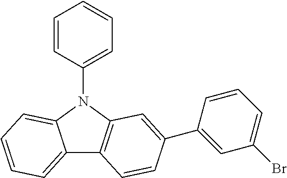 [SMILES: Brc1cccc(-c2ccc3c4ccccc4n(-c4ccccc4)c3c2)c1] | 67 |
| | 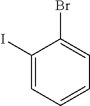 | 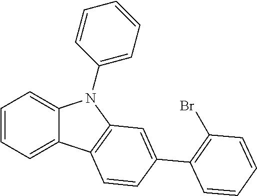 [SMILES: Brc1ccccc1-c1ccc2c3ccccc3n(-c3ccccc3)c2c1] | 63 |
| | 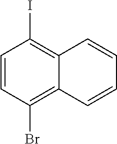 [SMILES: Brc1ccc(I)c2ccccc12] | 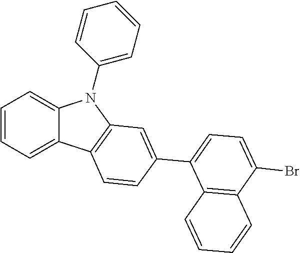 [SMILES: Brc1ccc(-c2ccc3c4ccccc4n(-c4ccccc4)c3c2)c2ccccc12] | 73 |
| | 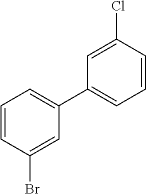 [SMILES: Clc1cccc(-c2cccc(Br)c2)c1] | 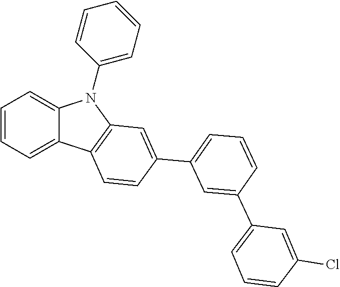 [SMILES: Clc1cccc(-c2cccc(-c3ccc4c5ccccc5n(-c5ccccc5)c4c3)c2)c1] | 66 |
| 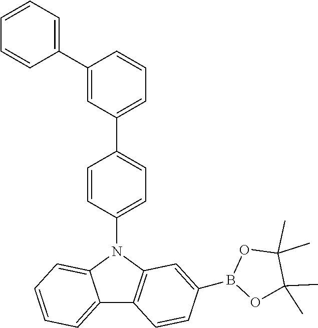 [SMILES: CC1(C)OB(c2ccc3c4ccccc4n(-c4ccc(-c5cccc(-c6ccccc6)c5)cc4)c3c2)OC1(C)C] | 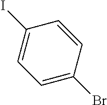 | 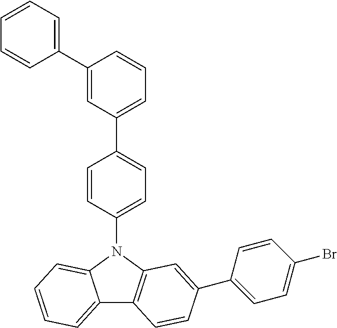 [SMILES: Brc1ccc(-c2ccc3c4ccccc4n(-c4ccc(-c5cccc(-c6ccccc6)c5)cc4)c3c2)cc1] | 69 |
| 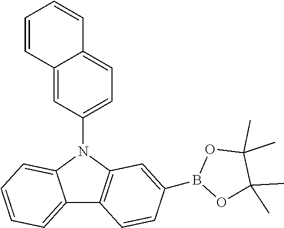 [SMILES: CC1(C)OB(c2ccc3c4ccccc4n(-c4ccc5ccccc5c4)c3c2)OC1(C)C] | 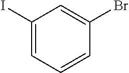 | 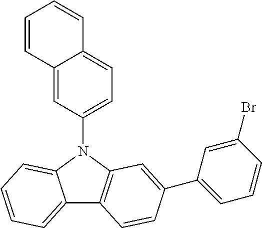 [SMILES: Brc1cccc(-c2ccc3c4ccccc4n(-c4ccc5ccccc5c4)c3c2)c1] | 71 |
| 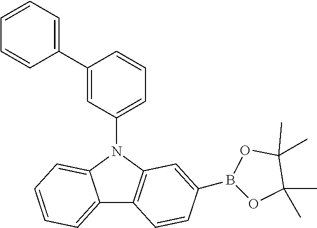 [SMILES: CC1(C)OB(c2ccc3c4ccccc4n(-c4cccc(-c5ccccc5)c4)c3c2)OC1(C)C] | 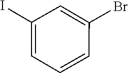 | 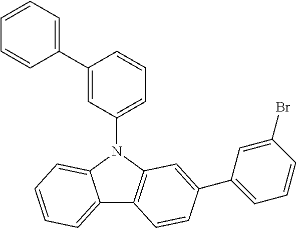 [SMILES: Brc1cccc(-c2ccc3c4ccccc4n(-c4cccc(-c5ccccc5)c4)c3c2)c1] | 73 |
| 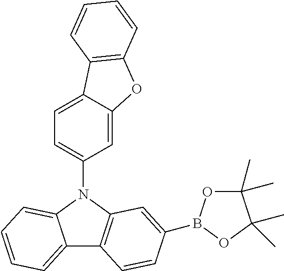 [SMILES: CC1(C)OB(c2ccc3c4ccccc4n(-c4ccc5c(c4)oc4ccccc45)c3c2)OC1(C)C] | 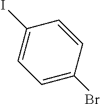 | 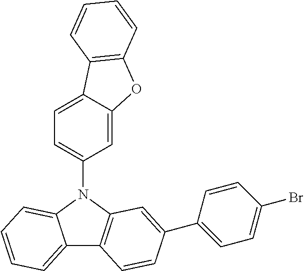 [SMILES: Brc1ccc(-c2ccc3c4ccccc4n(-c4ccc5c(c4)oc4ccccc45)c3c2)cc1] | 64 |
| 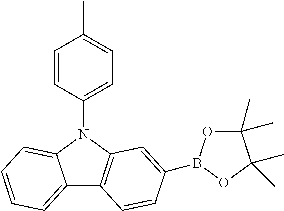 [SMILES: Cc1ccc(-n2c3ccccc3c3ccc(B4OC(C)(C)C(C)(C)O4)cc32)cc1] | 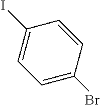 | 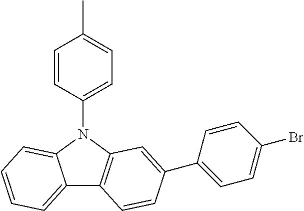 [SMILES: Cc1ccc(-n2c3ccccc3c3ccc(-c4ccc(Br)cc4)cc32)cc1] | 70 |
| 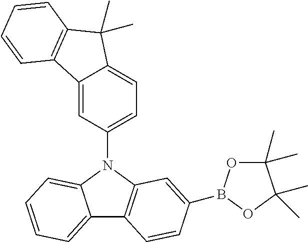 [SMILES: CC1(C)c2ccccc2-c2cc(-n3c4ccccc4c4ccc(B5OC(C)(C)C(C)(C)O5)cc43)ccc21] | 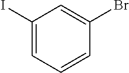 | 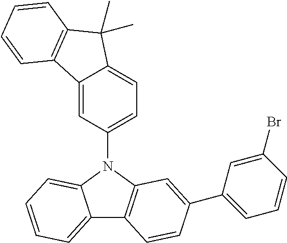 [SMILES: CC1(C)c2ccccc2-c2cc(-n3c4ccccc4c4ccc(-c5cccc(Br)c5)cc43)ccc21] | 62 |
| 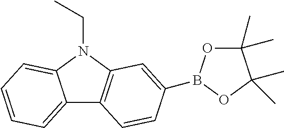 [SMILES: CCn1c2ccccc2c2ccc(B3OC(C)(C)C(C)(C)O3)cc21] | 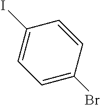 | 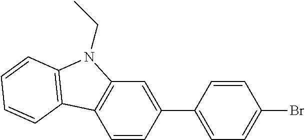 [SMILES: CCn1c2ccccc2c2ccc(-c3ccc(Br)cc3)cc21] | 68 |

### Synthesis of Intermediate IM a1-2

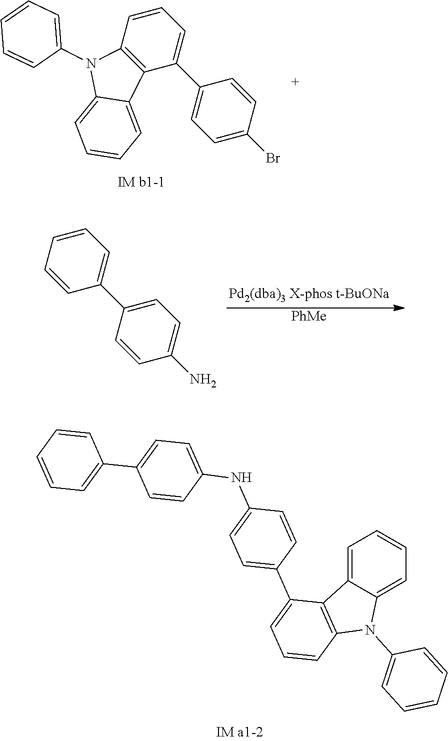 [SMILES: Brc1ccc(-c2cccc3c2c2ccccc2n3-c2ccccc2)cc1 ; Nc1ccc(-c2ccccc2)cc1 ; "PhMe" ; c1ccc(-c2ccc(Nc3ccc(-c4cccc5c4c4ccccc4n5-c4ccccc4)cc3)cc2)cc1]

IM b1-1 (5.0 g, 12.55 mmol), 4-aminobiphenyl (2.34 g, 13.81 mmol), tris(dibenzylideneacetone)dipalladium (0.11 g, 0.13 mmol), 2-dicyclohexylphosphine-2′,4′,6′-triisopropylbiphenyl (0.12 g, 0.25 mmol), sodium tert-butoxide (1.81 g, 18.83 mmol) and toluene (50 mL) were added to a round bottom flask, and stirred at 108° C. to react for 1 h under the protection of nitrogen. After completion of the reaction, the resulting reaction solution was cooled to room temperature, washed with water and then separated to obtain an organic phase, followed by drying the obtained organic phase with anhydrous magnesium sulfate, and then removed the solvent under reduced pressure to obtain a crude product. The obtained crude product was purified by silica gel column chromatography using a mixture of dichloromethane and n-heptane as an eluating agent to gain a white solid compound, namely IM a1-2 (5.2 g, yield: 85%).

The IM a1-X compounds listed in Table 3 were synthesized with reference to the synthesis method of IM a1-2, except that IM b1-1 was replaced with reactant D, and 4-aminobiphenyl was replaced with reactant E listed in Table 3.

TABLE 3

| | | | Yield |
|---|---|---|---|
| Reactant D | Reactant E | IM a1-X | (%) |
| 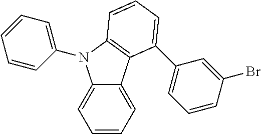 [SMILES: Brc1cccc(-c2cccc3c2c2ccccc2n3-c2ccccc2)c1] | 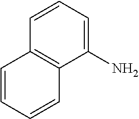 [SMILES: Nc1cccc2ccccc12] | 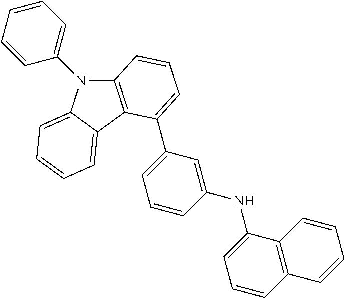 [SMILES: c1ccc(-n2c3ccccc3c3c(-c4cccc(Nc5cccc6ccccc56)c4)cccc32)cc1] | 88 |
| 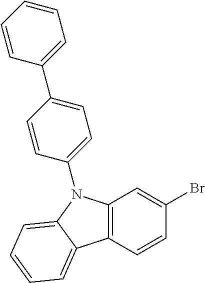 [SMILES: Brc1ccc2c3ccccc3n(-c3ccc(-c4ccccc4)cc3)c2c1] | 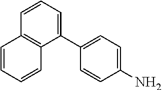 [SMILES: Nc1ccc(-c2cccc3ccccc23)cc1] | 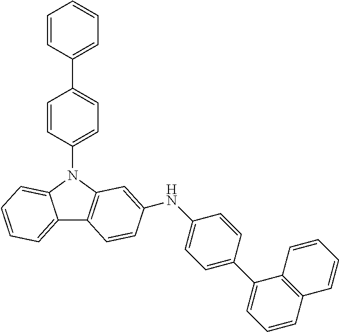 [SMILES: c1ccc(-c2ccc(-n3c4ccccc4c4ccc(Nc5ccc(-c6cccc7ccccc67)cc5)cc43)cc2)cc1] | 81 |
| 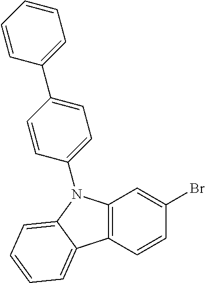 [SMILES: Brc1ccc2c3ccccc3n(-c3ccc(-c4ccccc4)cc3)c2c1] | 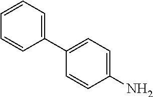 [SMILES: Nc1ccc(-c2ccccc2)cc1] | 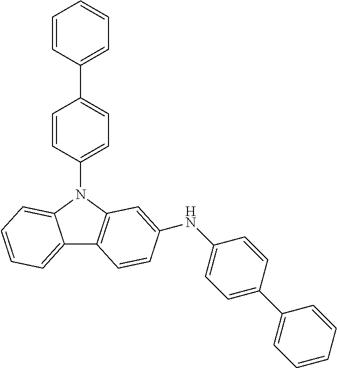 [SMILES: c1ccc(-c2ccc(Nc3ccc4c5ccccc5n(-c5ccc(-c6ccccc6)cc5)c4c3)cc2)cc1] | 85 |
| 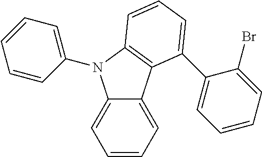 [SMILES: Brc1ccccc1-c1cccc2c1c1ccccc1n2-c1ccccc1] | 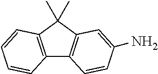 [SMILES: CC1(C)c2ccccc2-c2ccc(N)cc21] | 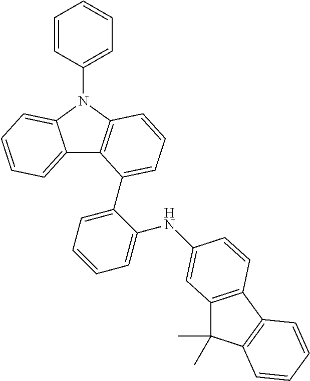 [SMILES: CC1(C)c2ccccc2-c2ccc(Nc3ccccc3-c3cccc4c3c3ccccc3n4-c3ccccc3)cc21] | 78 |
| 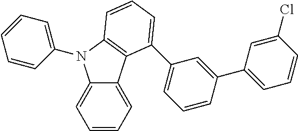 [SMILES: Clc1cccc(-c2cccc(-c3cccc4c3c3ccccc3n4-c3ccccc3)c2)c1] | 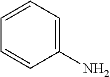 | 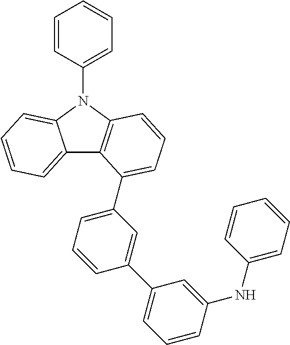 [SMILES: c1ccc(Nc2cccc(-c3cccc(-c4cccc5c4c4ccccc4n5-c4ccccc4)c3)c2)cc1] | 88 |
| 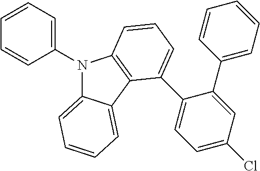 [SMILES: Clc1ccc(-c2cccc3c2c2ccccc2n3-c2ccccc2)c(-c2ccccc2)c1] | 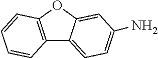 [SMILES: Nc1ccc2c(c1)oc1ccccc12] | 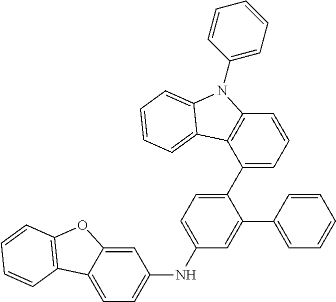 [SMILES: c1ccc(-c2cc(Nc3ccc4c(c3)oc3ccccc34)ccc2-c2cccc3c2c2ccccc2n3-c2ccccc2)cc1] | 75 |
| 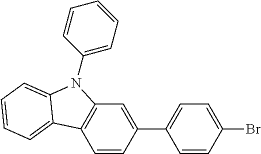 [SMILES: Brc1ccc(-c2ccc3c4ccccc4n(-c4ccccc4)c3c2)cc1] | 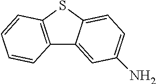 [SMILES: Nc1ccc2sc3ccccc3c2c1] | 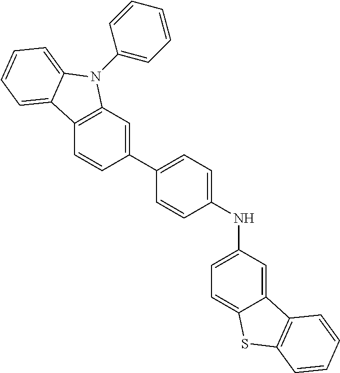 [SMILES: c1ccc(-n2c3ccccc3c3ccc(-c4ccc(Nc5ccc6sc7ccccc7c6c5)cc4)cc32)cc1] | 86 |
| 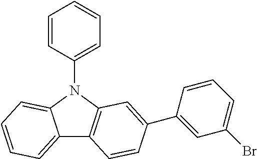 [SMILES: Brc1cccc(-c2ccc3c4ccccc4n(-c4ccccc4)c3c2)c1] | 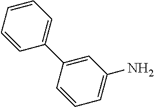 [SMILES: Nc1cccc(-c2ccccc2)c1] | 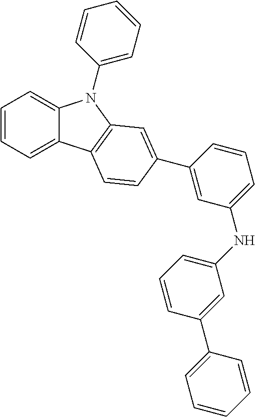 [SMILES: c1ccc(-c2cccc(Nc3cccc(-c4ccc5c6ccccc6n(-c6ccccc6)c5c4)c3)c2)cc1] | 87 |
| 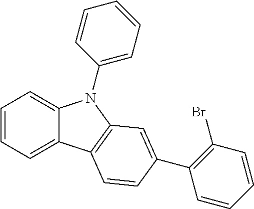 [SMILES: Brc1ccccc1-c1ccc2c3ccccc3n(-c3ccccc3)c2c1] | 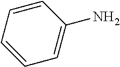 | 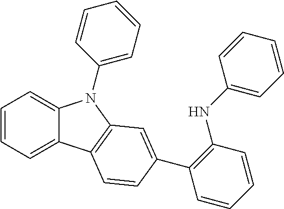 [SMILES: c1ccc(Nc2ccccc2-c2ccc3c4ccccc4n(-c4ccccc4)c3c2)cc1] | 78 |
| 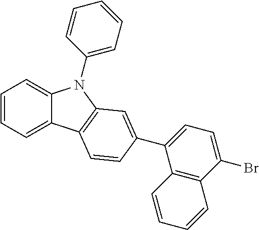 [SMILES: Brc1ccc(-c2ccc3c4ccccc4n(-c4ccccc4)c3c2)c2ccccc12] | 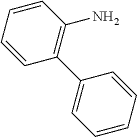 [SMILES: Nc1ccccc1-c1ccccc1] | 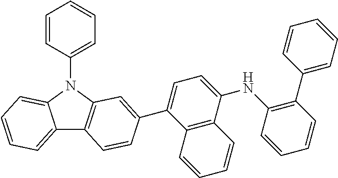 [SMILES: c1ccc(-c2ccccc2Nc2ccc(-c3ccc4c5ccccc5n(-c5ccccc5)c4c3)c3ccccc23)cc1] | 80 |
| 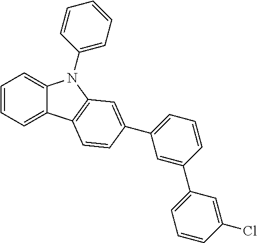 [SMILES: Clc1cccc(-c2cccc(-c3ccc4c5ccccc5n(-c5ccccc5)c4c3)c2)c1] | 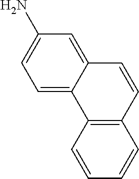 [SMILES: Nc1ccc2c(ccc3ccccc32)c1] | 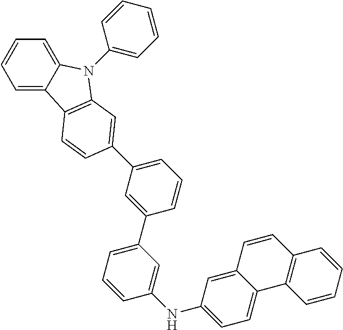 [SMILES: c1ccc(-n2c3ccccc3c3ccc(-c4cccc(-c5cccc(Nc6ccc7c(ccc8ccccc87)c6)c5)c4)cc32)cc1] | 85 |
| 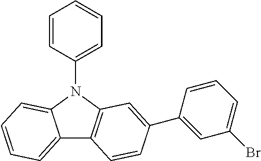 [SMILES: Brc1cccc(-c2ccc3c4ccccc4n(-c4ccccc4)c3c2)c1] | 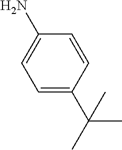 | 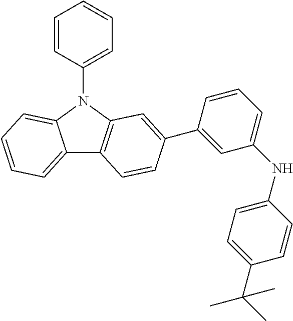 [SMILES: CC(C)(C)c1ccc(Nc2cccc(-c3ccc4c5ccccc5n(-c5ccccc5)c4c3)c2)cc1] | 88 |
| 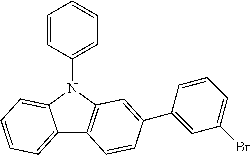 [SMILES: Brc1cccc(-c2ccc3c4ccccc4n(-c4ccccc4)c3c2)c1] | 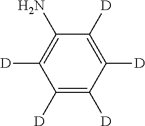 [SMILES: [2H]c1c([2H])c([2H])c(N)c([2H])c1[2H]] | 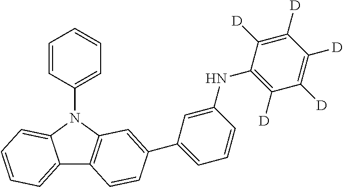 [SMILES: [2H]c1c([2H])c([2H])c(Nc2cccc(-c3ccc4c5ccccc5n(-c5ccccc5)c4c3)c2)c([2H])c1[2H]] | 83 |
| 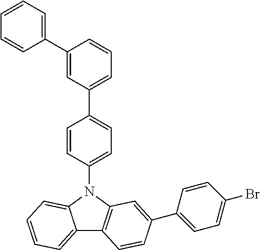 [SMILES: Brc1ccc(-c2ccc3c4ccccc4n(-c4ccc(-c5cccc(-c6ccccc6)c5)cc4)c3c2)cc1] | 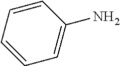 | 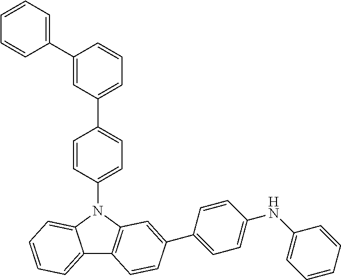 [SMILES: c1ccc(Nc2ccc(-c3ccc4c5ccccc5n(-c5ccc(-c6cccc(-c7ccccc7)c6)cc5)c4c3)cc2)cc1] | 79 |
| 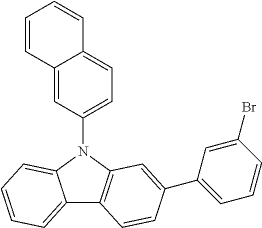 [SMILES: Brc1cccc(-c2ccc3c4ccccc4n(-c4ccc5ccccc5c4)c3c2)c1] | 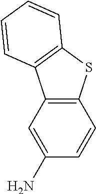 [SMILES: Nc1ccc2sc3ccccc3c2c1] | 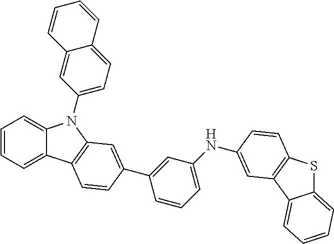 [SMILES: c1cc(Nc2ccc3sc4ccccc4c3c2)cc(-c2ccc3c4ccccc4n(-c4ccc5ccccc5c4)c3c2)c1] | 73 |
| 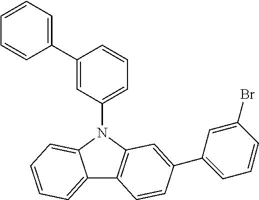 [SMILES: Brc1cccc(-c2ccc3c4ccccc4n(-c4cccc(-c5ccccc5)c4)c3c2)c1] | 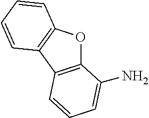 [SMILES: Nc1cccc2c1oc1ccccc12] | 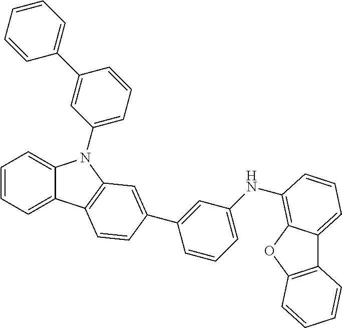 [SMILES: c1ccc(-c2cccc(-n3c4ccccc4c4ccc(-c5cccc(Nc6cccc7c6oc6ccccc67)c5)cc43)c2)cc1] | 76 |
| 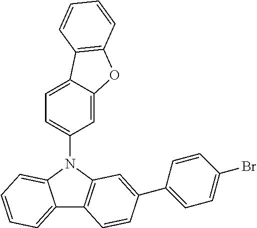 [SMILES: Brc1ccc(-c2ccc3c4ccccc4n(-c4ccc5c(c4)oc4ccccc45)c3c2)cc1] | 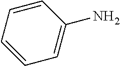 | 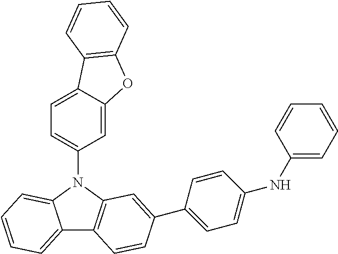 [SMILES: c1ccc(Nc2ccc(-c3ccc4c5ccccc5n(-c5ccc6c(c5)oc5ccccc56)c4c3)cc2)cc1] | 87 |
| 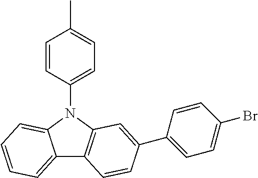 [SMILES: Cc1ccc(-n2c3ccccc3c3ccc(-c4ccc(Br)cc4)cc32)cc1] | 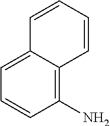 [SMILES: Nc1cccc2ccccc12] | 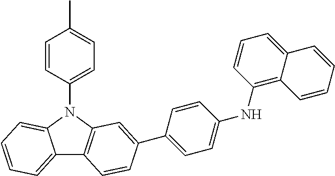 [SMILES: Cc1ccc(-n2c3ccccc3c3ccc(-c4ccc(Nc5cccc6ccccc56)cc4)cc32)cc1] | 85 |
| 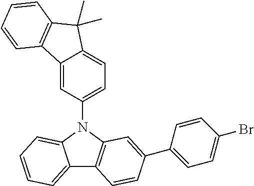 [SMILES: CC1(C)c2ccccc2-c2cc(-n3c4ccccc4c4ccc(-c5ccc(Br)cc5)cc43)ccc21] | 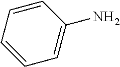 | 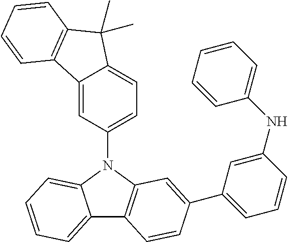 [SMILES: CC1(C)c2ccccc2-c2cc(-n3c4ccccc4c4ccc(-c5cccc(Nc6ccccc6)c5)cc43)ccc21] | 91 |
| 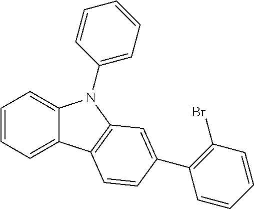 [SMILES: Brc1ccccc1-c1ccc2c3ccccc3n(-c3ccccc3)c2c1] | 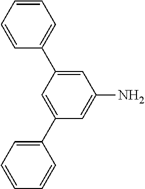 [SMILES: Nc1cc(-c2ccccc2)cc(-c2ccccc2)c1] | 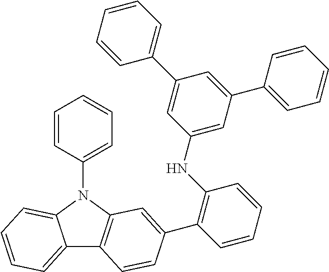 [SMILES: c1ccc(-c2cc(Nc3ccccc3-c3ccc4c5ccccc5n(-c5ccccc5)c4c3)cc(-c3ccccc3)c2)cc1] | 80 |
| 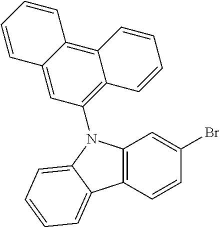 [SMILES: Brc1ccc2c3ccccc3n(-c3cc4ccccc4c4ccccc34)c2c1] | 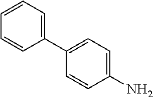 [SMILES: Nc1ccc(-c2ccccc2)cc1] | 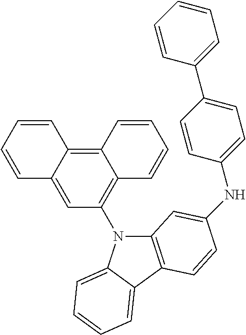 [SMILES: c1ccc(-c2ccc(Nc3ccc4c5ccccc5n(-c5cc6ccccc6c6ccccc56)c4c3)cc2)cc1] | 84 |
| 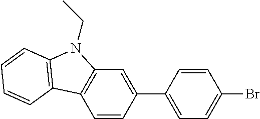 [SMILES: CCn1c2ccccc2c2ccc(-c3ccc(Br)cc3)cc21] | 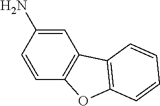 [SMILES: Nc1ccc2oc3ccccc3c2c1] | 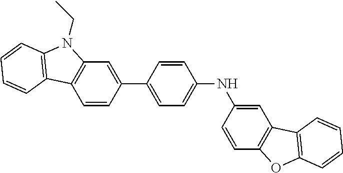 [SMILES: CCn1c2ccccc2c2ccc(-c3ccc(Nc4ccc5oc6ccccc6c5c4)cc3)cc21] | 81 |

### Synthesis Example 11: Synthesis of Compound 2-4

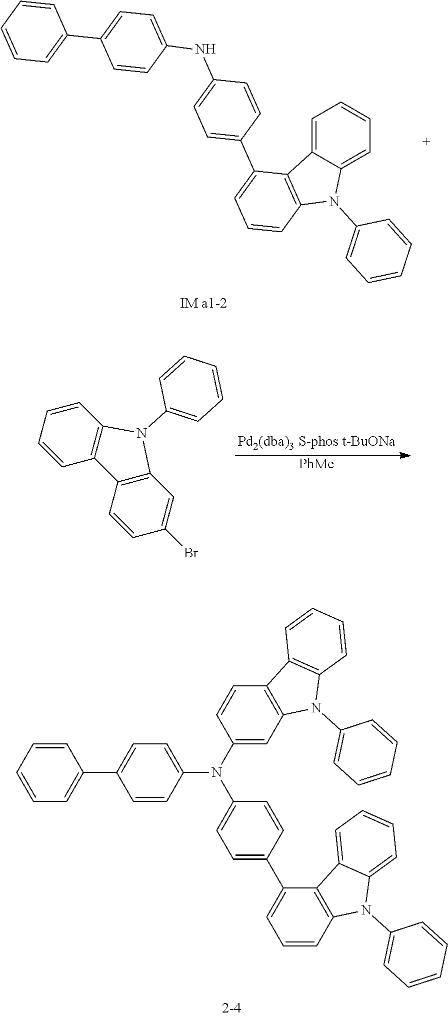 [SMILES: c1ccc(-c2ccc(Nc3ccc(-c4cccc5c4c4ccccc4n5-c4ccccc4)cc3)cc2)cc1 ; Brc1ccc2c3ccccc3n(-c3ccccc3)c2c1 ; "PhMe" ; c1ccc(-c2ccc(N(c3ccc(-c4cccc5c4c4ccccc4n5-c4ccccc4)cc3)c3ccc4c5ccccc5n(-c5ccccc5)c4c3)cc2)cc1]

IM a1-2 (5 g, 10.28 mmol), 2-bromo-9-phenylcarbazole (3.48 g, 10.79 mmol), tris(dibenzylideneacetone)dipalladium (0.09 g, 010 mmol), 2-dicyclohexylphosphino-2′,6′-dimethoxy-biphenyl (0.08 g, 0.21 mmol), sodium tert-butoxide (1.48 g, 15.4 mmol) and toluene (50 mL) were added to a round bottom flask, and stirred at 108° C. to react for 4 h under the protection of nitrogen. After completion of the reaction, the resulting reaction was cooled to room temperature, washed with water and then separated to obtain an organic phase, followed by drying the obtained organic phase with anhydrous magnesium sulfate, and then removed the solvent under reduced pressure to obtain a crude product. The obtained crude product was purified by silica gel column chromatography using a mixture of dichloromethane and ethyl acetate as the recrystallization solvent, to gain the white solid compound 2-4 (6.6 g, yield: 88%), mass spectrometry (m/z)=728.3 [M+H]+. NMR data for compounds 2-4: ¹H NMR (400 MHZ, CD2Cl2): 8.23 (d, 1H), 8.07 (d, 1H), 7.95 (d, 1H), 7.76-7.44 (m, 28H), 7.28-7.01 (m, 3H), 6.89 (d, 2H), 6.78 (s, 1H).

### Synthesis Example 12 to Synthesis Example 35

The following compounds were synthesized with reference to the synthesis method of compound 2-4, except that IM a1-2 was replaced with reactant F and 2-bromo-9-phenylcarbazole was replaced with reactant G. The synthesized compound, yield and mass spectrometry result thereof are shown in Table 4.

TABLE 4

| | | | | | Mass |
|---|---|---|---|---|---|
| Syn- | | | | | spectro- |
| thesis | | | | | metry |
| Ex- | | | | | (m/z), |
| am- | | | | Yield | [M + |
| ple | Reactant F | Reactant G | Compound | (%) | H]⁺ |
| 12 | 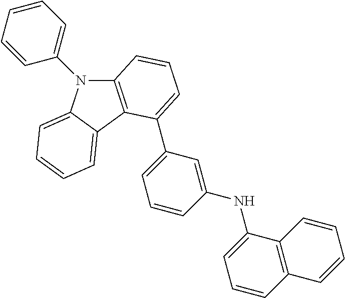 [SMILES: c1ccc(-n2c3ccccc3c3c(-c4cccc(Nc5cccc6ccccc56)c4)cccc32)cc1] | 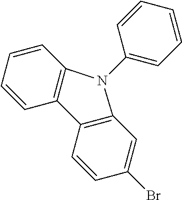 [SMILES: Brc1ccc2c3ccccc3n(-c3ccccc3)c2c1] | 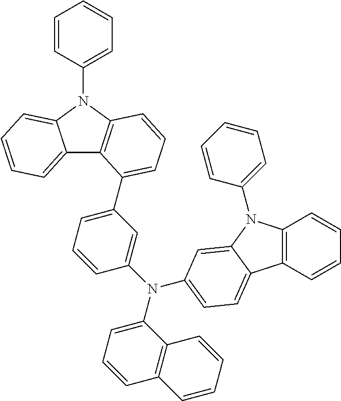 [SMILES: c1ccc(-n2c3ccccc3c3ccc(N(c4cccc(-c5cccc6c5c5ccccc5n6-c5ccccc5)c4)c4cccc5ccccc45)cc32)cc1] | 79 | 702.3 |
| 13 | 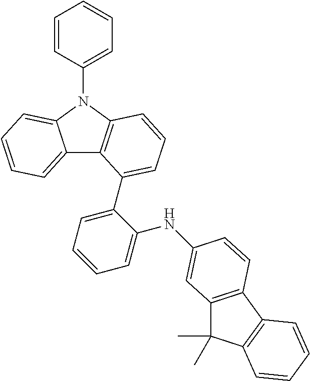 [SMILES: CC1(C)c2ccccc2-c2ccc(Nc3ccccc3-c3cccc4c3c3ccccc3n4-c3ccccc3)cc21] | 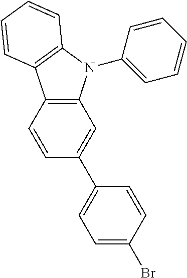 [SMILES: Brc1ccc(-c2ccc3c4ccccc4n(-c4ccccc4)c3c2)cc1] | 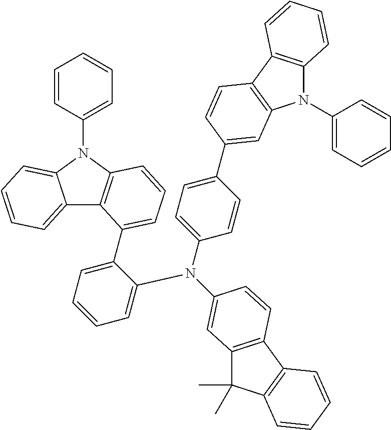 [SMILES: CC1(C)c2ccccc2-c2ccc(N(c3ccc(-c4ccc5c6ccccc6n(-c6ccccc6)c5c4)cc3)c3ccccc3-c3cccc4c3c3ccccc3n4-c3ccccc3)cc21] | 84 | 844.4 |
| 14 | 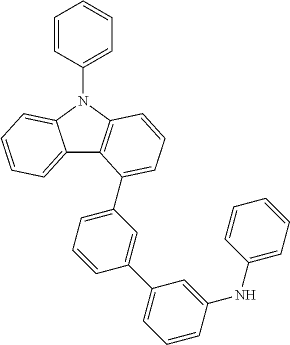 [SMILES: c1ccc(Nc2cccc(-c3cccc(-c4cccc5c4c4ccccc4n5-c4ccccc4)c3)c2)cc1] | 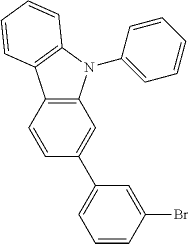 [SMILES: Brc1cccc(-c2ccc3c4ccccc4n(-c4ccccc4)c3c2)c1] | 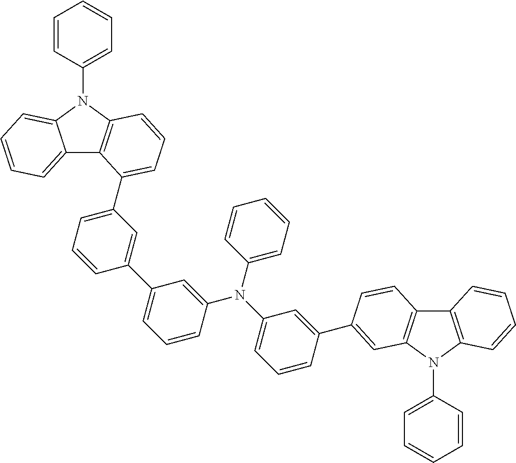 [SMILES: c1ccc(N(c2cccc(-c3cccc(-c4cccc5c4c4ccccc4n5-c4ccccc4)c3)c2)c2cccc(-c3ccc4c5ccccc5n(-c5ccccc5)c4c3)c2)cc1] | 84 | 804.3 |
| 15 | 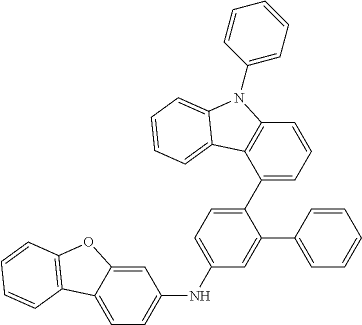 [SMILES: c1ccc(-c2cc(Nc3ccc4c(c3)oc3ccccc34)ccc2-c2cccc3c2c2ccccc2n3-c2ccccc2)cc1] | 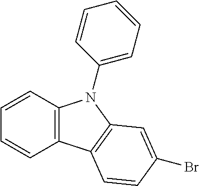 [SMILES: Brc1ccc2c3ccccc3n(-c3ccccc3)c2c1] | 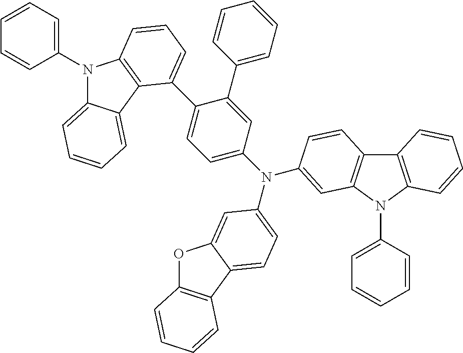 [SMILES: c1ccc(-c2cc(N(c3ccc4c(c3)oc3ccccc34)c3ccc4c5ccccc5n(-c5ccccc5)c4c3)ccc2-c2cccc3c2c2ccccc2n3-c2ccccc2)cc1] | 75 | 818.3 |
| 16 | 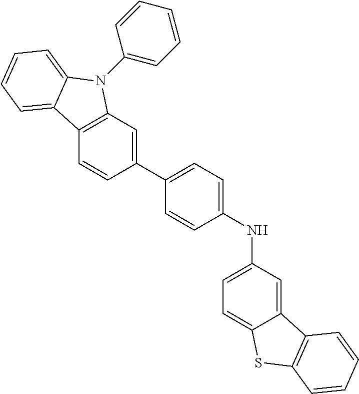 [SMILES: c1ccc(-n2c3ccccc3c3ccc(-c4ccc(Nc5ccc6sc7ccccc7c6c5)cc4)cc32)cc1] | 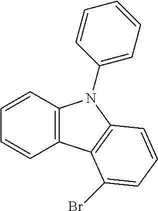 [SMILES: Brc1cccc2c1c1ccccc1n2-c1ccccc1] | 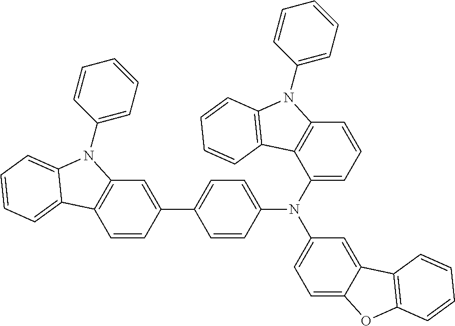 [SMILES: c1ccc(-n2c3ccccc3c3ccc(-c4ccc(N(c5ccc6oc7ccccc7c6c5)c5cccc6c5c5ccccc5n6-c5ccccc5)cc4)cc32)cc1] | 81 | 758.3 |
| 17 | 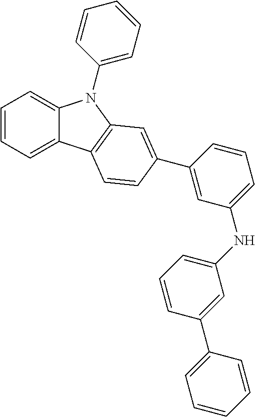 [SMILES: c1ccc(-c2cccc(Nc3cccc(-c4ccc5c6ccccc6n(-c6ccccc6)c5c4)c3)c2)cc1] | 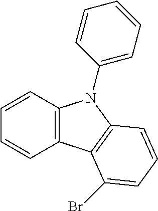 [SMILES: Brc1cccc2c1c1ccccc1n2-c1ccccc1] | 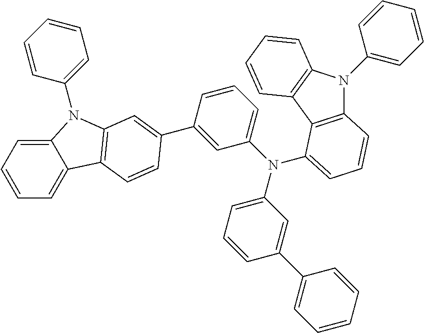 [SMILES: c1ccc(-c2cccc(N(c3cccc(-c4ccc5c6ccccc6n(-c6ccccc6)c5c4)c3)c3cccc4c3c3ccccc3n4-c3ccccc3)c2)cc1] | 87 | 728.3 |
| 18 19 | 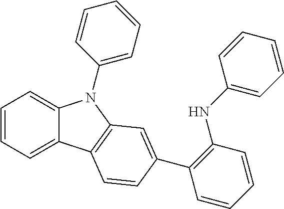 [SMILES: c1ccc(Nc2ccccc2-c2ccc3c4ccccc4n(-c4ccccc4)c3c2)cc1] 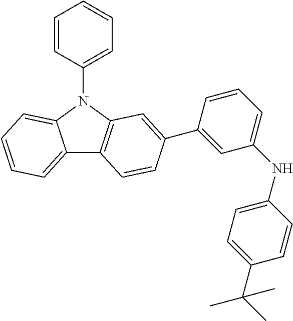 [SMILES: CC(C)(C)c1ccc(Nc2cccc(-c3ccc4c5ccccc5n(-c5ccccc5)c4c3)c2)cc1] | 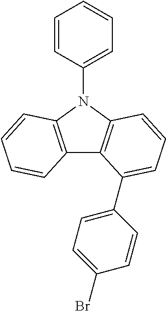 [SMILES: Brc1ccc(-c2cccc3c2c2ccccc2n3-c2ccccc2)cc1] | 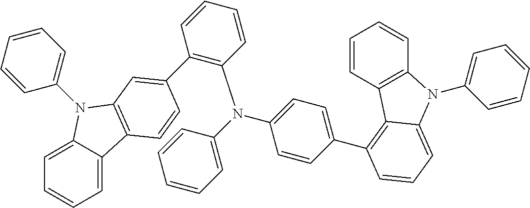 [SMILES: c1ccc(N(c2ccc(-c3cccc4c3c3ccccc3n4-c3ccccc3)cc2)c2ccccc2-c2ccc3c4ccccc4n(-c4ccccc4)c3c2)cc1] 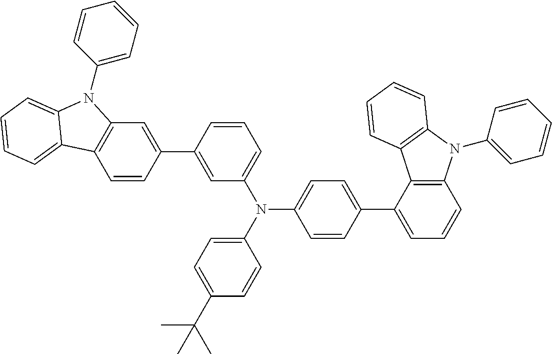 [SMILES: CC(C)(C)c1ccc(N(c2ccc(-c3cccc4c3c3ccccc3n4-c3ccccc3)cc2)c2cccc(-c3ccc4c5ccccc5n(-c5ccccc5)c4c3)c2)cc1] | 71 92 | 728.3 784.4 |
| 20 | 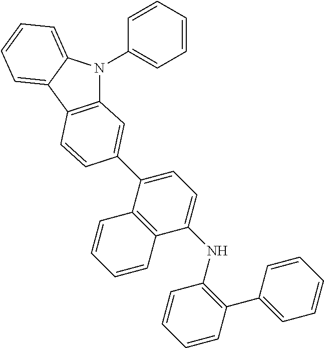 [SMILES: c1ccc(-c2ccccc2Nc2ccc(-c3ccc4c5ccccc5n(-c5ccccc5)c4c3)c3ccccc23)cc1] | 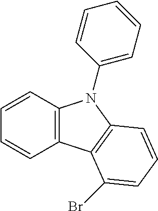 [SMILES: Brc1cccc2c1c1ccccc1n2-c1ccccc1] | 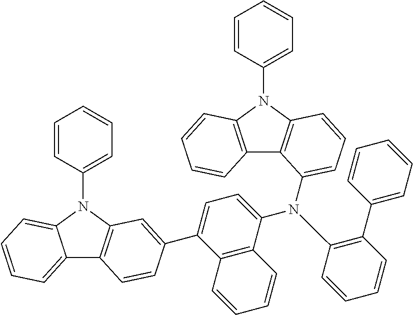 [SMILES: c1ccc(-c2ccccc2N(c2ccc(-c3ccc4c5ccccc5n(-c5ccccc5)c4c3)c3ccccc23)c2cccc3c2c2ccccc2n3-c2ccccc2)cc1] | 77 | 778.3 |
| 21 | 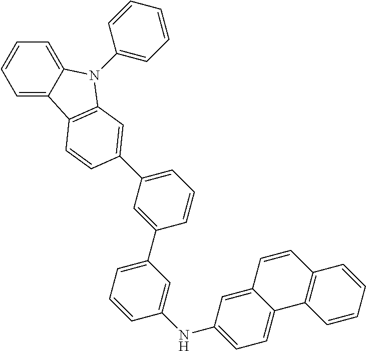 [SMILES: c1ccc(-n2c3ccccc3c3ccc(-c4cccc(-c5cccc(Nc6ccc7c(ccc8ccccc87)c6)c5)c4)cc32)cc1] | 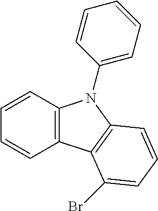 [SMILES: Brc1cccc2c1c1ccccc1n2-c1ccccc1] | 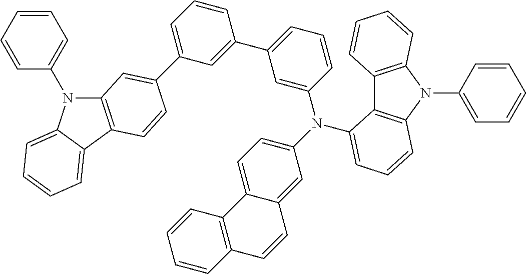 [SMILES: c1ccc(-n2c3ccccc3c3ccc(-c4cccc(-c5cccc(N(c6ccc7c(ccc8ccccc87)c6)c6cccc7c6c6ccccc6n7-c6ccccc6)c5)c4)cc32)cc1] | 85 | 828.3 |
| 22 | 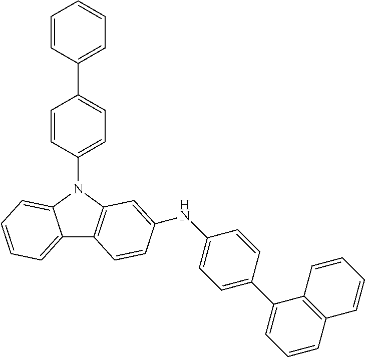 [SMILES: c1ccc(-c2ccc(-n3c4ccccc4c4ccc(Nc5ccc(-c6cccc7ccccc67)cc5)cc43)cc2)cc1] | 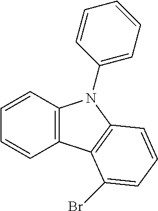 [SMILES: Brc1cccc2c1c1ccccc1n2-c1ccccc1] | 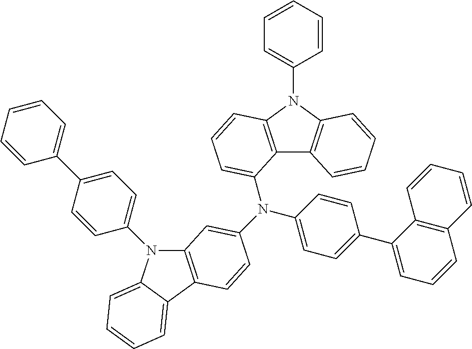 [SMILES: c1ccc(-c2ccc(-n3c4ccccc4c4ccc(N(c5ccc(-c6cccc7ccccc67)cc5)c5cccc6c5c5ccccc5n6-c5ccccc5)cc43)cc2)cc1] | 85 | 778.3 |
| 23 | 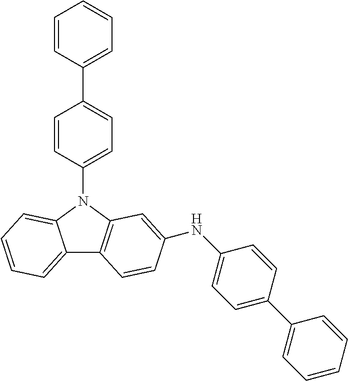 [SMILES: c1ccc(-c2ccc(Nc3ccc4c5ccccc5n(-c5ccc(-c6ccccc6)cc5)c4c3)cc2)cc1] | 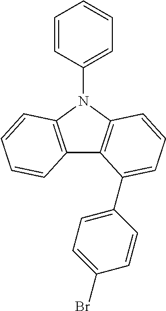 [SMILES: Brc1ccc(-c2cccc3c2c2ccccc2n3-c2ccccc2)cc1] | 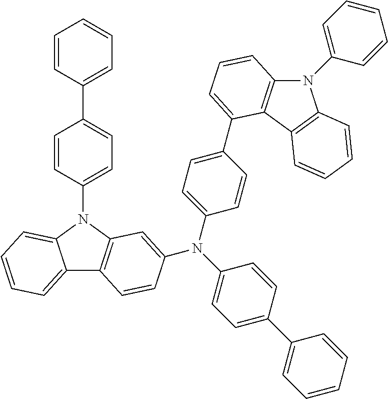 [SMILES: c1ccc(-c2ccc(N(c3ccc(-c4cccc5c4c4ccccc4n5-c4ccccc4)cc3)c3ccc4c5ccccc5n(-c5ccc(-c6ccccc6)cc5)c4c3)cc2)cc1] | 89 | 804.3 |
| 24 | 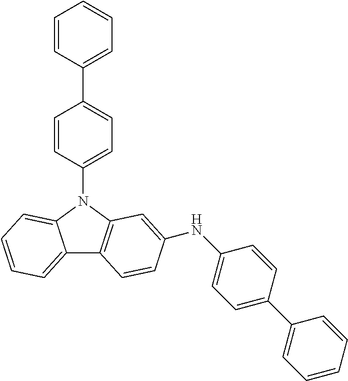 [SMILES: c1ccc(-c2ccc(Nc3ccc4c5ccccc5n(-c5ccc(-c6ccccc6)cc5)c4c3)cc2)cc1] | 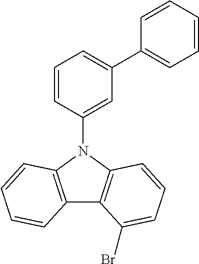 [SMILES: Brc1cccc2c1c1ccccc1n2-c1cccc(-c2ccccc2)c1] | 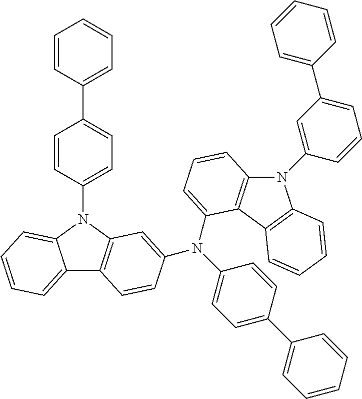 [SMILES: c1ccc(-c2ccc(N(c3ccc4c5ccccc5n(-c5ccc(-c6ccccc6)cc5)c4c3)c3cccc4c3c3ccccc3n4-c3cccc(-c4ccccc4)c3)cc2)cc1] | 79 | 804.3 |
| 25 | 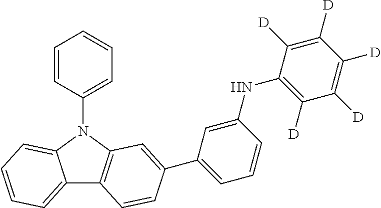 [SMILES: [2H]c1c([2H])c([2H])c(Nc2cccc(-c3ccc4c5ccccc5n(-c5ccccc5)c4c3)c2)c([2H])c1[2H]] | 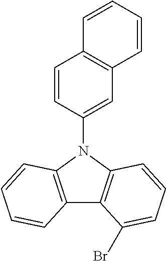 [SMILES: Brc1cccc2c1c1ccccc1n2-c1ccc2ccccc2c1] | 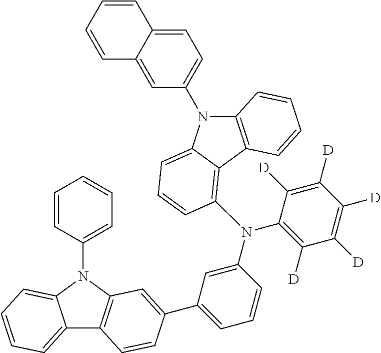 [SMILES: [2H]c1c([2H])c([2H])c(N(c2cccc(-c3ccc4c5ccccc5n(-c5ccccc5)c4c3)c2)c2cccc3c2c2ccccc2n3-c2ccc3ccccc3c2)c([2H])c1[2H]] | 81 | 707.3 |
| 26 | 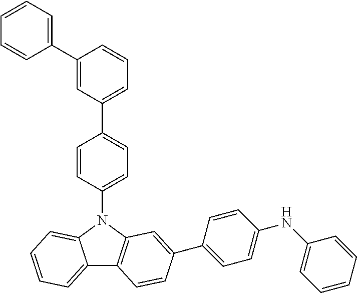 [SMILES: c1ccc(Nc2ccc(-c3ccc4c5ccccc5n(-c5ccc(-c6cccc(-c7ccccc7)c6)cc5)c4c3)cc2)cc1] | 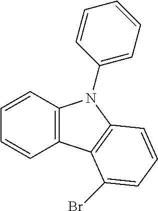 [SMILES: Brc1cccc2c1c1ccccc1n2-c1ccccc1] | 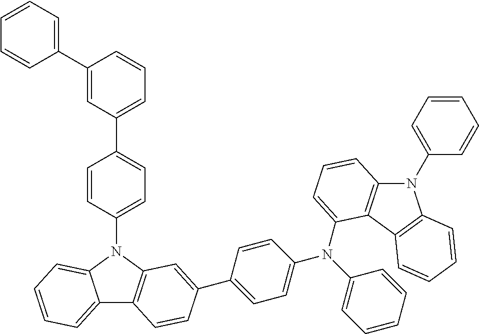 [SMILES: c1ccc(-c2cccc(-c3ccc(-n4c5ccccc5c5ccc(-c6ccc(N(c7ccccc7)c7cccc8c7c7ccccc7n8-c7ccccc7)cc6)cc54)cc3)c2)cc1] | 75 | 804.3 |
| 27 | 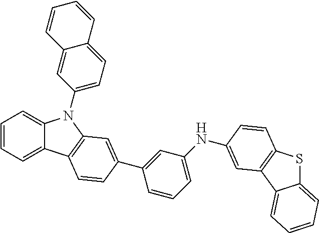 [SMILES: c1cc(Nc2ccc3sc4ccccc4c3c2)cc(-c2ccc3c4ccccc4n(-c4ccc5ccccc5c4)c3c2)c1] | 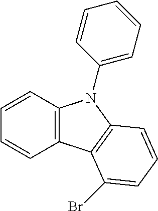 [SMILES: Brc1cccc2c1c1ccccc1n2-c1ccccc1] | 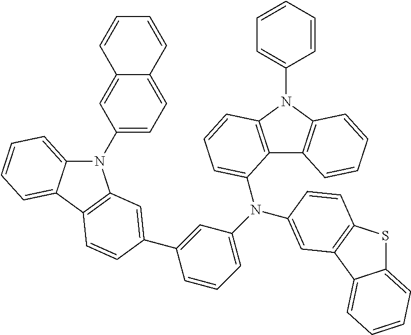 [SMILES: c1ccc(-n2c3ccccc3c3c(N(c4cccc(-c5ccc6c7ccccc7n(-c7ccc8ccccc8c7)c6c5)c4)c4ccc5sc6ccccc6c5c4)cccc32)cc1] | 78 | 808.3 |
| 28 | 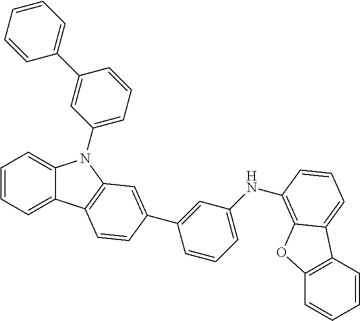 [SMILES: c1ccc(-c2cccc(-n3c4ccccc4c4ccc(-c5cccc(Nc6cccc7c6oc6ccccc67)c5)cc43)c2)cc1] | 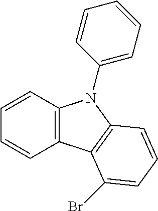 [SMILES: Brc1cccc2c1c1ccccc1n2-c1ccccc1] | 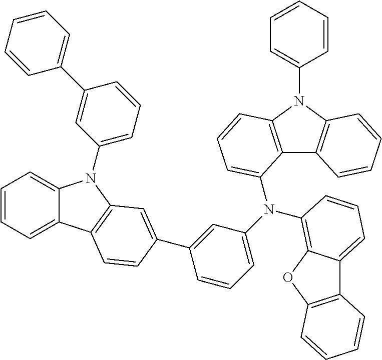 [SMILES: c1ccc(-c2cccc(-n3c4ccccc4c4ccc(-c5cccc(N(c6cccc7c6oc6ccccc67)c6cccc7c6c6ccccc6n7-c6ccccc6)c5)cc43)c2)cc1] | 82 | 818.3 |
| 29 | 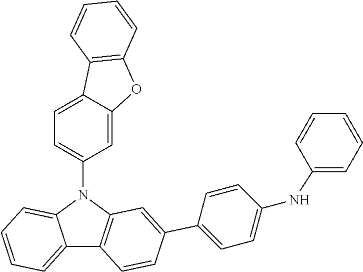 [SMILES: c1ccc(Nc2ccc(-c3ccc4c5ccccc5n(-c5ccc6c(c5)oc5ccccc56)c4c3)cc2)cc1] | 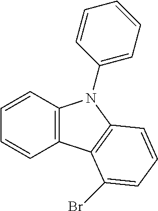 [SMILES: Brc1cccc2c1c1ccccc1n2-c1ccccc1] | 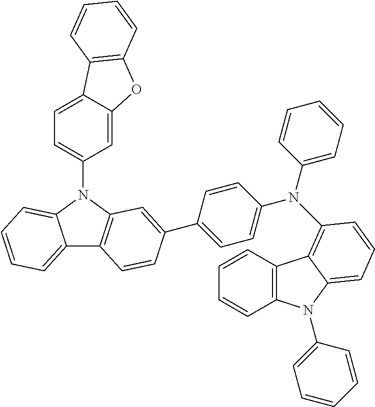 [SMILES: c1ccc(N(c2ccc(-c3ccc4c5ccccc5n(-c5ccc6c(c5)oc5ccccc56)c4c3)cc2)c2cccc3c2c2ccccc2n3-c2ccccc2)cc1] | 85 | 742.3 |
| 30 | 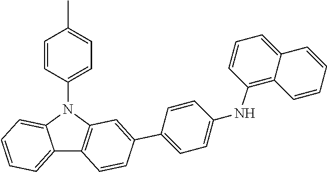 [SMILES: Cc1ccc(-n2c3ccccc3c3ccc(-c4ccc(Nc5cccc6ccccc56)cc4)cc32)cc1] | 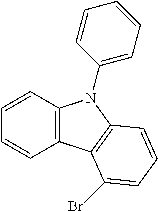 [SMILES: Brc1cccc2c1c1ccccc1n2-c1ccccc1] | 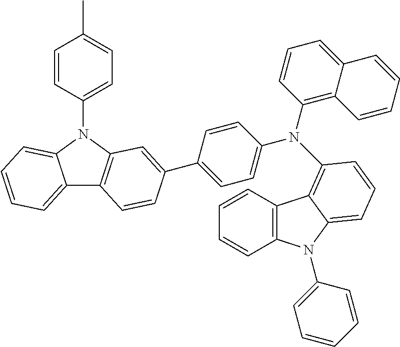 [SMILES: Cc1ccc(-n2c3ccccc3c3ccc(-c4ccc(N(c5cccc6ccccc56)c5cccc6c5c5ccccc5n6-c5ccccc5)cc4)cc32)cc1] | 87 | 716.3 |
| 31 | 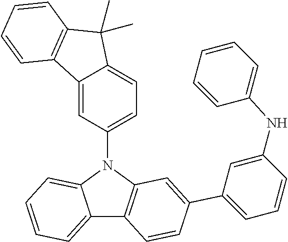 [SMILES: CC1(C)c2ccccc2-c2cc(-n3c4ccccc4c4ccc(-c5cccc(Nc6ccccc6)c5)cc43)ccc21] | 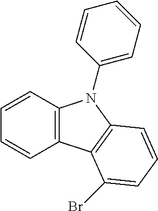 [SMILES: Brc1cccc2c1c1ccccc1n2-c1ccccc1] | 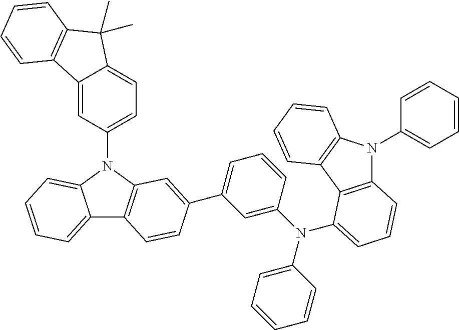 [SMILES: CC1(C)c2ccccc2-c2cc(-n3c4ccccc4c4ccc(-c5cccc(N(c6ccccc6)c6cccc7c6c6ccccc6n7-c6ccccc6)c5)cc43)ccc21] | 78 | 768.3 |
| 32 | 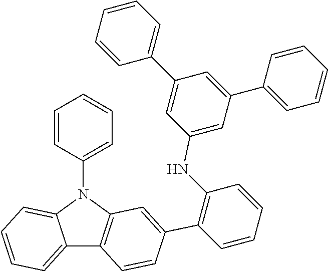 [SMILES: c1ccc(-c2cc(Nc3ccccc3-c3ccc4c5ccccc5n(-c5ccccc5)c4c3)cc(-c3ccccc3)c2)cc1] | 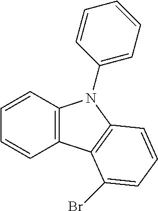 [SMILES: Brc1cccc2c1c1ccccc1n2-c1ccccc1] | 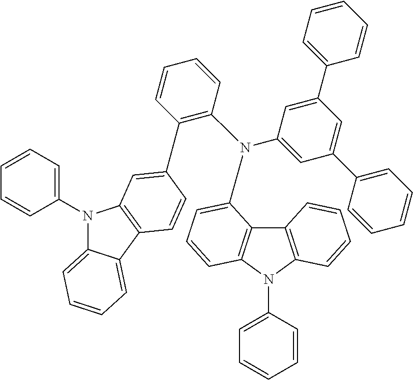 [SMILES: c1ccc(-c2cc(-c3ccccc3)cc(N(c3ccccc3-c3ccc4c5ccccc5n(-c5ccccc5)c4c3)c3cccc4c3c3ccccc3n4-c3ccccc3)c2)cc1] | 81 | 804.3 |
| 33 | 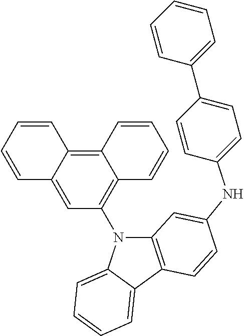 [SMILES: c1ccc(-c2ccc(Nc3ccc4c5ccccc5n(-c5cc6ccccc6c6ccccc56)c4c3)cc2)cc1] | 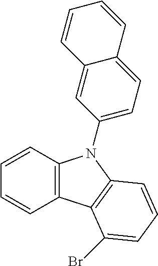 [SMILES: Brc1cccc2c1c1ccccc1n2-c1ccc2ccccc2c1] | 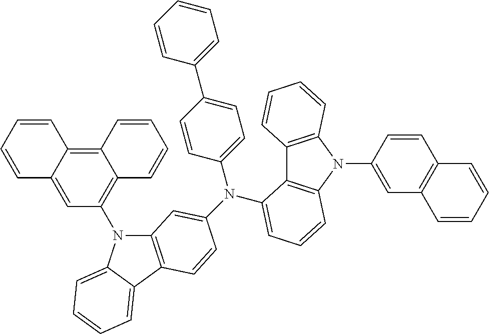 [SMILES: c1ccc(-c2ccc(N(c3ccc4c5ccccc5n(-c5cc6ccccc6c6ccccc56)c4c3)c3cccc4c3c3ccccc3n4-c3ccc4ccccc4c3)cc2)cc1] | 74 | 802.3 |
| 34 | 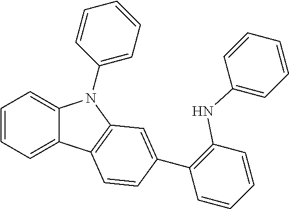 [SMILES: c1ccc(Nc2ccccc2-c2ccc3c4ccccc4n(-c4ccccc4)c3c2)cc1] | 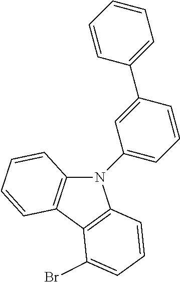 [SMILES: Brc1cccc2c1c1ccccc1n2-c1cccc(-c2ccccc2)c1] | 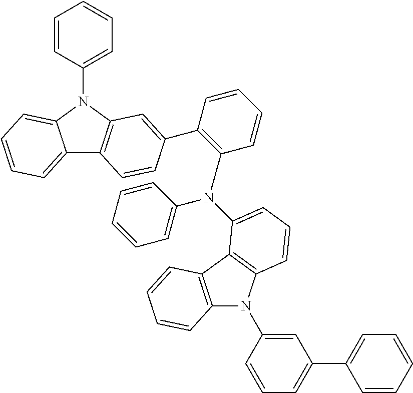 [SMILES: c1ccc(-c2cccc(-n3c4ccccc4c4c(N(c5ccccc5)c5ccccc5-c5ccc6c7ccccc7n(-c7ccccc7)c6c5)cccc43)c2)cc1] | 72 | 728.3 |
| 35 | 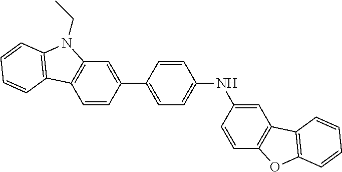 [SMILES: CCn1c2ccccc2c2ccc(-c3ccc(Nc4ccc5oc6ccccc6c5c4)cc3)cc21] | 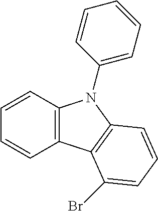 [SMILES: Brc1cccc2c1c1ccccc1n2-c1ccccc1] | 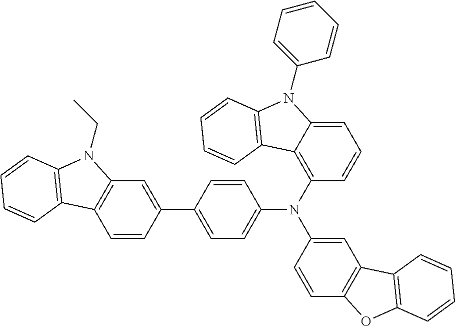 [SMILES: CCn1c2ccccc2c2ccc(-c3ccc(N(c4ccc5oc6ccccc6c5c4)c4cccc5c4c4ccccc4n5-c4ccccc4)cc3)cc21] | 79 | 694.3 |

### Example 1. Preparation of Blue Organic Electroluminescent Device

The anode was prepared by the following process: the ITO/AG/ITO substrate with a thickness of 100 Å/1200 Å/100 Å was cut into a size of 40 mm×40 mm×0.7 mm, from which the experimental substrate with a cathode, an anode and a insulation layer pattern was prepared by a photolithography process, and surface treatment was carried out using ultraviolet-ozone and O₂:N₂ plasma to increase the work function of the anode (experimental substrate) and remove scums.

F4-TCNQ was vacuum-evaporated on the experimental substrate (anode) to form a hole injection layer (HIL) with a thickness of 100 Å, and compound 1-1 was evaporated on the hole injection layer to form a hole transport layer (HTL) with a thickness of 980 Å.

EB-3 was evaporated on the hole transport layer to form an electronic barrier layer (EBL) with a thickness of 120 Å.

On the electronic barrier layer, PCAN and BD-1 were co-evaporated with a weight ratio of 98:2 to form a blue light organic electroluminescent layer (EML) with a thickness of 200 Å.

On the organic electroluminescent layer, BCP and LiQ were co-steamed with a weight ratio of 1:1 to form an electron transport layer (ETL) with a thickness of 300 Å.

Yb was evaporated on the electron transport layer to form an electron injection layer (EIL) with a thickness of 13 Å, and then magnesium and silver were vacuum-evaporated on the electron injection layer at an evaporation rate of 1:10 to form a cathode with a thickness of 128 Å.

In addition, CP-1 was evaporated on the above cathode to form an organic capping layer (CPL) with a thickness of 720 Å, thereby completing the manufacture of the organic electroluminescent device.

### Example 2 to Example 36

The organic electroluminescent device was prepared by the same method as that in Example 1, except that when the hole transport layer was formed, compound 1-1 was replaced with the remaining compounds listed in Table 5.

### Comparative Example 1 to Comparative Example 5

The organic electroluminescent device was prepared by the same method as that in Example 1, except that when the hole transport layer was formed, compound 1-1 was replaced with compound A, compound B, compound C, compound D and compound E in Comparative Example 1 to Comparative Example 5, respectively.

In the above Examples and Comparative Examples, the structure of the main materials used are shown below.

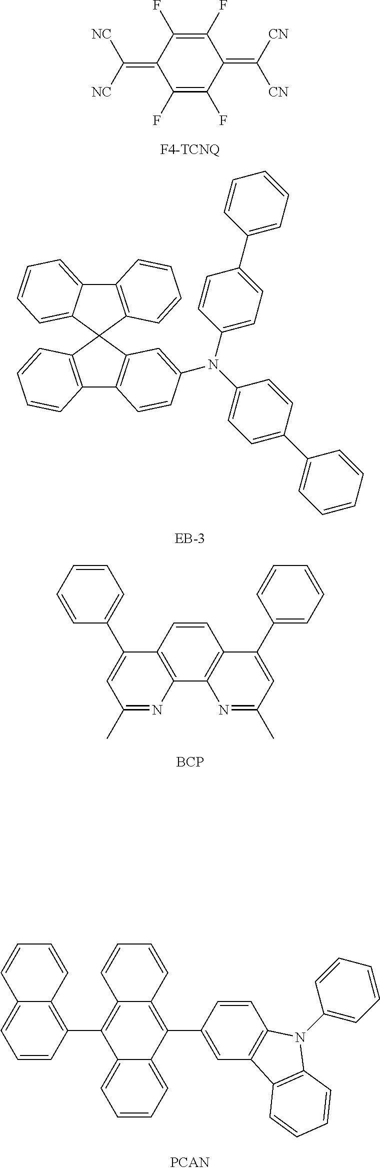

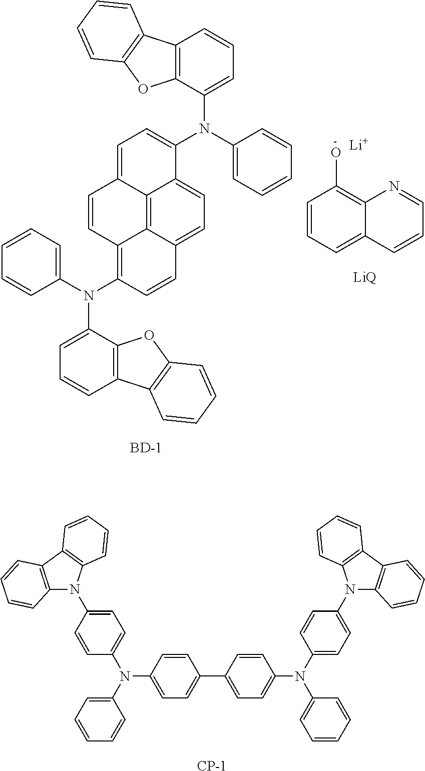

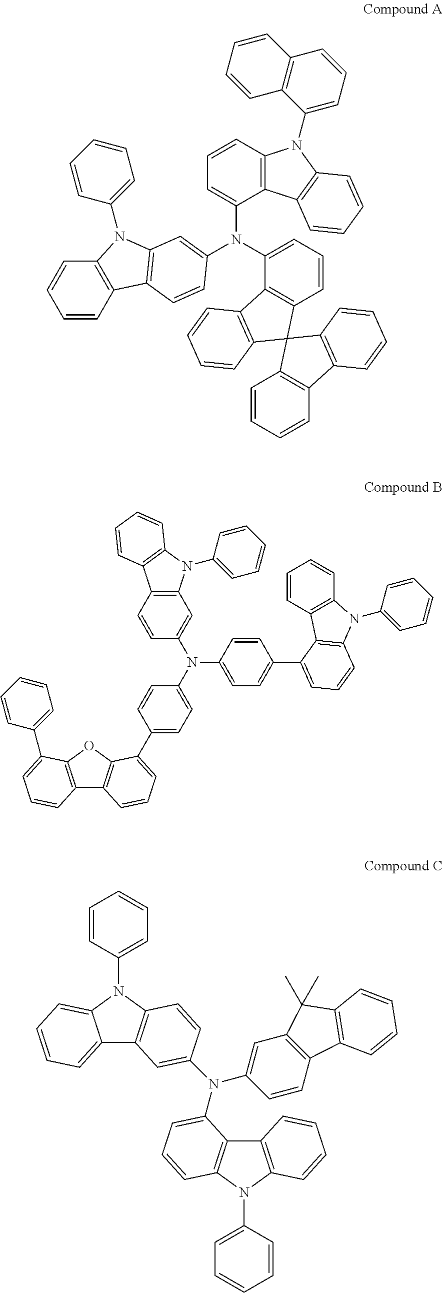 [SMILES: c1ccc(-n2c3ccccc3c3ccc(N(c4cccc5c4-c4ccccc4C54c5ccccc5-c5ccccc54)c4cccc5c4c4ccccc4n5-c4cccc5ccccc45)cc32)cc1 ; c1ccc(-c2cccc3c2oc2c(-c4ccc(N(c5ccc(-c6cccc7c6c6ccccc6n7-c6ccccc6)cc5)c5ccc6c7ccccc7n(-c7ccccc7)c6c5)cc4)cccc23)cc1 ; CC1(C)c2ccccc2-c2ccc(N(c3ccc4c(c3)c3ccccc3n4-c3ccccc3)c3cccc4c3c3ccccc3n4-c3ccccc3)cc21]

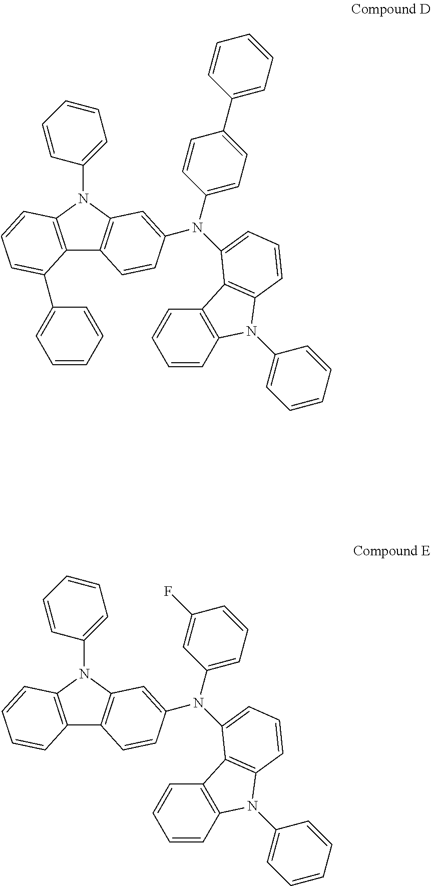 [SMILES: c1ccc(-c2ccc(N(c3ccc4c5c(-c6ccccc6)cccc5n(-c5ccccc5)c4c3)c3cccc4c3c3ccccc3n4-c3ccccc3)cc2)cc1 ; Fc1cccc(N(c2ccc3c4ccccc4n(-c4ccccc4)c3c2)c2cccc3c2c2ccccc2n3-c2ccccc2)c1]

For the organic electroluminescent devices prepared above, the photoelectric performance of the devices under the condition of 20 mA/cm′ was analyzed. The results are shown in Table 5.

TABLE 5

| | | Driving | Current | Power | Chromaticity | Chromaticity | T95 |
|---|---|---|---|---|---|---|---|
| | | voltage | efficiency | efficiency | coordinate, | coordinate, | Service |
| No. | HTL | (V) | (Cd/A) | (lm/W) | CIE-x | CIE-y | life (h) |
| Example 1 | Compound 1-1 | 4.06 | 6.60 | 5.24 | 0.138 | 0.045 | 203 |
| Example 2 | Compound 1-4 | 3.99 | 6.58 | 5.18 | 0.138 | 0.045 | 207 |
| Example 3 | Compound 1-6 | 3.98 | 6.48 | 5.11 | 0.138 | 0.045 | 213 |
| Example 4 | Compound 1-7 | 4.06 | 6.74 | 5.22 | 0.138 | 0.045 | 202 |
| Example 5 | Compound | 4.11 | 6.85 | 5.22 | 0.138 | 0.045 | 216 |
| | 1-12 | | | | | | |
| Example 6 | Compound | 4.04 | 6.63 | 5.16 | 0.138 | 0.045 | 209 |
| | 1-15 | | | | | | |
| Example 7 | Compound | 3.99 | 6.93 | 5.39 | 0.138 | 0.045 | 214 |
| | 1-19 | | | | | | |
| Example 8 | Compound | 4.12 | 6.36 | 4.85 | 0.138 | 0.045 | 205 |
| | 1-26 | | | | | | |
| Example 9 | Compound | 4.05 | 6.52 | 5.06 | 0.138 | 0.045 | 204 |
| | 1-43 | | | | | | |
| Example 10 | Compound | 4.00 | 6.49 | 5.01 | 0.138 | 0.045 | 212 |
| | 1-37 | | | | | | |
| Example 11 | Compound 2-4 | 4.03 | 6.49 | 5.00 | 0.138 | 0.045 | 210 |
| Example 12 | Compound | 4.09 | 6.68 | 5.13 | 0.138 | 0.045 | 206 |
| | 2-40 | | | | | | |
| Example 13 | Compound | 3.97 | 6.67 | 5.28 | 0.138 | 0.045 | 208 |
| | 2-41 | | | | | | |
| Example 14 | Compound | 4.01 | 6.67 | 5.23 | 0.138 | 0.045 | 216 |
| | 2-42 | | | | | | |
| Example 15 | Compound | 3.82 | 6.90 | 5.53 | 0.138 | 0.045 | 222 |
| | 2-43 | | | | | | |
| Example 16 | Compound | 3.89 | 6.84 | 5.50 | 0.138 | 0.045 | 250 |
| | 2-20 | | | | | | |
| Example 17 | Compound | 3.87 | 6.77 | 5.44 | 0.138 | 0.045 | 252 |
| | 2-16 | | | | | | |
| Example 18 | Compound | 3.97 | 6.49 | 5.14 | 0.138 | 0.045 | 202 |
| | 2-45 | | | | | | |
| Example 19 | Compound | 4.08 | 6.62 | 5.06 | 0.138 | 0.045 | 209 |
| | 2-46 | | | | | | |
| Example 20 | Compound | 3.94 | 6.71 | 5.35 | 0.138 | 0.045 | 233 |
| | 2-32 | | | | | | |
| Example 21 | Compound | 3.89 | 6.75 | 5.38 | 0.138 | 0.045 | 237 |
| | 2-34 | | | | | | |
| Example 22 | Compound | 4.02 | 6.88 | 5.26 | 0.138 | 0.045 | 214 |
| | 3-20 | | | | | | |
| Example 23 | Compound | 3.99 | 6.37 | 5.02 | 0.138 | 0.045 | 204 |
| | 4-21 | | | | | | |
| Example 24 | Compound | 4.04 | 6.69 | 5.20 | 0.138 | 0.045 | 211 |
| | 5-12 | | | | | | |
| Example 25 | Compound | 3.88 | 6.80 | 5.48 | 0.138 | 0.045 | 253 |
| | 2-56 | | | | | | |
| Example 26 | Compound | 3.84 | 6.78 | 5.41 | 0.138 | 0.045 | 254 |
| | 4-27 | | | | | | |
| Example 27 | Compound | 3.86 | 6.81 | 5.54 | 0.138 | 0.045 | 260 |
| | 4-33 | | | | | | |
| Example 28 | Compound | 3.90 | 6.85 | 5.52 | 0.138 | 0.045 | 258 |
| | 4-35 | | | | | | |
| Example 29 | Compound | 3.93 | 6.82 | 5.45 | 0.138 | 0.045 | 255 |
| | 4-41 | | | | | | |
| Example 30 | Compound | 3.87 | 6.85 | 5.53 | 0.138 | 0.045 | 251 |
| | 4-42 | | | | | | |
| Example 31 | Compound | 3.86 | 6.75 | 5.49 | 0.138 | 0.045 | 256 |
| | 4-43 | | | | | | |
| Example 32 | Compound | 3.91 | 6.90 | 5.60 | 0.138 | 0.045 | 262 |
| | 4-44 | | | | | | |
| Example 33 | Compound | 4.05 | 6.51 | 5.05 | 0.138 | 0.045 | 213 |
| | 5-20 | | | | | | |
| Example 34 | Compound | 3.88 | 6.74 | 5.46 | 0.138 | 0.045 | 249 |
| | 3-28 | | | | | | |
| Example 35 | Compound | 3.87 | 6.76 | 5.52 | 0.138 | 0.045 | 245 |
| | 2-78 | | | | | | |
| Example 36 | Compound | 4.02 | 6.89 | 5.27 | 0.138 | 0.045 | 217 |
| | 5-22 | | | | | | |
| Comparative | Compound A | 4.17 | 5.77 | 4.35 | 0.138 | 0.045 | 171 |
| Example 1 | | | | | | | |
| Comparative | Compound B | 4.23 | 5.81 | 4.31 | 0.138 | 0.045 | 164 |
| Example 2 | | | | | | | |
| Comparative | Compound C | 4.10 | 5.94 | 4.61 | 0.138 | 0.045 | 179 |
| Example 3 | | | | | | | |
| Comparative | Compound D | 4.07 | 6.01 | 4.63 | 0.138 | 0.045 | 185 |
| Example 4 | | | | | | | |
| Comparative | Compound E | 4.14 | 5.88 | 4.46 | 0.138 | 0.045 | 174 |
| Example 5 | | | | | | | |

It can be seen from Table 5 that, compared with the organic electroluminescent devices prepared by existing compounds as hole transport layer materials in Comparative Example 1 to Comparative Example 5, the service life of organic electroluminescent devices prepared by the organic compounds in Example 1 to Example 36 as hole transport layer materials has been significantly improved, with the service life increased by at least 9.2%. The device also has higher luminous efficiency and lower driving voltage. Furthermore, compared with Comparative Example 1 to Comparative Example 5, in preferred embodiments such as Examples 25 to 32, not only the service life of the prepared device is significantly improved, but also the luminous efficiency of the device is significantly improved, and the driving voltage is also lower.

1. An organic compound having the structure shown in Formula 1: 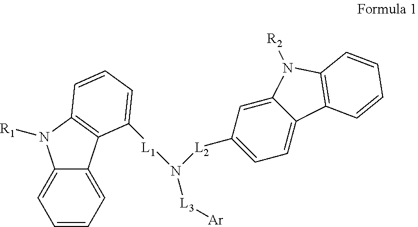
wherein R₁ and R₂ are the same or different, and are each independently selected from a substituted or unsubstituted aryl with 6 to 25 carbon atoms, a substituted or unsubstituted heteroaryl with 5 to 25 carbon atoms, an alkyl with 1 to 10 carbon atoms, or a cycloalkyl with 3 to 10 carbon atoms;
Ar is selected from a substituted or unsubstituted aryl with 6 to 21 carbon atoms, or a substituted or unsubstituted heteroaryl with 5 to 20 carbon atoms;
L₁, L₂ and L₃ are the same or different, and are each independently selected from a single bond, a substituted or unsubstituted arylene with 6 to 15 carbon atoms, or a heteroarylene with 5 to 15 carbon atoms;
the substituents of R₁, R₂, Ar, L₁, L₂ and L₃ are the same or different, and are each independently selected from deuterium, a cyano, an alkyl with 1 to 10 carbon atoms, a deuterated alkyl with 1 to 10 carbon atoms, an alkoxy with 1 to 10 carbon atoms, an alkylthio with 1 to 10 carbon atoms, an aryl with 6 to 12 carbon atoms, a heteroaryl with 5 to 12 carbon atoms, or a cycloalkyl with 3 to 10 carbon atoms; and
the total number of carbon atoms of L₃ and Ar is not more than 21.

2. The organic compound according to claim 1, wherein, the structure of the organic compound is selected from the following structural formulae: 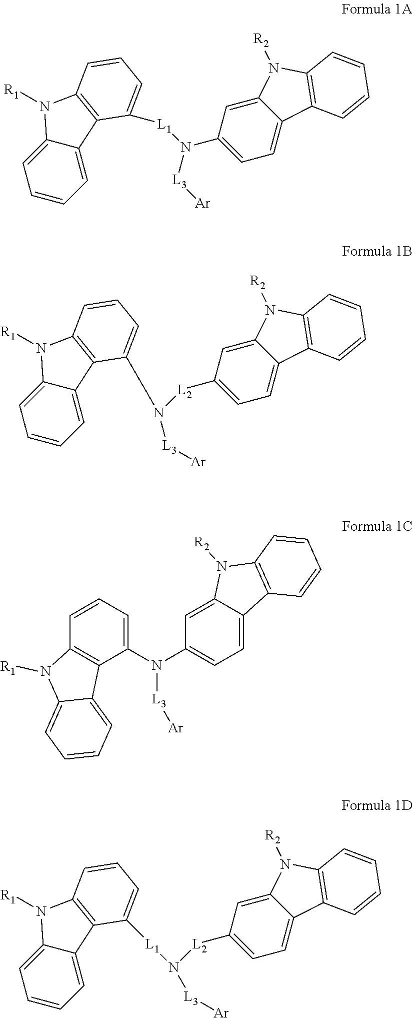
R₁, R₂, Ar, L₁, L₂ and L₃ in Formula 1A, Formula 1B, Formula 1C and Formula 1D have the same definitions as those in Formula 1.

3. The organic compound according to claim 1, wherein, R₁ and R₂ are the same or different, and are each independently selected from a substituted or unsubstituted aryl with 6 to 18 carbon atoms, a substituted or unsubstituted heteroaryl with 5 to 15 carbon atoms, an alkyl with 1 to 5 carbon atoms, or a cycloalkyl with 3 to 8 carbon atoms;
optionally, the substituents of R₁ and R₂ are the same or different, and are each independently selected from deuterium, a cyano, an alkyl with 1 to 4 carbon atoms, a deuterated alkyl with 1 to 4 carbon atoms, an alkoxy with 1 to 4 carbon atoms, an alkylthio with 1 to 4 carbon atoms, an aryl with 6 to 10 carbon atoms, or a cycloalkyl with 5 to 10 carbon atoms.

4. The organic compound according to claim 1, wherein, R₁ and R₂ are the same or different, and are each independently selected from a methyl, an ethyl, a n-propyl, an isopropyl, a n-butyl, an isobutyl, a tert-butyl, a cyclopentyl, a cyclohexyl, a substituted or unsubstituted phenyl, a substituted or unsubstituted biphenyl, a substituted or unsubstituted naphthyl, a substituted or unsubstituted fluorenyl, a substituted or unsubstituted phenanthryl, a substituted or unsubstituted terphenyl, a substituted or unsubstituted pyridyl, a substituted or unsubstituted dibenzofuranyl, a substituted or unsubstituted dibenzothienyl, or a substituted or unsubstituted carbazolyl;
optionally, each of the substituents of R₁ and R₂ is independently selected from deuterium, a cyano, a methyl, an ethyl, a n-propyl, an isopropyl, a tert-butyl, a trideuterated methyl, a methoxy, an ethoxy, a phenyl, a naphthyl, a cyclopentyl or a cyclohexyl.

5. The organic compound according to claim 1, wherein, R₁ and R₂ are the same or different, and are each independently selected from the group consisting of methyl, ethyl, n-propyl, isopropyl, n-butyl, isobutyl, tert-butyl, and the following groups:

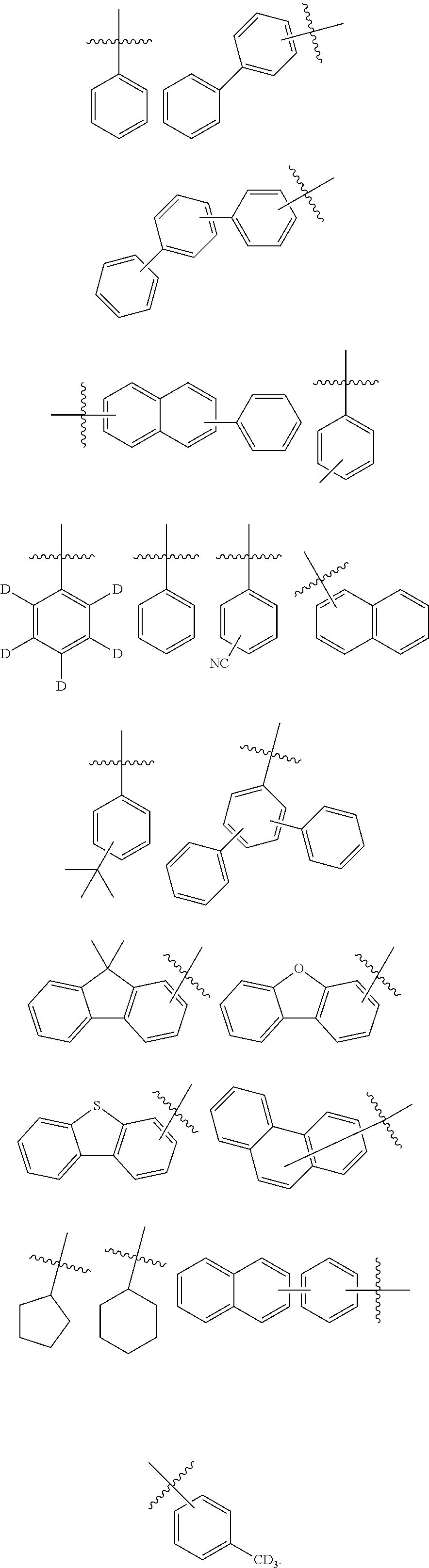

6. The organic compound according to claim 1, wherein, L₁, L₂ and L₃ are the same or different, and are each independently selected from a single bond, a substituted or unsubstituted arylene with 6 to 12 carbon atoms, or a substituted or unsubstituted heteroarylene with 5 to 12 carbon atoms;
optionally, the substituents of L₁, L₂ and L₃ are the same or different, and are each independently selected from deuterium, a cyano, an alkyl with 1 to 4 carbon atoms, a deuterated alkyl with 1 to 4 carbon atoms of 1 to 4, or a phenyl;
optionally, L₁ is a single bond, L₂ is selected from a substituted or unsubstituted arylene with 6 to 12 carbon atoms, L₃ is selected from a single bond, and a substituted or unsubstituted arylene with 6 to 12 f carbon atoms.

7. The organic compound according to claim 1, wherein, L₁, L₂ and L₃ are the same or different, and are each independently selected from a single bond, a substituted or unsubstituted phenylene, a substituted or unsubstituted naphthylene, a substituted or unsubstituted biphenylene, a substituted or unsubstituted pyridylidene, a substituted or unsubstituted dibenzofuranylene, or a substituted or unsubstituted dibenzothienylene;
optionally, the substituents of L₁, L₂ and L₃ are each independently selected from deuterium, a cyano, a methyl, an ethyl, an isopropyl, a tert-butyl, a trideuterated methyl, or a phenyl.

8. The organic compound according to claim 1, wherein, Ar is selected from a substituted or unsubstituted group Z, the unsubstituted group Z is selected from the group consisting of the following groups: 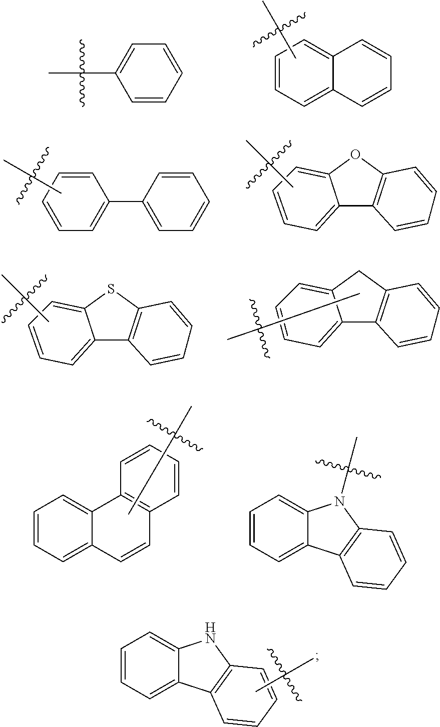
the substituted group Z has one or more substituents, each of the substituents is independently selected from the group consisting of deuterium, a cyano, a methyl, an ethyl, an isopropyl, a tert-butyl, a trideuterated methyl, a phenyl, and a naphthyl; when the number of the substituents is more than 1, the substituents are the same or different;
optionally, Ar is selected from the group consisting of the following groups:

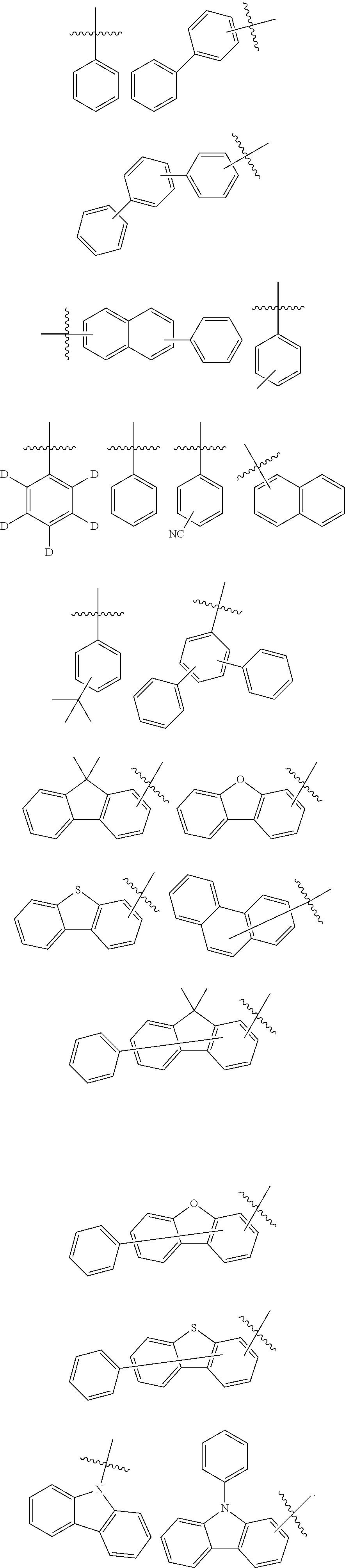

9. The organic compound according to claim 1, wherein, 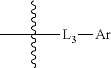
is selected from the group consisting of the following groups:

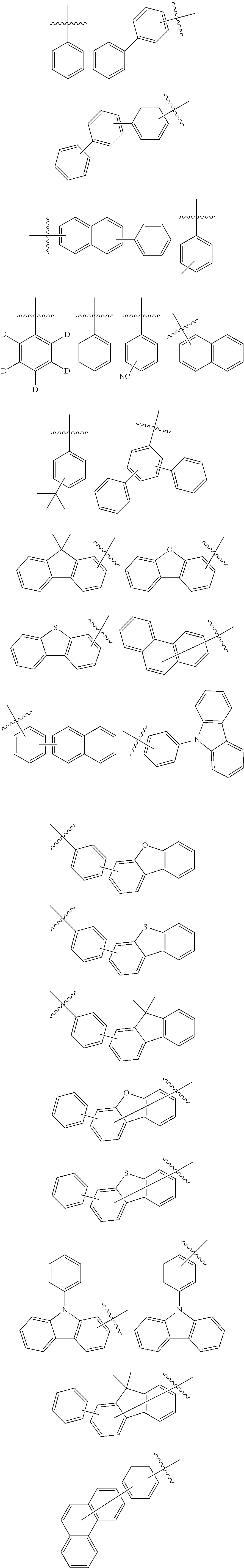

10. The organic compound according to claim 1, wherein, the organic compound is selected from the group consisting of the following compounds:

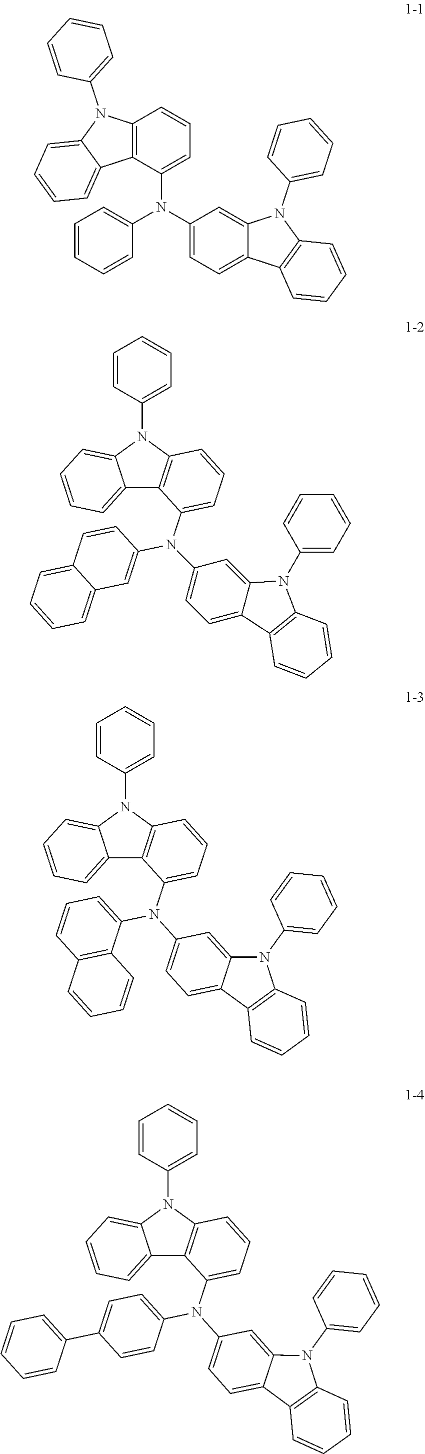 [SMILES: c1ccc(N(c2ccc3c4ccccc4n(-c4ccccc4)c3c2)c2cccc3c2c2ccccc2n3-c2ccccc2)cc1 ; c1ccc(-n2c3ccccc3c3ccc(N(c4ccc5ccccc5c4)c4cccc5c4c4ccccc4n5-c4ccccc4)cc32)cc1 ; c1ccc(-n2c3ccccc3c3ccc(N(c4cccc5ccccc45)c4cccc5c4c4ccccc4n5-c4ccccc4)cc32)cc1 ; c1ccc(-c2ccc(N(c3ccc4c5ccccc5n(-c5ccccc5)c4c3)c3cccc4c3c3ccccc3n4-c3ccccc3)cc2)cc1]

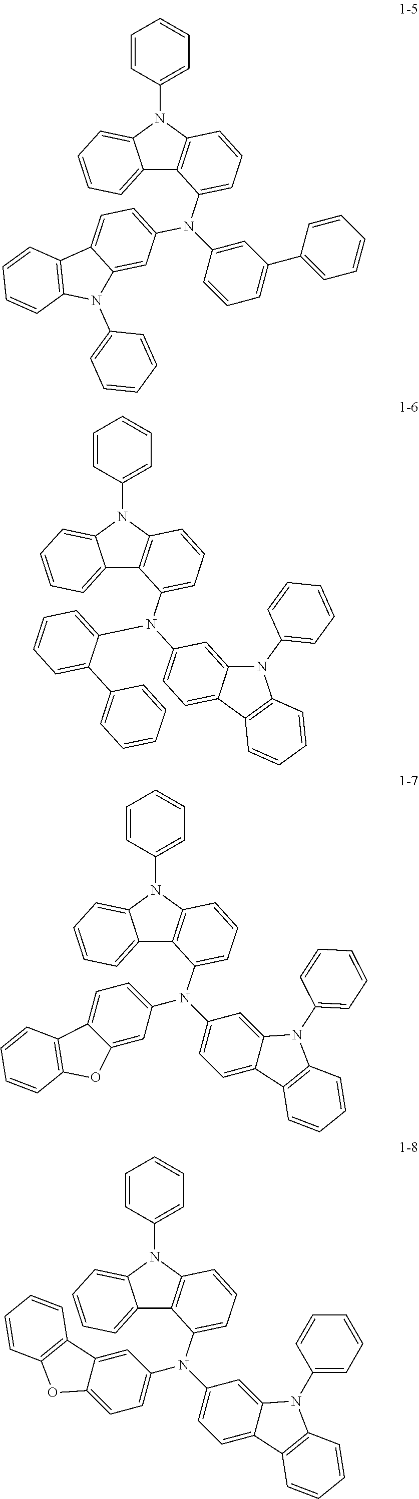

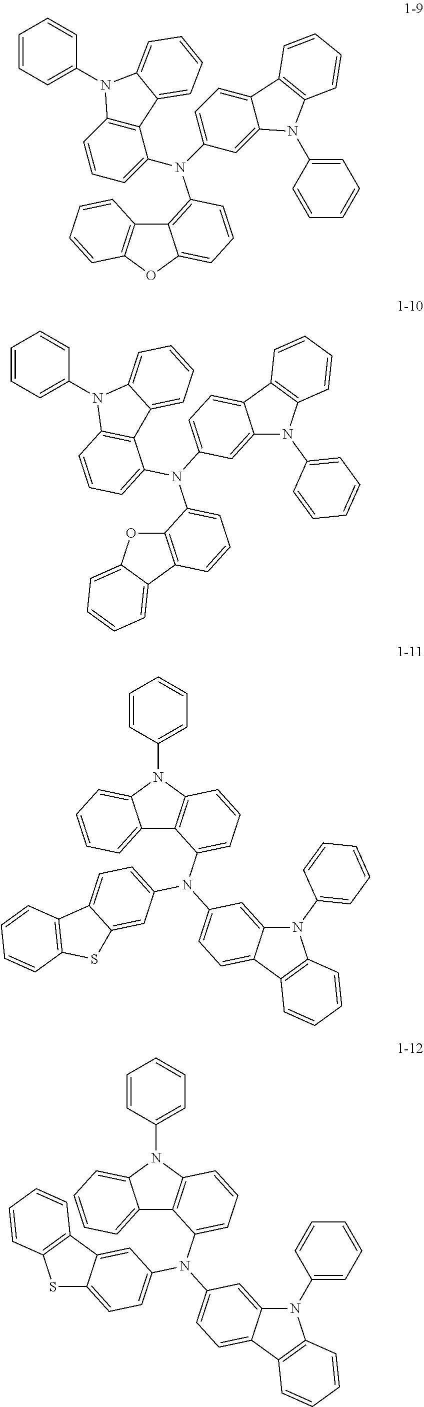 [SMILES: c1ccc(-n2c3ccccc3c3ccc(N(c4cccc5oc6ccccc6c45)c4cccc5c4c4ccccc4n5-c4ccccc4)cc32)cc1 ; c1ccc(-n2c3ccccc3c3ccc(N(c4cccc5c4oc4ccccc45)c4cccc5c4c4ccccc4n5-c4ccccc4)cc32)cc1 ; c1ccc(-n2c3ccccc3c3ccc(N(c4ccc5c(c4)sc4ccccc45)c4cccc5c4c4ccccc4n5-c4ccccc4)cc32)cc1 ; c1ccc(-n2c3ccccc3c3ccc(N(c4ccc5sc6ccccc6c5c4)c4cccc5c4c4ccccc4n5-c4ccccc4)cc32)cc1]

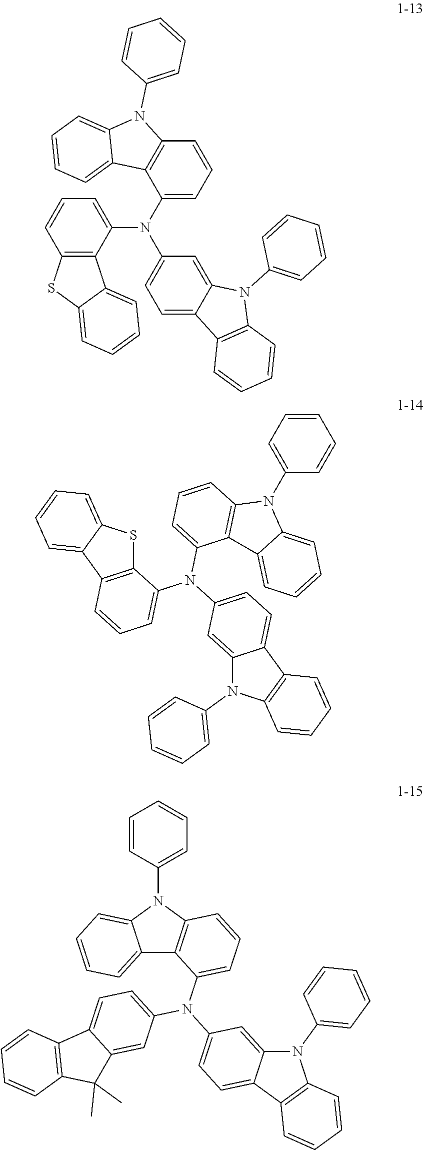

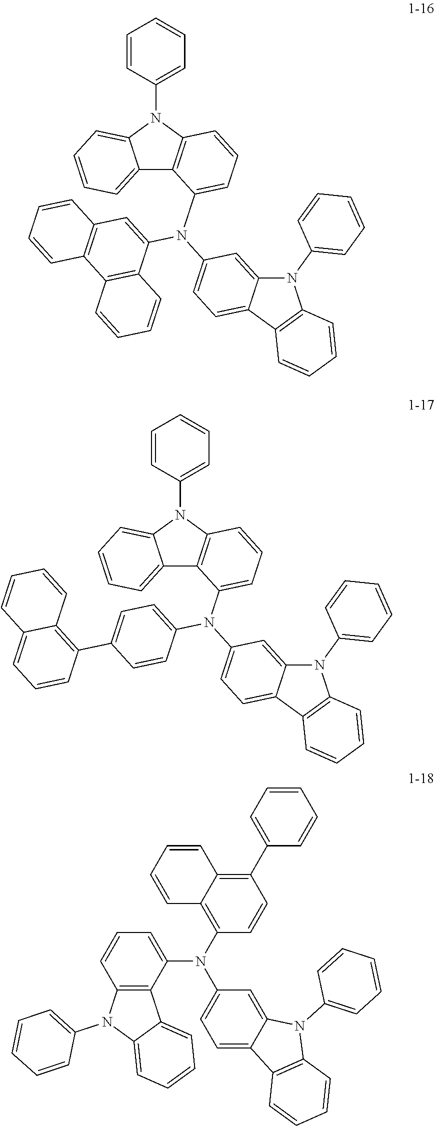

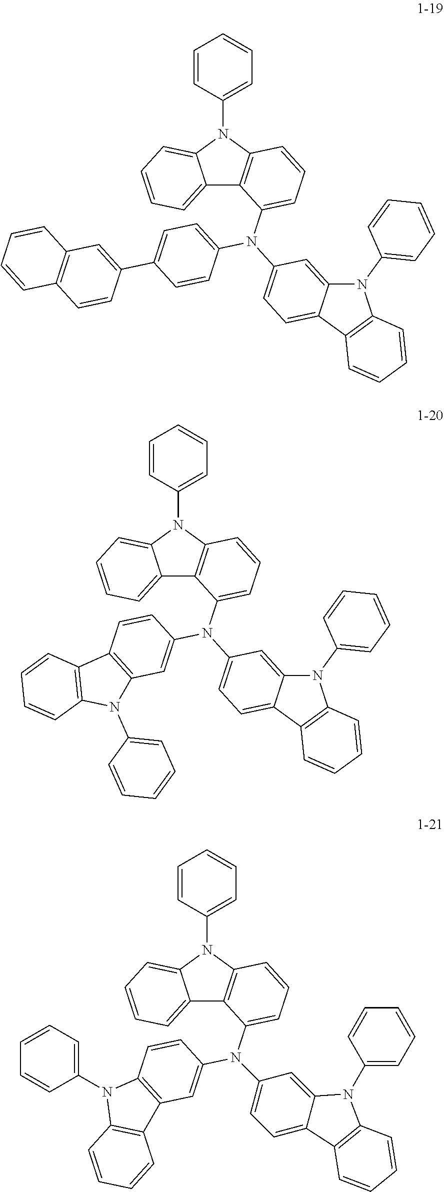 [SMILES: c1ccc(-n2c3ccccc3c3ccc(N(c4ccc(-c5ccc6ccccc6c5)cc4)c4cccc5c4c4ccccc4n5-c4ccccc4)cc32)cc1 ; c1ccc(-n2c3ccccc3c3ccc(N(c4ccc5c6ccccc6n(-c6ccccc6)c5c4)c4cccc5c4c4ccccc4n5-c4ccccc4)cc32)cc1 ; c1ccc(-n2c3ccccc3c3cc(N(c4ccc5c6ccccc6n(-c6ccccc6)c5c4)c4cccc5c4c4ccccc4n5-c4ccccc4)ccc32)cc1]

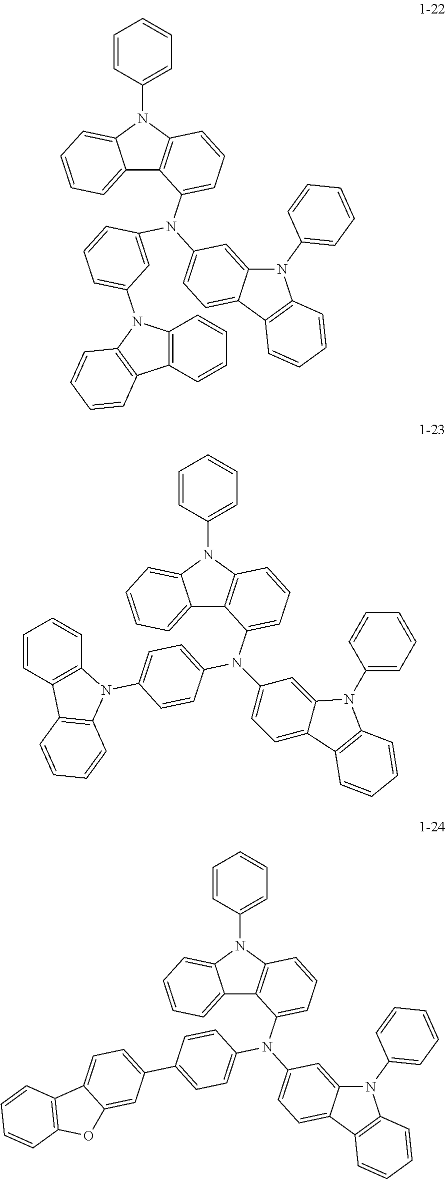 [SMILES: c1ccc(-n2c3ccccc3c3ccc(N(c4cccc(-n5c6ccccc6c6ccccc65)c4)c4cccc5c4c4ccccc4n5-c4ccccc4)cc32)cc1 ; c1ccc(-n2c3ccccc3c3ccc(N(c4ccc(-n5c6ccccc6c6ccccc65)cc4)c4cccc5c4c4ccccc4n5-c4ccccc4)cc32)cc1 ; c1ccc(-n2c3ccccc3c3ccc(N(c4ccc(-c5ccc6c(c5)oc5ccccc56)cc4)c4cccc5c4c4ccccc4n5-c4ccccc4)cc32)cc1]

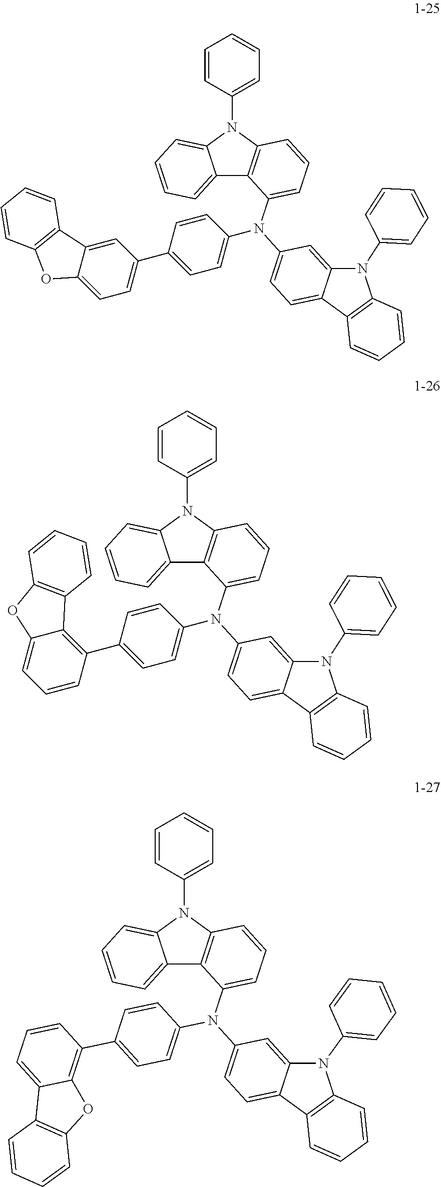 [SMILES: c1ccc(-n2c3ccccc3c3ccc(N(c4ccc(-c5ccc6oc7ccccc7c6c5)cc4)c4cccc5c4c4ccccc4n5-c4ccccc4)cc32)cc1 ; c1ccc(-n2c3ccccc3c3ccc(N(c4ccc(-c5cccc6oc7ccccc7c56)cc4)c4cccc5c4c4ccccc4n5-c4ccccc4)cc32)cc1 ; c1ccc(-n2c3ccccc3c3ccc(N(c4ccc(-c5cccc6c5oc5ccccc56)cc4)c4cccc5c4c4ccccc4n5-c4ccccc4)cc32)cc1]

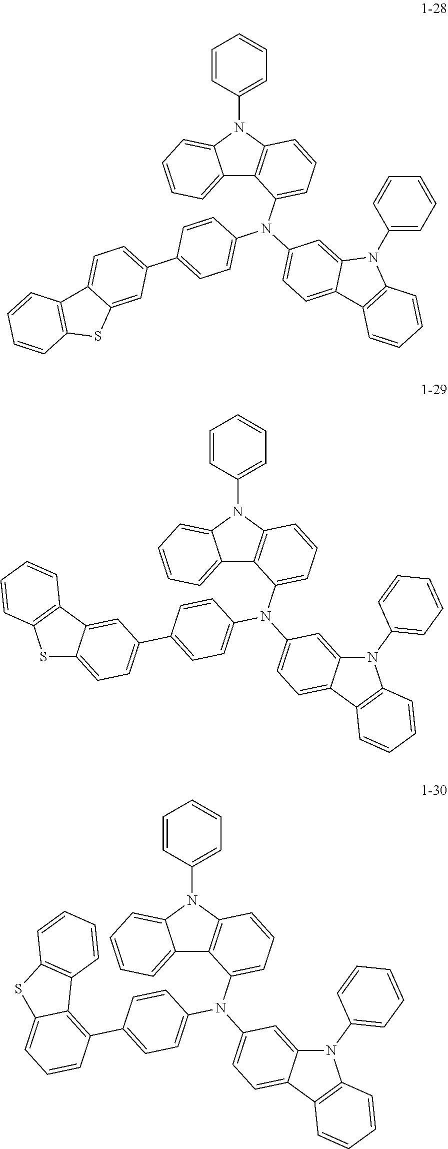 [SMILES: c1ccc(-n2c3ccccc3c3ccc(N(c4ccc(-c5ccc6c(c5)sc5ccccc56)cc4)c4cccc5c4c4ccccc4n5-c4ccccc4)cc32)cc1 ; c1ccc(-n2c3ccccc3c3ccc(N(c4ccc(-c5ccc6sc7ccccc7c6c5)cc4)c4cccc5c4c4ccccc4n5-c4ccccc4)cc32)cc1 ; c1ccc(-n2c3ccccc3c3ccc(N(c4ccc(-c5cccc6sc7ccccc7c56)cc4)c4cccc5c4c4ccccc4n5-c4ccccc4)cc32)cc1]

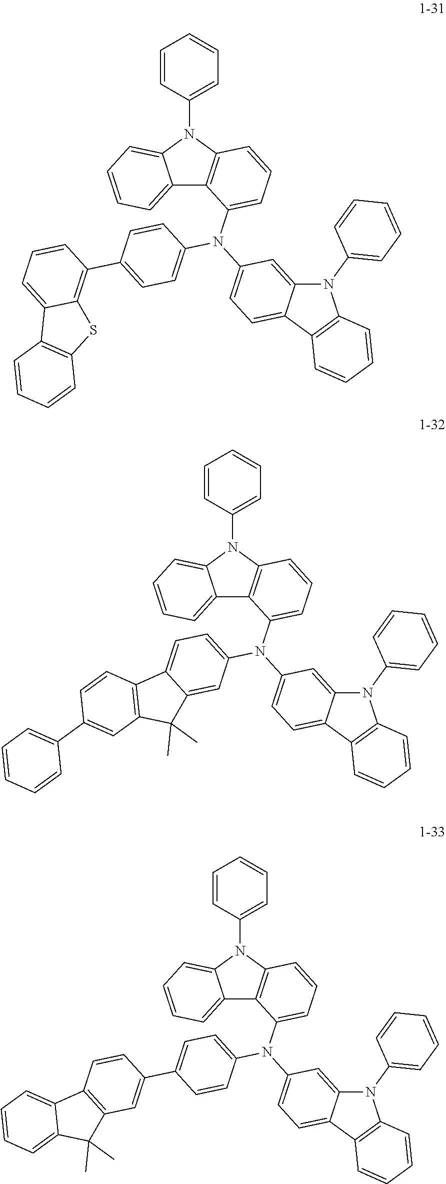 [SMILES: c1ccc(-n2c3ccccc3c3ccc(N(c4ccc(-c5cccc6c5sc5ccccc56)cc4)c4cccc5c4c4ccccc4n5-c4ccccc4)cc32)cc1 ; CC1(C)c2cc(-c3ccccc3)ccc2-c2ccc(N(c3ccc4c5ccccc5n(-c5ccccc5)c4c3)c3cccc4c3c3ccccc3n4-c3ccccc3)cc21 ; CC1(C)c2ccccc2-c2ccc(-c3ccc(N(c4ccc5c6ccccc6n(-c6ccccc6)c5c4)c4cccc5c4c4ccccc4n5-c4ccccc4)cc3)cc21]

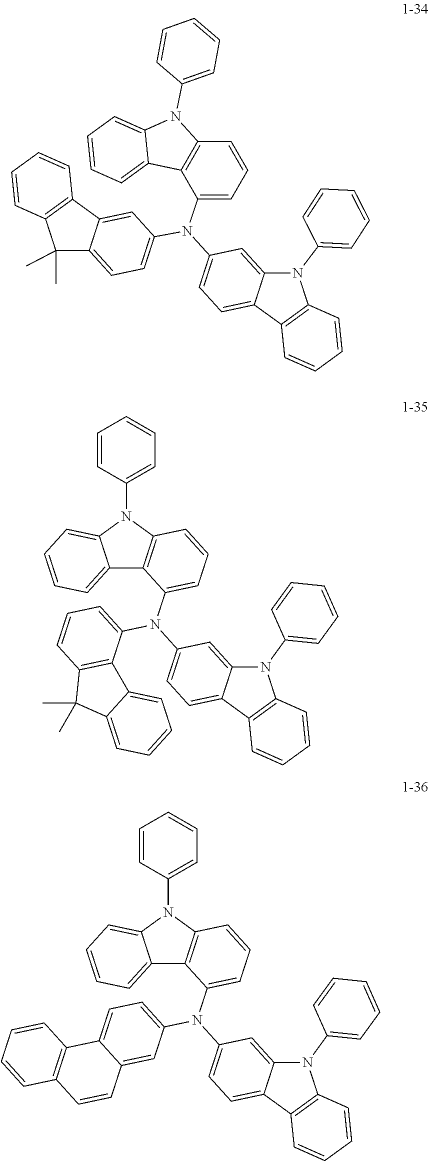 [SMILES: CC1(C)c2ccccc2-c2cc(N(c3ccc4c5ccccc5n(-c5ccccc5)c4c3)c3cccc4c3c3ccccc3n4-c3ccccc3)ccc21 ; CC1(C)c2ccccc2-c2c(N(c3ccc4c5ccccc5n(-c5ccccc5)c4c3)c3cccc4c3c3ccccc3n4-c3ccccc3)cccc21 ; c1ccc(-n2c3ccccc3c3ccc(N(c4ccc5c(ccc6ccccc65)c4)c4cccc5c4c4ccccc4n5-c4ccccc4)cc32)cc1]

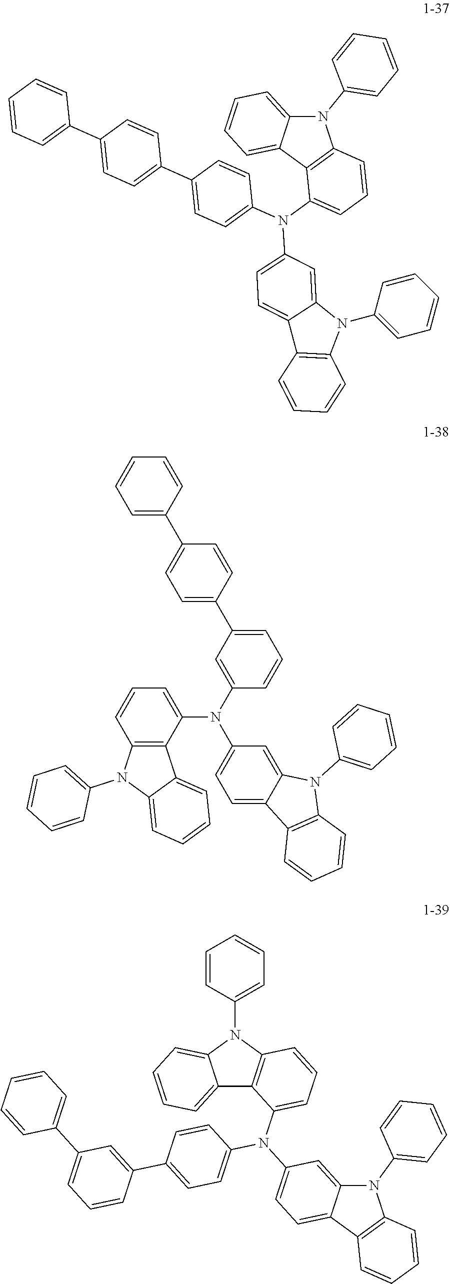 [SMILES: c1ccc(-c2ccc(-c3ccc(N(c4ccc5c6ccccc6n(-c6ccccc6)c5c4)c4cccc5c4c4ccccc4n5-c4ccccc4)cc3)cc2)cc1 ; c1ccc(-c2ccc(-c3cccc(N(c4ccc5c6ccccc6n(-c6ccccc6)c5c4)c4cccc5c4c4ccccc4n5-c4ccccc4)c3)cc2)cc1 ; c1ccc(-c2cccc(-c3ccc(N(c4ccc5c6ccccc6n(-c6ccccc6)c5c4)c4cccc5c4c4ccccc4n5-c4ccccc4)cc3)c2)cc1]

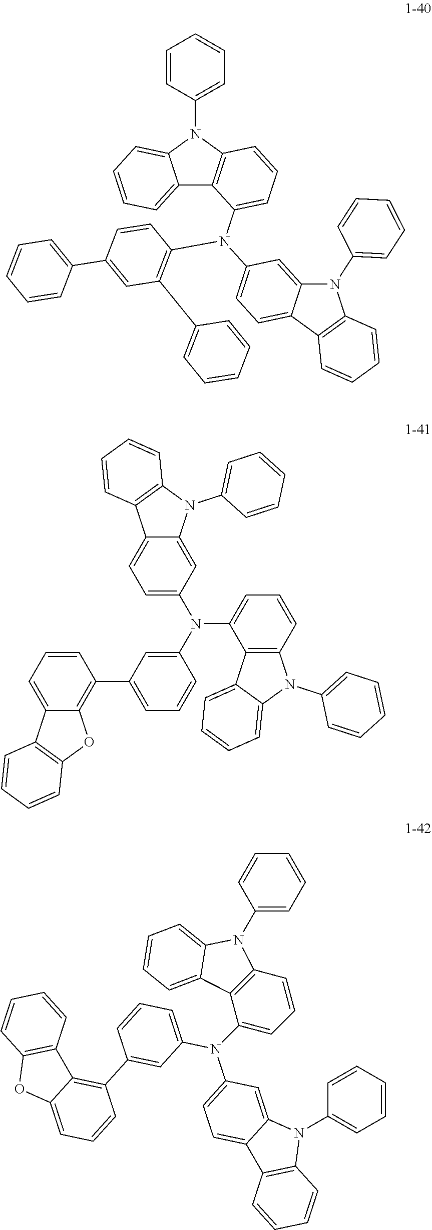 [SMILES: c1ccc(-c2ccc(N(c3ccc4c5ccccc5n(-c5ccccc5)c4c3)c3cccc4c3c3ccccc3n4-c3ccccc3)c(-c3ccccc3)c2)cc1 ; c1ccc(-n2c3ccccc3c3ccc(N(c4cccc(-c5cccc6c5oc5ccccc56)c4)c4cccc5c4c4ccccc4n5-c4ccccc4)cc32)cc1 ; c1ccc(-n2c3ccccc3c3ccc(N(c4cccc(-c5cccc6oc7ccccc7c56)c4)c4cccc5c4c4ccccc4n5-c4ccccc4)cc32)cc1]

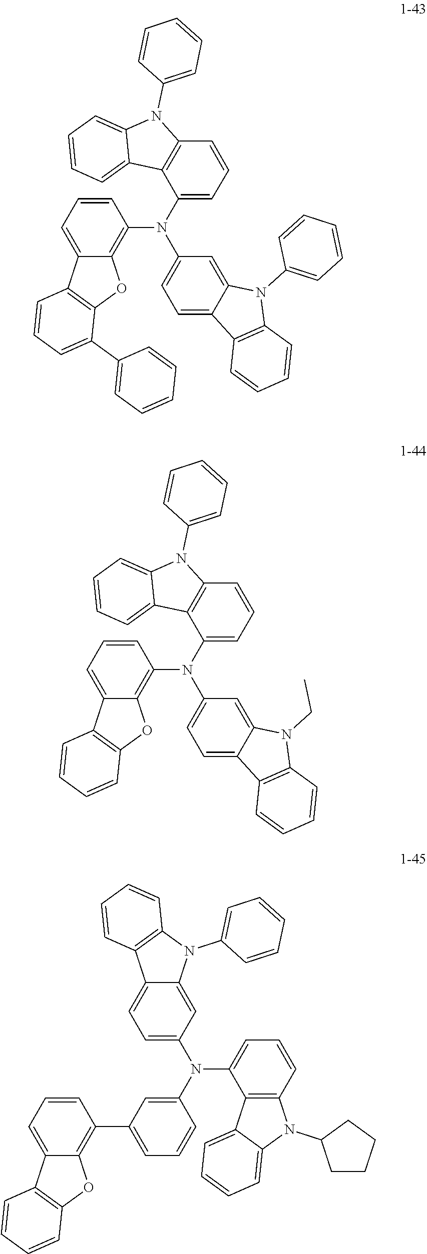 [SMILES: c1ccc(-c2cccc3c2oc2c(N(c4ccc5c6ccccc6n(-c6ccccc6)c5c4)c4cccc5c4c4ccccc4n5-c4ccccc4)cccc23)cc1 ; CCn1c2ccccc2c2ccc(N(c3cccc4c3oc3ccccc34)c3cccc4c3c3ccccc3n4-c3ccccc3)cc21 ; c1ccc(-n2c3ccccc3c3ccc(N(c4cccc(-c5cccc6c5oc5ccccc56)c4)c4cccc5c4c4ccccc4n5C4CCCC4)cc32)cc1]

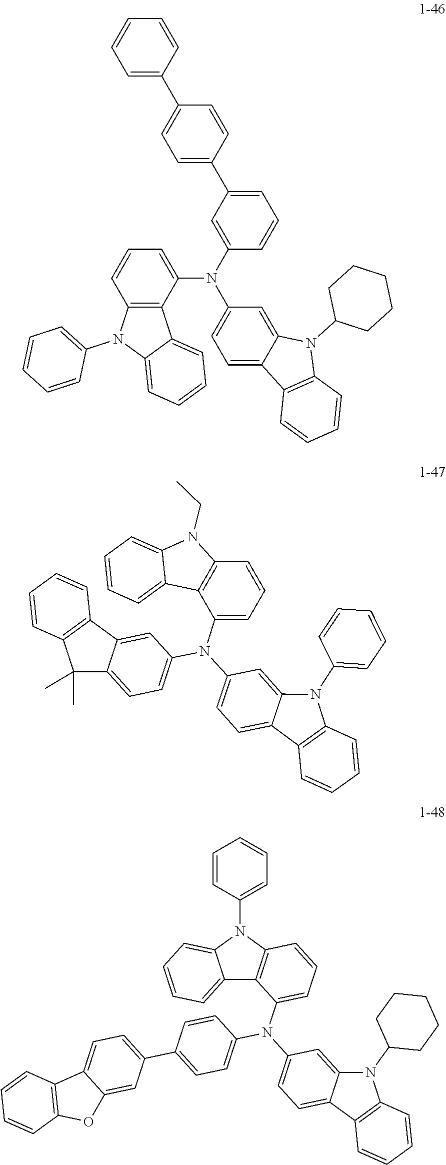 [SMILES: c1ccc(-c2ccc(-c3cccc(N(c4ccc5c6ccccc6n(C6CCCCC6)c5c4)c4cccc5c4c4ccccc4n5-c4ccccc4)c3)cc2)cc1 ; CCn1c2ccccc2c2c(N(c3ccc4c(c3)-c3ccccc3C4(C)C)c3ccc4c5ccccc5n(-c5ccccc5)c4c3)cccc21 ; c1ccc(-n2c3ccccc3c3c(N(c4ccc(-c5ccc6c(c5)oc5ccccc56)cc4)c4ccc5c6ccccc6n(C6CCCCC6)c5c4)cccc32)cc1]

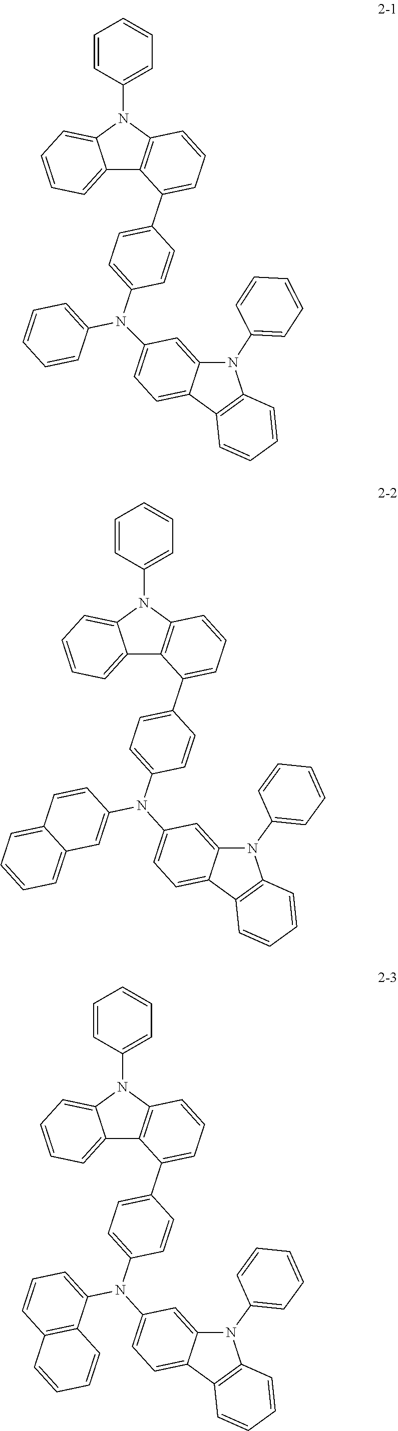 [SMILES: c1ccc(N(c2ccc(-c3cccc4c3c3ccccc3n4-c3ccccc3)cc2)c2ccc3c4ccccc4n(-c4ccccc4)c3c2)cc1 ; c1ccc(-n2c3ccccc3c3ccc(N(c4ccc(-c5cccc6c5c5ccccc5n6-c5ccccc5)cc4)c4ccc5ccccc5c4)cc32)cc1 ; c1ccc(-n2c3ccccc3c3ccc(N(c4ccc(-c5cccc6c5c5ccccc5n6-c5ccccc5)cc4)c4cccc5ccccc45)cc32)cc1]

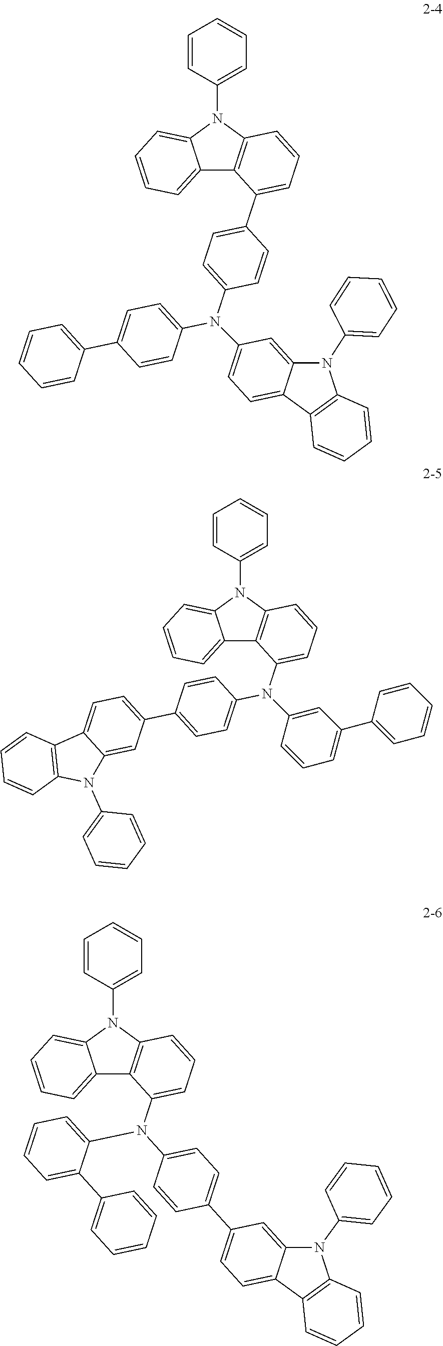 [SMILES: c1ccc(-c2ccc(N(c3ccc(-c4cccc5c4c4ccccc4n5-c4ccccc4)cc3)c3ccc4c5ccccc5n(-c5ccccc5)c4c3)cc2)cc1 ; c1ccc(-c2cccc(N(c3ccc(-c4ccc5c6ccccc6n(-c6ccccc6)c5c4)cc3)c3cccc4c3c3ccccc3n4-c3ccccc3)c2)cc1 ; c1ccc(-c2ccccc2N(c2ccc(-c3ccc4c5ccccc5n(-c5ccccc5)c4c3)cc2)c2cccc3c2c2ccccc2n3-c2ccccc2)cc1]

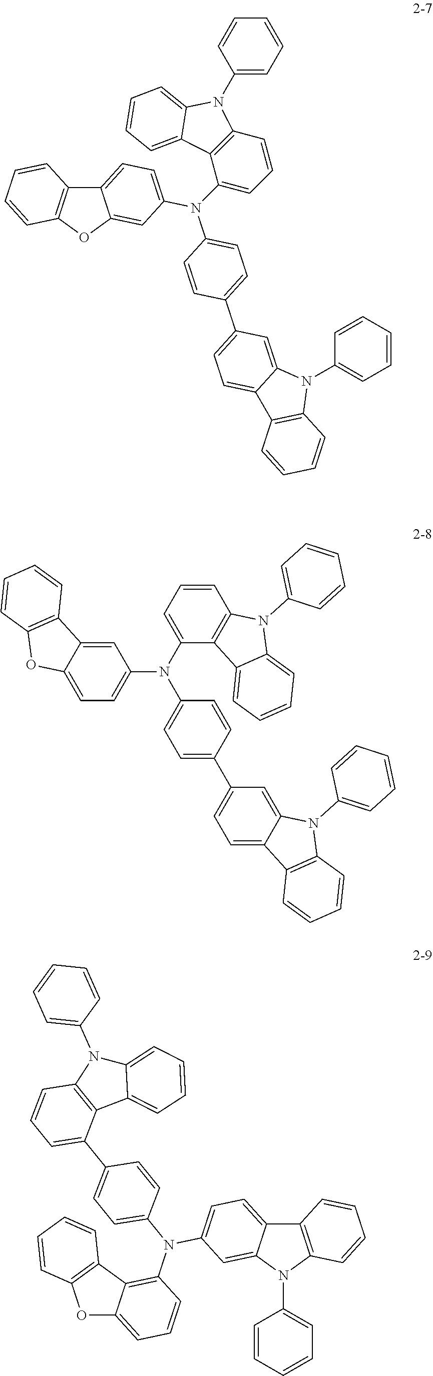 [SMILES: c1ccc(-n2c3ccccc3c3ccc(-c4ccc(N(c5ccc6c(c5)oc5ccccc56)c5cccc6c5c5ccccc5n6-c5ccccc5)cc4)cc32)cc1 ; c1ccc(-n2c3ccccc3c3ccc(-c4ccc(N(c5ccc6oc7ccccc7c6c5)c5cccc6c5c5ccccc5n6-c5ccccc5)cc4)cc32)cc1 ; c1ccc(-n2c3ccccc3c3ccc(N(c4ccc(-c5cccc6c5c5ccccc5n6-c5ccccc5)cc4)c4cccc5oc6ccccc6c45)cc32)cc1]

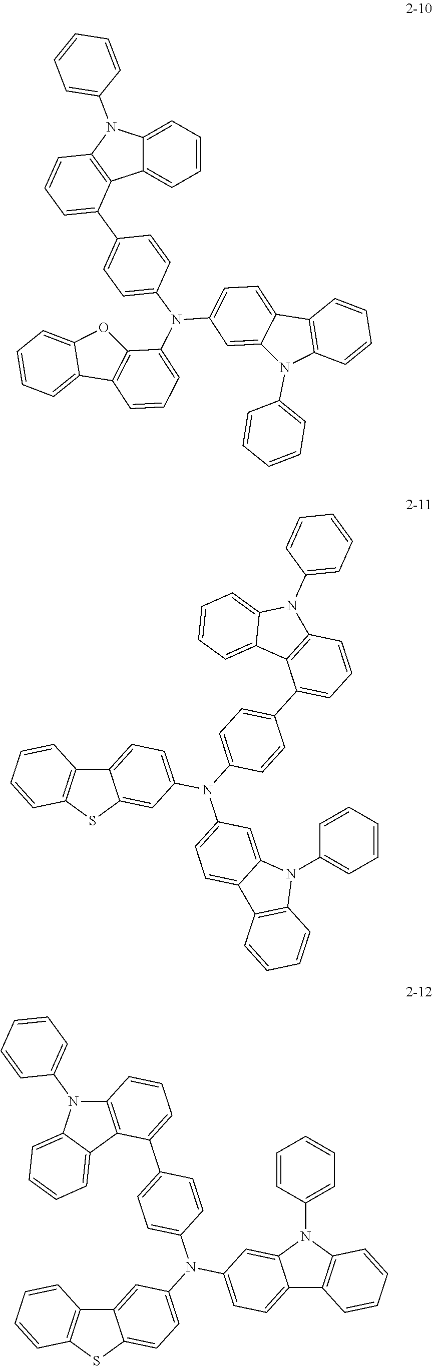 [SMILES: c1ccc(-n2c3ccccc3c3ccc(N(c4ccc(-c5cccc6c5c5ccccc5n6-c5ccccc5)cc4)c4cccc5c4oc4ccccc45)cc32)cc1 ; c1ccc(-n2c3ccccc3c3ccc(N(c4ccc(-c5cccc6c5c5ccccc5n6-c5ccccc5)cc4)c4ccc5c(c4)sc4ccccc45)cc32)cc1 ; c1ccc(-n2c3ccccc3c3ccc(N(c4ccc(-c5cccc6c5c5ccccc5n6-c5ccccc5)cc4)c4ccc5sc6ccccc6c5c4)cc32)cc1]

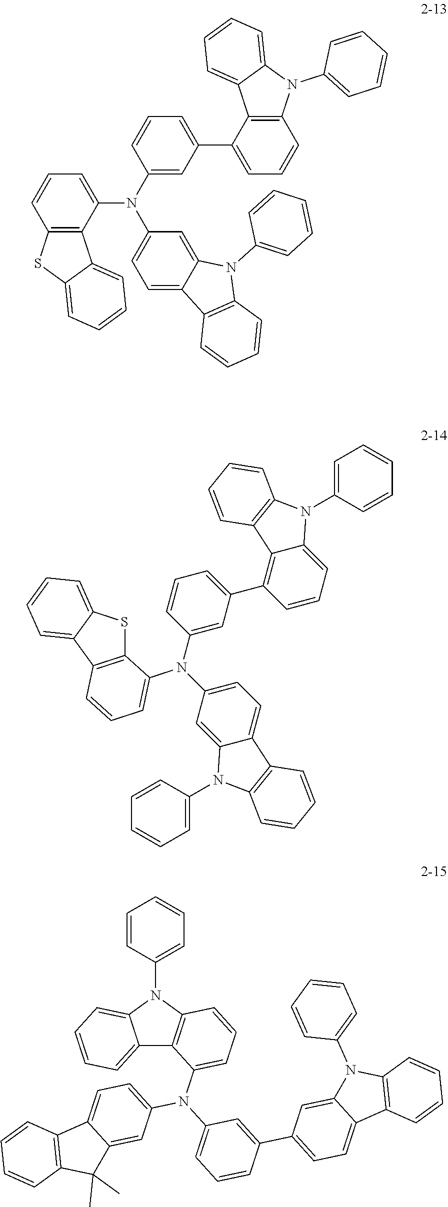 [SMILES: c1ccc(-n2c3ccccc3c3ccc(N(c4cccc(-c5cccc6c5c5ccccc5n6-c5ccccc5)c4)c4cccc5sc6ccccc6c45)cc32)cc1 ; c1ccc(-n2c3ccccc3c3ccc(N(c4cccc(-c5cccc6c5c5ccccc5n6-c5ccccc5)c4)c4cccc5c4sc4ccccc45)cc32)cc1 ; CC1(C)c2ccccc2-c2ccc(N(c3cccc(-c4ccc5c6ccccc6n(-c6ccccc6)c5c4)c3)c3cccc4c3c3ccccc3n4-c3ccccc3)cc21]

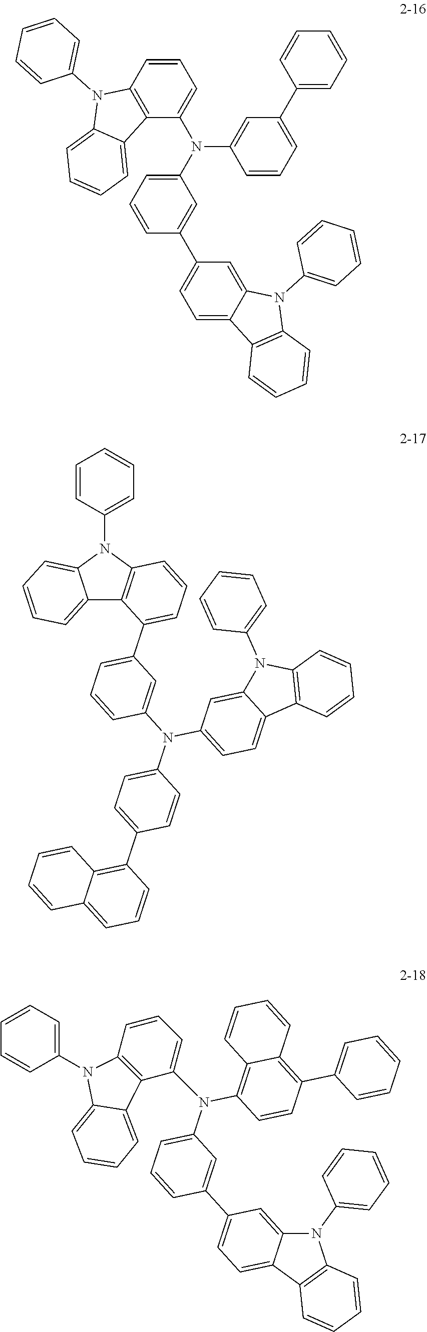 [SMILES: c1ccc(-c2cccc(N(c3cccc(-c4ccc5c6ccccc6n(-c6ccccc6)c5c4)c3)c3cccc4c3c3ccccc3n4-c3ccccc3)c2)cc1 ; c1ccc(-n2c3ccccc3c3ccc(N(c4ccc(-c5cccc6ccccc56)cc4)c4cccc(-c5cccc6c5c5ccccc5n6-c5ccccc5)c4)cc32)cc1 ; c1ccc(-c2ccc(N(c3cccc(-c4ccc5c6ccccc6n(-c6ccccc6)c5c4)c3)c3cccc4c3c3ccccc3n4-c3ccccc3)c3ccccc23)cc1]

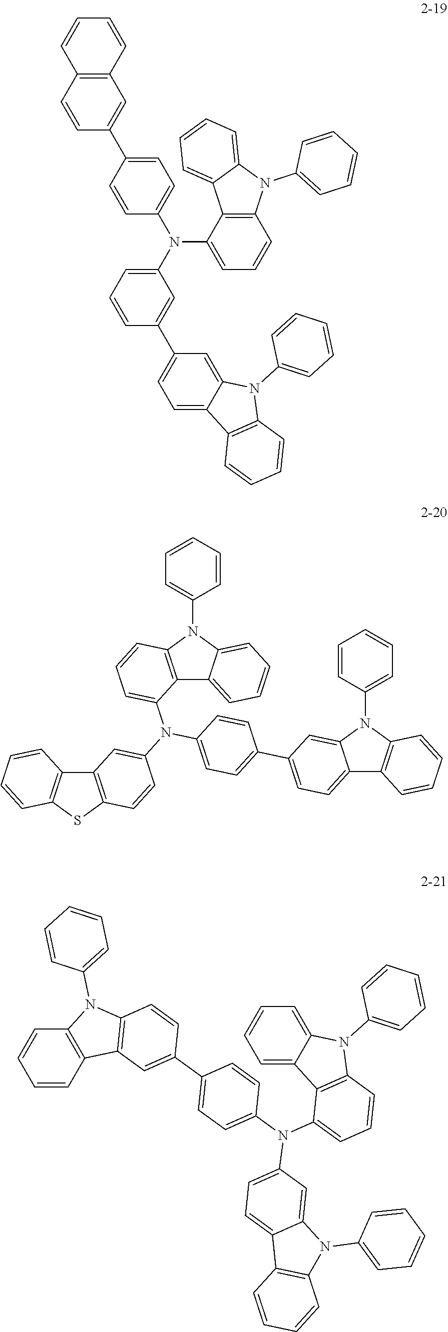 [SMILES: c1ccc(-n2c3ccccc3c3ccc(-c4cccc(N(c5ccc(-c6ccc7ccccc7c6)cc5)c5cccc6c5c5ccccc5n6-c5ccccc5)c4)cc32)cc1 ; c1ccc(-n2c3ccccc3c3ccc(-c4ccc(N(c5ccc6sc7ccccc7c6c5)c5cccc6c5c5ccccc5n6-c5ccccc5)cc4)cc32)cc1 ; c1ccc(-n2c3ccccc3c3cc(-c4ccc(N(c5ccc6c7ccccc7n(-c7ccccc7)c6c5)c5cccc6c5c5ccccc5n6-c5ccccc5)cc4)ccc32)cc1]

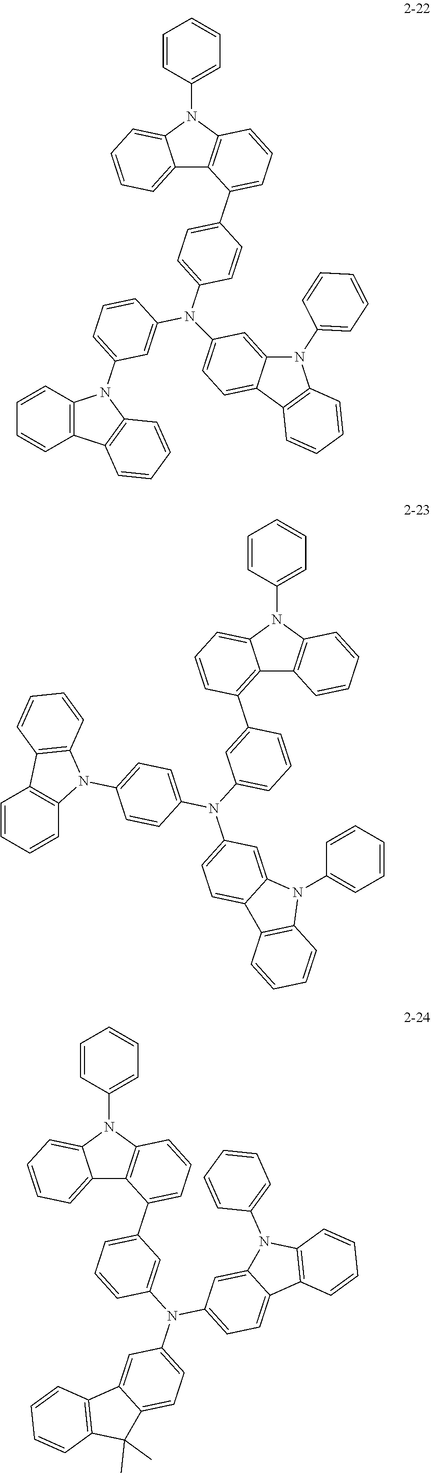 [SMILES: c1ccc(-n2c3ccccc3c3ccc(N(c4ccc(-c5cccc6c5c5ccccc5n6-c5ccccc5)cc4)c4cccc(-n5c6ccccc6c6ccccc65)c4)cc32)cc1 ; c1ccc(-n2c3ccccc3c3ccc(N(c4ccc(-n5c6ccccc6c6ccccc65)cc4)c4cccc(-c5cccc6c5c5ccccc5n6-c5ccccc5)c4)cc32)cc1 ; CC1(C)c2ccccc2-c2cc(N(c3cccc(-c4cccc5c4c4ccccc4n5-c4ccccc4)c3)c3ccc4c5ccccc5n(-c5ccccc5)c4c3)ccc21]

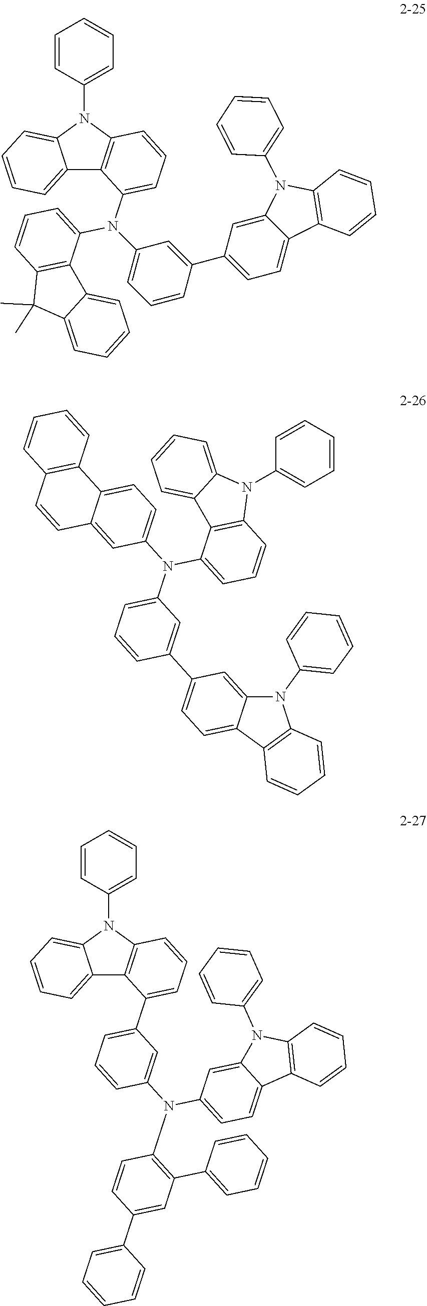 [SMILES: CC1(C)c2ccccc2-c2c(N(c3cccc(-c4ccc5c6ccccc6n(-c6ccccc6)c5c4)c3)c3cccc4c3c3ccccc3n4-c3ccccc3)cccc21 ; c1ccc(-n2c3ccccc3c3ccc(-c4cccc(N(c5ccc6c(ccc7ccccc76)c5)c5cccc6c5c5ccccc5n6-c5ccccc5)c4)cc32)cc1 ; c1ccc(-c2ccc(N(c3cccc(-c4cccc5c4c4ccccc4n5-c4ccccc4)c3)c3ccc4c5ccccc5n(-c5ccccc5)c4c3)c(-c3ccccc3)c2)cc1]

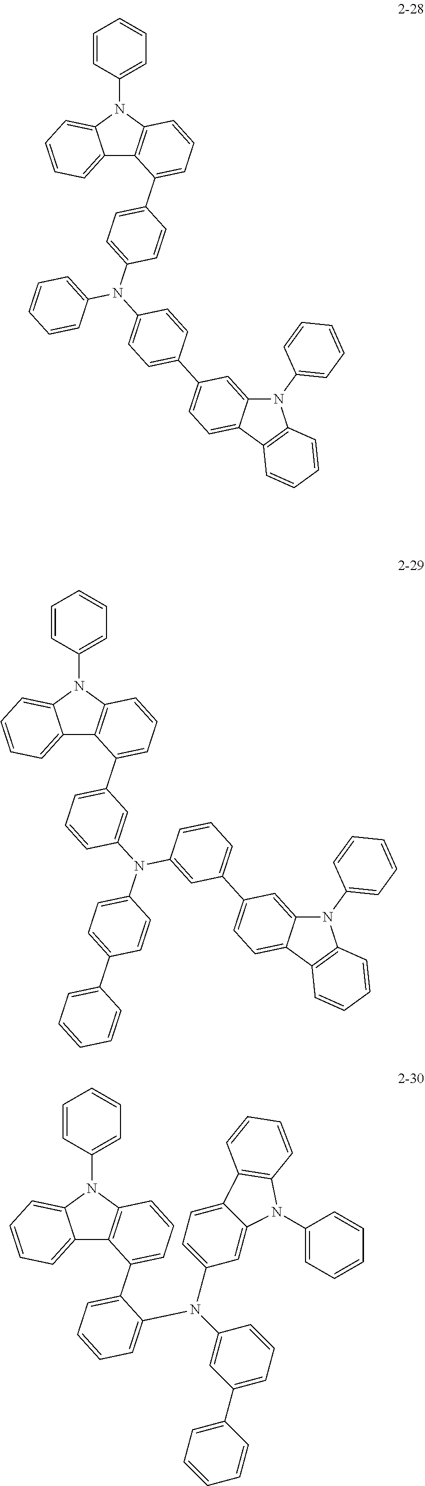

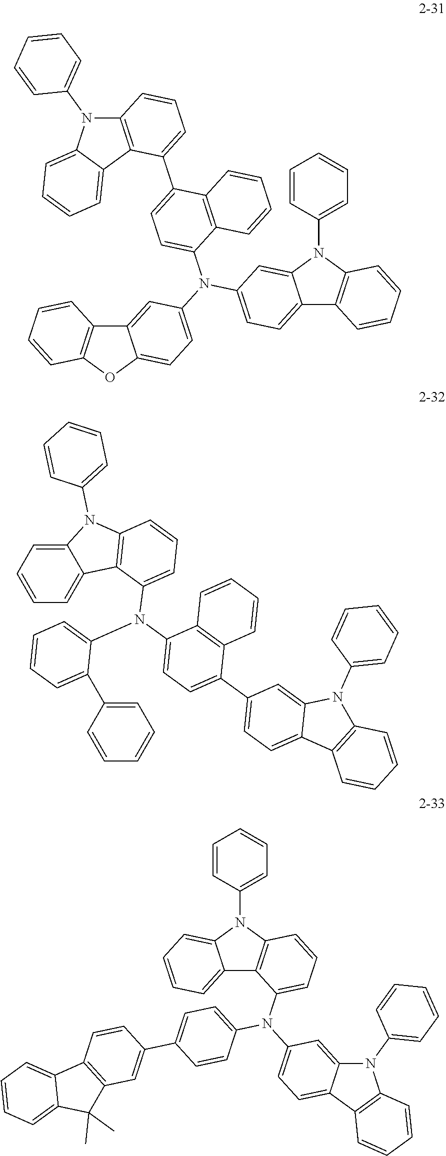 [SMILES: c1ccc(-n2c3ccccc3c3ccc(N(c4ccc5oc6ccccc6c5c4)c4ccc(-c5cccc6c5c5ccccc5n6-c5ccccc5)c5ccccc45)cc32)cc1 ; c1ccc(-c2ccccc2N(c2ccc(-c3ccc4c5ccccc5n(-c5ccccc5)c4c3)c3ccccc23)c2cccc3c2c2ccccc2n3-c2ccccc2)cc1 ; CC1(C)c2ccccc2-c2ccc(-c3ccc(N(c4ccc5c6ccccc6n(-c6ccccc6)c5c4)c4cccc5c4c4ccccc4n5-c4ccccc4)cc3)cc21]

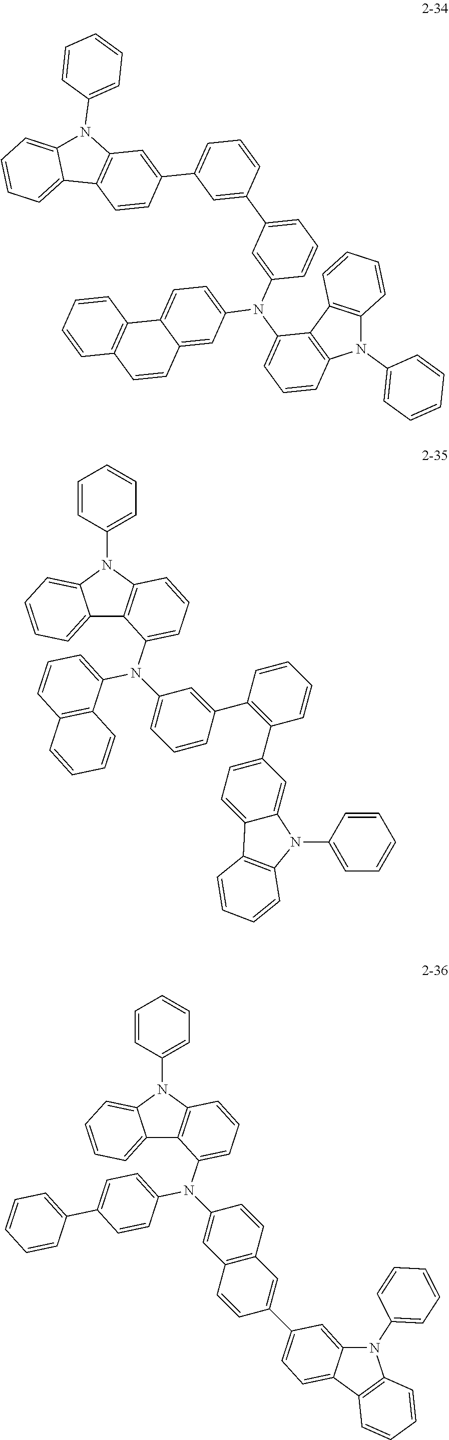 [SMILES: c1ccc(-n2c3ccccc3c3ccc(-c4cccc(-c5cccc(N(c6ccc7c(ccc8ccccc87)c6)c6cccc7c6c6ccccc6n7-c6ccccc6)c5)c4)cc32)cc1 ; c1ccc(-n2c3ccccc3c3ccc(-c4ccccc4-c4cccc(N(c5cccc6ccccc56)c5cccc6c5c5ccccc5n6-c5ccccc5)c4)cc32)cc1 ; c1ccc(-c2ccc(N(c3ccc4cc(-c5ccc6c7ccccc7n(-c7ccccc7)c6c5)ccc4c3)c3cccc4c3c3ccccc3n4-c3ccccc3)cc2)cc1]

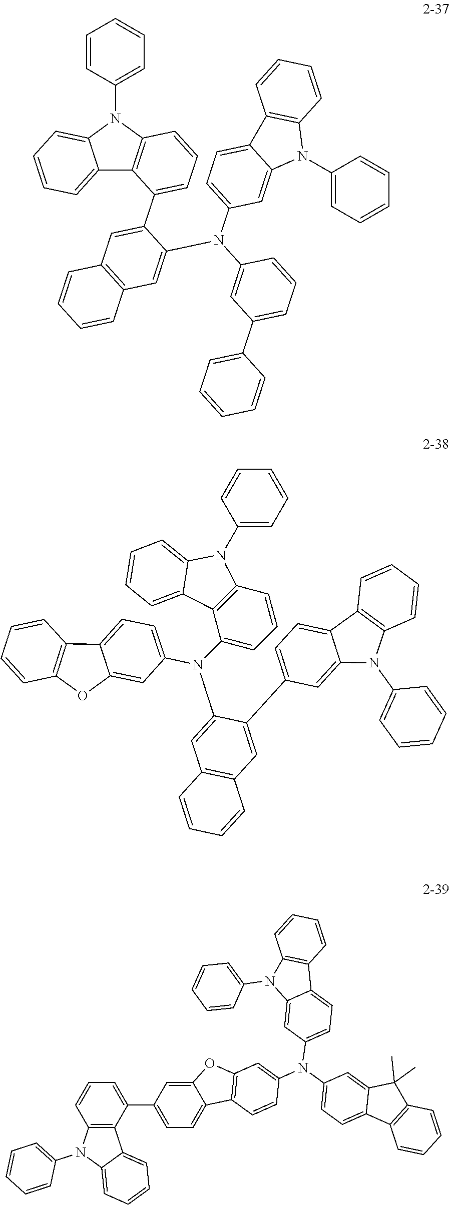 [SMILES: c1ccc(-c2cccc(N(c3ccc4c5ccccc5n(-c5ccccc5)c4c3)c3cc4ccccc4cc3-c3cccc4c3c3ccccc3n4-c3ccccc3)c2)cc1 ; c1ccc(-n2c3ccccc3c3ccc(-c4cc5ccccc5cc4N(c4ccc5c(c4)oc4ccccc45)c4cccc5c4c4ccccc4n5-c4ccccc4)cc32)cc1 ; CC1(C)c2ccccc2-c2ccc(N(c3ccc4c(c3)oc3cc(-c5cccc6c5c5ccccc5n6-c5ccccc5)ccc34)c3ccc4c5ccccc5n(-c5ccccc5)c4c3)cc21]

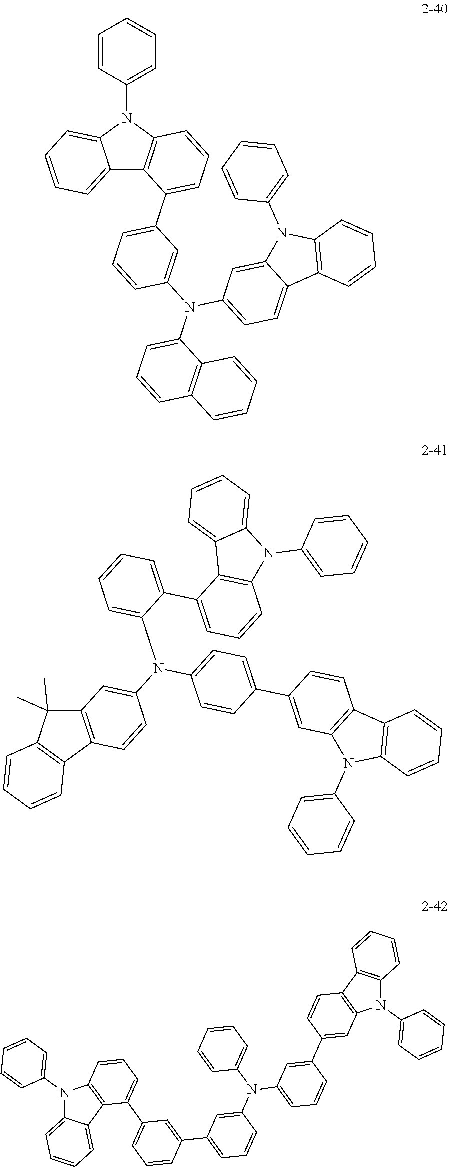 [SMILES: c1ccc(-n2c3ccccc3c3ccc(N(c4cccc(-c5cccc6c5c5ccccc5n6-c5ccccc5)c4)c4cccc5ccccc45)cc32)cc1 ; CC1(C)c2ccccc2-c2ccc(N(c3ccc(-c4ccc5c6ccccc6n(-c6ccccc6)c5c4)cc3)c3ccccc3-c3cccc4c3c3ccccc3n4-c3ccccc3)cc21 ; c1ccc(N(c2cccc(-c3cccc(-c4cccc5c4c4ccccc4n5-c4ccccc4)c3)c2)c2cccc(-c3ccc4c5ccccc5n(-c5ccccc5)c4c3)c2)cc1]

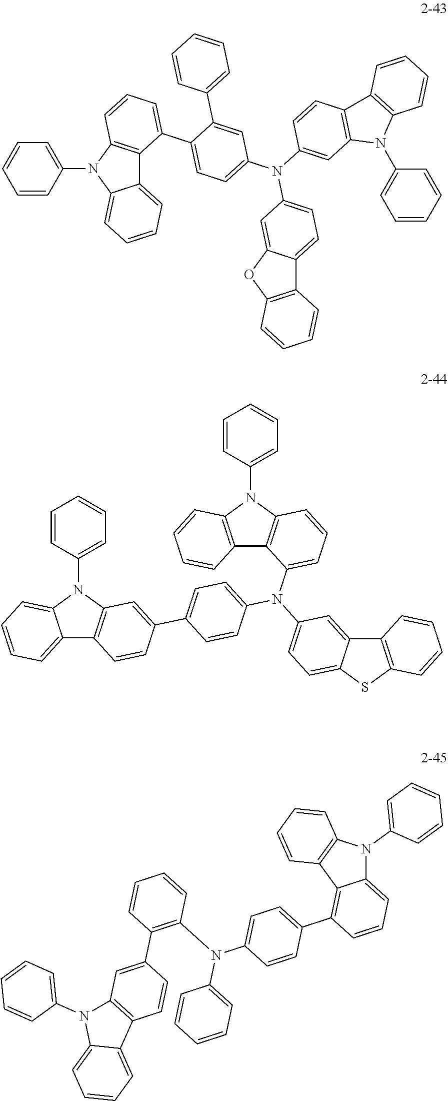 [SMILES: c1ccc(-c2cc(N(c3ccc4c(c3)oc3ccccc34)c3ccc4c5ccccc5n(-c5ccccc5)c4c3)ccc2-c2cccc3c2c2ccccc2n3-c2ccccc2)cc1 ; c1ccc(-n2c3ccccc3c3ccc(-c4ccc(N(c5ccc6sc7ccccc7c6c5)c5cccc6c5c5ccccc5n6-c5ccccc5)cc4)cc32)cc1 ; c1ccc(N(c2ccc(-c3cccc4c3c3ccccc3n4-c3ccccc3)cc2)c2ccccc2-c2ccc3c4ccccc4n(-c4ccccc4)c3c2)cc1]

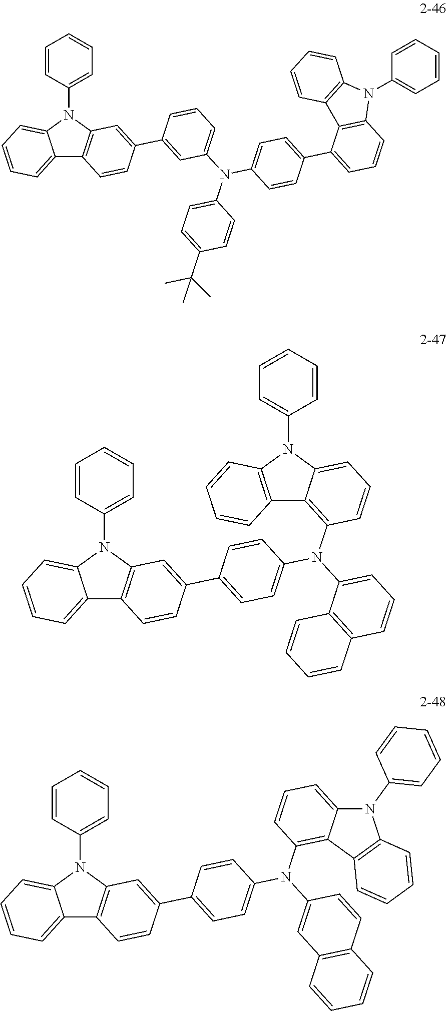 [SMILES: CC(C)(C)c1ccc(N(c2ccc(-c3cccc4c3c3ccccc3n4-c3ccccc3)cc2)c2cccc(-c3ccc4c5ccccc5n(-c5ccccc5)c4c3)c2)cc1 ; c1ccc(-n2c3ccccc3c3ccc(-c4ccc(N(c5cccc6ccccc56)c5cccc6c5c5ccccc5n6-c5ccccc5)cc4)cc32)cc1 ; c1ccc(-n2c3ccccc3c3ccc(-c4ccc(N(c5ccc6ccccc6c5)c5cccc6c5c5ccccc5n6-c5ccccc5)cc4)cc32)cc1]

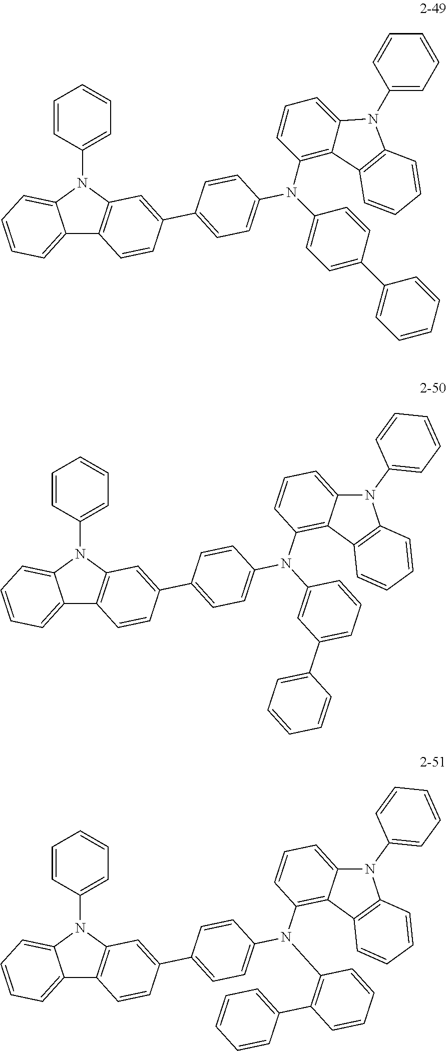 [SMILES: c1ccc(-c2ccc(N(c3ccc(-c4ccc5c6ccccc6n(-c6ccccc6)c5c4)cc3)c3cccc4c3c3ccccc3n4-c3ccccc3)cc2)cc1 ; c1ccc(-c2cccc(N(c3ccc(-c4ccc5c6ccccc6n(-c6ccccc6)c5c4)cc3)c3cccc4c3c3ccccc3n4-c3ccccc3)c2)cc1 ; c1ccc(-c2ccccc2N(c2ccc(-c3ccc4c5ccccc5n(-c5ccccc5)c4c3)cc2)c2cccc3c2c2ccccc2n3-c2ccccc2)cc1]

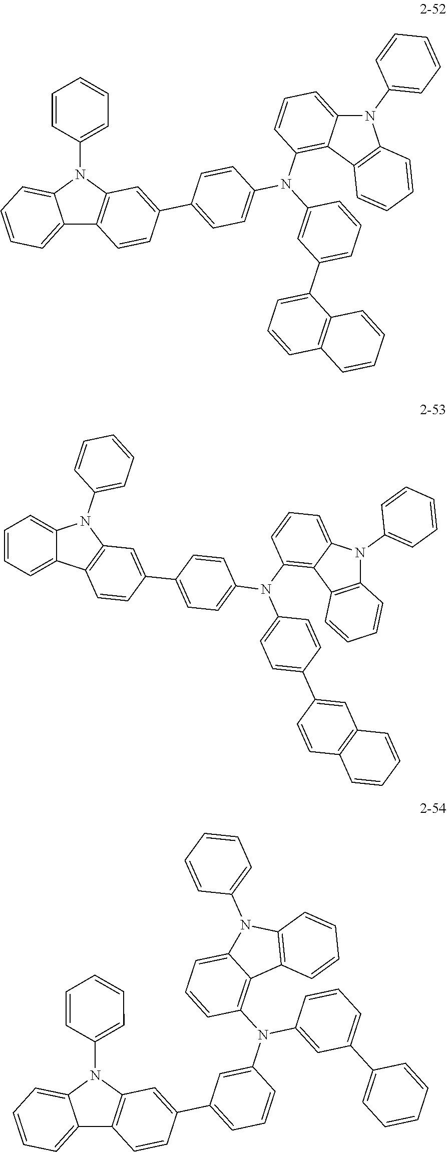 [SMILES: c1ccc(-n2c3ccccc3c3ccc(-c4ccc(N(c5cccc(-c6cccc7ccccc67)c5)c5cccc6c5c5ccccc5n6-c5ccccc5)cc4)cc32)cc1 ; c1ccc(-n2c3ccccc3c3ccc(-c4ccc(N(c5ccc(-c6ccc7ccccc7c6)cc5)c5cccc6c5c5ccccc5n6-c5ccccc5)cc4)cc32)cc1 ; c1ccc(-c2cccc(N(c3cccc(-c4ccc5c6ccccc6n(-c6ccccc6)c5c4)c3)c3cccc4c3c3ccccc3n4-c3ccccc3)c2)cc1]

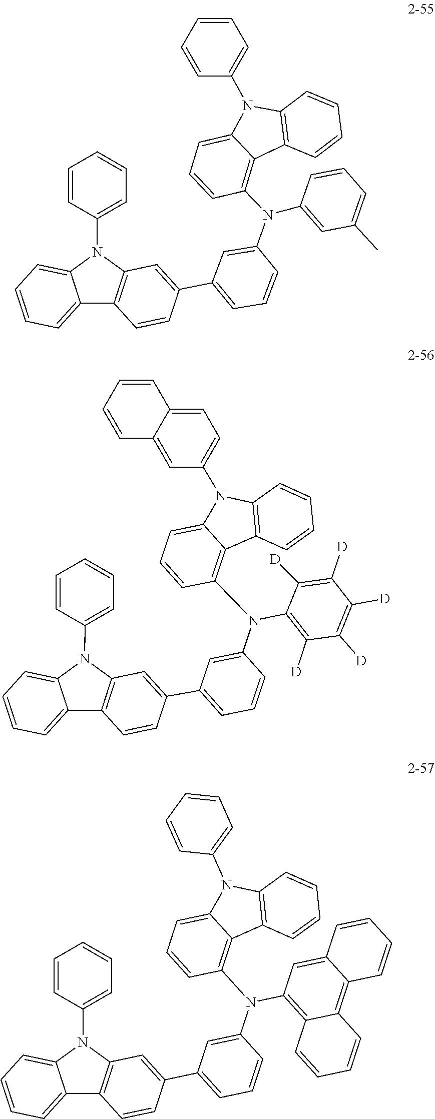 [SMILES: Cc1cccc(N(c2cccc(-c3ccc4c5ccccc5n(-c5ccccc5)c4c3)c2)c2cccc3c2c2ccccc2n3-c2ccccc2)c1 ; [2H]c1c([2H])c([2H])c(N(c2cccc(-c3ccc4c5ccccc5n(-c5ccccc5)c4c3)c2)c2cccc3c2c2ccccc2n3-c2ccc3ccccc3c2)c([2H])c1[2H] ; c1ccc(-n2c3ccccc3c3ccc(-c4cccc(N(c5cc6ccccc6c6ccccc56)c5cccc6c5c5ccccc5n6-c5ccccc5)c4)cc32)cc1]

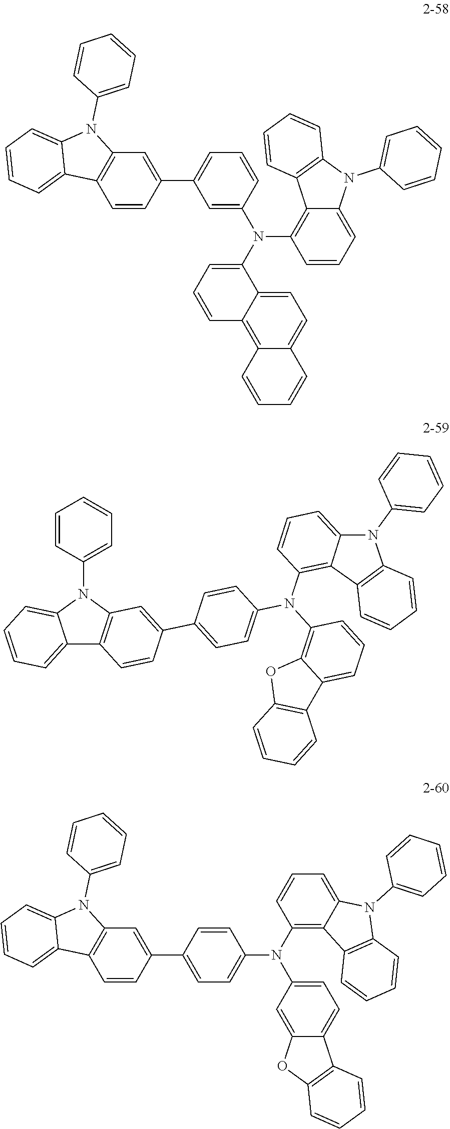 [SMILES: c1ccc(-n2c3ccccc3c3ccc(-c4cccc(N(c5cccc6c5ccc5ccccc56)c5cccc6c5c5ccccc5n6-c5ccccc5)c4)cc32)cc1 ; c1ccc(-n2c3ccccc3c3ccc(-c4ccc(N(c5cccc6c5oc5ccccc56)c5cccc6c5c5ccccc5n6-c5ccccc5)cc4)cc32)cc1 ; c1ccc(-n2c3ccccc3c3ccc(-c4ccc(N(c5ccc6c(c5)oc5ccccc56)c5cccc6c5c5ccccc5n6-c5ccccc5)cc4)cc32)cc1]

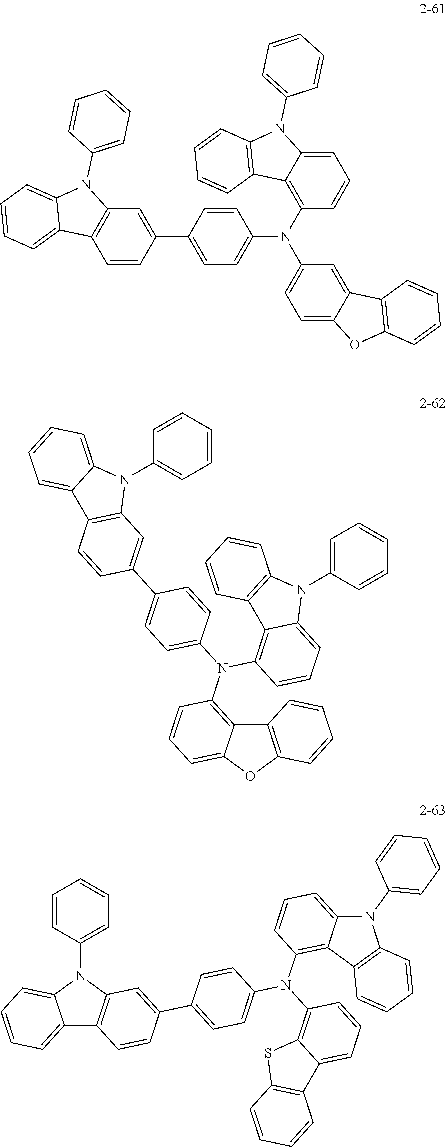 [SMILES: c1ccc(-n2c3ccccc3c3ccc(-c4ccc(N(c5ccc6oc7ccccc7c6c5)c5cccc6c5c5ccccc5n6-c5ccccc5)cc4)cc32)cc1 ; c1ccc(-n2c3ccccc3c3ccc(-c4ccc(N(c5cccc6oc7ccccc7c56)c5cccc6c5c5ccccc5n6-c5ccccc5)cc4)cc32)cc1 ; c1ccc(-n2c3ccccc3c3ccc(-c4ccc(N(c5cccc6c5sc5ccccc56)c5cccc6c5c5ccccc5n6-c5ccccc5)cc4)cc32)cc1]

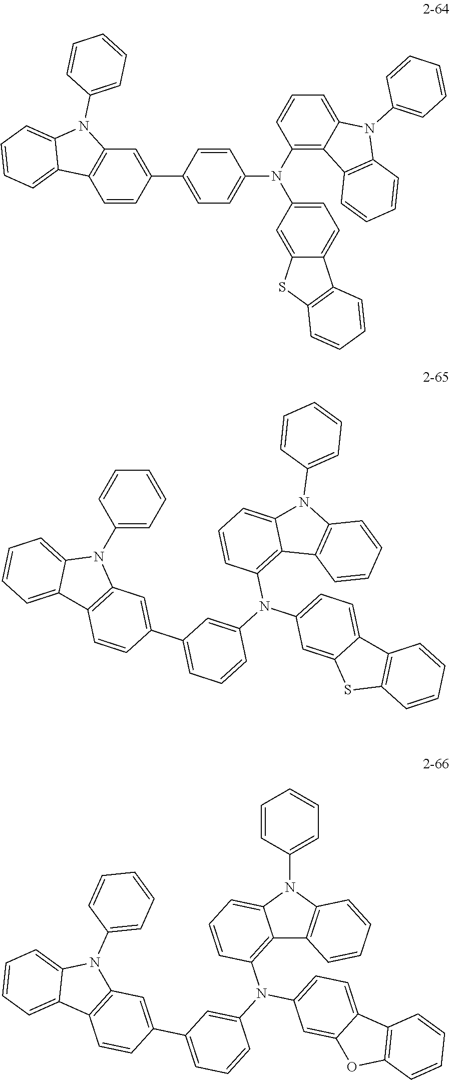 [SMILES: c1ccc(-n2c3ccccc3c3ccc(-c4ccc(N(c5ccc6c(c5)sc5ccccc56)c5cccc6c5c5ccccc5n6-c5ccccc5)cc4)cc32)cc1 ; c1ccc(-n2c3ccccc3c3ccc(-c4cccc(N(c5ccc6c(c5)sc5ccccc56)c5cccc6c5c5ccccc5n6-c5ccccc5)c4)cc32)cc1 ; c1ccc(-n2c3ccccc3c3ccc(-c4cccc(N(c5ccc6c(c5)oc5ccccc56)c5cccc6c5c5ccccc5n6-c5ccccc5)c4)cc32)cc1]

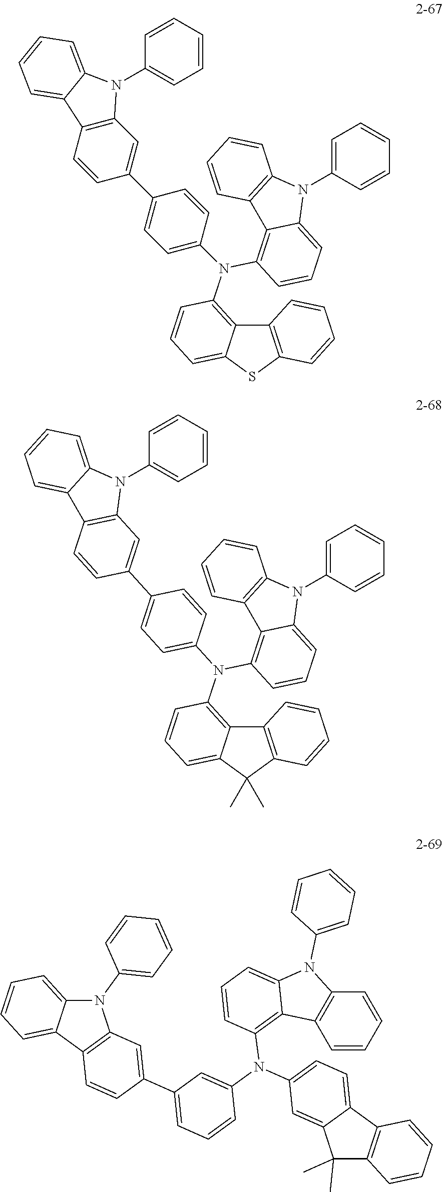 [SMILES: c1ccc(-n2c3ccccc3c3ccc(-c4ccc(N(c5cccc6sc7ccccc7c56)c5cccc6c5c5ccccc5n6-c5ccccc5)cc4)cc32)cc1 ; CC1(C)c2ccccc2-c2c(N(c3ccc(-c4ccc5c6ccccc6n(-c6ccccc6)c5c4)cc3)c3cccc4c3c3ccccc3n4-c3ccccc3)cccc21 ; CC1(C)c2ccccc2-c2ccc(N(c3cccc(-c4ccc5c6ccccc6n(-c6ccccc6)c5c4)c3)c3cccc4c3c3ccccc3n4-c3ccccc3)cc21]

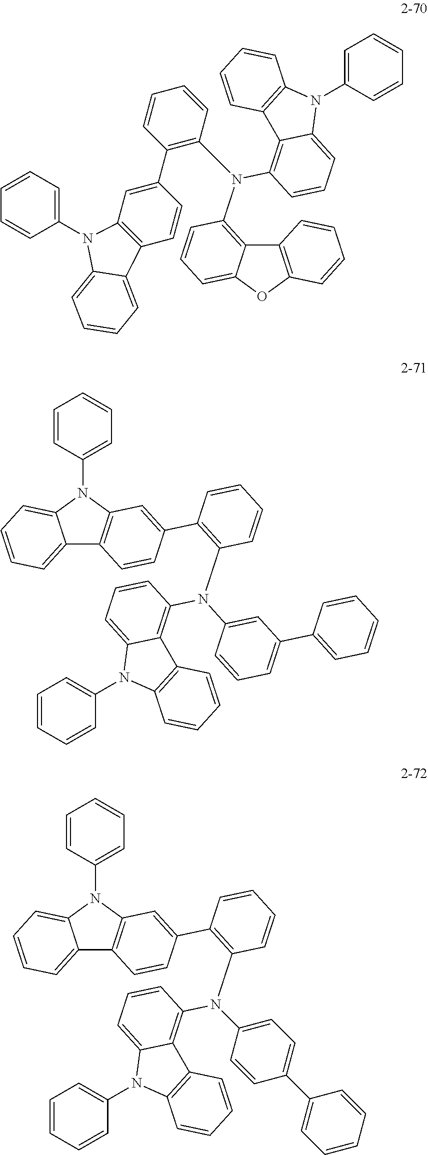 [SMILES: c1ccc(-n2c3ccccc3c3ccc(-c4ccccc4N(c4cccc5oc6ccccc6c45)c4cccc5c4c4ccccc4n5-c4ccccc4)cc32)cc1 ; c1ccc(-c2cccc(N(c3ccccc3-c3ccc4c5ccccc5n(-c5ccccc5)c4c3)c3cccc4c3c3ccccc3n4-c3ccccc3)c2)cc1 ; c1ccc(-c2ccc(N(c3ccccc3-c3ccc4c5ccccc5n(-c5ccccc5)c4c3)c3cccc4c3c3ccccc3n4-c3ccccc3)cc2)cc1]

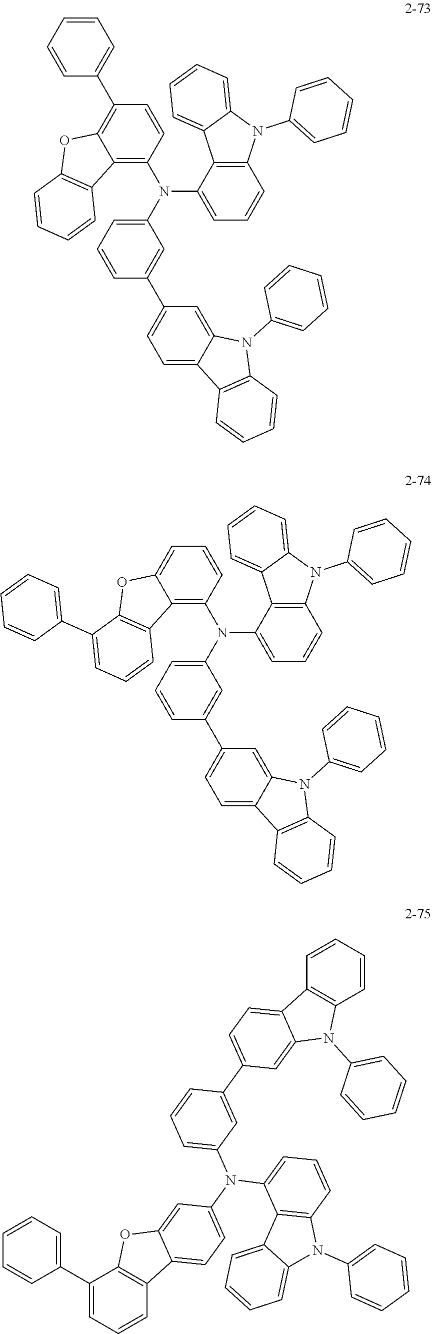 [SMILES: c1ccc(-c2ccc(N(c3cccc(-c4ccc5c6ccccc6n(-c6ccccc6)c5c4)c3)c3cccc4c3c3ccccc3n4-c3ccccc3)c3c2oc2ccccc23)cc1 ; c1ccc(-c2cccc3c2oc2cccc(N(c4cccc(-c5ccc6c7ccccc7n(-c7ccccc7)c6c5)c4)c4cccc5c4c4ccccc4n5-c4ccccc4)c23)cc1 ; c1ccc(-c2cccc3c2oc2cc(N(c4cccc(-c5ccc6c7ccccc7n(-c7ccccc7)c6c5)c4)c4cccc5c4c4ccccc4n5-c4ccccc4)ccc23)cc1]

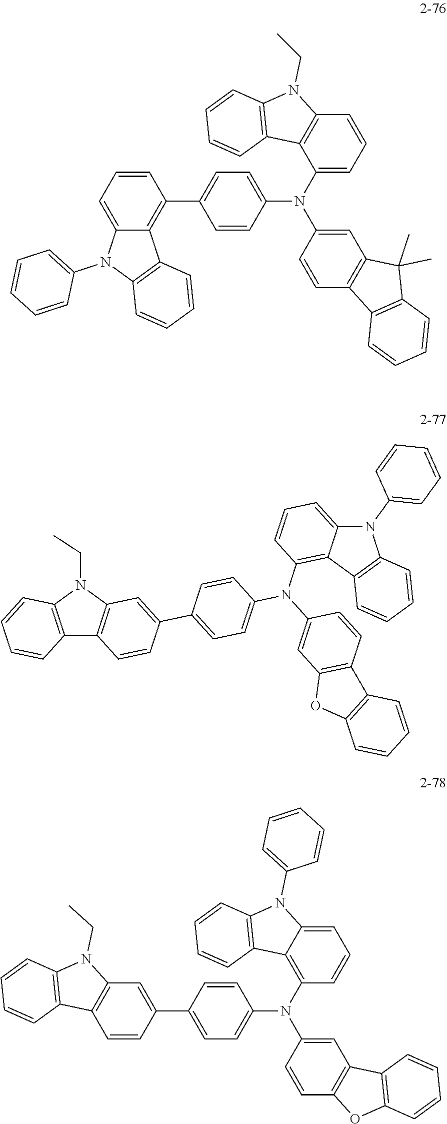 [SMILES: CCn1c2ccccc2c2c(N(c3ccc(-c4cccc5c4c4ccccc4n5-c4ccccc4)cc3)c3ccc4c(c3)C(C)(C)c3ccccc3-4)cccc21 ; CCn1c2ccccc2c2ccc(-c3ccc(N(c4ccc5c(c4)oc4ccccc45)c4cccc5c4c4ccccc4n5-c4ccccc4)cc3)cc21 ; CCn1c2ccccc2c2ccc(-c3ccc(N(c4ccc5oc6ccccc6c5c4)c4cccc5c4c4ccccc4n5-c4ccccc4)cc3)cc21]

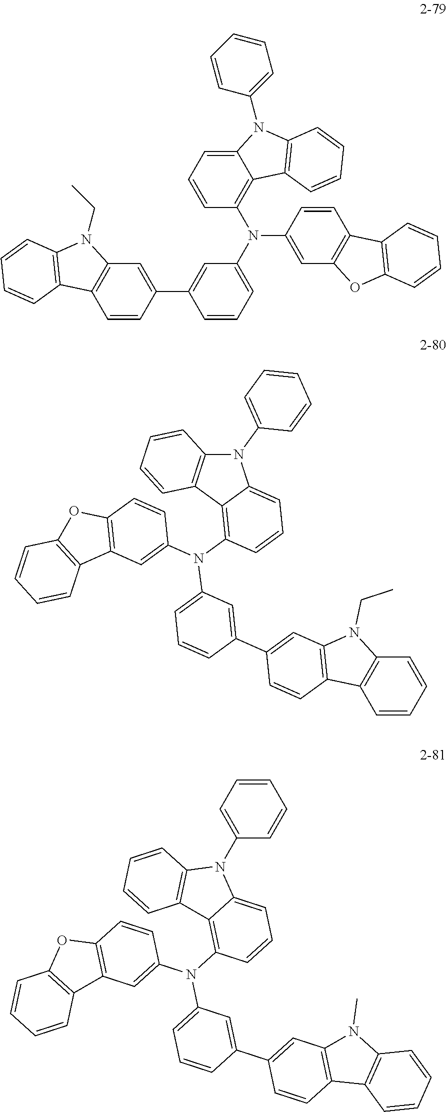 [SMILES: CCn1c2ccccc2c2ccc(-c3cccc(N(c4ccc5c(c4)oc4ccccc45)c4cccc5c4c4ccccc4n5-c4ccccc4)c3)cc21 ; CCn1c2ccccc2c2ccc(-c3cccc(N(c4ccc5oc6ccccc6c5c4)c4cccc5c4c4ccccc4n5-c4ccccc4)c3)cc21 ; Cn1c2ccccc2c2ccc(-c3cccc(N(c4ccc5oc6ccccc6c5c4)c4cccc5c4c4ccccc4n5-c4ccccc4)c3)cc21]

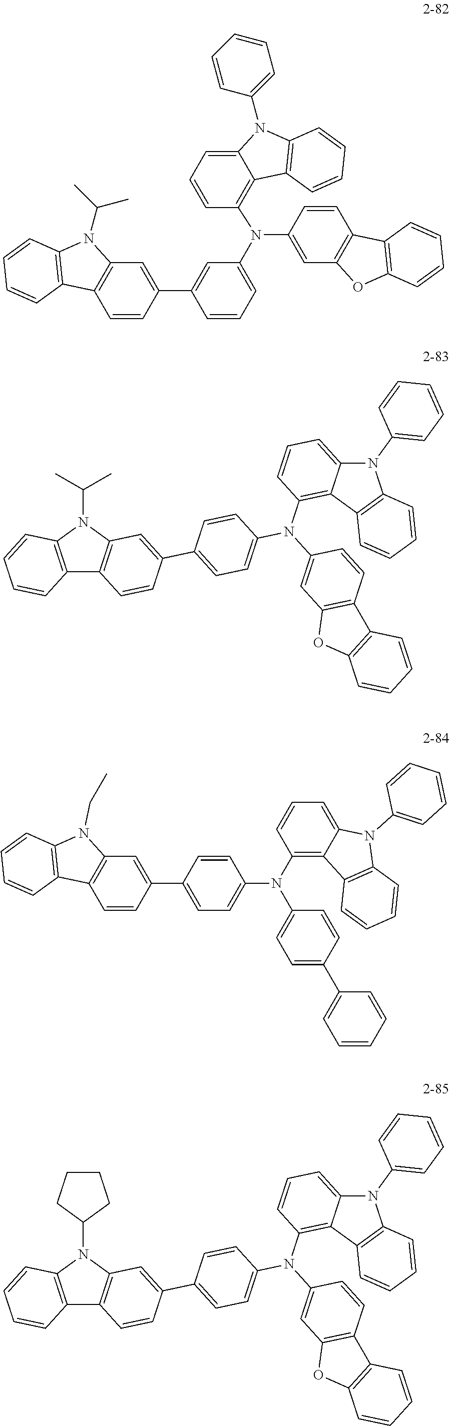 [SMILES: CC(C)n1c2ccccc2c2ccc(-c3cccc(N(c4ccc5c(c4)oc4ccccc45)c4cccc5c4c4ccccc4n5-c4ccccc4)c3)cc21 ; CC(C)n1c2ccccc2c2ccc(-c3ccc(N(c4ccc5c(c4)oc4ccccc45)c4cccc5c4c4ccccc4n5-c4ccccc4)cc3)cc21 ; CCn1c2ccccc2c2ccc(-c3ccc(N(c4ccc(-c5ccccc5)cc4)c4cccc5c4c4ccccc4n5-c4ccccc4)cc3)cc21 ; c1ccc(-n2c3ccccc3c3c(N(c4ccc(-c5ccc6c7ccccc7n(C7CCCC7)c6c5)cc4)c4ccc5c(c4)oc4ccccc45)cccc32)cc1]

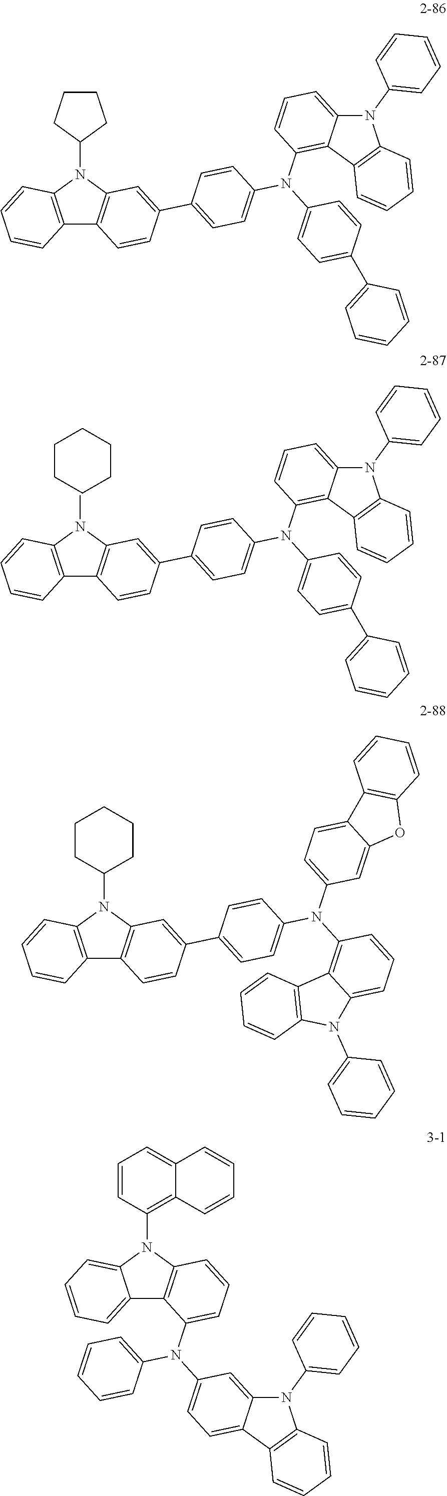

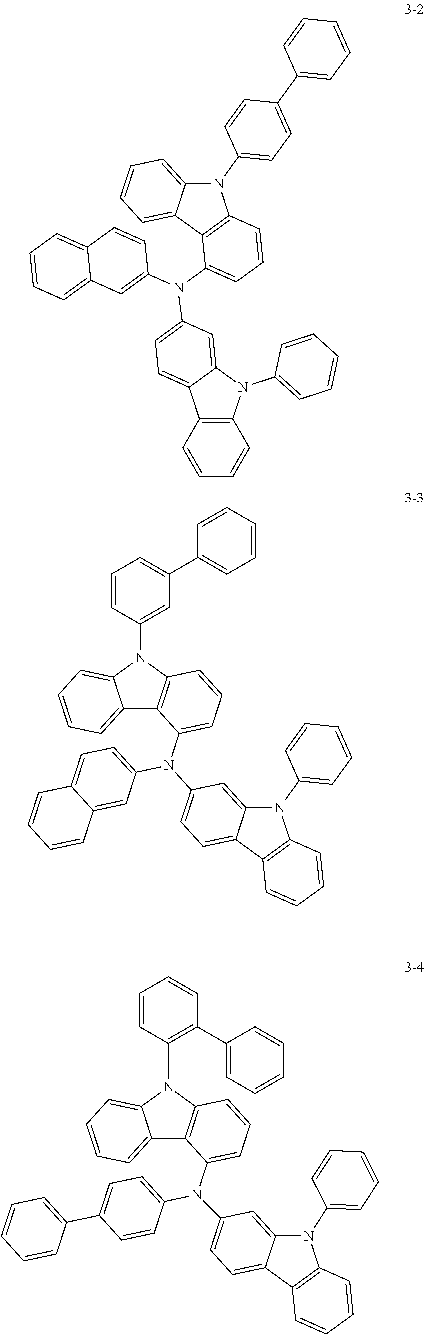

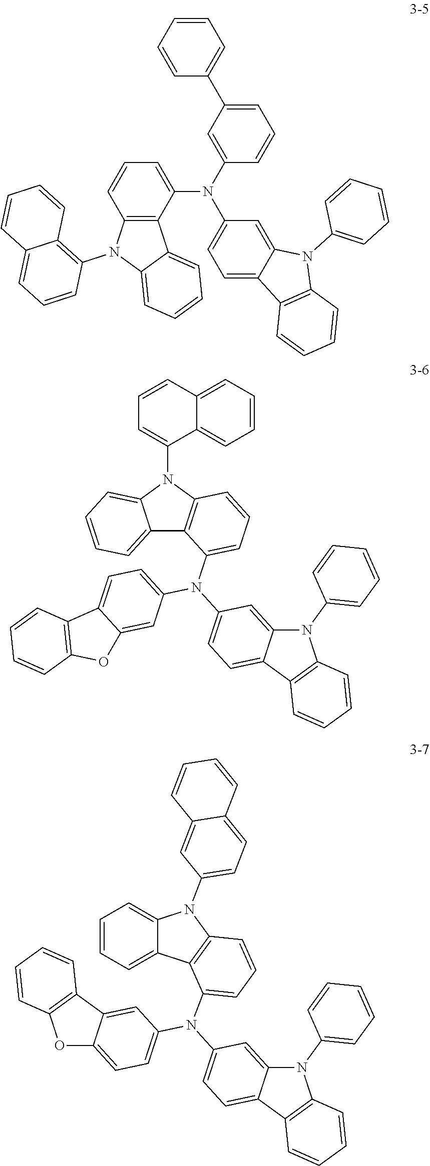 [SMILES: c1ccc(-c2cccc(N(c3ccc4c5ccccc5n(-c5ccccc5)c4c3)c3cccc4c3c3ccccc3n4-c3cccc4ccccc34)c2)cc1 ; c1ccc(-n2c3ccccc3c3ccc(N(c4ccc5c(c4)oc4ccccc45)c4cccc5c4c4ccccc4n5-c4cccc5ccccc45)cc32)cc1 ; c1ccc(-n2c3ccccc3c3ccc(N(c4ccc5oc6ccccc6c5c4)c4cccc5c4c4ccccc4n5-c4ccc5ccccc5c4)cc32)cc1]

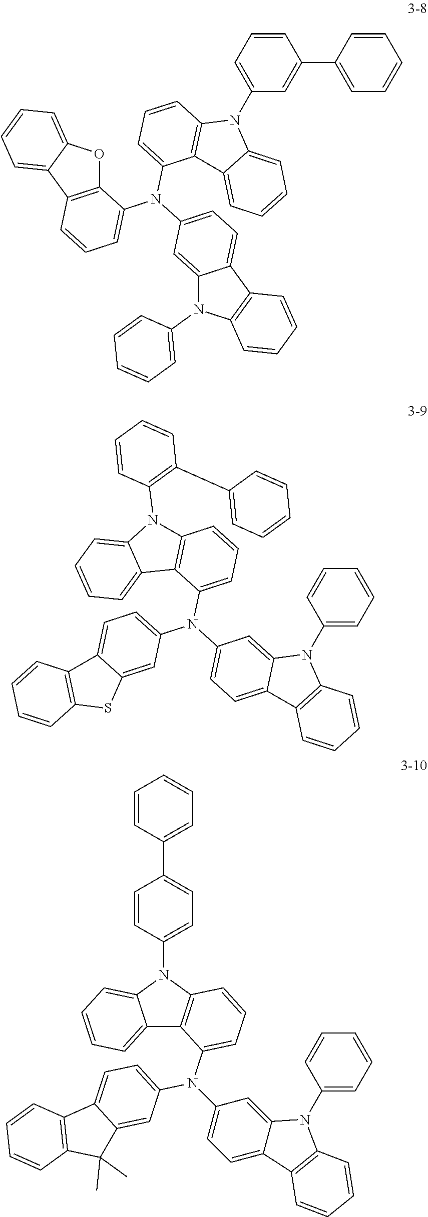

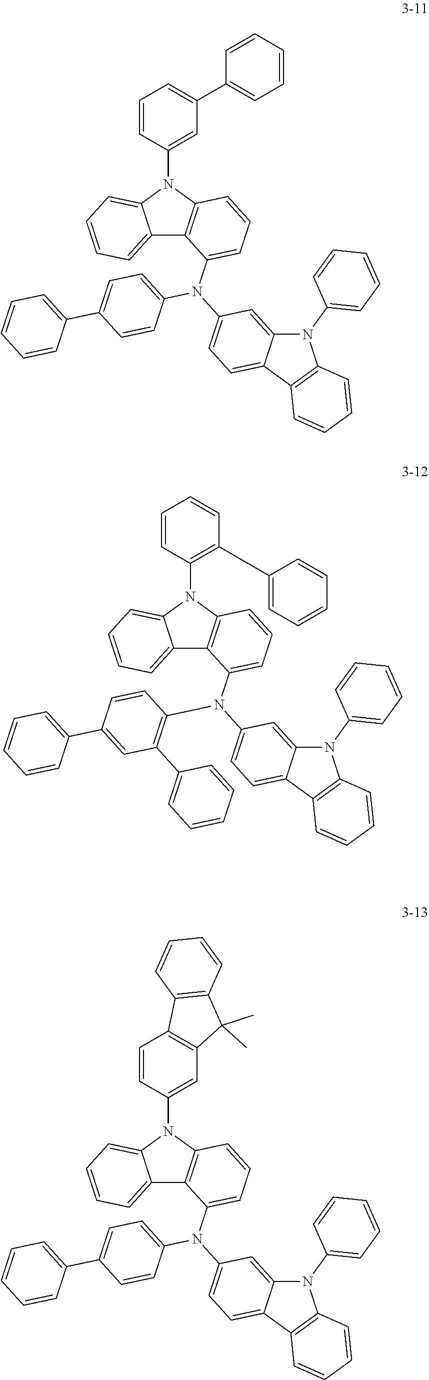 [SMILES: c1ccc(-c2ccc(N(c3ccc4c5ccccc5n(-c5ccccc5)c4c3)c3cccc4c3c3ccccc3n4-c3cccc(-c4ccccc4)c3)cc2)cc1 ; c1ccc(-c2ccc(N(c3ccc4c5ccccc5n(-c5ccccc5)c4c3)c3cccc4c3c3ccccc3n4-c3ccccc3-c3ccccc3)c(-c3ccccc3)c2)cc1 ; CC1(C)c2ccccc2-c2ccc(-n3c4ccccc4c4c(N(c5ccc(-c6ccccc6)cc5)c5ccc6c7ccccc7n(-c7ccccc7)c6c5)cccc43)cc21]

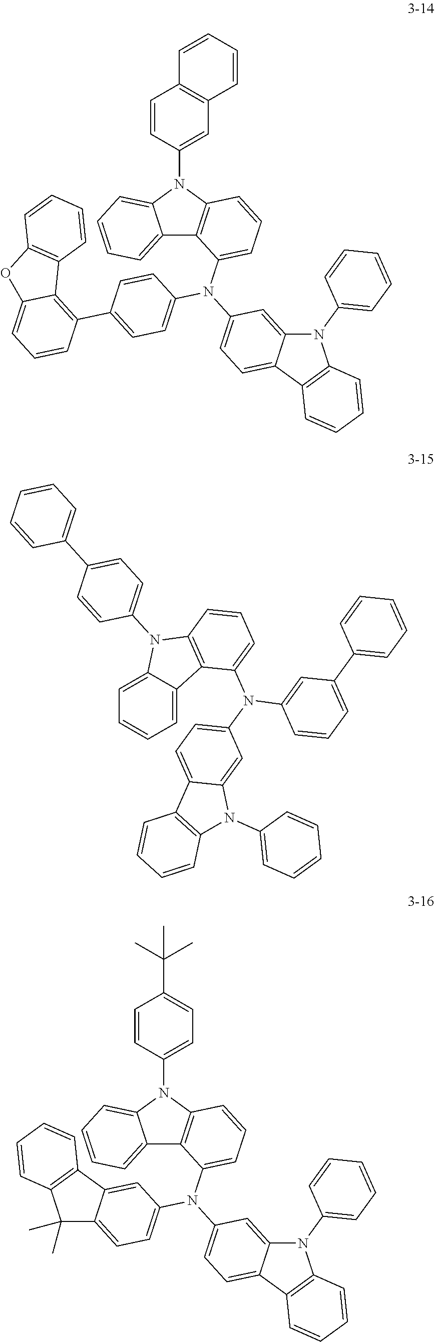 [SMILES: c1ccc(-n2c3ccccc3c3ccc(N(c4ccc(-c5cccc6oc7ccccc7c56)cc4)c4cccc5c4c4ccccc4n5-c4ccc5ccccc5c4)cc32)cc1 ; c1ccc(-c2ccc(-n3c4ccccc4c4c(N(c5cccc(-c6ccccc6)c5)c5ccc6c7ccccc7n(-c7ccccc7)c6c5)cccc43)cc2)cc1 ; CC(C)(C)c1ccc(-n2c3ccccc3c3c(N(c4ccc5c(c4)-c4ccccc4C5(C)C)c4ccc5c6ccccc6n(-c6ccccc6)c5c4)cccc32)cc1]

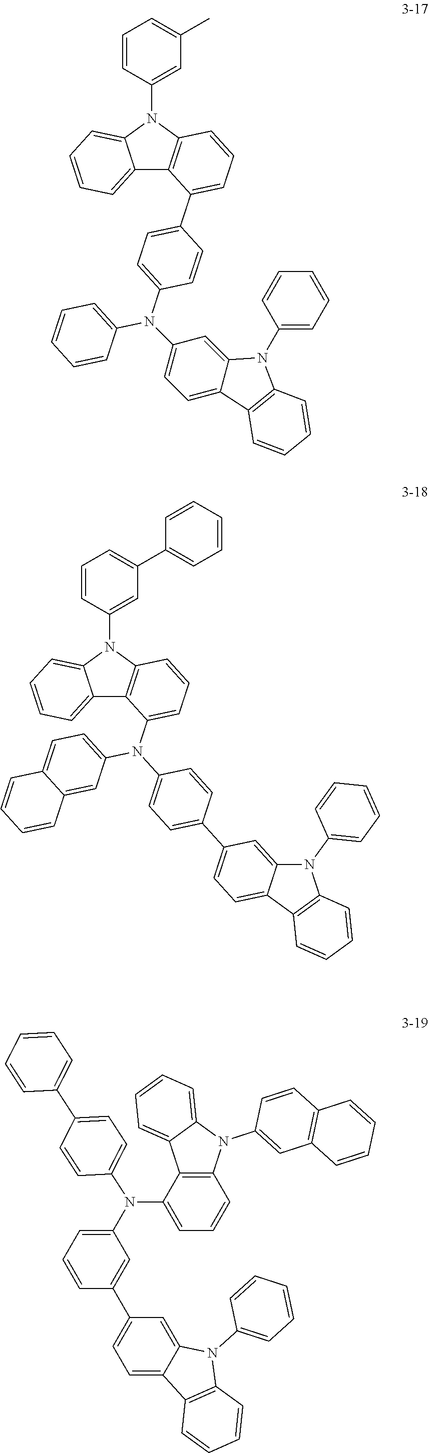 [SMILES: Cc1cccc(-n2c3ccccc3c3c(-c4ccc(N(c5ccccc5)c5ccc6c7ccccc7n(-c7ccccc7)c6c5)cc4)cccc32)c1 ; c1ccc(-c2cccc(-n3c4ccccc4c4c(N(c5ccc(-c6ccc7c8ccccc8n(-c8ccccc8)c7c6)cc5)c5ccc6ccccc6c5)cccc43)c2)cc1 ; c1ccc(-c2ccc(N(c3cccc(-c4ccc5c6ccccc6n(-c6ccccc6)c5c4)c3)c3cccc4c3c3ccccc3n4-c3ccc4ccccc4c3)cc2)cc1]

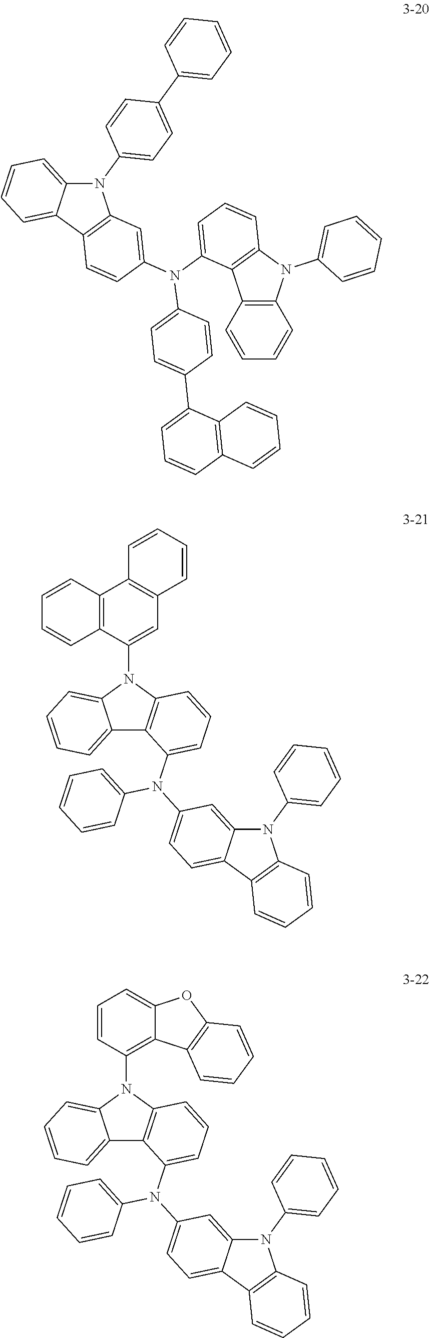 [SMILES: c1ccc(-c2ccc(-n3c4ccccc4c4ccc(N(c5ccc(-c6cccc7ccccc67)cc5)c5cccc6c5c5ccccc5n6-c5ccccc5)cc43)cc2)cc1 ; c1ccc(N(c2ccc3c4ccccc4n(-c4ccccc4)c3c2)c2cccc3c2c2ccccc2n3-c2cc3ccccc3c3ccccc23)cc1 ; c1ccc(N(c2ccc3c4ccccc4n(-c4ccccc4)c3c2)c2cccc3c2c2ccccc2n3-c2cccc3oc4ccccc4c23)cc1]

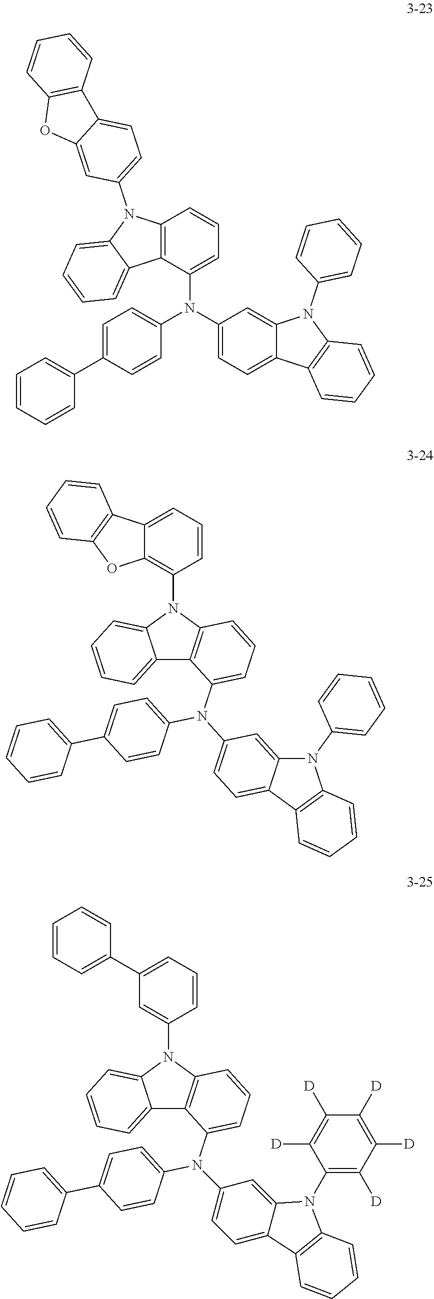 [SMILES: c1ccc(-c2ccc(N(c3ccc4c5ccccc5n(-c5ccccc5)c4c3)c3cccc4c3c3ccccc3n4-c3ccc4c(c3)oc3ccccc34)cc2)cc1 ; c1ccc(-c2ccc(N(c3ccc4c5ccccc5n(-c5ccccc5)c4c3)c3cccc4c3c3ccccc3n4-c3cccc4c3oc3ccccc34)cc2)cc1 ; [2H]c1c([2H])c([2H])c(-n2c3ccccc3c3ccc(N(c4ccc(-c5ccccc5)cc4)c4cccc5c4c4ccccc4n5-c4cccc(-c5ccccc5)c4)cc32)c([2H])c1[2H]]

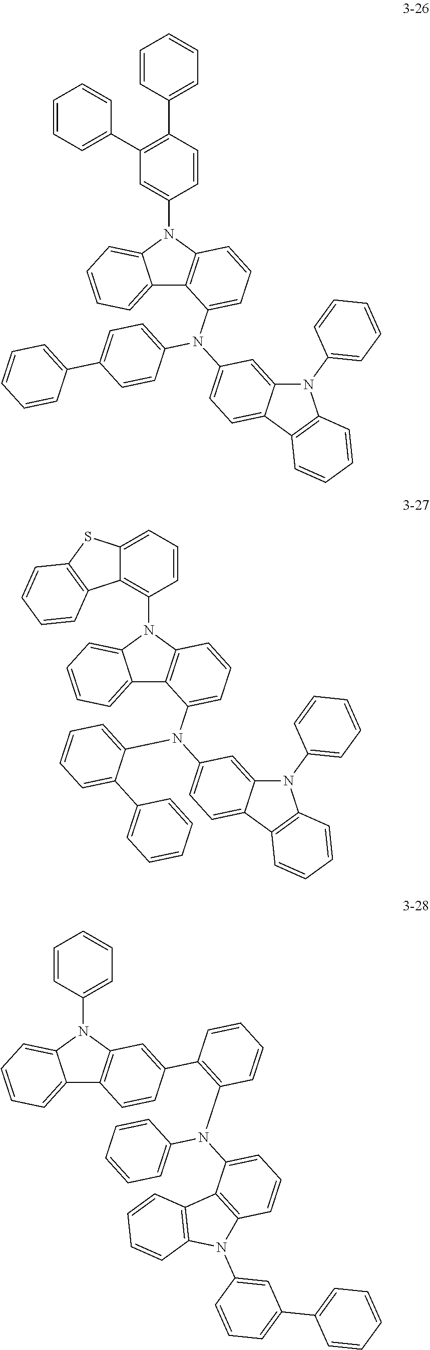 [SMILES: c1ccc(-c2ccc(N(c3ccc4c5ccccc5n(-c5ccccc5)c4c3)c3cccc4c3c3ccccc3n4-c3ccc(-c4ccccc4)c(-c4ccccc4)c3)cc2)cc1 ; c1ccc(-c2ccccc2N(c2ccc3c4ccccc4n(-c4ccccc4)c3c2)c2cccc3c2c2ccccc2n3-c2cccc3sc4ccccc4c23)cc1 ; c1ccc(-c2cccc(-n3c4ccccc4c4c(N(c5ccccc5)c5ccccc5-c5ccc6c7ccccc7n(-c7ccccc7)c6c5)cccc43)c2)cc1]

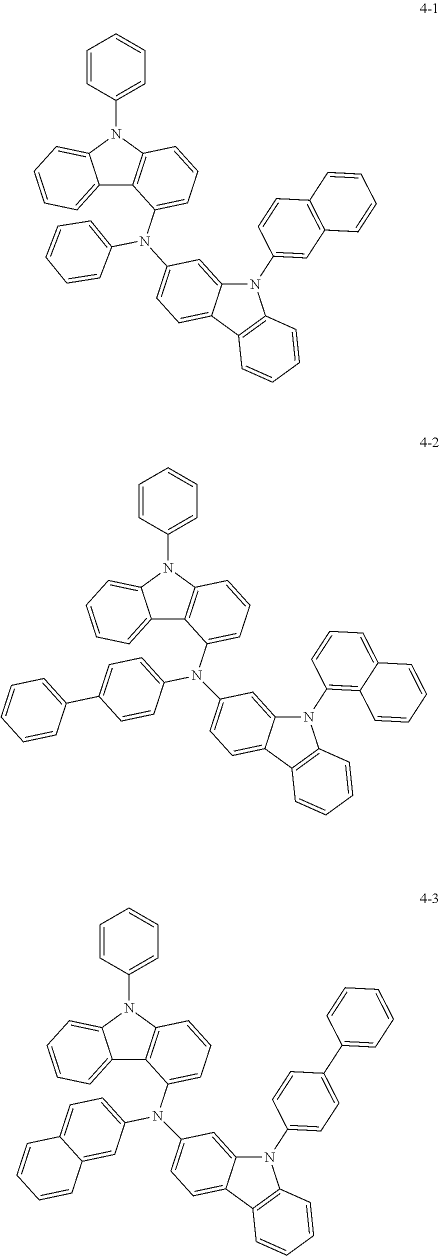 [SMILES: c1ccc(N(c2ccc3c4ccccc4n(-c4ccc5ccccc5c4)c3c2)c2cccc3c2c2ccccc2n3-c2ccccc2)cc1 ; c1ccc(-c2ccc(N(c3ccc4c5ccccc5n(-c5cccc6ccccc56)c4c3)c3cccc4c3c3ccccc3n4-c3ccccc3)cc2)cc1 ; c1ccc(-c2ccc(-n3c4ccccc4c4ccc(N(c5ccc6ccccc6c5)c5cccc6c5c5ccccc5n6-c5ccccc5)cc43)cc2)cc1]

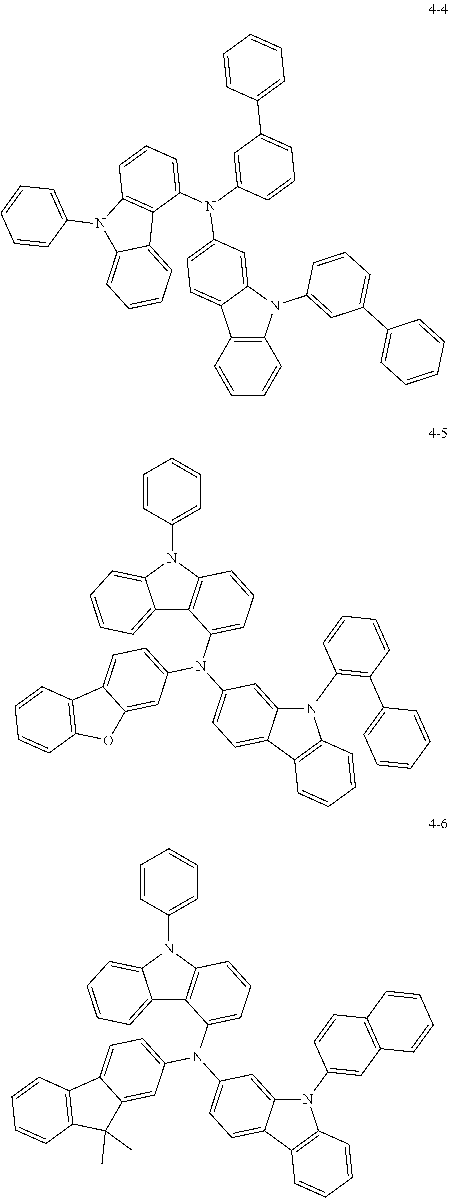 [SMILES: c1ccc(-c2cccc(N(c3ccc4c5ccccc5n(-c5cccc(-c6ccccc6)c5)c4c3)c3cccc4c3c3ccccc3n4-c3ccccc3)c2)cc1 ; c1ccc(-c2ccccc2-n2c3ccccc3c3ccc(N(c4ccc5c(c4)oc4ccccc45)c4cccc5c4c4ccccc4n5-c4ccccc4)cc32)cc1 ; CC1(C)c2ccccc2-c2ccc(N(c3ccc4c5ccccc5n(-c5ccc6ccccc6c5)c4c3)c3cccc4c3c3ccccc3n4-c3ccccc3)cc21]

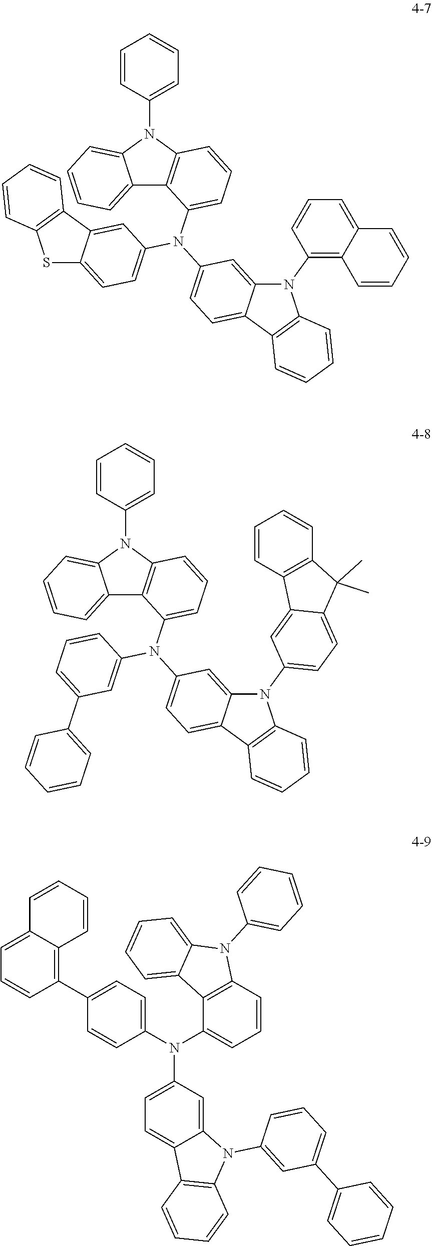 [SMILES: c1ccc(-n2c3ccccc3c3c(N(c4ccc5sc6ccccc6c5c4)c4ccc5c6ccccc6n(-c6cccc7ccccc67)c5c4)cccc32)cc1 ; CC1(C)c2ccccc2-c2cc(-n3c4ccccc4c4ccc(N(c5cccc(-c6ccccc6)c5)c5cccc6c5c5ccccc5n6-c5ccccc5)cc43)ccc21 ; c1ccc(-c2cccc(-n3c4ccccc4c4ccc(N(c5ccc(-c6cccc7ccccc67)cc5)c5cccc6c5c5ccccc5n6-c5ccccc5)cc43)c2)cc1]

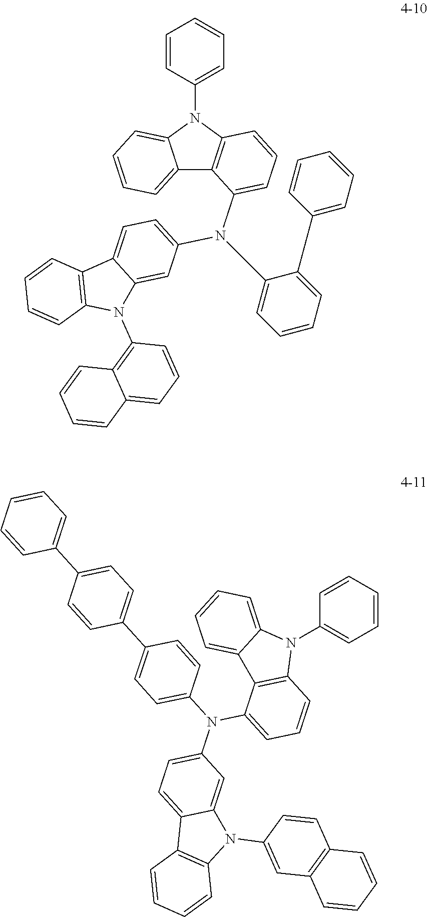 [SMILES: c1ccc(-c2ccccc2N(c2ccc3c4ccccc4n(-c4cccc5ccccc45)c3c2)c2cccc3c2c2ccccc2n3-c2ccccc2)cc1 ; c1ccc(-c2ccc(-c3ccc(N(c4ccc5c6ccccc6n(-c6ccc7ccccc7c6)c5c4)c4cccc5c4c4ccccc4n5-c4ccccc4)cc3)cc2)cc1]

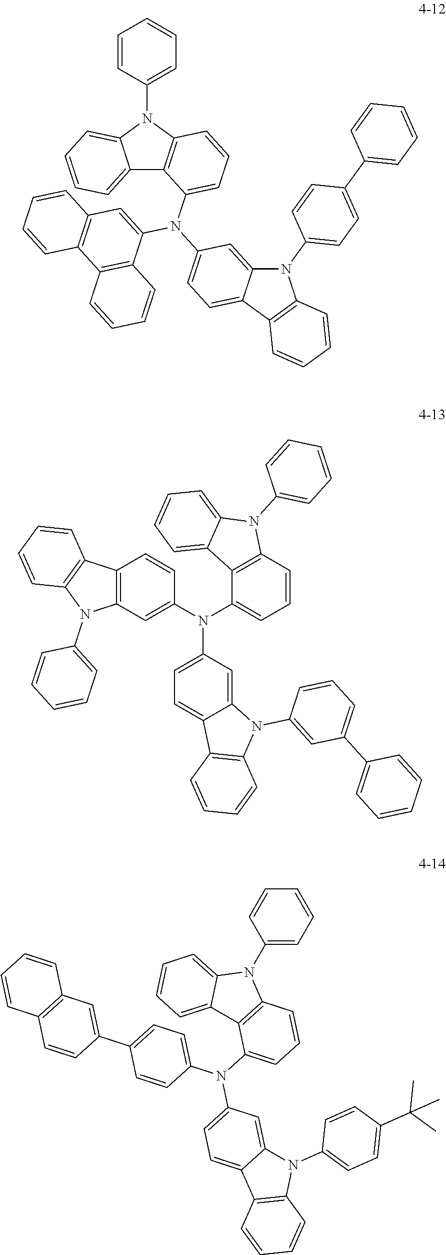 [SMILES: c1ccc(-c2ccc(-n3c4ccccc4c4ccc(N(c5cc6ccccc6c6ccccc56)c5cccc6c5c5ccccc5n6-c5ccccc5)cc43)cc2)cc1 ; c1ccc(-c2cccc(-n3c4ccccc4c4ccc(N(c5ccc6c7ccccc7n(-c7ccccc7)c6c5)c5cccc6c5c5ccccc5n6-c5ccccc5)cc43)c2)cc1 ; CC(C)(C)c1ccc(-n2c3ccccc3c3ccc(N(c4ccc(-c5ccc6ccccc6c5)cc4)c4cccc5c4c4ccccc4n5-c4ccccc4)cc32)cc1]

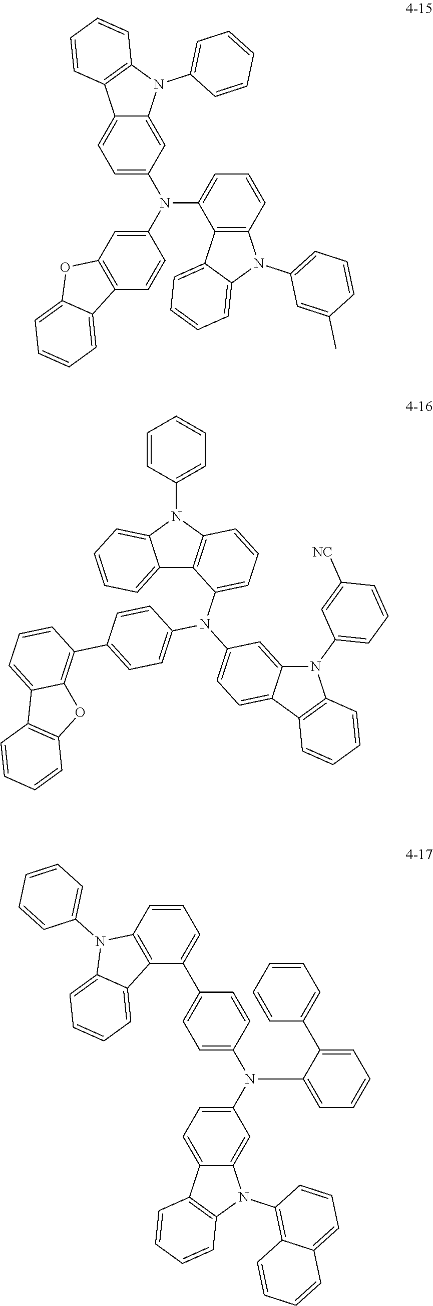 [SMILES: Cc1cccc(-n2c3ccccc3c3c(N(c4ccc5c(c4)oc4ccccc45)c4ccc5c6ccccc6n(-c6ccccc6)c5c4)cccc32)c1 ; N#Cc1cccc(-n2c3ccccc3c3ccc(N(c4ccc(-c5cccc6c5oc5ccccc56)cc4)c4cccc5c4c4ccccc4n5-c4ccccc4)cc32)c1 ; c1ccc(-c2ccccc2N(c2ccc(-c3cccc4c3c3ccccc3n4-c3ccccc3)cc2)c2ccc3c4ccccc4n(-c4cccc5ccccc45)c3c2)cc1]

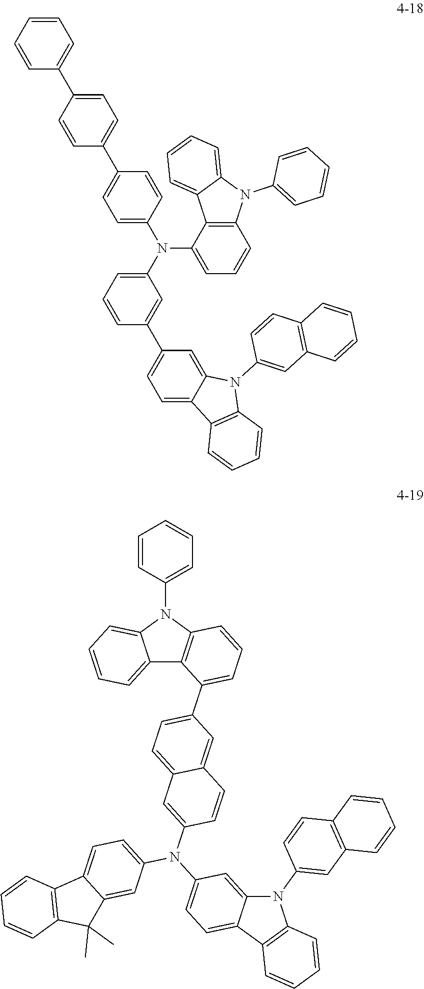 [SMILES: c1ccc(-c2ccc(-c3ccc(N(c4cccc(-c5ccc6c7ccccc7n(-c7ccc8ccccc8c7)c6c5)c4)c4cccc5c4c4ccccc4n5-c4ccccc4)cc3)cc2)cc1 ; CC1(C)c2ccccc2-c2ccc(N(c3ccc4cc(-c5cccc6c5c5ccccc5n6-c5ccccc5)ccc4c3)c3ccc4c5ccccc5n(-c5ccc6ccccc6c5)c4c3)cc21]

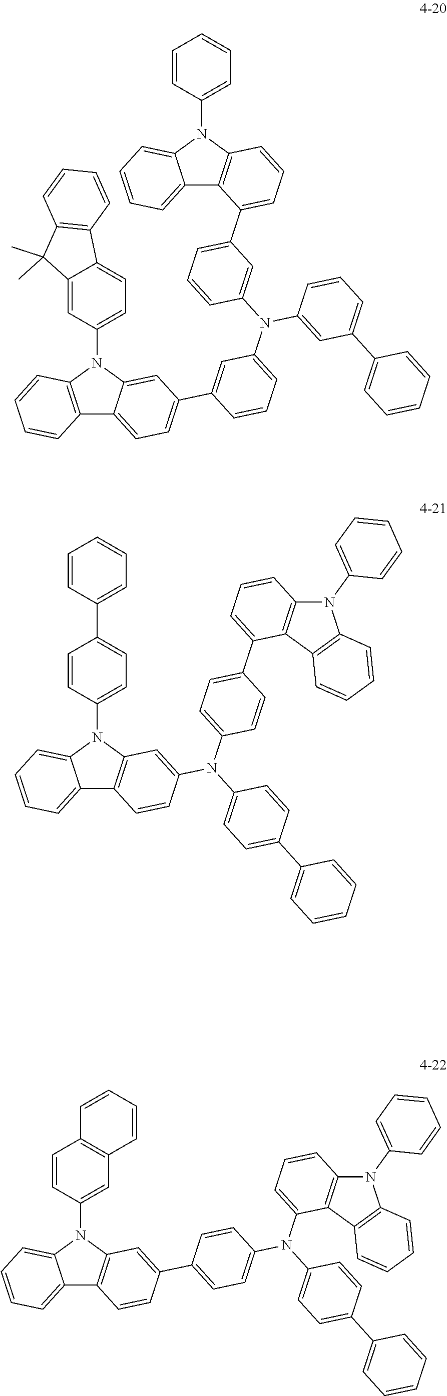 [SMILES: CC1(C)c2ccccc2-c2ccc(-n3c4ccccc4c4ccc(-c5cccc(N(c6cccc(-c7ccccc7)c6)c6cccc(-c7cccc8c7c7ccccc7n8-c7ccccc7)c6)c5)cc43)cc21 ; c1ccc(-c2ccc(N(c3ccc(-c4cccc5c4c4ccccc4n5-c4ccccc4)cc3)c3ccc4c5ccccc5n(-c5ccc(-c6ccccc6)cc5)c4c3)cc2)cc1 ; c1ccc(-c2ccc(N(c3ccc(-c4ccc5c6ccccc6n(-c6ccc7ccccc7c6)c5c4)cc3)c3cccc4c3c3ccccc3n4-c3ccccc3)cc2)cc1]

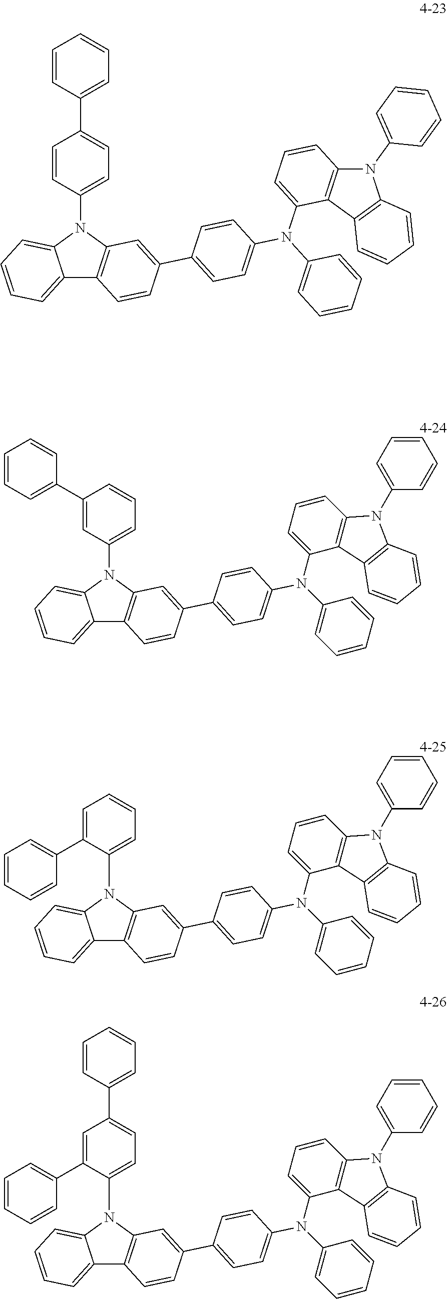 [SMILES: c1ccc(-c2ccc(-n3c4ccccc4c4ccc(-c5ccc(N(c6ccccc6)c6cccc7c6c6ccccc6n7-c6ccccc6)cc5)cc43)cc2)cc1 ; c1ccc(-c2cccc(-n3c4ccccc4c4ccc(-c5ccc(N(c6ccccc6)c6cccc7c6c6ccccc6n7-c6ccccc6)cc5)cc43)c2)cc1 ; c1ccc(-c2ccccc2-n2c3ccccc3c3ccc(-c4ccc(N(c5ccccc5)c5cccc6c5c5ccccc5n6-c5ccccc5)cc4)cc32)cc1 ; c1ccc(-c2ccc(-n3c4ccccc4c4ccc(-c5ccc(N(c6ccccc6)c6cccc7c6c6ccccc6n7-c6ccccc6)cc5)cc43)c(-c3ccccc3)c2)cc1]

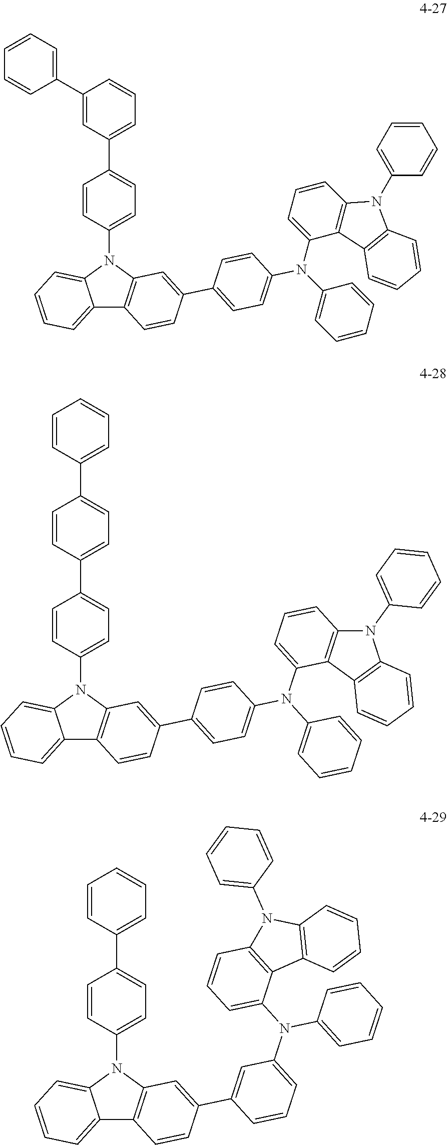 [SMILES: c1ccc(-c2cccc(-c3ccc(-n4c5ccccc5c5ccc(-c6ccc(N(c7ccccc7)c7cccc8c7c7ccccc7n8-c7ccccc7)cc6)cc54)cc3)c2)cc1 ; c1ccc(-c2ccc(-c3ccc(-n4c5ccccc5c5ccc(-c6ccc(N(c7ccccc7)c7cccc8c7c7ccccc7n8-c7ccccc7)cc6)cc54)cc3)cc2)cc1 ; c1ccc(-c2ccc(-n3c4ccccc4c4ccc(-c5cccc(N(c6ccccc6)c6cccc7c6c6ccccc6n7-c6ccccc6)c5)cc43)cc2)cc1]

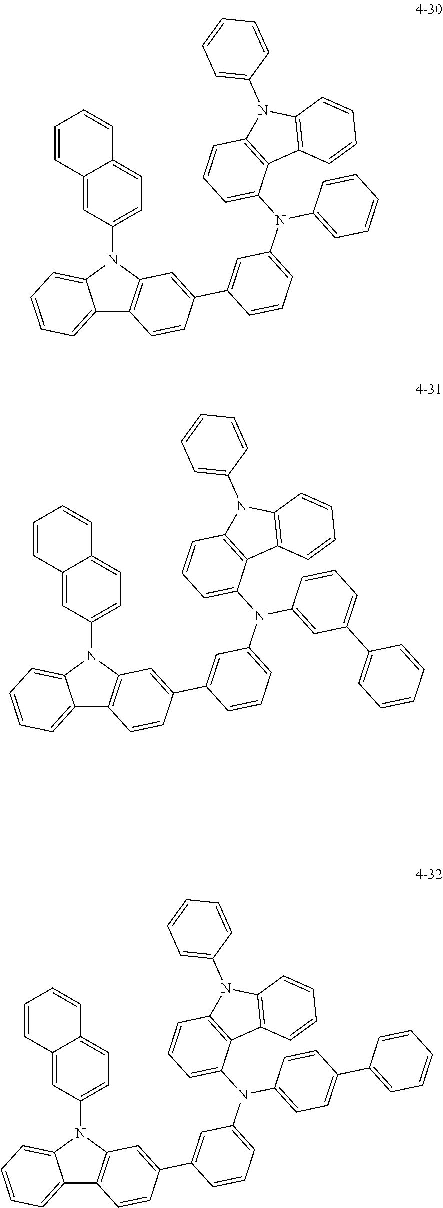 [SMILES: c1ccc(N(c2cccc(-c3ccc4c5ccccc5n(-c5ccc6ccccc6c5)c4c3)c2)c2cccc3c2c2ccccc2n3-c2ccccc2)cc1 ; c1ccc(-c2cccc(N(c3cccc(-c4ccc5c6ccccc6n(-c6ccc7ccccc7c6)c5c4)c3)c3cccc4c3c3ccccc3n4-c3ccccc3)c2)cc1 ; c1ccc(-c2ccc(N(c3cccc(-c4ccc5c6ccccc6n(-c6ccc7ccccc7c6)c5c4)c3)c3cccc4c3c3ccccc3n4-c3ccccc3)cc2)cc1]

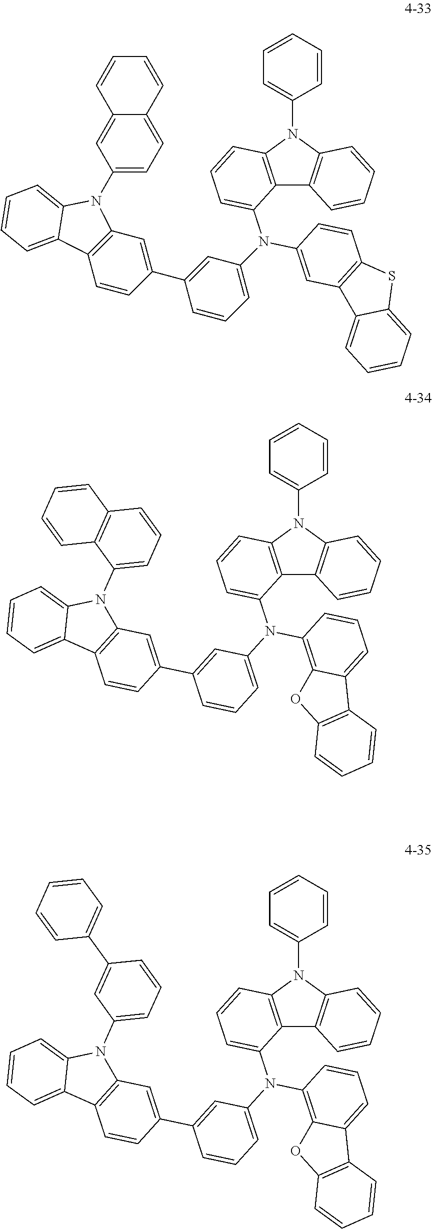 [SMILES: c1ccc(-n2c3ccccc3c3c(N(c4cccc(-c5ccc6c7ccccc7n(-c7ccc8ccccc8c7)c6c5)c4)c4ccc5sc6ccccc6c5c4)cccc32)cc1 ; c1ccc(-n2c3ccccc3c3c(N(c4cccc(-c5ccc6c7ccccc7n(-c7cccc8ccccc78)c6c5)c4)c4cccc5c4oc4ccccc45)cccc32)cc1 ; c1ccc(-c2cccc(-n3c4ccccc4c4ccc(-c5cccc(N(c6cccc7c6oc6ccccc67)c6cccc7c6c6ccccc6n7-c6ccccc6)c5)cc43)c2)cc1]

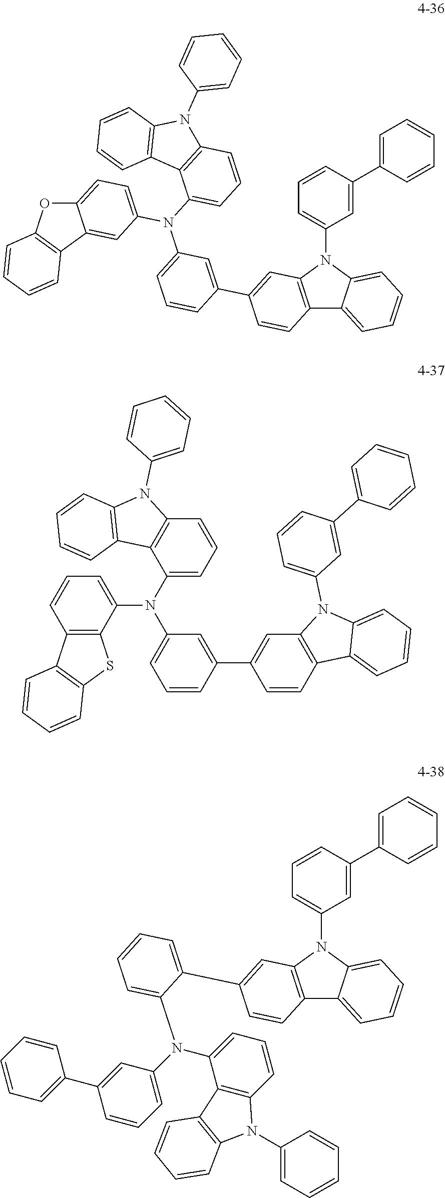 [SMILES: c1ccc(-c2cccc(-n3c4ccccc4c4ccc(-c5cccc(N(c6ccc7oc8ccccc8c7c6)c6cccc7c6c6ccccc6n7-c6ccccc6)c5)cc43)c2)cc1 ; c1ccc(-c2cccc(-n3c4ccccc4c4ccc(-c5cccc(N(c6cccc7c6sc6ccccc67)c6cccc7c6c6ccccc6n7-c6ccccc6)c5)cc43)c2)cc1 ; c1ccc(-c2cccc(N(c3ccccc3-c3ccc4c5ccccc5n(-c5cccc(-c6ccccc6)c5)c4c3)c3cccc4c3c3ccccc3n4-c3ccccc3)c2)cc1]

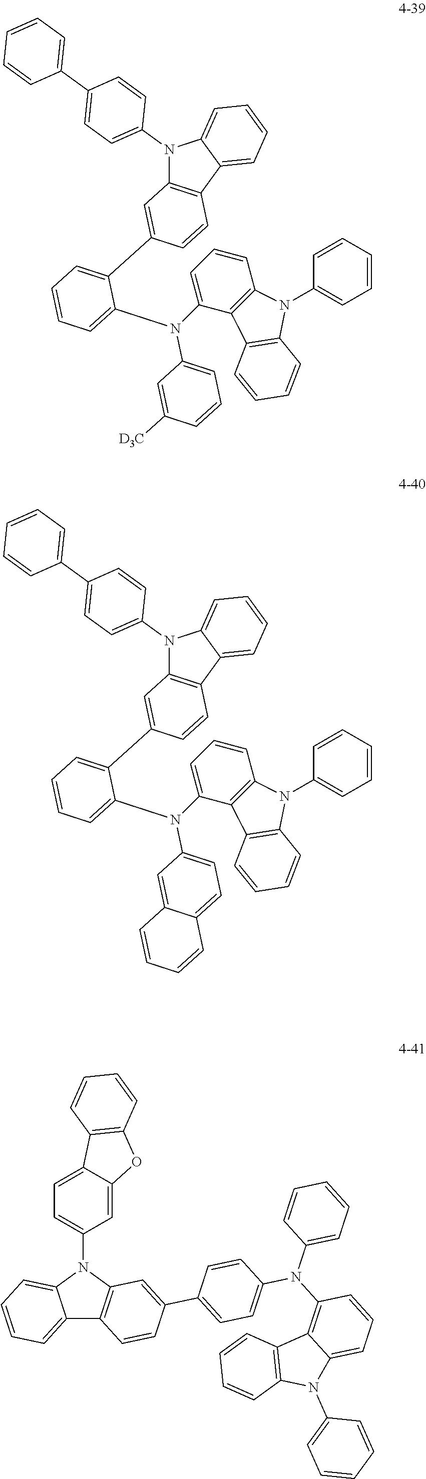 [SMILES: [2H]C([2H])([2H])c1cccc(N(c2ccccc2-c2ccc3c4ccccc4n(-c4ccc(-c5ccccc5)cc4)c3c2)c2cccc3c2c2ccccc2n3-c2ccccc2)c1 ; c1ccc(-c2ccc(-n3c4ccccc4c4ccc(-c5ccccc5N(c5ccc6ccccc6c5)c5cccc6c5c5ccccc5n6-c5ccccc5)cc43)cc2)cc1 ; c1ccc(N(c2ccc(-c3ccc4c5ccccc5n(-c5ccc6c(c5)oc5ccccc56)c4c3)cc2)c2cccc3c2c2ccccc2n3-c2ccccc2)cc1]

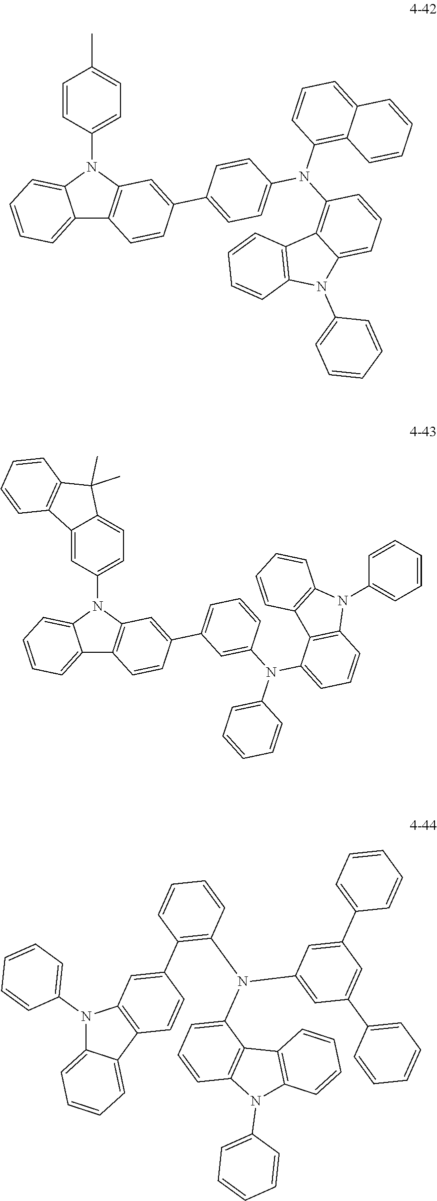 [SMILES: Cc1ccc(-n2c3ccccc3c3ccc(-c4ccc(N(c5cccc6ccccc56)c5cccc6c5c5ccccc5n6-c5ccccc5)cc4)cc32)cc1 ; CC1(C)c2ccccc2-c2cc(-n3c4ccccc4c4ccc(-c5cccc(N(c6ccccc6)c6cccc7c6c6ccccc6n7-c6ccccc6)c5)cc43)ccc21 ; c1ccc(-c2cc(-c3ccccc3)cc(N(c3ccccc3-c3ccc4c5ccccc5n(-c5ccccc5)c4c3)c3cccc4c3c3ccccc3n4-c3ccccc3)c2)cc1]

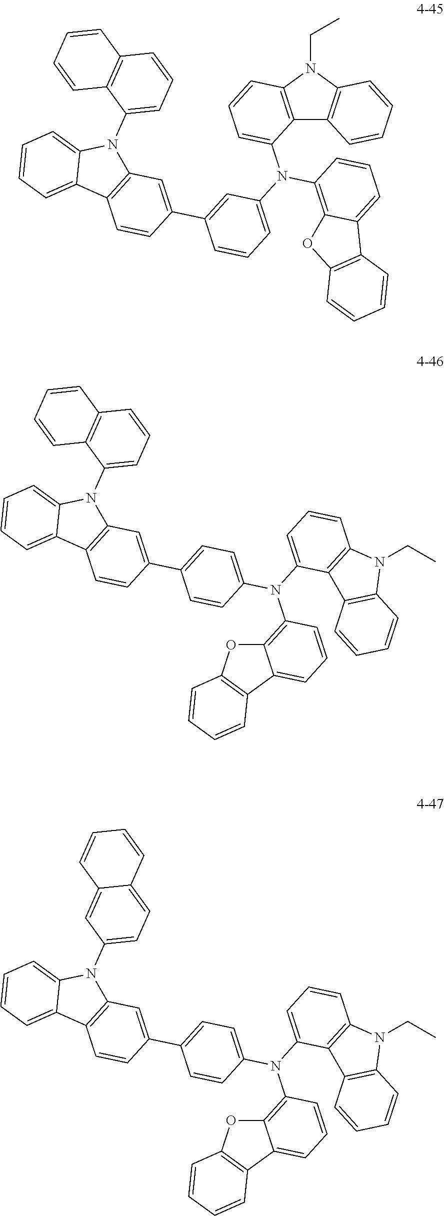 [SMILES: CCn1c2ccccc2c2c(N(c3cccc(-c4ccc5c6ccccc6n(-c6cccc7ccccc67)c5c4)c3)c3cccc4c3oc3ccccc34)cccc21 ; CCn1c2ccccc2c2c(N(c3ccc(-c4ccc5c6ccccc6n(-c6cccc7ccccc67)c5c4)cc3)c3cccc4c3oc3ccccc34)cccc21 ; CCn1c2ccccc2c2c(N(c3ccc(-c4ccc5c6ccccc6n(-c6ccc7ccccc7c6)c5c4)cc3)c3cccc4c3oc3ccccc34)cccc21]

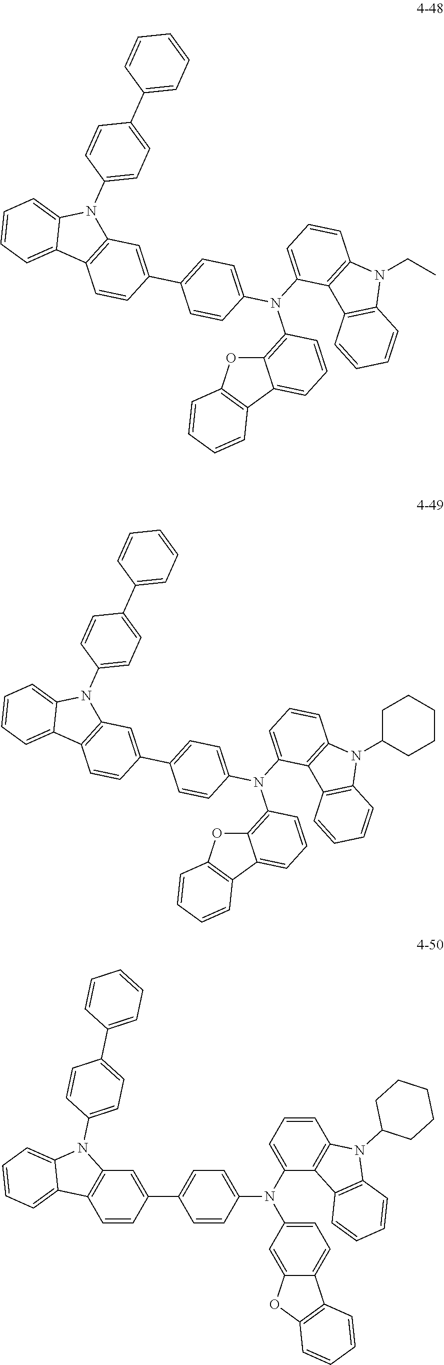 [SMILES: CCn1c2ccccc2c2c(N(c3ccc(-c4ccc5c6ccccc6n(-c6ccc(-c7ccccc7)cc6)c5c4)cc3)c3cccc4c3oc3ccccc34)cccc21 ; c1ccc(-c2ccc(-n3c4ccccc4c4ccc(-c5ccc(N(c6cccc7c6oc6ccccc67)c6cccc7c6c6ccccc6n7C6CCCCC6)cc5)cc43)cc2)cc1 ; c1ccc(-c2ccc(-n3c4ccccc4c4ccc(-c5ccc(N(c6ccc7c(c6)oc6ccccc67)c6cccc7c6c6ccccc6n7C6CCCCC6)cc5)cc43)cc2)cc1]

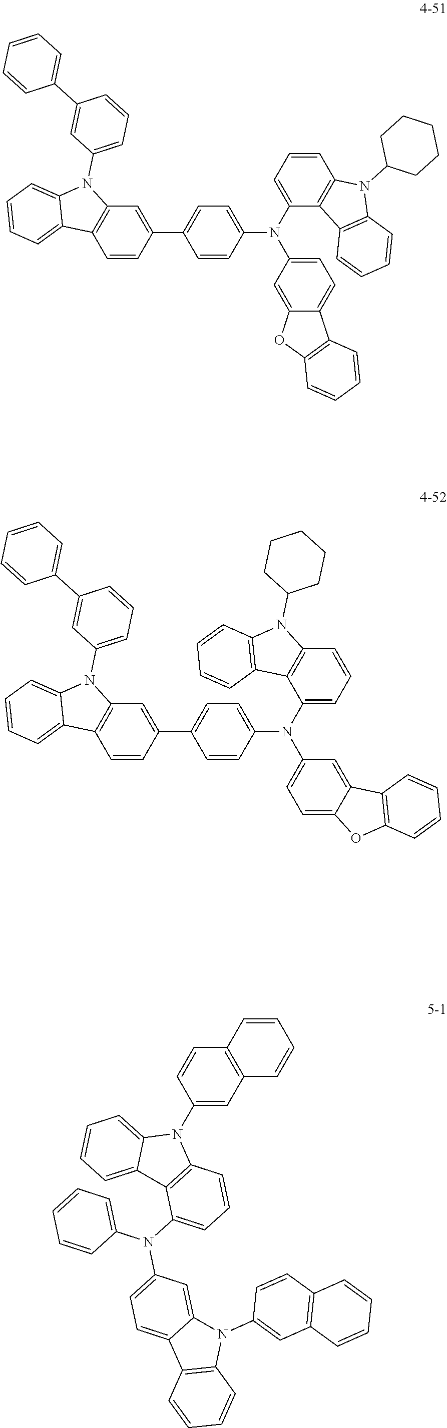 [SMILES: c1ccc(-c2cccc(-n3c4ccccc4c4ccc(-c5ccc(N(c6ccc7c(c6)oc6ccccc67)c6cccc7c6c6ccccc6n7C6CCCCC6)cc5)cc43)c2)cc1 ; c1ccc(-c2cccc(-n3c4ccccc4c4ccc(-c5ccc(N(c6ccc7oc8ccccc8c7c6)c6cccc7c6c6ccccc6n7C6CCCCC6)cc5)cc43)c2)cc1 ; c1ccc(N(c2ccc3c4ccccc4n(-c4ccc5ccccc5c4)c3c2)c2cccc3c2c2ccccc2n3-c2ccc3ccccc3c2)cc1]

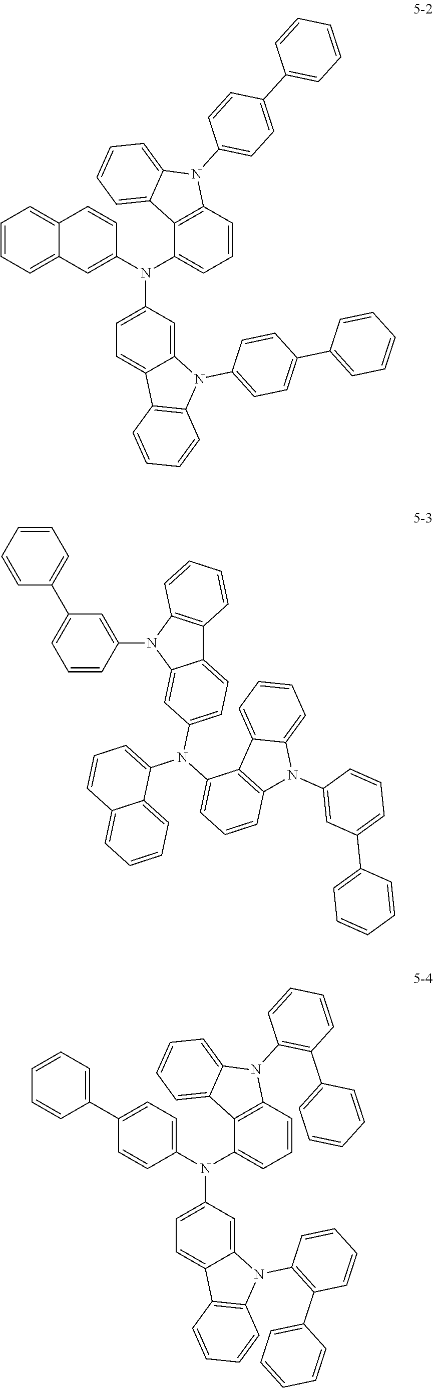 [SMILES: c1ccc(-c2ccc(-n3c4ccccc4c4ccc(N(c5ccc6ccccc6c5)c5cccc6c5c5ccccc5n6-c5ccc(-c6ccccc6)cc5)cc43)cc2)cc1 ; c1ccc(-c2cccc(-n3c4ccccc4c4ccc(N(c5cccc6ccccc56)c5cccc6c5c5ccccc5n6-c5cccc(-c6ccccc6)c5)cc43)c2)cc1 ; c1ccc(-c2ccc(N(c3ccc4c5ccccc5n(-c5ccccc5-c5ccccc5)c4c3)c3cccc4c3c3ccccc3n4-c3ccccc3-c3ccccc3)cc2)cc1]

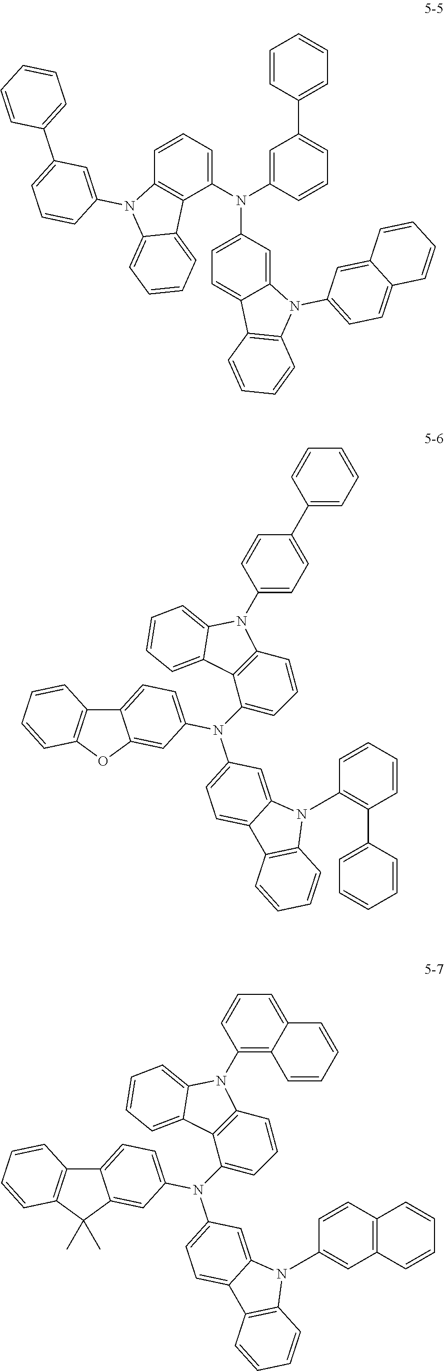 [SMILES: c1ccc(-c2cccc(N(c3ccc4c5ccccc5n(-c5ccc6ccccc6c5)c4c3)c3cccc4c3c3ccccc3n4-c3cccc(-c4ccccc4)c3)c2)cc1 ; c1ccc(-c2ccc(-n3c4ccccc4c4c(N(c5ccc6c(c5)oc5ccccc56)c5ccc6c7ccccc7n(-c7ccccc7-c7ccccc7)c6c5)cccc43)cc2)cc1 ; CC1(C)c2ccccc2-c2ccc(N(c3ccc4c5ccccc5n(-c5ccc6ccccc6c5)c4c3)c3cccc4c3c3ccccc3n4-c3cccc4ccccc34)cc21]

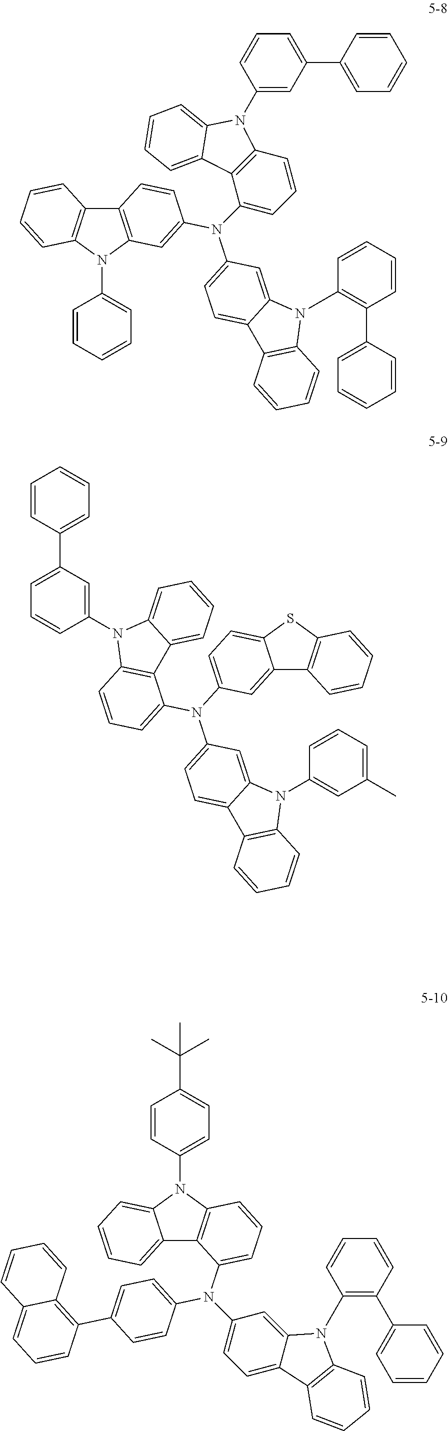 [SMILES: c1ccc(-c2cccc(-n3c4ccccc4c4c(N(c5ccc6c7ccccc7n(-c7ccccc7)c6c5)c5ccc6c7ccccc7n(-c7ccccc7-c7ccccc7)c6c5)cccc43)c2)cc1 ; Cc1cccc(-n2c3ccccc3c3ccc(N(c4ccc5sc6ccccc6c5c4)c4cccc5c4c4ccccc4n5-c4cccc(-c5ccccc5)c4)cc32)c1 ; CC(C)(C)c1ccc(-n2c3ccccc3c3c(N(c4ccc(-c5cccc6ccccc56)cc4)c4ccc5c6ccccc6n(-c6ccccc6-c6ccccc6)c5c4)cccc32)cc1]

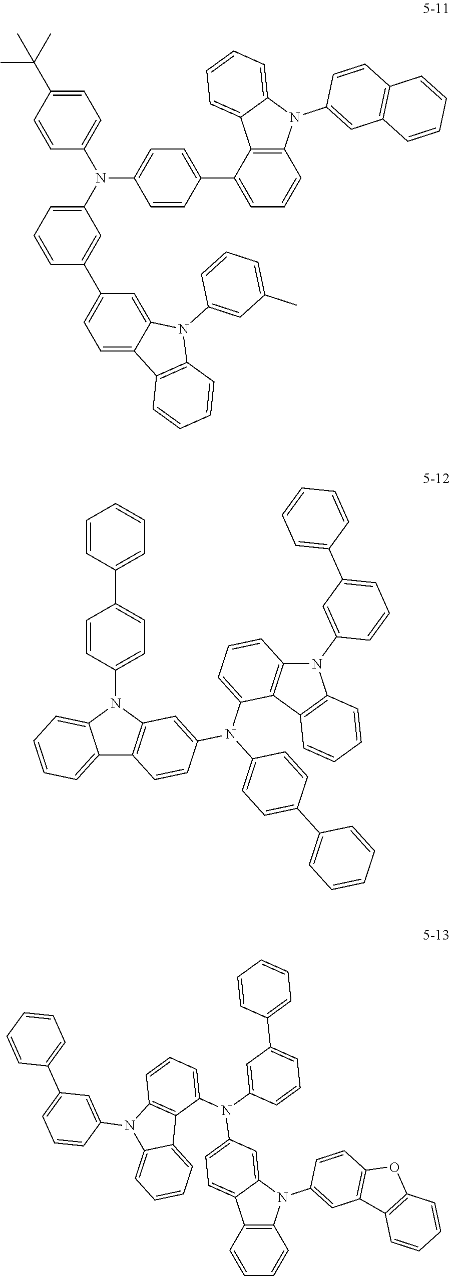 [SMILES: Cc1cccc(-n2c3ccccc3c3ccc(-c4cccc(N(c5ccc(-c6cccc7c6c6ccccc6n7-c6ccc7ccccc7c6)cc5)c5ccc(C(C)(C)C)cc5)c4)cc32)c1 ; c1ccc(-c2ccc(N(c3ccc4c5ccccc5n(-c5ccc(-c6ccccc6)cc5)c4c3)c3cccc4c3c3ccccc3n4-c3cccc(-c4ccccc4)c3)cc2)cc1 ; c1ccc(-c2cccc(N(c3ccc4c5ccccc5n(-c5ccc6oc7ccccc7c6c5)c4c3)c3cccc4c3c3ccccc3n4-c3cccc(-c4ccccc4)c3)c2)cc1]

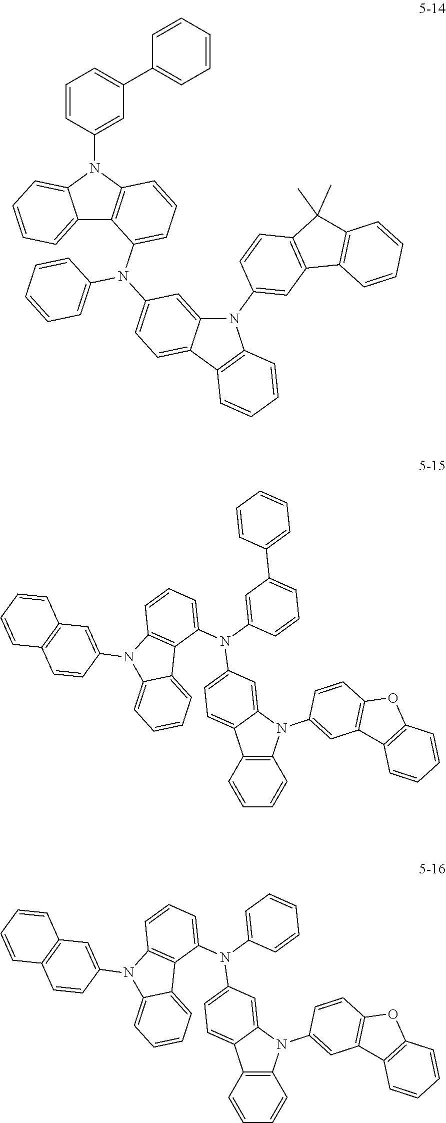 [SMILES: CC1(C)c2ccccc2-c2cc(-n3c4ccccc4c4ccc(N(c5ccccc5)c5cccc6c5c5ccccc5n6-c5cccc(-c6ccccc6)c5)cc43)ccc21 ; c1ccc(-c2cccc(N(c3ccc4c5ccccc5n(-c5ccc6oc7ccccc7c6c5)c4c3)c3cccc4c3c3ccccc3n4-c3ccc4ccccc4c3)c2)cc1 ; c1ccc(N(c2ccc3c4ccccc4n(-c4ccc5oc6ccccc6c5c4)c3c2)c2cccc3c2c2ccccc2n3-c2ccc3ccccc3c2)cc1]

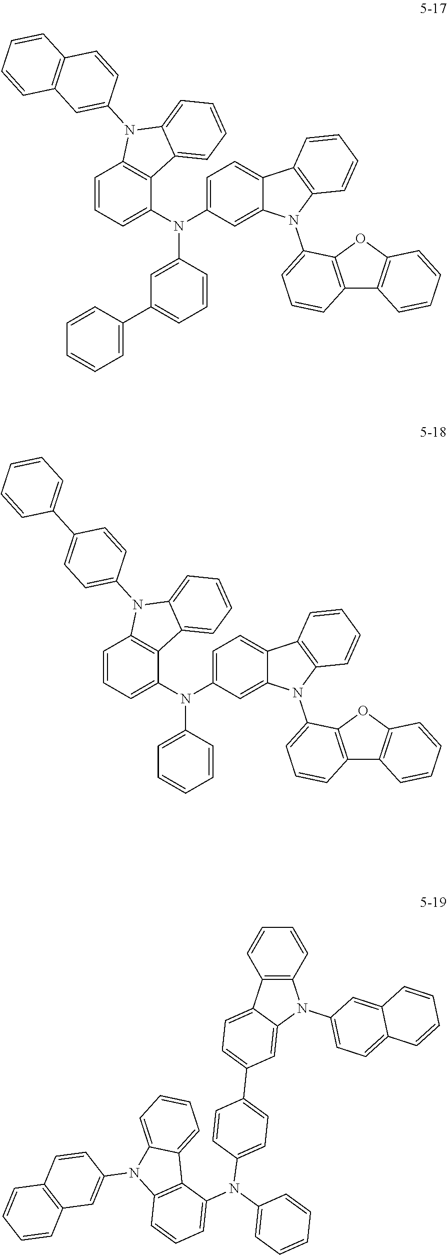 [SMILES: c1ccc(-c2cccc(N(c3ccc4c5ccccc5n(-c5cccc6c5oc5ccccc56)c4c3)c3cccc4c3c3ccccc3n4-c3ccc4ccccc4c3)c2)cc1 ; c1ccc(-c2ccc(-n3c4ccccc4c4c(N(c5ccccc5)c5ccc6c7ccccc7n(-c7cccc8c7oc7ccccc78)c6c5)cccc43)cc2)cc1 ; c1ccc(N(c2ccc(-c3ccc4c5ccccc5n(-c5ccc6ccccc6c5)c4c3)cc2)c2cccc3c2c2ccccc2n3-c2ccc3ccccc3c2)cc1]

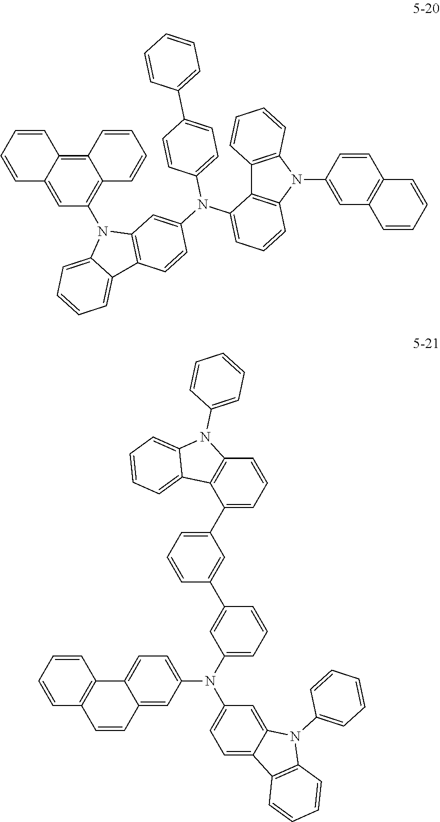 [SMILES: c1ccc(-c2ccc(N(c3ccc4c5ccccc5n(-c5cc6ccccc6c6ccccc56)c4c3)c3cccc4c3c3ccccc3n4-c3ccc4ccccc4c3)cc2)cc1 ; c1ccc(-n2c3ccccc3c3ccc(N(c4cccc(-c5cccc(-c6cccc7c6c6ccccc6n7-c6ccccc6)c5)c4)c4ccc5c(ccc6ccccc65)c4)cc32)cc1]

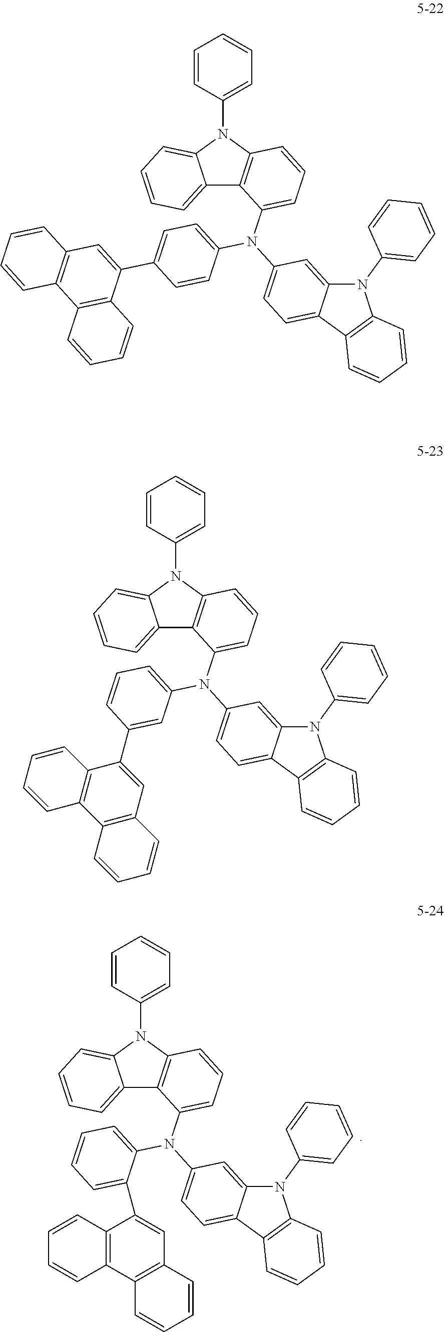 [SMILES: c1ccc(-n2c3ccccc3c3ccc(N(c4ccc(-c5cc6ccccc6c6ccccc56)cc4)c4cccc5c4c4ccccc4n5-c4ccccc4)cc32)cc1 ; c1ccc(-n2c3ccccc3c3ccc(N(c4cccc(-c5cc6ccccc6c6ccccc56)c4)c4cccc5c4c4ccccc4n5-c4ccccc4)cc32)cc1 ; c1ccc(-n2c3ccccc3c3ccc(N(c4ccccc4-c4cc5ccccc5c5ccccc45)c4cccc5c4c4ccccc4n5-c4ccccc4)cc32)cc1]

11. An electronic element, comprising an anode, a cathode, and a functional layer disposed between the anode and the cathode, wherein, the functional layer comprises the organic compound according to claim 1.

12. The electronic element according to claim 11, wherein, the functional layer comprises a hole transport layer, and the hole transport layer comprises the organic compound;
optionally, the electronic element is an organic electroluminescent device or a photoelectric conversion device.

13. An electronic apparatus, comprising the electronic element according to claim 11.
